(19) United States
(12) Patent Application Publication
(10) Pub. No.: US 2013/0102631 A1
(43) Pub. Date: Apr. 25, 2013

(54) NOVEL MICROBIOCIDAL DIOXIME ETHER DERIVATIVES

(71) Applicants: Kurt Nebel, Stein (CH); Daniel Stierli, Stein (CH); Werner Zambach, Stein (CH); Andrea Bortolato, Stein (CH)

(72) Inventors: Kurt Nebel, Stein (CH); Daniel Stierli, Stein (CH); Werner Zambach, Stein (CH); Andrea Bortolato, Stein (CH)

(73) Assignee: SYNGENTA CROP PROTECTION LLC, Greensboro, NC (US)

(21) Appl. No.: 13/808,096

(22) PCT Filed: Jun. 29, 2011
(86) PCT No.: PCT/EP2011/060904

§ 371 Date: Jan. 2, 2013

(30) Foreign Application Priority Data
Jul. 2, 2010 (EP) 10168236.7
Jul. 29, 2010 (EP) 10171257.8
Jan. 11, 2011 (EP) 11150650.7

(51) Int. Cl. C07D 401/12; C07D 401/04; C07D 219/06; A01N 43/42; C07D 215/40

(52) U.S. Cl. CPC C07D 401/12; A01N 43/42; C07D 215/40; C07D 219/06; C07D 401/04
USPC 514/312; 546/171; 514/314; 546/153; 546/178; 546/102

(57) ABSTRACT

The present invention provides compounds of formula (I) wherein R¹, A¹, X, Y⁶, Y⁷, Y⁸, G¹, G², G³ and p are as defined in the claims. The invention further provides intermediates used in the preparation of these compounds, to compositions which comprise these compounds and to their use in agriculture or horticulture for controlling or preventing infestation of plants by phytopathogenic microorganisms, preferably fungi.

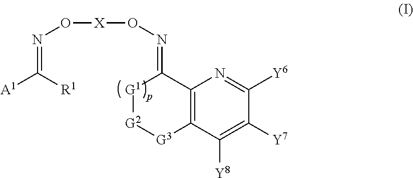

The present invention relates to novel microbiocidally active, in particular fungicidally active, cyclic bisoxime derivatives. It further relates to intermediates used in the preparation of these compounds, to compositions which comprise these compounds and to their use in agriculture or horticulture for controlling or preventing infestation of plants by phytopathogenic microorganisms, preferably fungi.

Fungicidally active bisoximes are described in WO08074418.

Surprisingly, it has been found that novel bisoxime derivatives based on a bicyclic fragment have microbiocidal activity.

The present invention accordingly relates to bisoxime derivatives of formula (I)

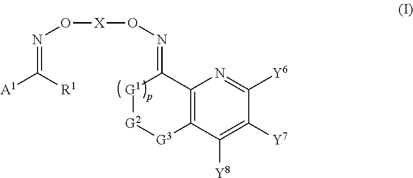

wherein R¹ represents hydrogen, halogen, CN, OH, SH, C₁-C₈ alkylthio, C₁-C₈ alkylsulphinyl, C₁-C₈ alkylsulphonyl, NH₂, C₁-C₁₀ alkyl, C₃-C₈ cycloalkyl, C₂-C₈ alkenyl, C₂-C₈ alkynyl, (C₁-C₄ alkyloxycarbonyl) C₁-C₄-alkyl, (C₁-C₄ alkyl)O₂C, phenyl or pyridyl, wherein the alkyl, cycloalkyl, alkenyl, alkynyl, phenyl and pyridyl are optionally substituted by one or more groups independently selected from halogen, CN, NH₂, NO₂, OH, C₁-C₄ alkyl, C₁-C₄ haloalkyl, C₁-C₄ alkoxy, C₁-C₄ haloalkoxy, C₃-C₆ cycloalkyl and a 5- or 6-membered heterocycle containing one to three heteroatoms independently selected from O, S and N, providing that the heterocycle does not contain adjacent oxygen atoms, adjacent sulphur atoms, or adjacent sulphur and oxygen atoms;

A¹ represents cycle A-2, A-4, or A-5:

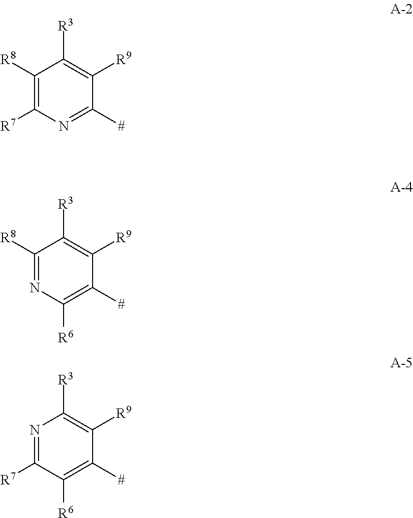

R³, R⁶, R⁷, R⁸ and R⁹ independently of one another represent hydrogen, halogen, CN, NO₂, C₁-C₈ alkyl, C₁-C₄-alkoxy-C₁-C₄-alkyl, C₁-C₄-alkoxy-C₁-C₄-alkoxy-C₁-C₄-alkyl, C₃-C₈ cycloalkyl, C₂-C₈ alkenyl, C₂-C₈ alkynyl, phenyl, a 5- or 6-membered heterocycle containing one to three heteroatoms independently selected from O, S and N, providing that the heterocycle does not contain adjacent oxygen atoms, adjacent sulphur atoms, or adjacent sulphur and oxygen atoms, COR¹³, OR¹¹, SH, C₁-C₈ alkylthio, C₁-C₈ alkylsulphinyl, C₁-C₈ alkylsulphonyl, N(R¹²)², CO₂R¹¹, O(CO)R¹³, CON(R¹²)₂, NR¹²COR¹³ or CR¹³N—OR¹¹, wherein the alkyl, alkoxy, cycloalkyl, alkenyl, alkynyl, phenyl and heterocycle are optionally substituted by one or more groups independently selected from halogen, CN, NH₂, NO₂, OH, C₁-C₄ alkyl, C₁-C₄-haloalkyl, C₁-C₄ alkoxy and C₁-C₄ haloalkoxy;

or R⁶ and R⁷, R⁷ and R⁸, R³ and R⁸, or R³ and R⁹ together with the fragment of the pyridyl ring to which they are attached may form a partially or fully unsaturated 5- to 7-membered carbocyclic ring or a 5- to 7-membered heterocyclic ring containing one to three heteroatoms independently selected from O, S, N and N(R¹²), providing that the heterocycle does not contain adjacent oxygen atoms, adjacent sulphur atoms, or adjacent sulphur and oxygen atoms, and wherein the ring formed by R⁶ and R⁷, R⁷ and R⁸, R³ and R⁸, or R³ and R⁹ is optionally substituted by one or more groups independently selected from halogen, CN, NH₂, NO₂, OH, C₁-C₄ alkyl, C₁-C₄ haloalkyl, C₁-C₄ alkoxy and C₁-C₄ haloalkoxy;

X represents X-2, X-3, X-4 or X-5:

#—Z²—Z³—# X-2

#—Z⁴—Z⁵—Z⁶—# X-3

#—Z⁷—Z⁸—Z⁹—Z¹⁰—# X-4

#—Z¹¹—Z¹²—Z¹³—Z¹⁴—Z¹⁵—# X-5

Z², Z³, Z⁴, Z⁶, Z⁷, Z⁸, Z⁹, Z¹⁰, Z¹¹, Z¹², Z¹⁴ and Z¹⁵ independently of one another represent CR¹⁴R¹⁵, C═O or C═CR¹⁹R²⁰;

Z⁵ and Z¹³ independently of one another represent CR^{14′}R^{15′}, SiR¹⁶R¹⁷, C═O or C═CR¹⁹R²⁰;

each R¹⁴ and R¹⁵ independently of one another represent hydrogen, halogen, OH, C₁-C₄ alkyl, C₁-C₄ haloalkyl, phenyl or CN, wherein the phenyl is optionally substituted by one or more groups independently selected from halogen, CN, C₁-C₄ alkyl, C₁-C₄ haloalkyl, C₁-C₄ alkoxy and C₁-C₄ haloalkyoxy;

or R¹⁴ and R¹⁵ together with the carbon atom to which they are attached may form a C₃-C₆ cycloalkyl group or a C₃-C₆ halocycloalkyl group;

each R¹⁹ and R²⁰ independently of one another represent hydrogen, halogen, C₁-C₄ alkyl or C₁-C₄ haloalkyl;

each R^{14′}, R^{15′}, R¹⁶ and R¹⁷ independently of one another represent hydrogen, halogen, OH, C₁-C₄ alkyl, C₁-C₄ haloalkyl, phenyl or CN, wherein phenyl is optionally substituted by one or more groups independently selected from halogen, CN, C₁-C₄ alkyl, C₁-C₄ haloalkyl, C₁-C₄ alkoxy and C₁-C₄ haloalkyoxy;

or R^{14′} and R^{15′} together with the carbon atom to which they are attached may form a C₃-C₆ cycloalkyl group or a C₃-C₆ halocycloalkyl group;

and wherein the groupings X-2, X-3, X-4 and X-5 contain at most one ring (i.e. a cycloalkyl group or halocycloalkyl group) which contains either only one of the radicals Z² to Z¹⁵ or two radicals Z² to Z¹⁵ or three radicals Z² to Z¹⁵ or four radicals Z² to Z¹⁵ as ring members; and wherein radicals Z², Z³, Z⁴, Z⁶, Z⁷, Z¹⁰, Z¹¹ and Z¹⁵ are not substituted by OH;

Y⁶, Y⁷ and Y⁸ independently of one another represent hydrogen, halogen, CN, NO₂, C₁-C₈ alkyl, C₁-C₄-alkoxy-C₁-C₄-alkyl, C₁-C₄-alkoxy-C₁-C₄-alkoxy-C₁-C₄-alkyl, C₃-C₈ cycloalkyl, C₂-C₈ alkenyl, C₂-C₈ alkynyl, phenyl, pyridyl, COR¹³, OR²², SH, C₁-C₈ alkylthio, C₁-C₈ alkylsulphinyl, C₁-C₈ alkylsulphonyl, N(R²³)₂, CO₂R²², O(CO)R¹³, CON(R²³)₂, NR²³COR¹³ or CR¹³N—OR²², wherein the alkyl, alkoxy, cycloalkyl, alkenyl, alkynyl, phenyl and pyridyl are optionally substituted by one or more groups independently selected from halogen, CN, NH₂, NO₂, OH, C₁-C₄ alkyl, C₁-C₄ haloalkyl, C₁-C₄ alkoxy and C₁-C₄ haloalkoxy;

or Y⁶ and Y⁷ or Y⁷ and Y⁸ together with the fragment of the pyridyl ring to which they are attached may form a partially or fully unsaturated 5- to 7-membered carbocyclic ring or a 5- to 7-membered heterocyclic ring containing one to three heteroatoms independently selected from O, S, N and N(R¹²), providing that the heterocycle does not contain adjacent oxygen atoms, adjacent sulphur atoms, or adjacent sulphur and oxygen atoms, and wherein the ring formed by Y⁶ and Y⁷ or Y⁷ and Y⁸ is optionally substituted by one or more groups independently selected from halogen, CN, NH₂, NO₂, OH, C₁-C₄ alkyl, C₁-C₄ haloalkyl, C₁-C₄ alkoxy and C₁-C₄ haloalkoxy;

each R¹¹ and R²² independently of one another represent hydrogen, C₁-C₈ alkyl, C₃-C₈ cycloalkyl, C₃-C₈ alkenyl, C₃-C₈ alkynyl, benzyl, phenyl or pyridyl, wherein the alkyl, cycloalkyl, alkenyl, alkynyl, phenyl, benzyl and pyridyl are optionally substituted by one or more groups independently selected from halogen, CN, NH₂, NO₂, OH, C₁-C₄ alkyl, C₁-C₄ haloalkyl, C₁-C₄ alkoxy, C₁-C₄ haloalkoxy and C₁-C₄-alkoxy-C₁-C₄-alkyl;

each R¹² and R²³ independently of one another represent hydrogen, OH, C₁-C₈ alkyl, C₁-C₈ alkoxy, C₁-C₈-alkoxy-C₁-C₄-alkyl, C₃-C₈ alkenyl, C₃-C₈ alkynyl, or COR¹³, wherein the alkyl, alkoxy, alkenyl and alkynyl are optionally substituted by one or more halogen;

wherein when two radicals R¹² or two radicals R²³ are attached to the same nitrogen atom, these radicals can be identical or different;

wherein when two radicals R¹² or two radicals R²³ are attached to the same nitrogen atom, both of these radicals cannot be OH, C₁-C₄ alkoxy or C₁-C₄ haloalkoxy;

and wherein when two radicals R¹² or two radicals R²³ are attached to the same nitrogen atom, these two radicals together with the nitrogen atom to which they are attached may form a cycle B-1, B-2, B-3, B-4, B-5, B-6, B-7 or B-8:

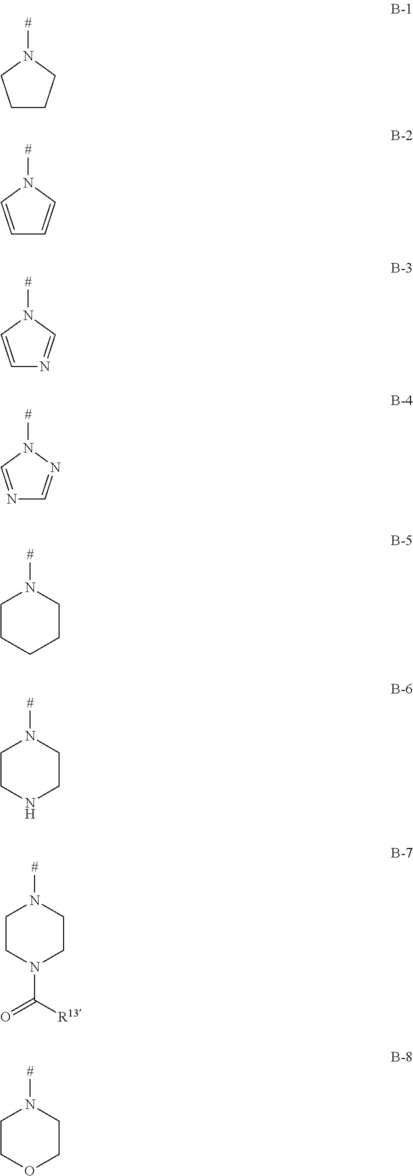

wherein the cycle formed is optionally substituted by one or more groups independently selected from halogen, CN, NH₂, NO₂, OH, C₁-C₄ alkyl, C₁-C₄ haloalkyl, C₁-C₄ alkoxy and C₁-C₄ haloalkoxy;

each R¹³ and R^{13′} independently of one another represent hydrogen, C₁-C₈ alkyl, C₃-C₈ cycloalkyl, C₂-C₈ alkenyl, C₂-C₈ alkynyl, benzyl, phenyl or pyridyl, wherein the alkyl, cycloalkyl, alkenyl, alkynyl, phenyl, benzyl and pyridyl are optionally substituted by one or more groups independently selected from halogen, CN, NH₂, NO₂, OH, C₁-C₄ alkyl, C₁-C₄ haloalkyl, C₁-C₄ alkoxy and C₁-C₄ haloalkoxy;

G¹ and G² independently of one another represent —C(R²⁴R²⁵)—;

G³ represents —C(R²⁴R²⁵)—, O, N(R²⁶) or S;

or G¹ and G², or G² and G³, or G¹ and G¹ together represent —CR²⁴═CR²⁵—;

each R²⁴ and R²⁵ independently of one another represent hydrogen, halogen, C₁-C₄ alkyl, C₁-C₄ haloalkyl, C₁-C₄ alkoxy or C₁-C₄ haloalkoxy;

R²⁶ represents hydrogen, OH, C₁-C₄ alkyl, C₁-C₄ alkoxy, C₁-C₈ alkylcarbonyl or C₁-C₈ haloalkylcarbonyl; and

p is 1 or 2;

or a salt or an N-oxide thereof.

Halogen, either as a lone substituent or in combination with another substituent (e.g. haloalkyl) is generally fluorine, chlorine, bromine or iodine, and usually fluorine, chlorine or bromine.

Each alkyl moiety (including the alkyl moiety of alkoxy, alkylthio, etc.) is a straight or branched chain and, depending on the number of carbon atoms it contains, is, for example, methyl, ethyl, n-propyl, n-butyl, n-pentyl, n-hexyl, iso-propyl, sec-butyl, iso-butyl, tert-butyl, neo-pentyl, n-heptyl or 1,3-dimethylbutyl, and usually methyl or ethyl.

The alkenyl and alkynyl groups can be mono- or di-unsaturated and are examples thereof are derived from the above mentioned alkyl groups.

Haloalkyl moieties are alkyl moieties which are substituted by one or more of the same or different halogen atoms and are, for example, monofluoromethyl, difluoromethyl, trifluoromethyl, monochloromethyl, dichloromethyl, trichloromethyl, 2,2,2-trifluoroethyl, 2,2-difluoroethyl, 2-fluoroethyl, 1,1-difluoroethyl, 1-fluoroethyl, 2-chloroethyl, pentafluoroethyl, 1,1-difluoro-2,2,2-trichloroethyl, 2,2,3,3-tetrafluoroethyl and 2,2,2-trichloroethyl, and typically trichloromethyl, difluorochloromethyl, difluoromethyl, trifluoromethyl and dichlorofluoromethyl.

Alkoxy is, for example, methoxy, ethoxy, propoxy, iso-propoxy, n-butoxy, iso-butoxy, sec-butoxy and tert-butoxy, and usually methoxy or ethoxy.

Haloalkoxy is, for example, fluoromethoxy, difluoromethoxy, trifluoromethoxy, 2,2,2-trifluoroethoxy, 1,1,2,2-tetrafluoroethoxy, 2-fluoroethoxy, 2-chloroethoxy, 2,2-difluoroethmy and 2,2,2-trichloroethoxy, and usually difluoromethoxy, 2-chloroethoxy and trifluoromethoxy.

Alkylthio is, for example, methylthio, ethylthio, propylthio, iso-propylthio, n-butylthio, iso-butylthio, sec-butylthio or tert-butylthio, and usually methylthio or ethylthio.

Alkylsulphonyl is, for example, methylsulphonyl, ethylsulphonyl, propylsulphonyl, iso-propylsulphonyl, n-butylsulphonyl, iso-butylsulphonyl, sec-butylsulphonyl or tert-butylsulphonyl, and usually methylsulphonyl or ethylsulphonyl.

Alkylsulphinyl is, for example, methylsulphinyl, ethylsulphinyl, propylsulphinyl, iso-propylsulphinyl, n-butylsulphinyl, iso-butylsulphinyl, sec-butylsulphinyl or tert-butylsulphinyl, and usually methylsulphinyl or ethylsulphinyl

Cycloalkyl may be saturated or partially unsaturated, preferably fully saturated, and is, for example, cyclopropyl, cyclobutyl, cyclopentyl or cyclohexyl.

Alkoxyalkyl is, for example, methoxymethyl, methoxyethyl, ethoxymethyl, ethoxyethyl, n-propoxymethyl, n-propoxyethyl, iso-propoxymethyl or iso-propoxyethyl.

Aryl includes phenyl, naphthyl, anthracyl, fluorenyl and indanyl, but is usually phenyl.

Carbocycle includes cycloalkyl groups and aryl groups.

Heterocycloalkyl is a non-aromatic ring that may be saturated or partially unsaturated, preferably fully saturated, containing carbon atoms as ring members and at least one heteroatom selected from O, S and N as ring members. Examples include oxiranyl, oxetanyl, tetrahydrofuranyl, tetrahydropyranyl, 1,3-dioxolanyl, 1,4-dioxanyl, aziridinyl, azetidinyl, pyrrolidinyl, piperidinyl, oxazinanyl, morpholinyl, thiomorpholinyl, imidazolidinyl, pyrazolidinyl and piperazinyl, preferably morpholinyl, pyrrolidinyl, piperidinyl and piperazinyl, more preferably morpholinyl and pyrollidinyl.

Heteroaryl is, for example, a monovalent monocyclic or bicyclic aromatic hydrocarbon radical. Examples of monocyclic groups include pyridyl, pyridazinyl, pyrimidinyl, pyrazinyl, pyrrolyl, pyrazolyl, imidazolyl, triazolyl, tetrazolyl, furanyl, thiophenyl, oxazolyl, isoxazolyl, oxadiazolyl, thiazolyl, isothiazolyl, and thiadiazolyl. Examples of bicyclic groups include quinolinyl, cinnolinyl, quinoxalinyl, benzimidazolyl, benzothiophenyl, and benzothiadiazolyl. Monocyclic heteroaryl groups are preferred, preferably pyridyl, pyrrolyl, imidazolyl and triazolyl, e.g. 1,2,4 triazolyl, pyridyl and imidazolyl being most preferred.

The terms “heterocycle” and “heterocyclic ring” are used interchangeably and are defined to include heterocycloalkyl and heteroaryl groups. Any reference herein to a heterocycle or heterocyclic ring preferably refers to the specific examples given under the definition of heteroaryl and heterocycloalkyl above, and are preferably morpholinyl, pyrrolidinyl, piperidinyl, piperazinyl pyridyl, pyrrolyl, imidazolyl and triazolyl, e.g. 1,2,4 triazolyl, more preferably morpholinyl, pyrollidinyl, pyridyl and imidazolyl.

Where a moiety is indicated as being (optionally) substituted, e.g. alkyl, this includes those moieties where they are part of a larger group, e.g. the alkyl in the alkylthio group. Where a moiety is indicated as being optionally substituted by one or more other groups, preferably there are one to five optional substituents, more preferably one to three optional substituents.

R¹ represents hydrogen, halogen, CN, OH, SH, C₁-C₈ alkylthio, C₁-C₈ alkylsulphinyl, C₁-C₈ alkylsulphonyl, NH₂, C₁-C₁₀ alkyl, C₃-C₈ cycloalkyl, C₂-C₈ alkenyl, C₂-C₈ alkynyl, (R¹¹O)carbonyl(C₁-C₄-alkyl), phenyl or pyridyl, wherein the alkyl, cycloalkyl, alkenyl, alkynyl, phenyl and pyridyl are optionally substituted by one or more groups, e.g. one to five groups, independently selected from halogen, CN, NH₂, NO₂, OH, C₁-C₄ alkyl, C₁-C₄ haloalkyl, C₁-C₄ alkoxy, C₁-C₄ haloalkoxy, C₃-C₆ cycloalkyl and a 5- or 6-membered heterocycle containing one to three heteroatoms independently selected from O, S and N, providing that the heterocycle does not contain adjacent oxygen atoms, adjacent sulphur atoms, or adjacent sulphur and oxygen atoms. The heterocycle is preferably one as defined herein, preferably morpholinyl, pyrrolidinyl, piperidinyl, piperazinyl, pyridyl, pyrrolyl, imidazolyl or triazolyl, e.g. 1,2,4 triazolyl, more preferably morpholinyl, pyrollidinyl, pyridyl or imidazolyl.

Preferably R¹ represents hydrogen, C₁-C₈ alkyl, C₃-C₈ cycloalkyl, phenyl or pyridyl, wherein the alkyl, cycloalkyl, phenyl and pyridyl are optionally substituted by one or more groups, e.g. one to five groups, independently selected from halogen, CN, C₁-C₄ alkyl, C₁-C₄ haloalkyl, OH, C₁-C₄ alkoxy, C₁-C₄ haloalkoxy and C₃-C₆ cycloalkyl.

More preferably R¹ represents hydrogen, C₁-C₄ alkyl, phenyl or pyridyl, wherein alkyl is optionally substituted by one or more groups, e.g. one to five groups, independently selected from halogen, OH, C₁-C₄ alkoxy and C₁-C₄ haloalkoxy, and wherein phenyl and pyridyl are optionally substituted by one or more groups, e.g. one to five groups, independently selected from halogen, CN, C₁-C₄ alkyl, C₁-C₄ haloalkyl, OH, C₁-C₄ alkoxy, C₁-C₄ haloalkoxy and C₃-C₆ cycloalkyl.

Even more preferably R¹ represents hydrogen, C₁-C₄ alkyl, C₁-C₄ haloalkyl, phenyl or pyridin-2-yl, wherein the phenyl and pyridin-2-yl are optionally substituted by one or more groups, e.g. one to five groups, independently selected from halogen, CN, methyl, halomethyl, methoxy and halomethoxy.

In one preferred group of compounds R¹ represents pyridyl, optionally substituted by one or more groups independently selected from halogen, CN, NH₂, NO₂, OH, C₁-C₄-alkyl, C₁-C₄-haloalkyl, C₁-C₄-alkoxy, C₁-C₄-haloalkoxy, C₃-C₆ cycloalkyl and a 5 or 6-membered heterocycle containing one to three heteroatoms independently selected from O, S and N, providing that the heterocycle does not contain adjacent oxygen atoms, adjacent sulphur atoms, or adjacent sulphur and oxygen atoms. The heterocycle is preferably one as defined herein, preferably morpholinyl, pyrrolidinyl, piperidinyl, piperazinyl, pyridyl, pyrrolyl, imidazolyl or triazolyl, e.g. 1,2,4 triazolyl, more preferably morpholinyl, pyrollidinyl, pyridyl or imidazolyl.

In this preferred group of compounds R¹ preferably represents pyridin-2-yl, optionally substituted by one or more groups independently selected from halogen, CN, NH₂, NO₂, OH, C₁-C₄ alkyl, C₁-C₄ haloalkyl, C₁-C₄ alkoxy, C₁-C₄ haloalkoxy, C₃-C₆ cycloalkyl and a 5 or 6-membered heterocycle containing one to three heteroatoms independently selected from O, S and N, providing that the heterocycle does not contain adjacent oxygen atoms, adjacent sulphur atoms, or adjacent sulphur and oxygen atoms. The heterocycle is preferably one as defined herein, preferably morpholinyl, pyrrolidinyl, piperidinyl, piperazinyl, pyridyl, pyrrolyl, imidazolyl or triazolyl, e.g. 1,2,4 triazolyl, more preferably morpholinyl, pyrollidinyl, pyridyl or imidazolyl.

In another group of compounds R¹ represents hydrogen, C₁-C₄ alkyl, C₂-C₄ alkenyl, phenyl or pyridyl, wherein the alkyl, alkenyl, phenyl and pyridyl are optionally substituted by one or more groups independently selected from C₁-C₄ alkyl, C₁-C₄ haloalkyl, halogen, CN, C₁-C₄ alkoxy and C₁-C₄ haloalkoxy.

In another preferred group of compounds, R¹ represents hydrogen, halogen, CN, OH, SH, C₁-C₈ alkylthio, C₁-C₈ alkylsulphinyl, C₁-C₈ alkylsulphonyl, NH₂, C₁-C₁₀ alkyl, C₃-C₈ cycloalkyl, C₂-C₈ alkenyl, C₂-C₈ alkynyl, (C₁-C₄ alkyloxycarbonyl) C₁-C₄-alkyl, (C₁-C₄ alkyl)O₂C, phenyl or pyridyl, wherein the alkyl, cycloalkyl, alkenyl, alkynyl, phenyl and pyridyl are optionally substituted by one or more groups independently selected from halogen, CN, NH₂, NO₂, OH, C₁-C₄ alkyl, C₁-C₄ haloalkyl, C₁-C₄ alkoxy, C₁-C₄ haloalkoxy, C₃-C₆ cycloalkyl and a 5- or 6-membered heterocycle containing one to three heteroatoms independently selected from O, S and N, providing that the heterocycle does not contain adjacent oxygen atoms, adjacent sulphur atoms, or adjacent sulphur and oxygen atoms.

In another preferred group of compounds, R¹ represents hydrogen, (C₁-C₄ alkyl)O₂C, C₁-C₁₀ alkyl, phenyl or pyridyl, wherein the alkyl, phenyl and pyridyl are optionally substituted by one or more groups independently selected from halogen, CN, C₁-C₄ alkyl, C₁-C₄ haloalkyl, C₃-C₆ cycloalkyl and a 5- or 6-membered heterocycle containing one to three nitrogen atoms.

A¹ represents cycle A-2, A-4, or A-5:

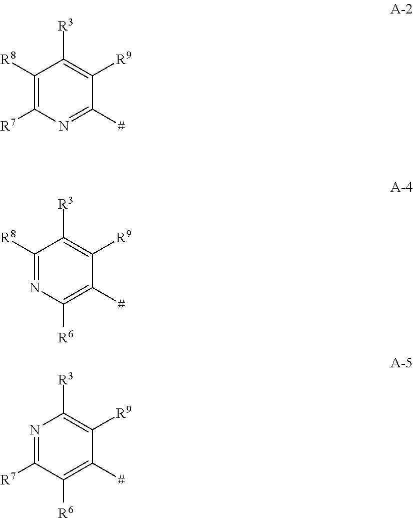

Preferably A¹ represents cycle A-2.

More preferably, A¹ represent pyridin-2-yl, optionally substituted by one or more groups independently selected from halogen, CN, NH₂, NO₂, OH, C₁-C₄ alkyl, C₁-C₄ haloalkyl, C₁-C₄ alkoxy, C₁-C₄ haloalkoxy, C₃-C₆ cycloalkyl and a 5 or 6-membered heterocycle containing one to three heteroatoms independently selected from O, S and N, providing that the heterocycle does not contain adjacent oxygen atoms, adjacent sulphur atoms, or adjacent sulphur and oxygen atoms. The heterocycle is preferably one as defined herein, preferably morpholinyl, pyrrolidinyl, piperidinyl, piperazinyl, pyridyl, pyrrolyl, imidazolyl or triazolyl, e.g. 1,2,4 triazolyl, more preferably morpholinyl, pyrollidinyl, pyridyl or imidazolyl.

R³, R⁶, R⁷, R⁸ and R⁹ independently of one another represent hydrogen, halogen, CN, NO₂, C₁-C₈ alkyl, C₁-C₄-alkoxy-C₁-C₄-alkyl, C₁-C₄-alkoxy-C₁-C₄-alkoxy-C₁-C₄-alkyl, C₃-C₈ cycloalkyl, C₂-C₈ alkenyl, C₂-C₈ alkynyl, phenyl, a 5- or 6-membered heterocycle containing one to three heteroatoms independently selected from O, S and N, providing that the heterocycle does not contain adjacent oxygen atoms, adjacent sulphur atoms, or adjacent sulphur and oxygen atoms (e.g. a heterocycle as defined herein, preferably morpholinyl, pyrrolidinyl, piperidinyl, piperazinyl, pyridyl, pyrrolyl, imidazolyl or triazolyl, e.g. 1,2,4 triazolyl, more preferably morpholinyl, pyrollidinyl, pyridyl or imidazolyl), COR¹³, OR¹¹, SH, C₁-C₈ alkylthio, C₁-C₈ alkylsulphinyl, C₁-C₈ alkylsulphonyl, N(R¹²)₂, CO₂R¹¹, O(CO)R¹³, CON(R¹²)₂, NR¹²COR¹³ or CR¹³N—OR¹¹, wherein the alkyl, alkoxy, cycloalkyl, alkenyl, alkynyl, phenyl and heterocycle are optionally substituted by one or more groups, e.g. one to five groups, independently selected from halogen, CN, NH₂, NO₂, OH, C₁-C₄ alkyl, C₁-C₄ haloalkyl, C₁-C₄ alkoxy and C₁-C₄ haloalkoxy; or R⁶ and R⁷, R⁷ and R⁸, R³ and R⁸, or R³ and R⁹ together with the fragment of the pyridyl ring to which they are attached may form a partially or fully unsaturated 5- to 7-membered carbocyclic ring (e.g. an aryl or cycloalkyl ring as defined herein, e.g. cyclopentenyl, cyclohexenyl or phenyl) or a 5- to 7-membered heterocyclic ring containing one to three heteroatoms independently selected from O, S, N and N(R¹²), providing that the heterocycle does not contain adjacent oxygen atoms, adjacent sulphur atoms, or adjacent sulphur and oxygen atoms (e.g. a heterocycle as defined herein, preferably the single double-bond unsaturated equivalent of any of morpholine, pyrrolidine, piperidine, piperazine pyridine and pyrrole, or imidazole or triazole, e.g. 1,2,4 triazole, more preferably the single double-bond unsaturated equivalent of either morpholine or pyrollidine, or pyridine or imidazole), and wherein the ring formed by R⁶ and R⁷, R⁷ and R⁸, R³ and R⁸, or R³ and R⁹ is optionally substituted by one or more groups, e.g. one to five groups, independently selected from halogen, CN, NH₂, NO₂, OH, C₁-C₄ alkyl, C₁-C₄ haloalkyl, C₁-C₄ alkoxy and C₁-C₄ haloalkoxy.

Preferably R³, R⁶, R⁷, R⁸ and R⁹ independently of one another represent hydrogen, halogen, OH, CN, C₁-C₈ alkyl, C₁-C₈ haloalkyl, C₁-C₈ alkoxy, C₁-C₈ haloalkoxy, C₃-C₈ cycloalkyl, phenyl, pyridyl, N(R¹²)₂ or NR¹²COR¹³, wherein the phenyl and pyridyl are optionally substituted by one or more groups, e.g. one to five groups, independently selected from halogen, CN, C₁-C₄ alkyl, C₁-C₄ haloalkyl, OH, C₁-C₄ alkoxy and C₁-C₄ haloalkoxy; or R⁶ and R⁷, R⁷ and R⁸, R³ and R⁸, or R³ and R⁹, together with the fragment of the pyridyl ring to which they are attached may form a partially or fully unsaturated 5- to 7-membered carboyclic ring (e.g. an aryl or cycloalkyl ring as defined herein, e.g. cyclopentenyl, cyclohexenyl or phenyl) or a 5- to 7-membered heterocyclic ring containing one to three heteroatoms independently selected from O, S, N and N(R¹²), providing that the heterocycle does not contain adjacent oxygen atoms, adjacent sulphur atoms, or adjacent sulphur and oxygen atoms (e.g. a heterocycle as defined herein, preferably the single double-bond unsaturated equivalent of any of morpholine, pyrrolidine, piperidine, piperazine pyridine and pyrrole, and imidazole or triazole, e.g. 1,2,4 triazole, more preferably the single double-bond unsaturated equivalent of either morpholine or pyrollidine, or pyridine or imidazole), wherein the ring formed by R⁶ and R⁷, R⁷ and R⁸, R³ and R⁸, or R³ and R⁹ is optionally substituted by one or more groups, e.g. one to five groups, independently selected from halogen, C₁-C₄ alkyl, C₁-C₄ haloalkyl, C₁-C₄ alkoxy and C₁-C₄ haloalkoxy.

More preferably R³, R⁶, R⁷, R⁸ and R⁹ independently of one another represent hydrogen, halogen, C₁-C₄ alkyl, C₁-C₄ haloalkyl, C₁-C₄ alkoxy, C₁-C₄ haloalkoxy, C₃-C₆ cycloalkyl, N(R¹²)₂ or NR¹²COR¹³; or R⁶ and R⁷, R⁷ and R⁸, R³ and R⁸, or R³ and R⁹, together with the fragment of the pyridyl ring to which they are attached may form a fully or partially unsaturated 6-membered carbocyclic ring, optionally substituted by halogen, methyl and halomethyl.

Even more preferably R³, R⁶, R⁷, R⁸ and R⁹ independently of one another represent hydrogen, halogen, C₁-C₄ alkyl, C₁-C₄ haloalkyl, C₁-C₄ alkoxy, C₁-C₄ haloalkoxy, C₃-C₆ cycloalkyl or N(R¹²)₂; or R⁶ and R⁷, R⁷ and R⁸, R³ and R⁸, or R³ and R⁹, together with the fragment of the pyridyl ring to which they are attached may form a fully or partially unsaturated 6-membered carbocyclic ring optionally substituted by one or more groups, e.g. one to five groups, selected from halogen, methyl and halomethyl.

In one group of compounds R³, R⁶, R⁷, R⁸ and R⁹ independently of one another represent hydrogen, C₁-C₄ alkyl, CN or C₁-C₄ alkoxy, wherein the alkyl and alkoxy are optionally substituted by one or more groups independently selected from halogen, CN, C₁-C₄ alkoxy and C₁-C₄ haloalkoxy.

In another group of compounds, R³, R⁶, R⁷, R⁸ and R⁹ independently of one another represent hydrogen, halogen, CN, C₁-C₈ alkyl, C₃-C₈ cycloalkyl, C₂-C₈ alkenyl, C₂-C₈ alkynyl, phenyl, a 5- or 6-membered heterocycle containing one to three nitrogen atoms, OR¹¹, SH, C₁-C₈-alkylthio, C₁-C₈-alkylsulphinyl, C₁-C₈-alkylsulphonyl, CO₂R¹¹, CON(R¹²)₂, or wherein the alkyl, cycloalkyl, alkenyl, alkynyl, phenyl and heterocycle are optionally substituted by one or more groups independently selected from halogen, CN, NH₂, NO₂, OH, C₁-C₄ alkyl, C₁-C₄ haloalkyl, C₁-C₄ alkoxy and C₁-C₄ haloalkoxy;

or R⁶ and R⁷, R⁷ and R⁸, R³ and R⁸, or R³ and R⁹ together with the fragment of the pyridyl ring to which they are attached may form a partially or fully unsaturated 5- to 7-membered carbocyclic ring, and wherein the ring formed by R⁶ and R⁷, R⁷ and R⁸, R³ and R⁸, or R³ and R⁹ is optionally substituted by one or more groups independently selected from halogen, CN, NH₂, NO₂, OH, C₁-C₄ alkyl, C₁-C₄ haloalkyl, C₁-C₄ alkoxy and C₁-C₄ haloalkoxy;

in this group of compounds, each R¹¹ independently of one another represent hydrogen, C₁-C₄ alkyl, C₃-C₈ alkenyl, C₃-C₈ alkynyl, benzyl, or phenyl, wherein the alkyl, cycloalkyl, alkenyl, alkynyl, phenyl and benzyl are optionally substituted by one or more groups independently selected from halogen, CN, NH₂, NO₂, OH, C₁-C₄ alkyl, C₁-C₄-haloalkyl, C₁-C₄ alkoxy and C₁-C₄ haloalkoxy;

each R¹² independently of one another represent hydrogen or C₁-C₈ alkyl.

In this group, preferably R³, R⁶, R⁷, R⁸ and R⁹ independently of one another represent hydrogen, halogen, CN, C₁-C₄ alkyl, C₂-C₆ alkenyl, C₂-C₆ alkynyl, phenyl, OR¹¹, SH, C₁-C₄-alkylthio, C₁-C₄-alkylsulphinyl, C₁-C₄-alkylsulphonyl, wherein the alkyl, alkoxy, alkenyl, alkynyl and phenyl are optionally substituted by one or more groups independently selected from halogen, CN, NH₂, NO₂, OH, C₁-C₄ alkyl, C₁-C₄ haloalkyl, C₁-C₄ alkoxy and C₁-C₄ haloalkoxy;

or R⁶ and R⁷, R⁷ and R⁸, R³ and R⁸, or R³ and R⁹ together with the fragment of the pyridyl ring to which they are attached may form a partially or fully unsaturated 5- to 7-membered carbocyclic ring;

each R¹¹ independently of one another represent hydrogen, C₁-C₄ alkyl, C₃-C₆ alkenyl, C₃-C₆ alkynyl or phenyl;

each R¹² independently of one another represent hydrogen or C₁-C₈ alkyl.

X represents X-2, X-3, X-4 or X-5:

#—Z²—Z³—# X-2

#—Z⁴—Z⁵—Z⁶—# X-3

#—Z⁷—Z⁸—Z⁹—Z¹⁰—# X-4

#—Z¹¹—Z¹²—Z¹³—Z¹⁴—Z¹⁵—# X-5

Preferably X represents X-3 or X-5. More preferably X represents X-3. Z², Z³, Z⁴, Z⁶, Z⁷, Z⁸, Z⁹, Z¹⁰, Z¹¹, Z¹², Z¹⁴ and Z¹⁵ independently of one another represent CR¹⁴R¹⁵, C═O or C═CR¹⁹R²⁰; Z⁵ and Z¹³ independently of one another represent CR^{14′}R^{15′}, SiR¹⁶R¹⁷, C═O or C═CR¹⁹R²⁰.

Preferably Z², Z³, Z⁴, Z⁶, Z⁷, Z⁸, Z⁹, Z¹⁰, Z¹¹, Z¹², Z¹⁴ and Z¹⁵ independently of one another represent methylene or halomethylene; Z⁵ and Z¹³ independently of one another represent CR^{14′}R^{15′} or C═CR¹⁹C²⁰.

More preferably Z², Z³, Z⁴, Z⁶, Z⁷, Z⁸, Z⁹, Z¹⁰, Z¹¹, Z¹², Z¹⁴ and Z¹⁵ independently of one another represent methylene; Z⁵ and Z¹³ independently of one another represent CR^{14′}R^{15′} or C═CR¹⁹R²⁰.

Each R¹⁴ and R¹⁵ independently of one another represent hydrogen, halogen, OH, C₁-C₄ alkyl, C₁-C₄ haloalkyl, phenyl or CN, wherein the phenyl is optionally substituted by one or more groups, e.g. one to five groups, independently selected from halogen, CN, C₁-C₄ alkyl, C₁-C₄ haloalkyl, C₁-C₄ alkoxy and C₁-C₄ haloalkyoxy; or R¹⁴ and R¹⁵ together with the carbon atom to which they are attached may form a C₃-C₆ cycloalkyl group or a C₃-C₆ halocycloalkyl group.

Each R¹⁹ and R²⁰ independently of one another represent hydrogen, halogen, C₁-C₄ alkyl or C₁-C₄ haloalkyl.

Preferably each R¹⁹ and R²⁰ independently of one another represent hydrogen, halogen, methyl or halomethyl.

Each R^{14′}, R^{15′}, R¹⁶ and R¹⁷ independently of one another represent hydrogen, halogen, OH, C₁-C₄ alkyl, C₁-C₄ haloalkyl, phenyl or CN, wherein phenyl is optionally substituted by one or more groups, e.g. one to five groups, independently selected from halogen, CN, C₁-C₄ alkyl, C₁-C₄ haloalkyl, C₁-C₄ alkoxy and C₁-C₄ haloalkyoxy; or R^{14′} and R^{15′} together with the carbon atom to which they are attached may form a C₃-C₆ cycloalkyl group or a C₃-C₆ halocycloalkyl group.

Preferably, each R^{14′} and R^{15′} independently of one another represent hydrogen, halogen, OH, C₁-C₄ alkyl, C₁-C₄ haloalkyl, phenyl or CN, wherein the phenyl is optionally substituted by one or more groups independently selected from halogen, CN, methyl, halomethyl, methoxy and halomethoxy; or R^{14′} and R^{15′} together with the carbon atom to which they are attached may form a C₃-C₆ cycloalkyl group or a C₃-C₆ halocycloalkyl group.

Y⁶, Y⁷, and Y⁸ independently of one another represent hydrogen, halogen, CN, NO₂, C₁-C₈ alkyl, C₁-C₄-alkoxy-C₁-C₄-alkyl, C₁-C₄-alkoxy-C₁-C₄-alkoxy-C₁-C₄-alkyl, C₃-C₈ cycloalkyl, C₂-C₈ alkenyl, C₂-C₈ alkynyl, phenyl, pyridyl, COR¹³, OR²², SH, C₁-C₈-alkylthio, C₁-C₈ alkylsulphinyl, C₁-C₈ alkylsulphonyl, N(R²³)₂, CO₂R²², O(CO)R¹³, CON(R²³)₂, NR²³COR¹³ or CR¹³N—OR²², wherein the alkyl, alkoxy, cycloalkyl, alkenyl, alkynyl, phenyl and pyridyl are optionally substituted by one or more groups, e.g. one to five groups, independently selected from halogen, CN, NH₂, NO₂, OH, C₁-C₄ alkyl, C₁-C₄ haloalkyl, C₁-C₄ alkoxy and C₁-C₄ haloalkoxy.

Preferably, Y⁶, Y⁷ and Y⁸ independently of one another represent hydrogen, halogen, N(R²³)₂CN, NO₂, C₁-C₈ alkyl, C₁-C₆-alkoxy-C₁-C₄-alkyl, C₃-C₈ cycloalkyl, C₂-C₆ alkenyl, C₂-C₆ alkynyl, phenyl, pyridyl, OR²², SH, C₁-C₈ alkylthio, C₁-C₈ alkylsulphinyl or C₁-C₈ alkylsulphonyl, wherein the alkyl, alkoxy, cycloalkyl, alkenyl, alkynyl, phenyl and pyridyl are optionally substituted by one or more groups independently selected from halogen, CN, NH₂, NO₂, OH, C₁-C₄ alkyl, C₁-C₄ haloalkyl, C₁-C₄ alkoxy and C₁-C₄ haloalkoxy;

or Y⁶ and Y⁷ or Y⁷ and Y⁸ together with the fragment of the pyridyl ring to which they are attached may form a partially or fully unsaturated 5- to 7-membered carbocyclic ring, wherein the ring formed by Y⁶ and Y⁷ or Y⁷ and Y⁸ is optionally substituted by one or more groups independently selected from halogen, CN, NH₂, NO₂, OH, C₁-C₄ alkyl, C₁-C₄ haloalkyl, C₁-C₄ alkoxy and C₁-C₄ haloalkoxy;

wherein each R²² independently of one another represent hydrogen, C₁-C₄ alkyl, C₃-C₆ cycloalkyl, C₃-C₆ alkenyl, C₃-C₆ alkynyl, benzyl, phenyl or pyridyl, wherein the alkyl, cycloalkyl, alkenyl, alkynyl, phenyl, benzyl and pyridyl are optionally substituted by one or more groups independently selected from halogen, CN, NH₂, NO₂, OH, C₁-C₄ alkyl, C₁-C₄-haloalkyl, C₁-C₄ alkoxy, C₁-C₄ haloalkoxy and C₁-C₄-alkoxy-C₁-C₄-alkyl;

each R²³ independently of one another represent hydrogen or C₁-C₈ alkyl, wherein the alkyl, is optionally substituted by one or more halogen;

wherein when two radicals R²³ are attached to the same nitrogen atom, these radicals can be identical or different;

and wherein when two radicals R²³ are attached to the same nitrogen atom, these two radicals together with the nitrogen atom to which they are attached may form a cycle B-1, B-2, B-3, B-4 or B-5 wherein the cycle formed is optionally substituted by one or more groups independently selected from halogen, methyl and halomethyl.

Preferably, Y⁶, Y⁷ and Y⁸ independently of one another represent hydrogen, halogen, N(R²³)₂CN, NO₂, C₁-C₆ alkyl, C₁-C₄-alkoxy-C₁-C₄-alkyl, C₃-C₆ cycloalkyl, C₂-C₆ alkenyl, C₂-C₆ alkynyl, phenyl, pyridyl, C₁-C₄-alkoxy, C₁-C₄-alkenoxy, C₁-C₄-alkynoxy, phenoxy, SH, C₁-C₈ alkylthio, C₁-C₈ alkylsulphinyl or C₁-C₈ alkylsulphonyl, wherein the alkyl, alkoxy, alkenoxy, alkynoxy, phenoxy cycloalkyl, alkenyl, alkynyl, phenyl and pyridyl are optionally substituted by one or more groups independently selected from halogen, CN, NH₂, NO₂, OH, methyl and halomethyl;

or Y⁶ and Y⁷ or Y⁷ and Y⁸ together with the fragment of the pyridyl ring to which they are attached may form a partially or fully unsaturated 5- to 7-membered carbocyclic ring, wherein the ring formed by Y⁶ and Y⁷ or Y⁷ and Y⁸ is optionally substituted by one or more groups independently selected from halogen, CN, NH₂, NO₂, OH, methyl and halomethyl;

wherein each R²³ independently of one another represent hydrogen or C₁-C₈ alkyl, the alkyl, is optionally substituted by one or more halogen;

wherein when two radicals R²³ are attached to the same nitrogen atom, these radicals can be identical or different;

and wherein when two radicals R²³ are attached to the same nitrogen atom, these two radicals together with the nitrogen atom to which they are attached may form a cycle B-1, B-2, B-3, B-4 or B-5 wherein the cycle formed is optionally substituted by one or more groups independently selected from halogen, methyl and halomethyl.

In another group of compounds, Y⁶, Y⁷ and Y⁸ independently of one another represent hydrogen, halogen, OH, CN, C₁-C₈ alkyl, C₁-C₈ haloalkyl, C₁-C₈ alkoxy, C₁-C₈ haloalkoxy, C₃-C₈ cycloalkyl, phenyl, pyridyl, N(R²³)₂ or NR¹²COR¹³, wherein phenyl and pyridyl are optionally substituted by one or more groups, e.g. one to five groups, independently selected from halogen, CN, C₁-C₄ alkyl, C₁-C₄ haloalkyl, C₁-C₄ alkoxy and C₁-C₄ haloalkoxy.

In another group of compounds, Y⁶, Y⁷ and Y⁸ independently of one another represent hydrogen, CN, OH, halogen, C₁-C₄ alkyl, C₁-C₄ haloalkyl, C₁-C₄ alkoxy, C₁-C₄ haloalkoxy, C₃-C₆ cycloalkyl, N(R²³)₂, NR²³COR¹³ or phenyl, wherein phenyl is optionally substituted by one or more groups, e.g. one to five groups, independently selected from halogen, CN, C₁-C₄ alkyl, C₁-C₄ haloalkyl, C₁-C₄ alkoxy and C₁-C₄ haloalkoxy.

In another group of compounds, Y⁶, Y⁷, and Y⁸ independently of one another represent hydrogen, CN, OH, NH₂, halogen, C₁-C₄ alkyl, C₁-C₄ haloalkyl, C₁-C₄ alkoxy, C₁-C₄ haloalkoxy, C₃-C₆ cycloalkyl, N(R²³)₂, NR²³COR¹³ or phenyl, wherein phenyl is optionally substituted by one or more groups, e.g. one to five groups, independently selected from halogen, methyl, CN, methoxy, halomethyl and halomethoxy.

In one group of compounds Y⁶ and Y⁷ independently of one another represent hydrogen, C₁-C₄ alkyl, CN or C₁-C₄ alkoxy, wherein the alkyl and alkoxy are optionally substituted by one or more groups independently selected from halogen, CN, C₁-C₄ alkoxy and C₁-C₄ haloalkoxy. In another group of compounds Y⁶, Y⁷ and Y⁸ independently of one another represent hydrogen, C₁-C₄ alkyl, CN or C₁-C₄ alkoxy, wherein the alkyl and alkoxy are optionally substituted by one or more groups independently selected from halogen, CN, C₁-C₄ alkoxy and C₁-C₄ haloalkoxy.

In one group of compounds, Y⁶ and Y⁷ or Y⁷ and Y⁸ together with the fragment of the pyridyl ring to which they are attached may form a partially or fully unsaturated 5- to 7-membered carbocyclic ring or a 5- to 7-membered heterocyclic ring containing one to three heteroatoms independently selected from O, S, N and N(R¹²), providing that the heterocycle does not contain adjacent oxygen atoms, adjacent sulphur atoms, or adjacent sulphur and oxygen atoms, and wherein the ring formed by Y⁶ and Y⁷ or Y⁷ and Y⁸ is optionally substituted by one or more groups independently selected from halogen, CN, NH₂, NO₂, OH, C₁-C₄ alkyl, C₁-C₄ haloalkyl, C₁-C₄ alkoxy and C₁-C₄ haloalkoxy; preferably Y⁶ and Y⁷ or Y⁷ and Y⁸ together with the fragment of the pyridyl ring to which they are attached may form a partially or fully unsaturated 5- to 7-membered carbocyclic ring or a 5- to 7-membered heterocyclic ring containing one to three heteroatoms independently selected from N and N(R¹²), and wherein the ring formed by Y⁶ and Y⁷ or Y⁷ and Y⁸ is optionally substituted by one or more groups independently selected from halogen, CN, NH₂, NO₂, OH, C₁-C₄ alkyl, C₁-C₄ haloalkyl, C₁-C₄ alkoxy and C₁-C₄ haloalkoxy.

In one group of compounds Y⁷ is preferably hydrogen.

Each R¹¹ and R²² independently of one another represent hydrogen, C₁-C₈ alkyl, C₃-C₈ cycloalkyl, C₃-C₈ alkenyl, C₃-C₈ alkynyl, benzyl, phenyl or pyridyl, wherein the alkyl, cycloalkyl, alkenyl, alkynyl, phenyl, benzyl and pyridyl are optionally substituted by one or more groups, e.g. one to five groups, independently selected from halogen, CN, NH₂, NO₂, OH, C₁-C₄-alkyl, C₁-C₄-haloalkyl, C₁-C₄-alkoxy, C₁-C₄-haloalkoxy and C₁-C₄-alkoxy-C₁-C₄-alkyl.

Each R¹² and R²³ independently of one another represent hydrogen, OH, C₁-C₈ alkyl, C₁-C₈ alkoxy, C₁-C₈-alkoxy-C₁-C₄-alkyl, C₃-C₈ alkenyl, C₃-C₈ alkynyl, or COR¹³, wherein the alkyl, alkoxy, alkenyl and alkynyl are optionally substituted by one or more halogen; wherein when two radicals R¹² or two radicals R²³ are attached to the same nitrogen atom, these radicals can be identical or different; wherein when two radicals R¹² or two radicals R²³ are attached to the same nitrogen atom, both of these radicals cannot be OH or C₁-C₄ alkoxy or C₁-C₄ haloalkoxy; and wherein when two radicals R¹² or two radicals R²³ are attached to the same nitrogen atom, these two radicals together with the nitrogen atom to which they are attached may form a cycle B-1, B-2, B-3, B-4, B-5, B-6, B-7 or B-8:

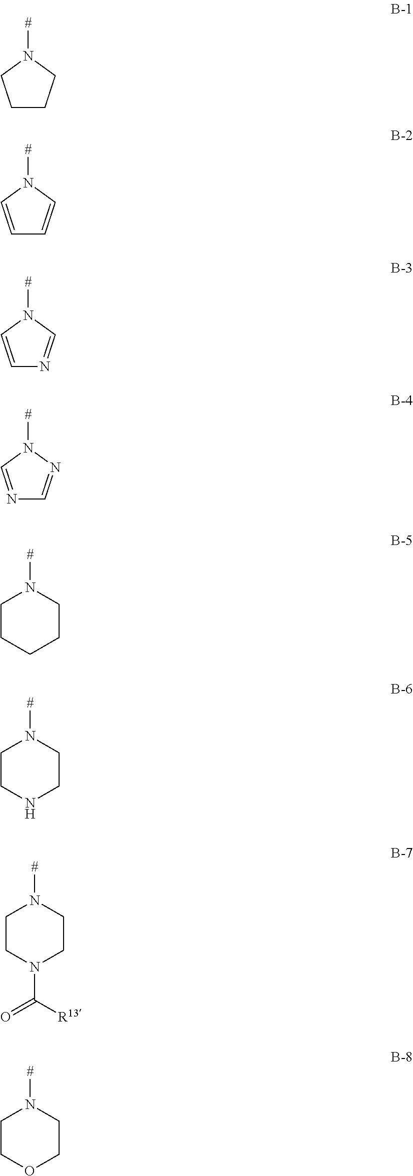

wherein the cycle formed is optionally substituted by one or more groups, e.g. one to five groups, independently selected from halogen, CN, NH₂, NO₂, OH, C₁-C₄-alkyl, C₁-C₄-haloalkyl, C₁-C₄-alkoxy and C₁-C₄-haloalkoxy.

Preferably each R¹² and R²³ independently of one another represent hydrogen, C₁-C₈ alkyl or COR¹³; wherein when two radicals R¹² or two radicals R²³ are attached to the same nitrogen atom, these radicals can be identical or different; and wherein when two radicals R¹² or two radicals R²³ are attached to the same nitrogen atom, these two radicals together with the nitrogen atom to which they are attached may form a cycle B-1, B-2, B-3, B-4 or B-5 wherein the cycle formed is optionally substituted by one or more groups, e.g. one to five groups independently selected from halogen, methyl and halomethyl.

More preferably each R¹² and R²³ independently of one another represent hydrogen or C₁-C₄ alkyl; wherein when two radicals R¹² or two radicals R²³ are attached to the same nitrogen atom, these radicals can be identical or different; and wherein when two radicals R¹² are attached to the same nitrogen atom, these two radicals together with the nitrogen atom to which they are attached may form a cycle B-1, B-2, B-3, B-4 or B-5 wherein the cycle formed is optionally substituted by one or more groups, e.g. one to five groups, independently selected from halogen, methyl and halomethyl.

Each R¹³ and R^{13′} independently of one another represent hydrogen, C₁-C₈ alkyl, C₃-C₈ cycloalkyl, C₂-C₈ alkenyl, C₂-C₈ alkynyl, benzyl, phenyl or pyridyl, wherein the alkyl, cycloalkyl, alkenyl, alkynyl, phenyl, benzyl and pyridyl are optionally substituted by one or more groups, e.g. one to five groups, independently selected from halogen, CN, NH₂, NO₂, OH, C₁-C₄ alkyl, C₁-C₄ haloalkyl, C₁-C₄ alkoxy and C₁-C₄ haloalkoxy.

Preferably each R¹³ and R^{13′} independently of one another represent C₁-C₈ alkyl or C₁-C₈ haloalkyl, more preferably C₁-C₄ alkyl or C₁-C₄ haloalkyl.

G¹ and G² independently of one another represent —C(R²⁴R²⁵)—; G³ represents —C(R²⁴R²⁵)—, O, N(R²⁶) or S; or G¹ and G², or G² and G³, or G¹ and G¹ together represent —CR²⁴═CR²⁵—;

Preferably G¹, G² and G³ independently of one another represent —C(R²⁴R²⁵)—.

Even more preferably G¹, G² and G³ represent methylene.

Each R²⁴ and R²⁵ independently of one another represent hydrogen, halogen, C₁-C₄ alkyl, C₁-C₄ haloalkyl, C₁-C₄ alkoxy or C₁-C₄ haloalkoxy.

R²⁶

represents hydrogen, OH, C₁-C₄ alkyl, C₁-C₄ alkoxy, C₁-C₈ alkylcarbonyl or C₁-C₈ haloalkylcarbonyl.

p is 1 or 2.

More preferably p is 1.

In a preferred group of compounds X represents X-3;

Z⁴ and Z⁶ represent methylene;

Z⁵ represents CR^{14′}R^{15′} or C═CR¹⁹R²⁰;

each R^{14′} and R^{15′} independently of one another represent hydrogen, halogen, C₁-C₄ alkyl, C₁-C₄ haloalkyl or phenyl, wherein the phenyl is optionally substituted by one or more groups independently selected from halogen, CN, methyl, halomethyl, methoxy and halomethoxy;

or R^{14′} and R^{15′} together with the carbon atom to which they are attached may form a C₃-C₆ cycloalkyl group optionally substituted by halogen; and

each R¹⁹ and R²⁰ independently of one another represent hydrogen, halogen, methyl or halomethyl.

In yet another group of compounds R¹ and A¹ represent identical substituents.

In another group of compounds, R¹ represents hydrogen, (C₁-C₄ alkyl)O₂C, C₁-C₁₀ alkyl, phenyl or pyridyl, wherein the alkyl, phenyl and pyridyl are optionally substituted by one or more groups independently selected from halogen, CN, C₁-C₄ alkyl, C₁-C₄ haloalkyl, C₃-C₆ cycloalkyl and a 5- or 6-membered heterocycle containing one to three nitrogen atoms;

A¹ represents cycle A-2, A-4, or A-5;

R³, R⁶, R⁷, R⁸ and R⁹ independently of one another represent hydrogen, halogen, CN, C₁-C₈ alkyl, C₃-C₈ cycloalkyl, C₂-C₈ alkenyl, C₂-C₈ alkynyl, phenyl, a 5- or 6-membered heterocycle containing one to three nitrogen atoms, OR¹¹, SH, C₁-C₈-alkylthio, C₁-C₈-alkylsulphinyl, C₁-C₈-alkylsulphonyl, CO₂R¹¹, CON(R¹²)₂, or wherein the alkyl, cycloalkyl, alkenyl, alkynyl, phenyl and heterocycle are optionally substituted by one or more groups independently selected from halogen, CN, NH₂, NO₂, OH, C₁-C₄ alkyl, C₁-C₄ haloalkyl, C₁-C₄ alkoxy and C₁-C₄ haloalkoxy;

or R⁶ and R⁷, R⁷ and R⁸, R³ and R⁸, or R³ and R⁹ together with the fragment of the pyridyl ring to which they are attached may form a partially or fully unsaturated 5- to 7-membered carbocyclic ring, and wherein the ring formed by R⁶ and R⁷, R⁷ and R⁸, R³ and R⁸, or R³ and R⁹ is optionally substituted by one or more groups independently selected from halogen, CN, NH₂, NO₂, OH, C₁-C₄ alkyl, C₁-C₄ haloalkyl, C₁-C₄ alkoxy and C₁-C₄ haloalkoxy;

each R¹¹ independently of one another represent hydrogen, C₁-C₄ alkyl, C₃-C₈ alkenyl, C₃-C₈ alkynyl, benzyl, or phenyl, wherein the alkyl, cycloalkyl, alkenyl, alkynyl, phenyl and benzyl are optionally substituted by one or more groups independently selected from halogen, CN, NH₂, NO₂, OH, C₁-C₄ alkyl, C₁-C₄-haloalkyl, C₁-C₄ alkoxy and C₁-C₄ haloalkoxy;

each R¹² independently of one another represent hydrogen or C₁-C₈ alkyl.

In another group of compounds R¹ represents hydrogen, halogen, (C₁-C₄ alkyl)O₂C, C₁-C₁₀ alkyl, phenyl or pyridyl, wherein the alkyl, phenyl and pyridyl are optionally substituted by one or more groups independently selected from halogen, CN, C₁-C₄ alkyl, C₁-C₄ haloalkyl, C₃-C₆ cycloalkyl and a 5- or 6-membered heterocycle containing one to three nitrogen atoms;

A¹ represents cycle A-2, A-4, or A-5;

R³, R⁶, R⁷, R⁸ and R⁹ independently of one another represent hydrogen, halogen, CN, C₁-C₄ alkyl, C₂-C₆ alkenyl, C₂-C₆ alkynyl, phenyl, OR¹¹, SH, C₁-C₄-alkylthio, C₁-C₄-alkylsulphinyl, C₁-C₄-alkylsulphonyl, wherein the alkyl, alkoxy, cycloalkyl, alkenyl, alkynyl, phenyl and heterocycle are optionally substituted by one or more groups independently selected from halogen, CN, NH₂, NO₂, OH, C₁-C₄ alkyl, C₁-C₄ haloalkyl, C₁-C₄ alkoxy and C₁-C₄ haloalkoxy;

or R⁶ and R⁷, R⁷ and R⁸, R³ and R⁸, or R³ and R⁹ together with the fragment of the pyridyl ring to which they are attached may form a partially or fully unsaturated 5- to 7-membered carbocyclic ring;

each R¹¹ independently of one another represent hydrogen, C₁-C₄ alkyl, C₃-C₆ alkenyl, C₃-C₆ alkynyl or phenyl;

each R¹² independently of one another represent hydrogen or C₁-C₈ alkyl.

In a further group of preferred compounds R¹ represents hydrogen, halogen, CN, OH, SH, C₁-C₈ alkylthio, C₁-C₈ alkylsulphinyl, C₁-C₈ alkylsulphonyl, NH₂, C₁-C₁₀ alkyl, C₃-C₈ cycloalkyl, C₂-C₈ alkenyl, C₂-C₈ alkynyl, (C₁-C₄ alkyloxycarbonyl) C₁-C₄-alkyl, (C₁-C₄ alkyl)O₂C, phenyl or pyridyl, wherein the alkyl, cycloalkyl, alkenyl, alkynyl, phenyl and pyridyl are optionally substituted by one or more groups independently selected from halogen, CN, NH₂, NO₂, OH, C₁-C₄ alkyl, C₁-C₄ haloalkyl, C₁-C₄ alkoxy, C₁-C₄ haloalkoxy, C₃-C₆ cycloalkyl and a 5- or 6-membered heterocycle containing one to three heteroatoms independently selected from O, S and N, providing that the heterocycle does not contain adjacent oxygen atoms, adjacent sulphur atoms, or adjacent sulphur and oxygen atoms;

A¹ represents cycle A-2;

R³, R⁷, R⁸ and R⁹ independently of one another represent hydrogen, halogen, CN, C₁-C₈ alkyl, C₃-C₈ cycloalkyl, C₂-C₈ alkenyl, C₂-C₈ alkynyl, phenyl, a 5- or 6-membered heterocycle containing one to three nitrogen atoms, OR¹¹, SH, C₁-C₈-alkylthio, C₁-C₈-alkylsulphinyl, C₁-C₈-alkylsulphonyl, CO₂R¹¹ or CON(R¹²)₂ wherein the alkyl, cycloalkyl, alkenyl, alkynyl, phenyl and heterocycle are optionally substituted by one or more groups independently selected from halogen, CN, NH₂, NO₂, OH, C₁-C₄ alkyl, C₁-C₄ haloalkyl, C₁-C₄ alkoxy and C₁-C₄ haloalkoxy;

or R⁷ and R⁸, R³ and R⁸, or R³ and R⁹ together with the fragment of the pyridyl ring to which they are attached may form a partially or fully unsaturated 5- to 7-membered carbocyclic ring, and wherein the ring formed by R⁷ and R⁸, R³ and R⁸, or R³ and R⁹ is optionally substituted by one or more groups independently selected from halogen, CN, NH₂, NO₂, OH, C₁-C₄ alkyl, C₁-C₄ haloalkyl, C₁-C₄ alkoxy and C₁-C₄ haloalkoxy;

each R¹¹ independently of one another represent hydrogen, C₁-C₄ alkyl, C₃-C₈ alkenyl, C₃-C₈ alkynyl, benzyl, or phenyl, wherein the alkyl, cycloalkyl, alkenyl, alkynyl, phenyl and benzyl are optionally substituted by one or more groups independently selected from halogen, CN, NH₂, NO₂, OH, C₁-C₄ alkyl, C₁-C₄-haloalkyl, C₁-C₄ alkoxy and C₁-C₄ haloalkoxy;

each R¹² independently of one another represent hydrogen or C₁-C₈ alkyl;

X represents X-3;

Z⁴ and Z⁶ represent methylene;

Z⁵ represents CR^{14′}R^{15′} or C═CR¹⁹R²⁰;

each R^{14′} and R^{15′} independently of one another represent hydrogen, halogen, C₁-C₄ alkyl, C₁-C₄ haloalkyl or phenyl, wherein the phenyl is optionally substituted by one or more groups independently selected from halogen, CN, methyl, halomethyl, methoxy and halomethoxy;

or R^{14′} and R^{15′} together with the carbon atom to which they are attached may form a C₃-C₆ cycloalkyl group optionally substituted by halogen; and

each R¹⁹ and R²⁰ independently of one another represent hydrogen, halogen, methyl or halomethyl;

Y⁶, Y⁷ and Y⁸ independently of one another represent hydrogen, halogen, N(R²³)₂CN, NO₂, C₁-C₈ alkyl, C₁-C₆-alkoxy-C₁-C₄-alkyl, C₃-C₈ cycloalkyl, C₂-C₆ alkenyl, C₂-C₆ alkynyl, phenyl, pyridyl, OR²², SH, C₁-C₈ alkylthio, C₁-C₈ alkylsulphinyl or C₁-C₈ alkylsulphonyl, wherein the alkyl, alkoxy, cycloalkyl, alkenyl, alkynyl, phenyl and pyridyl are optionally substituted by one or more groups independently selected from halogen, CN, NH₂, NO₂, OH, C₁-C₄ alkyl, C₁-C₄ haloalkyl, C₁-C₄ alkoxy and C₁-C₄ haloalkoxy;

or Y⁶ and Y⁷ or Y⁷ and Y⁸ together with the fragment of the pyridyl ring to which they are attached may form a partially or fully unsaturated 5- to 7-membered carbocyclic ring, wherein the ring formed by Y⁶ and Y⁷ or Y⁷ and Y⁸ is optionally substituted by one or more groups independently selected from halogen, CN, NH₂, NO₂, OH, C₁-C₄ alkyl, C₁-C₄ haloalkyl, C₁-C₄ alkoxy and C₁-C₄ haloalkoxy;

each R²² independently of one another represent hydrogen, C₁-C₄ alkyl, C₃-C₄ cycloalkyl, C₃-C₆ alkenyl, C₃-C₆ alkynyl, benzyl, phenyl or pyridyl, wherein the alkyl, cycloalkyl, alkenyl, alkynyl, phenyl, benzyl and pyridyl are optionally substituted by one or more groups independently selected from halogen, CN, NH₂, NO₂, OH, C₁-C₄ alkyl, C₁-C₄-haloalkyl, C₁-C₄ alkoxy, C₁-C₄ haloalkoxy and C₁-C₄-alkoxy-C₁-C₄-alkyl;

each R²³ independently of one another represent hydrogen or C₁-C₈ alkyl, wherein the alkyl is optionally substituted by one or more halogen;

wherein when two radicals R²³ are attached to the same nitrogen atom, these radicals can be identical or different;

and wherein when two radicals R²³ are attached to the same nitrogen atom, these two radicals together with the nitrogen atom to which they are attached may form a cycle B-1, B-2, B-3, B-4 or B-5 wherein the cycle formed is optionally substituted by one or more groups independently selected from halogen, methyl and halomethyl;

G¹, G² and G³ represent methylene; and

p is 1 or 2.

In yet another preferred group of compounds R¹ represents hydrogen, (C₁-C₄ alkyl)O₂C, C₁-C₁₀ alkyl, phenyl or pyridyl, wherein the alkyl, phenyl and pyridyl are optionally substituted by one or more groups independently selected from halogen, CN, C₁-C₄ alkyl, C₁-C₄ haloalkyl, C₃-C₆ cycloalkyl and a 5- or 6-membered heterocycle containing one to three nitrogen atoms;

A¹ represents cycle A-2;

R³, R⁷, R⁸ and R⁹ independently of one another represent hydrogen, halogen, CN, C₁-C₄ alkyl, C₂-C₆ alkenyl, C₂-C₆ alkynyl, phenyl, OR¹¹, SH, C₁-C₄-alkylthio, C₁-C₄-alkylsulphinyl, C₁-C₄-alkylsulphonyl, wherein the alkyl, alkoxy, cycloalkyl, alkenyl, alkynyl and phenyl are optionally substituted by one or more groups independently selected from halogen, CN, NH₂, NO₂, OH, C₁-C₄ alkyl, C₁-C₄ haloalkyl, C₁-C₄ alkoxy and C₁-C₄ haloalkoxy;

or R⁷ and R⁸, R³ and R⁸, or R³ and R⁹ together with the fragment of the pyridyl ring to which they are attached may form a partially or fully unsaturated 5- to 7-membered carbocyclic ring;

each R¹¹ independently of one another represent hydrogen, C₁-C₄ alkyl, C₃-C₆ alkenyl, C₃-C₆ alkynyl or phenyl;

each R¹² independently of one another represent hydrogen or C₁-C₈ alkyl;

X represents X-3;

Z⁴ and Z⁶ represent methylene;

Z⁵ represents CR^{14′}R^{15′} or C═CR¹⁹R²⁰;

each R^{14′} and R^{15′} independently of one another represent hydrogen, halogen, C₁-C₄ alkyl, C₁-C₄ haloalkyl or phenyl, wherein the phenyl is optionally substituted by one or more groups independently selected from halogen, CN, methyl, halomethyl, methoxy and halomethoxy;

or R^{14′} and R^{15′} together with the carbon atom to which they are attached may form a C₃-C₆ cycloalkyl group optionally substituted by halogen; and

each R¹⁹ and R²⁰ independently of one another represent hydrogen, halogen, methyl or halomethyl;

Y⁶, Y⁷ and Y⁸ independently of one another represent hydrogen, halogen, N(R²³)₂CN, NO₂, C₁-C₆ alkyl, C₁-C₄-alkoxy-C₁-C₄-alkyl, C₃-C₆ cycloalkyl, C₂-C₆ alkenyl, C₂-C₆ alkynyl, phenyl, pyridyl, C₁-C₄-alkoxy, C₁-C₄-alkenoxy, C₁-C₄-alkynoxy, phenoxy, SH, C₁-C₈ alkylthio, C₁-C₈ alkylsulphinyl or C₁-C₈ alkylsulphonyl, wherein the alkyl, alkoxy, alkenoxy, alkynoxy, phenoxy, cycloalkyl, alkenyl, alkynyl, phenyl and pyridyl are optionally substituted by one or more groups independently selected from halogen, CN, NH₂, NO₂, OH, methyl and halomethyl;

or Y⁶ and Y⁷ or Y⁷ and Y⁸ together with the fragment of the pyridyl ring to which they are attached may form a partially or fully unsaturated 5- to 7-membered carbocyclic ring, wherein the ring formed by Y⁶ and Y⁷ or Y⁷ and Y⁸ is optionally substituted by one or more groups independently selected from halogen, CN, NH₂, NO₂, OH, methyl and halomethyl;

each R²³ independently of one another represent hydrogen or C₁-C₈ alkyl, wherein the alkyl, is optionally substituted by one or more halogen;

wherein when two radicals R²³ are attached to the same nitrogen atom, these radicals can be identical or different;

and wherein when or two radicals R²³ are attached to the same nitrogen atom, these two radicals together with the nitrogen atom to which they are attached may form a cycle B-1, B-2, B-3, B-4 or B-5 wherein the cycle formed is optionally substituted by one or more groups independently selected from halogen, methyl and halomethyl;

G¹, G² and G³ represent methylene; and

p is 1.

The invention also provides compounds of formula (I-C), an embodiment of compounds of formula (I) wherein R¹ represents hydrogen, halogen, CN, OH, SH, C₁-C₈ alkylthio, C₁-C₈ alkylsulphinyl, C₁-C₈ alkylsulphonyl, NH₂, C₁-C₁₀ alkyl, C₃-C₈ cycloalkyl, C₂-C₈ alkenyl, C₂-C₈ alkynyl, (R¹¹O)carbonyl(C₁-C₄-alkyl), phenyl or pyridyl, wherein the alkyl, cycloalkyl, alkenyl, alkynyl, phenyl and pyridyl are optionally substituted by one or more groups independently selected from halogen, CN, NH₂, NO₂, OH, C₁-C₄ alkyl, C₁-C₄ haloalkyl, C₁-C₄ alkoxy, C₁-C₄ haloalkoxy, C₃-C₆ cycloalkyl and a 5- or 6-membered heterocycle containing one to three heteroatoms independently selected from O, S and N, providing that the heterocycle does not contain adjacent oxygen atoms, adjacent sulphur atoms, or adjacent sulphur and oxygen atoms;

A¹ represents cycle A-2, A-4, or A-5;

R³, R⁶, R⁷, R⁸ and R⁹ independently of one another represent hydrogen, halogen, CN, NO₂, C₁-C₈ alkyl, C₁-C₄-alkoxy-C₁-C₄-alkyl, C₁-C₄-alkoxy-C₁-C₄-alkoxy-C₁-C₄-alkyl, C₃-C₈ cycloalkyl, C₂-C₈ alkenyl, C₂-C₈ alkynyl, phenyl, a 5- or 6-membered heterocycle containing one to three heteroatoms independently selected from O, S and N, providing that the heterocycle does not contain adjacent oxygen atoms, adjacent sulphur atoms, or adjacent sulphur and oxygen atoms, COR¹³, OR¹¹, SH, C₁-C₈-alkylthio, C₁-C₈-alkylsulphinyl, C₁-C₈-alkylsulphonyl, N(R¹²)₂, CO₂R¹¹, O(CO)R¹³, CON(R¹²)₂, NR¹²COR¹³ or CR¹³N—OR¹¹, wherein the alkyl, alkoxy, cycloalkyl, alkenyl, alkynyl, phenyl and heterocycle are optionally substituted by one or more groups independently selected from halogen, CN, NH₂, NO₂, OH, C₁-C₄ alkyl, C₁-C₄ haloalkyl, C₁-C₄ alkoxy and C₁-C₄ haloalkoxy;

or R⁶ and R⁷, R⁷ and R⁸, R³ and R⁸, or R³ and R⁹ together with the fragment of the pyridyl ring to which they are attached may form a partially or fully unsaturated 5- to 7-membered carbocyclic ring or a 5- to 7-membered heterocyclic ring containing one to three heteroatoms independently selected from O, S, N and N(R¹²), providing that the heterocycle does not contain adjacent oxygen atoms, adjacent sulphur atoms, or adjacent sulphur and oxygen atoms, and wherein the ring formed by R⁶ and R⁷, R⁷ and R⁸, R³ and R⁸, or R³ and R⁹ is optionally substituted by one or more groups independently selected from halogen, CN, NH₂, NO₂, OH, C₁-C₄ alkyl, C₁-C₄ haloalkyl, C₁-C₄ alkoxy and C₁-C₄ haloalkoxy;

X represents X-2, X-3, X-4 or X-5;

Z², Z³, Z⁴, Z⁶, Z⁷, Z⁸, Z⁹, Z¹⁰, Z¹¹, Z¹², Z¹⁴ and Z¹⁵ independently of one another represent CR¹⁴R¹⁵, C═O or C═CR¹⁹R²⁰;

Z⁵ and Z¹³ independently of one another represent CR^{14′}R^{15′}, SiR¹⁶R¹⁷, C═O or C═CR¹⁹R²⁰;

each R¹⁴ and R¹⁵ independently of one another represent hydrogen, halogen, OH, C₁-C₄ alkyl, C₁-C₄ haloalkyl, phenyl or CN, wherein the phenyl is optionally substituted by one or more groups independently selected from halogen, CN, C₁-C₄ alkyl, C₁-C₄ haloalkyl, C₁-C₄ alkoxy and C₁-C₄ haloalkyoxy;

or R¹⁴ and R¹⁵ together with the carbon atom to which they are attached may form a C₃-C₆ cycloalkyl group or a C₃-C₆ halocycloalkyl group;

each R¹⁹ and R²⁰ independently of one another represent hydrogen, halogen, C₁-C₄ alkyl or C₁-C₄ haloalkyl;

each R^{14′}, R^{15′}, R¹⁶ and R¹⁷ independently of one another represent hydrogen, halogen, OH, C₁-C₄ alkyl, C₁-C₄ haloalkyl, phenyl or CN, wherein phenyl is optionally substituted by one or more groups independently selected from halogen, CN, C₁-C₄ alkyl, C₁-C₄ haloalkyl, C₁-C₄ alkoxy and C₁-C₄ haloalkyoxy;

or R^{14′} and R^{15′} together with the carbon atom to which they are attached may form a C₃-C₆ cycloalkyl group or a C₃-C₆ halocycloalkyl group;

wherein the groupings X-2, X-3, X-4 and X-5 contain at most one ring which contains either only one of the radicals Z² to Z¹⁵ or two radicals Z² to Z¹⁵ or three radicals Z² to Z¹⁵ or four radicals Z² to Z¹⁵ as ring members; and wherein radicals Z², Z³, Z⁴, Z⁶, Z⁷; Z¹⁰, Z¹¹ and Z¹⁵ are not substituted by OH;

Y⁶, Y⁷ and Y⁸ independently of one another represent hydrogen, halogen, CN, NO₂, C₁-C₈ alkyl, C₁-C₄-alkoxy-C₁-C₄-alkyl, C₁-C₄-alkoxy-C₁-C₄-alkoxy-C₁-C₄-alkyl, C₃-C₈ cycloalkyl, C₂-C₈ alkenyl, C₂-C₈ alkynyl, phenyl, pyridyl, COR¹³, OR²², SH, C₁-C₈ alkylthio, C₁-C₈ alkylsulphinyl, C₁-C₈ alkylsulphonyl, N(R²³)₂, CO₂R²², O(CO)R¹³, CON(R²³)₂, NR²³COR¹³ or CR¹³N—OR²², wherein the alkyl, alkoxy, cycloalkyl, alkenyl, alkynyl, phenyl and pyridyl are optionally substituted by one or more groups independently selected from halogen, CN, NH₂, NO₂, OH, C₁-C₄ alkyl, C₁-C₄ haloalkyl, C₁-C₄ alkoxy and C₁-C₄ haloalkoxy;

or Y⁶ and Y⁷ or Y⁷ and Y⁸ together with the fragment of the pyridyl ring to which they are attached may form a partially or fully unsaturated 5- to 7-membered carbocyclic ring or a 5- to 7-membered heterocyclic ring containing one to three heteroatoms independently selected from O, S, N and N(R¹²), providing that the heterocycle does not contain adjacent oxygen atoms, adjacent sulphur atoms, or adjacent sulphur and oxygen atoms, and wherein the ring formed by Y⁶ and Y⁷ or Y⁷ and Y⁸ is optionally substituted by one or more groups independently selected from halogen, CN, NH₂, NO₂, OH, C₁-C₄ alkyl, C₁-C₄ haloalkyl, C₁-C₄ alkoxy and C₁-C₄ haloalkoxy;

each R¹¹ and R²² independently of one another represent hydrogen, C₁-C₈ alkyl, C₃-C₈ cycloalkyl, C₃-C₈ alkenyl, C₃-C₈ alkynyl, benzyl, phenyl or pyridyl, wherein the alkyl, cycloalkyl, alkenyl, alkynyl, phenyl, benzyl and pyridyl are optionally substituted by one or more groups independently selected from halogen, CN, NH₂, NO₂, OH, C₁-C₄ alkyl, C₁-C₄-haloalkyl, C₁-C₄ alkoxy, C₁-C₄ haloalkoxy and C₁-C₄-alkoxy-C₁-C₄-alkyl;

each R¹² and R²³ independently of one another represent hydrogen, OH, C₁-C₈ alkyl, C₁-C₈ alkoxy, C₁-C₈-alkoxy-C₁-C₄-alkyl, C₃-C₈ alkenyl, C₃-C₈ alkynyl, or COR¹³, wherein the alkyl, alkoxy, alkenyl and alkynyl are optionally substituted by one or more halogen;

wherein when two radicals R¹² or two radicals R²³ are attached to the same nitrogen atom, these radicals can be identical or different;

wherein when two radicals R¹² or two radicals R²³ are attached to the same nitrogen atom, both of these radicals cannot be OH, C₁-C₄ alkoxy or C₁-C₄ haloalkoxy;

and wherein when two radicals R¹² or two radicals R²³ are attached to the same nitrogen atom, these two radicals together with the nitrogen atom to which they are attached may form a cycle B-1, B-2, B-3, B-4, B-5, B-6, B-7 or B-8;

wherein the cycle formed is optionally substituted by one or more groups independently selected from halogen, CN, NH₂, NO₂, OH, C₁-C₄ alkyl, C₁-C₄ haloalkyl, C₁-C₄ alkoxy and C₁-C₄ haloalkoxy;

each R¹³ and R^{13′} independently of one another represent hydrogen, C₁-C₈ alkyl, C₃-C₈ cycloalkyl, C₂-C₈ alkenyl, C₂-C₈ alkynyl, benzyl, phenyl or pyridyl, wherein the alkyl, cycloalkyl, alkenyl, alkynyl, phenyl, benzyl and pyridyl are optionally substituted by one or more groups independently selected from halogen, CN, NH₂, NO₂, OH, C₁-C₄ alkyl, C₁-C₄ haloalkyl, C₁-C₄ alkoxy and C₁-C₄ haloalkoxy;

G¹ and G² independently of one another represent —C(R²⁴R²⁵)—;

G³ represents —C(R²⁴R²⁵)—, O, N(R²⁶) or S;

or G¹ and G², or G² and G³, or G¹ and G¹ together represent —CR²⁴═CR²⁵—;

each R²⁴ and R²⁵ independently of one another represent hydrogen, halogen, C₁-C₄ alkyl, C₁-C₄ haloalkyl, C₁-C₄ alkoxy or C₁-C₄ haloalkoxy;

R²⁶ represents hydrogen, OH, C₁-C₄ alkyl, C₁-C₄ alkoxy, C₁-C₈ alkylcarbonyl or C₁-C₈ haloalkylcarbonyl; and

p is 0, 1 or 2.

In one preferred group of compounds of embodiment (I-C), R¹ represents hydrogen, C₁-C₈ alkyl, C₃-C₈ cycloalkyl, phenyl or pyridyl, wherein the alkyl, cycloalkyl, phenyl and pyridyl are optionally substituted by one or more groups independently selected from halogen, CN, C₁-C₄ alkyl, C₁-C₄ haloalkyl, OH, C₁-C₄ alkoxy, C₁-C₄ haloalkoxy and C₃-C₆ cycloalkyl;

A¹ represents cycle A-2, A-4 or A-5;

R³, R⁶, R⁷, R⁸ and R⁹ independently of one another represent hydrogen, halogen, OH, CN, C₁-C₈ alkyl, C₁-C₈ haloalkyl, C₁-C₈ alkoxy, C₁-C₈ haloalkoxy, C₃-C₈ cycloalkyl, phenyl, pyridyl, N(R¹²)₂ or NR¹²COR¹³, wherein the phenyl and pyridyl are optionally substituted by one or more groups independently selected from halogen, CN, C₁-C₄ alkyl, C₁-C₄ haloalkyl, OH, C₁-C₄ alkoxy and C₁-C₄ haloalkoxy;

or R⁶ and R⁷, R⁷ and R⁸, R³ and R⁸, or R³ and R⁹, together with the fragment of the pyridyl ring to which they are attached may form a partially or fully unsaturated 5- to 7-membered carboyclic ring or a 5- to 7-membered heterocyclic ring containing one to three heteroatoms independently selected from O, S, N and N(R¹²), providing that the heterocycle does not contain adjacent oxygen atoms, adjacent sulphur atoms, or adjacent sulphur and oxygen atoms, wherein the ring formed by R⁶ and R⁷, R⁷ and R⁸, R³ and R⁸, or R³ and R⁹ is optionally substituted by one or more groups independently selected from halogen, C₁-C₄ alkyl, C₁-C₄ haloalkyl, C₁-C₄ alkoxy and C₁-C₄ haloalkoxy;

X represents X-3;

Z⁴ and Z⁶ independently of one another represent methylene or halomethylene;

Z⁵ represents CR^{14′}R^{15′} or C═CR¹⁹R²⁰;

each R^{14′} and R^{15′} independently of one another represent hydrogen, halogen, OH, C₁-C₄ alkyl, C₁-C₄ haloalkyl, phenyl or CN, wherein the phenyl is optionally substituted by one or more groups independently selected from halogen, CN, methyl, halomethyl, methoxy or halomethoxy;

or R^{14′} and R^{15′} together with the carbon atom to which they are attached may form a C₃-C₆ cycloalkyl group or a C₃-C₆ halocycloalkyl group;

each R¹⁹ and R²⁰ independently of one another represent hydrogen, halogen, methyl or halomethyl;

Y⁶, Y⁷ and Y⁸ independently of one another represent hydrogen, halogen, OH, CN, C₁-C₈ alkyl, C₁-C₈ haloalkyl, C₁-C₈ alkoxy, C₁-C₈ haloalkoxy, C₃-C₈ cycloalkyl, phenyl, pyridyl, N(R²³)₂ or NR¹²COR¹³, wherein phenyl and pyridyl are optionally substituted by one or more groups independently selected from halogen, CN, C₁-C₄ alkyl, C₁-C₄ haloalkyl, C₁-C₄ alkoxy and C₁-C₄ haloalkoxy;

or Y⁶ and Y⁷ or Y⁷ and Y⁸ together with the fragment of the pyridyl ring to which they are attached may form a partially or fully unsaturated 5- to 7-membered carbocyclic ring or a 5- to 7-membered heterocyclic ring containing one to three heteroatoms independently selected from N and N(R¹²), and wherein the ring formed by Y⁶ and Y⁷ or Y⁷ and Y⁸ is optionally substituted by one or more groups independently selected from halogen, CN, NH₂, NO₂, OH, C₁-C₄ alkyl, C₁-C₄ haloalkyl, C₁-C₄ alkoxy and C₁-C₄ haloalkoxy;

each R¹² and R²³ independently of one another represent hydrogen, C₁-C₈ alkyl or COR¹³;

wherein when two radicals R¹² or two radicals R²³ are attached to the same nitrogen atom, these radicals can be identical or different;

and wherein when two radicals R¹² or two radicals R²³ are attached to the same nitrogen atom, these two radicals together with the nitrogen atom to which they are attached may form a cycle B-1, B-2, B-3, B-4 or B-5 wherein the cycle formed is optionally substituted by one or more groups independently selected from halogen, methyl and halomethyl;

each R¹³ independently represents C₁-C₈ alkyl or C₁-C₈ haloalkyl;

G¹, G² and G³ independently of one another represent —C(R²⁴R²⁵)—;

each R²⁴ and R²⁵ independently of one another represent hydrogen, halogen, C₁-C₄ alkyl, C₁-C₄ haloalkyl, C₁-C₄ alkoxy or C₁-C₄ haloalkoxy; and

p is 0, 1 or 2

In yet another group of preferred compounds of embodiment (I-C), R¹ represents hydrogen, C₁-C₄ alkyl, phenyl or pyridyl, wherein alkyl is optionally substituted by one or more groups independently selected from halogen, OH, C₁-C₄ alkoxy and C₁-C₄ haloalkoxy, and wherein phenyl and pyridyl are optionally substituted by one or more groups independently selected from halogen, CN, C₁-C₄ alkyl, C₁-C₄ haloalkyl, OH, C₁-C₄ alkoxy, C₁-C₄ haloalkoxy and C₃-C₆ cycloalkyl;

A¹ represents cycle A-2;

R³, R⁷, R⁸ and R⁹ independently of one another represent hydrogen, halogen, C₁-C₄ alkyl, C₁-C₄ haloalkyl, C₁-C₄ alkoxy, C₁-C₄ haloalkoxy, C₃-C₆ cycloalkyl, N(R¹²)₂ or NR¹²COR¹³;

or R⁷ and R⁸, R³ and R⁸, or R³ and R⁹, together with the fragment of the pyridyl ring to which they are attached may form a fully or partially unsaturated 6-membered carbocyclic ring, optionally substituted by one or more groups independently selected from halogen, methyl and halomethyl;

X represents X-3;

Z⁴ and Z⁶ represent methylene;

Z⁵ represents CR^{14′}R^{15′} or C═CR¹⁹R²⁰;

R^{14′} and R^{15′} independently of one another represent hydrogen, halogen, OH, C₁-C₄ alkyl, C₁-C₄ haloalkyl, phenyl or CN, wherein the phenyl is optionally substituted by one or more groups independently selected from halogen, CN, methyl, halomethyl, methoxy and halomethoxy;

or R¹⁴ and R¹⁵ together with the carbon atom to which they are attached may form a C₃-C₆ cycloalkyl group or a C₃-C₆ halocycloalkyl group;

R¹⁹ and R²⁰ independently of one another represent hydrogen, halogen, methyl or halomethyl;

Y⁶, Y⁷ and Y⁸ independently of one another represent hydrogen, CN, OH, halogen, C₁-C₄ alkyl, C₁-C₄ haloalkyl, C₁-C₄ alkoxy, C₁-C₄ haloalkoxy, C₃-C₆ cycloalkyl, N(R²³)₂, NR²³COR¹³ or phenyl, wherein phenyl is optionally substituted by one or more groups independently selected from halogen, CN, C₁-C₄ alkyl, C₁-C₄ haloalkyl, C₁-C₄ alkoxy and C₁-C₄ haloalkoxy;

each R¹² and R²³ independently of one another represent hydrogen or C₁-C₄ alkyl;

wherein when two radicals R¹² or two radicals R²³ are attached to the same nitrogen atom, these radicals can be identical or different;

and wherein when two radicals R¹² are attached to the same nitrogen atom, these two radicals together with the nitrogen atom to which they are attached may form a cycle B-1, B-2, B-3, B-4 or B-5 wherein the cycle formed is optionally substituted by one or more groups independently selected from halogen, methyl and halomethyl;

each R¹³ independently represents C₁-C₄-alkyl or C₁-C₄ haloalkyl;

G¹, G² and G³ represent methylene;

p is 0, 1 or 2.

In yet another preferred group of compounds of embodiment (I-C), R¹ represents hydrogen, C₁-C₄ alkyl, C₁-C₄ haloalkyl, phenyl or pyridin-2-yl, wherein the phenyl and pyridin-2-yl are optionally substituted by one or more groups independently selected from halogen, CN, methyl, halomethyl and methoxy;

A¹ represents cycle A-2;

R³, R⁷, R⁸ and R⁹ independently of one another represent hydrogen, halogen, C₁-C₄ alkyl, C₁-C₄ haloalkyl, C₁-C₄ alkoxy, C₁-C₄ haloalkoxy, C₃-C₆ cycloalkyl or N(R¹²)₂;

or R⁷ and R⁸, R³ and R⁸, or R³ and R⁹, together with the fragment of the pyridyl ring to which they are attached may form a fully or partially unsaturated 6-membered carbocyclic ring optionally substituted by one or more groups independently selected from halogen, methyl, halomethyl and halomethoxy;

X represents X-3;

Z⁴ and Z⁶ represent methylene;

Z⁵ represents CR^{14′}R^{15′} or C═CR¹⁹R²⁰;

each R^{14′} and R^{15′} independently of one another represent hydrogen, halogen, OH, C₁-C₄ alkyl, C₁-C₄ haloalkyl, phenyl or CN, wherein the phenyl is optionally substituted by one or more groups independently selected from halogen, CN, methyl, halomethyl, methoxy and halomethoxy;

or R^{14′} and R^{15′} together with the carbon atom they are attached may form a C₃-C₆ cycloalkyl group or a C₃-C₆ halocycloalkyl group;

R¹⁹ and R²⁰ independently of one another represent hydrogen, halogen, methyl or halomethyl;

Y⁶, Y⁷, and Y⁸ independently of one another represent hydrogen, CN, OH, NH₂, halogen, C₁-C₄ alkyl, C₁-C₄ haloalkyl, C₁-C₄ alkoxy, C₁-C₄ haloalkoxy, C₃-C₆ cycloalkyl, N(R²³)₂, NR²³COR¹³ or phenyl, wherein phenyl is optionally substituted by one or more groups independently selected from halogen, methyl, CN, methoxy, halomethyl and halomethoxy;

each R¹² and R²³ independently of one another represent hydrogen or C₁-C₄ alkyl;

wherein when two radicals R¹² or two radicals R²³ are attached to the same nitrogen atom, these radicals can be identical or different;

and wherein when two radicals R¹² are attached to the same nitrogen atom, these two radicals together with the nitrogen atom to which they are attached may form a cycle B-1, B-2, B-3, B-4 or B-5, wherein the cycle formed is optionally substituted by one or more groups independently selected from halogen, methyl and halomethyl;

each R¹³ independently represents C₁-C₄-alkyl or C₁-C₄ haloalkyl;

G¹, G² and G³ represent methylene;

p is 0, 1 or 2.

The preferred definitions of compounds of formula (I) as described herein also apply to the compounds of embodiment (I-C).

In one embodiment the invention relates to compounds of the formula (IA)

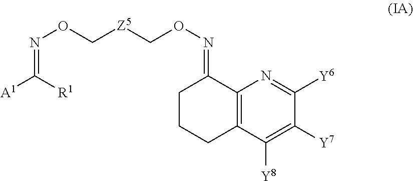

wherein A¹, R¹, Z⁵, Y⁶, Y⁷ and Y⁸ are as defined for a compound of formula (I). The preferred definitions of A¹, R¹, Z⁵, Y⁶, Y⁷ and Y⁸ defined in respect of compounds of formula (I) also apply to compounds of formula (IA).

In a further embodiment the invention relates to compounds of the formula (IB)

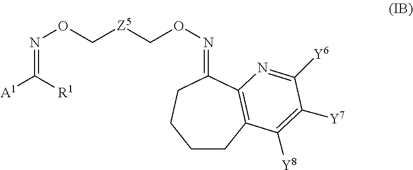

wherein A¹, R¹, Z⁵, Y⁶, Y⁷ and Y⁸ are as defined for a compound of formula (I). The preferred definitions of A¹, R¹, Z⁵, Y⁶, Y⁷ and Y⁸ defined in respect of compounds of formula (I) also apply to compounds of formula (IB).

Where it is stated above that R⁶ and R⁷, R⁷ and R⁸, R³ and R⁸, or R³ and R⁹ together with the fragment of the pyridyl ring to which they are attached may form a ring, for example a partially or fully unsaturated 5- to 7-membered carbocyclic ring or a 5- to 7-membered heterocyclic ring, then compounds with a ring formed by R⁷ and R⁸ together with the fragment of the pyridyl ring to which they are attached are preferred.

Where it is stated above that Y⁶ and Y⁷ or Y⁷ and Y⁸ together with the fragment of the pyridyl ring to which they are attached may form a partially or fully unsaturated 5- to 7-membered carbocyclic ring or a 5- to 7-membered heterocyclic ring then compounds with a ring formed by Y⁶ and Y⁷ together with the fragment of the pyridyl ring to which they are attached are preferred.

Certain intermediates that can be used to prepare compounds of formula (I) are novel and as such also form part of the present invention.

Accordingly, in a further aspect the invention provides a compound of formula (II)

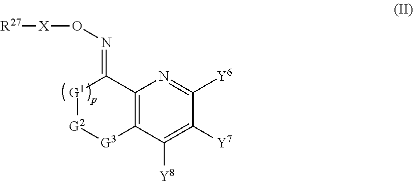

wherein R²⁷ represents —ONH₂, halogen, —O—SO₂—R²⁸ or one of the groups LG, C-1, C-2A or C-2B:

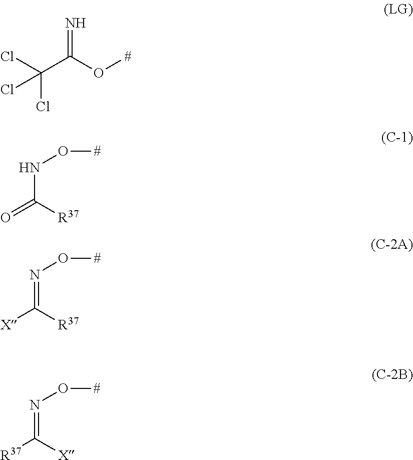

R²⁸ represents C₁-C₄ alkyl, C₁-C₄ haloalkyl or phenyl, wherein the phenyl is optionally substituted by one or two substituents independently selected from methyl, trihalomethyl, NO₂, CN, C₁-C₇ alkoxycarbonyl;

X″ represents halogen;

R³⁷ represents either A¹ or R¹ as defined herein for compounds of formula (I); and

X, G¹, G², G³, Y⁶, Y⁷, Y⁸ and p are as defined for the compound of formula (I);

or a salt or N-oxide thereof.

The preferred definitions of A¹, R¹, X, G¹, G², G³, Y⁶, Y⁷, Y⁸ and p defined in respect of compounds of formula (I) also apply to compounds of formula (II).

Preferably R²⁷ represents —ONH₂, —O—SO₂—R²⁸ or one of the groups LG, C-1 or C-2.

Even more preferably R²⁷ represents —ONH₂, tosylate, mesylate, triflate or one of the groups LG, C-1 or C-2.

In a further aspect the invention provides a compound of formula (III)

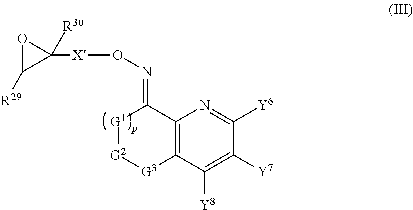

wherein X′ represents one of the groupings X′-1, X′-2 or X′-3:

#—Z⁶—# X′-1

#—Z⁹—Z¹⁰—# X′-2

#—Z¹³—Z¹⁴—Z¹⁵—# X′-3

Z⁶, Z⁹, Z¹⁰, Z¹³, Z¹⁴ and Z¹⁵ are as defined for a compound of formula (I);

R²⁹ and R³⁰ independently of one another represent hydrogen, halogen, C₁-C₄ alkyl, C₁-C₄ haloalkyl, phenyl or CN, wherein phenyl is optionally substituted by one or more groups, e.g. one to five groups, independently selected from halogen, CN, C₁-C₄ alkyl, C₁-C₄ haloalkyl, C₁-C₄ alkoxy and C₁-C₄ haloalkyoxy; and

G¹, G², G³, Y⁶, Y⁷, Y⁸ and p are as defined for a compound of formula (I);

or a salt or N-oxide thereof.

Preferably, R²⁹ and R³⁰ independently of one another represent hydrogen, halogen, C₁-C₄ alkyl, C₁-C₄ haloalkyl, phenyl or CN, wherein the phenyl is optionally substituted by one or more groups independently selected from halogen, CN, methyl, halomethyl, methoxy and halomethoxy.

The preferred definitions of Z⁶, Z⁹, Z¹⁰, Z¹³, Z¹⁴, Z¹⁵, G¹, G², G³, Y⁶, Y⁷, Y⁸ and p defined in respect of compounds of formula (I) above also apply to compounds of formula (III).

Preferably X′ represents X′-1.

In a further aspect the invention provides a compound of formula (VIII)

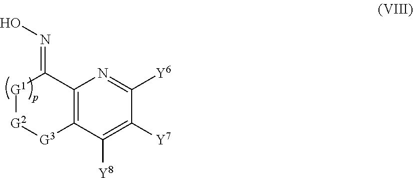

wherein G¹, G², G³, Y⁶, Y², Y⁸ and p are as defined for a compound of formula (I) providing that:

when p is 1 and G¹, G², G³ are —CH₂—, then Y⁶, Y² and Y⁸ are not all H;

when p is 1, G¹, G², G³ are —CH₂—, and Y² and Y⁸ are H, then Y⁶ is not methoxy;

when p is 1, G¹, G², G³ are —CH₂—, and Y⁶ and Y⁸ are H, then Y² is not methyl;

when p is 1, G¹, G² are —CH₂—, and Y⁶, Y² and Y⁸ are H, then G³ is not 0;

when p is 1, G¹ and G² together form CH═CH, Y⁶, Y² and Y⁸ are H, then G³ is not C(CHCl₂)(CH₃);

when p is 2 and G¹, G², G³ are —CH₂—, then Y⁶, Y² and Y⁸ are not all H;

or a salt or N-oxide thereof.

The preferred definitions of G¹, G², G³, Y⁶, Y², Y⁸ and p are as defined in respect of compounds of formula (I) above also apply to compounds of formula (VIII).

In one preferred group of compounds of formula (VIII):

Y⁶ represents halogen, CN, NO₂, C₁-C₈ alkyl, C₁-C₄-alkoxy-C₁-C₄-alkyl, C₁-C₄-alkoxy-C₁-C₄-alkoxy-C₁-C₄-alkyl, C₃-C₈ cycloalkyl, C₂-C₈ alkenyl, C₂-C₈ alkynyl, phenyl, pyridyl, COR¹³, OR^{22′}, SH, C₁-C₈ alkylthio, C₁-C₈ alkylsulphinyl, C₁-C₈ alkylsulphonyl, N(R²³)₂, CO₂R²², O(CO)R¹³, CON(R²³)₂, NR²³COR¹³ or CR¹³N—OR²², wherein the alkyl, alkoxy, cycloalkyl, alkenyl, alkynyl, phenyl and pyridyl are optionally substituted by one or more groups independently selected from halogen, CN, NH₂, NO₂, OH, C₁-C₄ alkyl, C₁-C₄ haloalkyl, C₁-C₄ alkoxy and C₁-C₄ haloalkoxy;

R^{22′} represents hydrogen, C₂-C₈ alkyl, C₃-C₈ cycloalkyl, C₃-C₈ alkenyl, C₃-C₈ alkynyl, benzyl, phenyl or pyridyl, wherein the alkyl, cycloalkyl, alkenyl, alkynyl, phenyl and pyridyl are optionally substituted by one or more groups independently selected from halogen, CN, NH₂, NO₂, OH, C₁-C₄ alkyl, C₁-C₄ haloalkyl, C₁-C₄ alkoxy, C₁-C₄ haloalkoxy and C₁-C₄-alkoxy-C₁-C₄-alkyl; and

R¹³, R²², R²³, G¹, G², G³, Y⁷, Y⁸ and p are as defined for a compound of formula (I).

According to this preferred embodiment of compounds of formula (VIII) preferred definitions of R¹³, R²², R²³, G¹, G², G³, Y⁷, Y⁸ and p are as defined for compounds of formula. Preferred definitions of Y⁶ are as set out below.

Preferably Y⁶ represents halogen, OH, CN, C₁-C₈ alkyl, C₁-C₈ haloalkyl, C₁-C₈ alkoxy, C₁-C₈ haloalkoxy, C₃-C₈ cycloalkyl, phenyl, pyridyl, N(R²³)₂ or NR¹²COR¹³, wherein phenyl and pyridyl are optionally substituted by one or more groups, e.g. one to five groups, independently selected from halogen, CN, C₁-C₄ alkyl, C₁-C₄ haloalkyl, C₁-C₄ alkoxy and C₁-C₄ haloalkoxy.

More preferably Y⁶ represents CN, OH, halogen, C₁-C₄ alkyl, C₁-C₄ haloalkyl, C₁-C₄ alkoxy, C₁-C₄ haloalkoxy, C₃-C₆ cycloalkyl, N(R²³)₂, NR²³COR¹³ or phenyl, wherein phenyl is optionally substituted by one or more groups, e.g. one to five groups, independently selected from halogen, CN, C₁-C₄ alkyl, C₁-C₄ haloalkyl, C₁-C₄ alkoxy and C₁-C₄ haloalkoxy.

Even more preferably Y⁶ represents CN, OH, NH₂, halogen, C₁-C₄ alkyl, C₁-C₄ haloalkyl, C₁-C₄ alkoxy, C₁-C₄ haloalkoxy, C₃-C₆ cycloalkyl, N(R²³)₂, NR²³COR¹³ or phenyl, wherein phenyl is optionally substituted by one or more groups, e.g. one to five groups, independently selected from halogen, methyl, CN, methoxy, halomethyl and halomethoxy.

In a further aspect the invention provides a compound of formula IVa

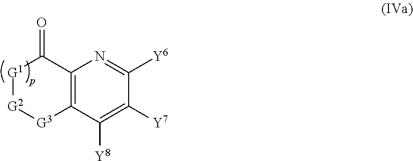

wherein G¹, G², G³, p, Y⁶, Y⁷ and Y⁸ are as defined for a compound of formula I, or a salt or N-oxide thereof, wherein the compound is not one of the compounds indicated in the claims. The preferred definitions of G¹, G², G³, Y⁶, Y⁷, Y⁸ and p are as defined in respect of compounds of formula (I) above also apply to compounds of formula (IVa).

In one preferred group of compounds of formula IVa

Y⁶ is C₁-C₆ alkyl, halogen, NH₂, C₁-C₆ haloalkyl, C₁-C₆ haloalkoxy, or C₁-C₆ haloalkoxy;

Y⁷ and Y⁸ independently of one another represent hydrogen, halogen, CN, NO₂, C₁-C₈ alkyl, C₁-C₄-alkoxy-C₁-C₄-alkyl, C₁-C₄-alkoxy-C₁-C₄-alkoxy-C₁-C₄-alkyl, C₃-C₈ cycloalkyl, C₂-C₈ alkenyl, C₂-C₈ alkynyl, phenyl, pyridyl, COR¹³, OR²², SH, C₁-C₈ alkylthio, C₁-C₈ alkylsulphinyl, C₁-C₈ alkylsulphonyl, N(R²³)₂, CO₂R²², O(CO)R¹³, CON(R²³)₂, NR²³COR¹³ or CR¹³N—OR²², wherein the alkyl, alkoxy, cycloalkyl, alkenyl, alkynyl, phenyl and pyridyl are optionally substituted by one or more groups independently selected from halogen, CN, NH₂, NO₂, OH, C₁-C₄ alkyl, C₁-C₄ haloalkyl, C₁-C₄ alkoxy and C₁-C₄ haloalkoxy;

G¹ and G² independently represent —C(R²⁴)(R²⁵)—;

G³ represents —C(R²⁴)(R²⁵)—, O, N(R²⁶) or S;

each R²⁴ and R²⁵ independently of one another represent hydrogen, halogen, C₁-C₄ alkyl, C₁-C₄ haloalkyl, C₁-C₄ alkoxy, or C₁-C₄ haloalkyl;

R²⁶ is hydrogen, OH, C₁-C₄ alkyl or C₁-C₄ aloxy;

p is 0, 1 or 2. Preferably, p is 1 or 2.

In another group of preferred compounds of formula IVa, Y⁶ is hydrogen, methyl or halomethyl.

In another group of preferred compounds of formula IVa, Y⁶ is methyl or halomethyl.

Preferred individual compounds of formula IVa are shown in the claims.

In a further aspect the invention provides a compound of formula XVI.

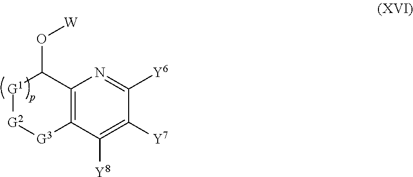

wherein G¹, G², G³, p, Y⁶, Y⁷ and Y⁸ are as defined herein for a compound of formula I, and wherein W is hydrogen, CO—CH₃, CO—CH₂CH₃, CO—CH₂CH₂CH₃, CO—CH(CH₃)₂, CO—CF₃, CO—CF₂CF₃, or a salt or N-oxide thereof, wherein the compound is not one of the compounds indicated in the claims. Preferred individual compounds of formula XVI are shown in the claims.

In a further aspect the invention provides a process for the production of a compound of formula (I) comprising reacting a compound of formula (IIb) with a compound of formula (X) as shown in scheme X

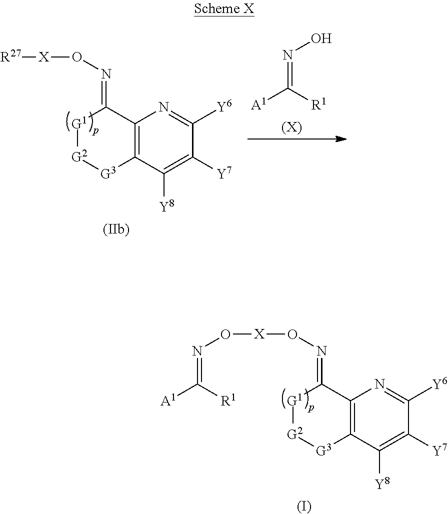

wherein R²⁷ represents halogen, —O—SO₂—R²⁸ or group LG:

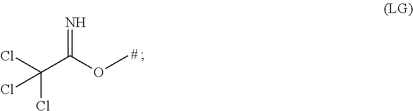

R²⁸ represents C₁-C₄-alkyl, C₁-C₄-haloalkyl or phenyl, wherein the phenyl is optionally substituted by one or two substituents independently selected from methyl, trihalomethyl, NO₂, CN, C₁-C₂-alkoxycarbonyl; and

X, G¹, G², G³, Y⁶, Y⁷, Y⁸, p, A¹ and R¹ are as defined for the compound of formula (I).

The preferred definitions of A¹, R¹, G¹, G², G³, Y⁶, Y⁷, Y⁸ and p defined in respect of compounds of formula (I) above also apply to compound (Iib) and (X). The compounds of formula (I) may exist as different geometric or optical isomers or in different tautomeric forms. These may be separated and isolated by well-known (usually chromatographic) techniques, and all such isomers and tautomers and mixtures thereof in all proportions as well as isotopic forms, such as deuterated compounds, are part of the present invention. In particular, the carbon-nitrogen double bonds of the compound of formula (I) allow the four cis/trans isomers (i)-(iv) shown below:

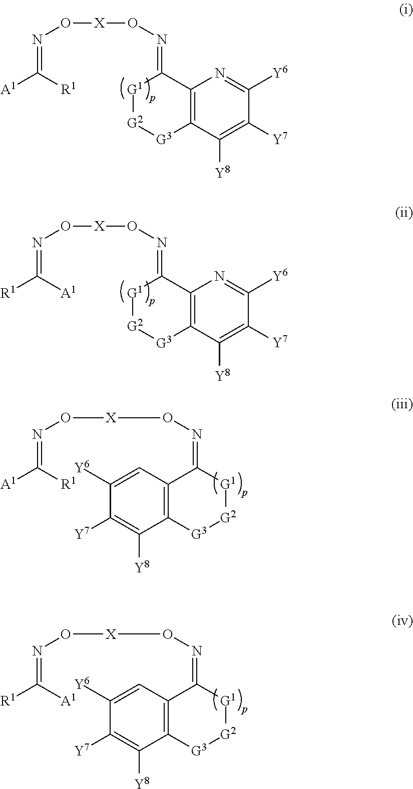

The present invention includes each of these isomers. The invention may provide a compound of formula (I) as just one of these isomers or as a mixture of one or more isomers in any ratio. Preferred compounds are those of the isomer (i).

Likewise, the invention also includes the corresponding isomers of the intermediates described herein, e.g. compounds (II), (III) and (VIII). In addition, where a reaction scheme depicts synthesis of one geometric isomer, the scheme also includes synthesis of the other geometric isomers where possible. For example Scheme X shown above encompasses the reactions

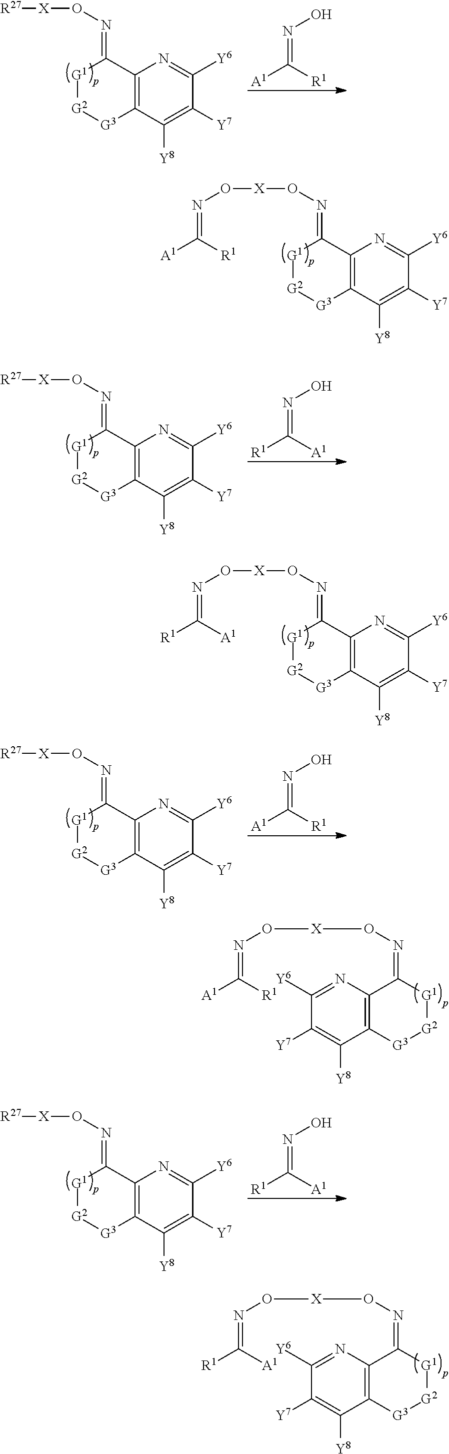

The compounds in tables 1 to 15 illustrate compounds of formula (I).

Table X represents Table 1 (when X is 1), Table 2 (when X is 2), Table 3 (when X is 3), Table 4 (when X is 4), Table 5 (when X is 5), Table 6 (when X is 6), Table 7 (when X is 7), Table 8 (when X is 8), Table 9 (when X is 9), Table 10 (when X is 10), Table 11 (when X is 11), Table 12 (when X is 12), Table 13 (when X is 13), Table 14 (when X is 14), Table 15 (when X is 15).

TABLE X

| Compound | A¹ | R¹ | Y⁶ | Y⁷ | Y⁸ |
|---|---|---|---|---|---|
| X.001 | 6-methylpyridin-2-yl | H | CH₃ | H | H |
| X.002 | 6-methylpyridin-2-yl | CH₃ | CH₃ | H | H |
| X.003 | 6-methylpyridin-2-yl | CH₃ | H | H | H |
| X.004 | 6-methylpyridin-2-yl | CH₃ | CH₂CH₃ | H | H |
| X.005 | 6-methylpyridin-2-yl | CH₃ | CH(CH₃)₂ | H | H |
| X.006 | 6-methylpyridin-2-yl | CH₃ | cyclopropyl | H | H |
| X.007 | 6-methylpyridin-2-yl | CH₃ | cyclohexyl | H | H |
| X.008 | 6-methylpyridin-2-yl | CH₃ | CH₂Cl | H | H |
| X.009 | 6-methylpyridin-2-yl | CH₃ | CH₂F | H | H |
| X.010 | 6-methylpyridin-2-yl | CH₃ | CF₃ | H | H |
| X.011 | 6-methylpyridin-2-yl | CH₃ | phenyl | H | H |
| X.012 | 6-methylpyridin-2-yl | CH₃ | 3-F-phenyl | H | H |
| X.013 | 6-methylpyridin-2-yl | CH₃ | 4-Cl-phenyl | H | H |
| X.014 | 6-methylpyridin-2-yl | CH₃ | 2-(CH₃)-phenyl | H | H |
| X.015 | 6-methylpyridin-2-yl | CH₃ | 2,4-di-Cl-phenyl | H | H |
| X.016 | 6-methylpyridin-2-yl | CH₃ | 3,5-di-F-phenyl | H | H |
| X.017 | 6-methylpyridin-2-yl | CH₃ | CN | H | H |
| X.018 | 6-methylpyridin-2-yl | CH₃ | Br | H | H |
| X.019 | 6-methylpyridin-2-yl | CH₃ | OH | H | H |
| X.020 | 6-methylpyridin-2-yl | CH₃ | OCH₃ | H | H |
| X.021 | 6-methylpyridin-2-yl | CH₃ | OCH₂CH₃ | H | H |
| X.022 | 6-methylpyridin-2-yl | CH₃ | OCH(CH₃)₂ | H | H |
| X.023 | 6-methylpyridin-2-yl | CH₃ | NH₂ | H | H |
| X.024 | 6-methylpyridin-2-yl | CH₃ | NH(CH₃) | H | H |
| X.025 | 6-methylpyridin-2-yl | CH₃ | N(CH₃)₂ | H | H |
| X.026 | 6-methylpyridin-2-yl | CH₃ | NH(CH₂CH═CH₂) | H | H |
| X.027 | 6-methylpyridin-2-yl | CH₃ | NH(CH₂CH₃) | H | H |
| X.028 | 6-methylpyridin-2-yl | CH₃ | 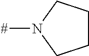 | H | H |
| X.029 | 6-methylpyridin-2-yl | CH₃ | 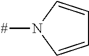 | H | H |
| X.030 | 6-methylpyridin-2-yl | CH₃ | 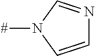 | H | H |
| X.031 | 6-methylpyridin-2-yl | CH₃ | 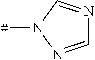 | H | H |
| X.032 | 6-methylpyridin-2-yl | CH₃ | 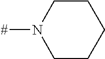 | H | H |
| X.033 | 6-methylpyridin-2-yl | CH₃ | NH(COCH₃) | H | H |
| X.034 | 6-methylpyridin-2-yl | CH₃ | NH(COCH(CH₃)₂) | H | H |
| X.035 | 6-methylpyridin-2-yl | CH₃ | H | H | CH₃ |
| X.036 | 6-methylpyridin-2-yl | CH₃ | CH₃ | H | CH₃ |
| X.037 | 6-methylpyridin-2-yl | CH₃ | CH₂CH₃ | H | CH₃ |
| X.038 | 6-methylpyridin-2-yl | CH₃ | CH(CH₃)₂ | H | CH₃ |
| X.039 | 6-methylpyridin-2-yl | CH₃ | cyclopropyl | H | CH₃ |
| X.040 | 6-methylpyridin-2-yl | CH₃ | cyclohexyl | H | CH₃ |
| X.041 | 6-methylpyridin-2-yl | CH₃ | CH₂Cl | H | CH₃ |
| X.042 | 6-methylpyridin-2-yl | CH₃ | CH₂F | H | CH₃ |
| X.043 | 6-methylpyridin-2-yl | CH₃ | CF₃ | H | CH₃ |
| X.044 | 6-methylpyridin-2-yl | CH₃ | phenyl | H | CH₃ |
| X.045 | 6-methylpyridin-2-yl | CH₃ | 3-F-phenyl | H | CH₃ |
| X.046 | 6-methylpyridin-2-yl | CH₃ | 4-Cl-phenyl | H | CH₃ |
| X.047 | 6-methylpyridin-2-yl | CH₃ | 2-(CH₃)-phenyl | H | CH₃ |
| X.048 | 6-methylpyridin-2-yl | CH₃ | 2,4-di-Cl-phenyl | H | CH₃ |
| X.049 | 6-methylpyridin-2-yl | CH₃ | 3,5-di-F-phenyl | H | CH₃ |
| X.050 | 6-methylpyridin-2-yl | CH₃ | CN | H | CH₃ |
| X.051 | 6-methylpyridin-2-yl | CH₃ | Br | H | CH₃ |
| X.052 | 6-methylpyridin-2-yl | CH₃ | OH | H | CH₃ |
| X.053 | 6-methylpyridin-2-yl | CH₃ | OCH₃ | H | CH₃ |
| X.054 | 6-methylpyridin-2-yl | CH₃ | OCH₂CH₃ | H | CH₃ |
| X.055 | 6-methylpyridin-2-yl | CH₃ | OCH(CH₃)₂ | H | CH₃ |
| X.056 | 6-methylpyridin-2-yl | CH₃ | NH₂ | H | CH₃ |
| X.057 | 6-methylpyridin-2-yl | CH₃ | NH(CH₃) | H | CH₃ |
| X.058 | 6-methylpyridin-2-yl | CH₃ | N(CH₃)₂ | H | CH₃ |
| X.059 | 6-methylpyridin-2-yl | CH₃ | NH(CH₂CH═CH₂) | H | CH₃ |
| X.060 | 6-methylpyridin-2-yl | CH₃ | NH(CH₂CH₃) | H | CH₃ |
| X.061 | 6-methylpyridin-2-yl | CH₃ | 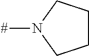 | H | CH₃ |
| X.062 | 6-methylpyridin-2-yl | CH₃ | 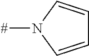 | H | CH₃ |
| X.063 | 6-methylpyridin-2-yl | CH₃ | 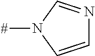 | H | CH₃ |
| X.064 | 6-methylpyridin-2-yl | CH₃ | 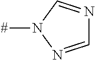 | H | CH₃ |
| X.065 | 6-methylpyridin-2-yl | CH₃ | 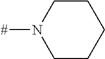 | H | CH₃ |
| X.066 | 6-methylpyridin-2-yl | CH₃ | NH(COCH₃) | H | CH₃ |
| X.067 | 6-methylpyridin-2-yl | CH₃ | NH(COCH(CH₃)₂) | H | CH₃ |
| X.068 | 6-methylpyridin-2-yl | CH₃ | H | CH₃ | H |
| X.069 | 6-methylpyridin-2-yl | CH₃ | CH₃ | CH₃ | H |
| X.070 | 6-methylpyridin-2-yl | CH₃ | CH₂CH₃ | CH₃ | H |
| X.071 | 6-methylpyridin-2-yl | CH₃ | CH(CH₃)₂ | CH₃ | H |
| X.072 | 6-methylpyridin-2-yl | CH₃ | cyclopropyl | CH₃ | H |
| X.073 | 6-methylpyridin-2-yl | CH₃ | cyclohexyl | CH₃ | H |
| X.074 | 6-methylpyridin-2-yl | CH₃ | CH₂Cl | CH₃ | H |
| X.075 | 6-methylpyridin-2-yl | CH₃ | CH₂F | CH₃ | H |
| X.076 | 6-methylpyridin-2-yl | CH₃ | CF₃ | CH₃ | H |
| X.077 | 6-methylpyridin-2-yl | CH₃ | phenyl | CH₃ | H |
| X.078 | 6-methylpyridin-2-yl | CH₃ | 3-F-phenyl | CH₃ | H |
| X.079 | 6-methylpyridin-2-yl | CH₃ | 4-Cl-phenyl | CH₃ | H |
| X.080 | 6-methylpyridin-2-yl | CH₃ | 2-(CH₃)-phenyl | CH₃ | H |
| X.081 | 6-methylpyridin-2-yl | CH₃ | 2,4-di-Cl-phenyl | CH₃ | H |
| X.082 | 6-methylpyridin-2-yl | CH₃ | 3,5-di-F-phenyl | CH₃ | H |
| X.083 | 6-methylpyridin-2-yl | CH₃ | CN | CH₃ | H |
| X.084 | 6-methylpyridin-2-yl | CH₃ | Br | CH₃ | H |
| X.085 | 6-methylpyridin-2-yl | CH₃ | OH | CH₃ | H |
| X.086 | 6-methylpyridin-2-yl | CH₃ | OCH₃ | CH₃ | H |
| X.087 | 6-methylpyridin-2-yl | CH₃ | OCH₂CH₃ | CH₃ | H |
| X.088 | 6-methylpyridin-2-yl | CH₃ | OCH(CH₃)₂ | CH₃ | H |
| X.089 | 6-methylpyridin-2-yl | CH₃ | NH₂ | CH₃ | H |
| X.090 | 6-methylpyridin-2-yl | CH₃ | NH(CH₃) | CH₃ | H |
| X.091 | 6-methylpyridin-2-yl | CH₃ | N(CH₃)₂ | CH₃ | H |
| X.092 | 6-methylpyridin-2-yl | CH₃ | NH(CH₂CH═CH₂) | CH₃ | H |
| X.093 | 6-methylpyridin-2-yl | CH₃ | NH(CH₂CH₃) | CH₃ | H |
| X.094 | 6-methylpyridin-2-yl | CH₃ | 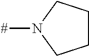 | CH₃ | H |
| X.095 | 6-methylpyridin-2-yl | CH₃ | 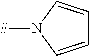 | CH₃ | H |
| X.096 | 6-methylpyridin-2-yl | CH₃ | 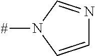 | CH₃ | H |
| X.097 | 6-methylpyridin-2-yl | CH₃ | 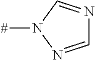 | CH₃ | H |
| X.098 | 6-methylpyridin-2-yl | CH₃ | 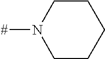 | CH₃ | H |
| X.099 | 6-methylpyridin-2-yl | CH₃ | NH(COCH₃) | CH₃ | H |
| X.100 | 6-methylpyridin-2-yl | CH₃ | NH(COCH(CH₃)₂) | CH₃ | H |
| X.101 | 6-methylpyridin-2-yl | CH₃ | CH₃ | H | OCH₃ |
| X.102 | 6-methylpyridin-2-yl | CH₃ | CH₂CH₃ | H | OCH₃ |
| X.103 | 6-methylpyridin-2-yl | CH₃ | cyclopropyl | H | OCH₃ |
| X.104 | 6-methylpyridin-2-yl | CH₃ | CF₃ | H | OCH₃ |
| X.105 | 6-methylpyridin-2-yl | CH₃ | phenyl | H | OCH₃ |
| X.106 | 6-methylpyridin-2-yl | CH₃ | CN | H | OCH₃ |
| X.107 | 6-methylpyridin-2-yl | CH₃ | Br | H | OCH₃ |
| X.108 | 6-methylpyridin-2-yl | CH₃ | OH | H | OCH₃ |
| X.109 | 6-methylpyridin-2-yl | CH₃ | OCH₃ | H | OCH₃ |
| X.110 | 6-methylpyridin-2-yl | CH₃ | NH₂ | H | OCH₃ |
| X.111 | 6-methylpyridin-2-yl | CH₃ | NH(CH₃) | H | OCH₃ |
| X.112 | 6-methylpyridin-2-yl | CH₃ | N(CH₃)₂ | H | OCH₃ |
| X.113 | 6-methylpyridin-2-yl | CH₃ | 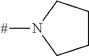 | H | OCH₃ |
| X.114 | 6-methylpyridin-2-yl | CH₃ | 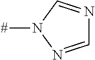 | H | OCH₃ |
| X.115 | 6-methylpyridin-2-yl | CH₃ | NH(COCH₃) | H | OCH₃ |
| X.116 | 6-methylpyridin-2-yl | CH₃ | CH₃ | OCH₃ | H |
| X.117 | 6-methylpyridin-2-yl | CH₃ | CH₂CH₃ | OCH₃ | H |
| X.118 | 6-methylpyridin-2-yl | CH₃ | cyclopropyl | OCH₃ | H |
| X.119 | 6-methylpyridin-2-yl | CH₃ | CF₃ | OCH₃ | H |
| X.120 | 6-methylpyridin-2-yl | CH₃ | phenyl | OCH₃ | H |
| X.121 | 6-methylpyridin-2-yl | CH₃ | OH | OCH₃ | H |
| X.122 | 6-methylpyridin-2-yl | CH₃ | OCH₃ | OCH₃ | H |
| X.123 | 6-methylpyridin-2-yl | CH₃ | NH₂ | OCH₃ | H |
| X.124 | 6-methylpyridin-2-yl | CH₃ | NH(CH₃) | OCH₃ | H |
| X.125 | 6-methylpyridin-2-yl | CH₃ | N(CH₃)₂ | OCH₃ | H |
| X.126 | 6-methylpyridin-2-yl | CH₃ | 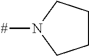 | OCH₃ | H |
| X.127 | 6-methylpyridin-2-yl | CH₃ | 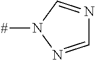 | OCH₃ | H |
| X.128 | 6-methylpyridin-2-yl | CH₃ | CH₃ | CH₃ | CH₃ |
| X.129 | 6-methylpyridin-2-yl | CH₃ | CH₂CH₃ | CH₃ | CH₃ |
| X.130 | 6-methylpyridin-2-yl | CH₃ | cyclopropyl | CH₃ | CH₃ |
| X.131 | 6-methylpyridin-2-yl | CH₃ | CF₃ | CH₃ | CH₃ |
| X.132 | 6-methylpyridin-2-yl | CH₃ | phenyl | CH₃ | CH₃ |
| X.133 | 6-methylpyridin-2-yl | CH₃ | OH | CH₃ | CH₃ |
| X.134 | 6-methylpyridin-2-yl | CH₃ | OCH₃ | CH₃ | CH₃ |
| X.135 | 6-methylpyridin-2-yl | CH₃ | NH(CH₃) | CH₃ | CH₃ |
| X.136 | 6-methylpyridin-2-yl | CH₃ | N(CH₃)₂ | CH₃ | CH₃ |
| X.137 | 6-methylpyridin-2-yl | CH₃ | CH₃ | CH₃ | OCH₃ |
| X.138 | 6-methylpyridin-2-yl | CH₃ | CH₂CH₃ | CH₃ | OCH₃ |
| X.139 | 6-methylpyridin-2-yl | CH₃ | cyclopropyl | CH₃ | OCH₃ |
| X.140 | 6-methylpyridin-2-yl | CH₃ | CF₃ | CH₃ | OCH₃ |
| X.141 | 6-methylpyridin-2-yl | CH₃ | phenyl | CH₃ | OCH₃ |
| X.142 | 6-methylpyridin-2-yl | CH₃ | OH | CH₃ | OCH₃ |
| X.143 | 6-methylpyridin-2-yl | CH₃ | OCH₃ | CH₃ | OCH₃ |
| X.144 | 6-methylpyridin-2-yl | CH₃ | NH(CH₃) | CH₃ | OCH₃ |
| X.145 | 6-methylpyridin-2-yl | CH₃ | N(CH₃)₂ | CH₃ | OCH₃ |
| X.146 | 6-methylpyridin-2-yl | CH₃ | CH₃ | H | N(CH₃)₂ |
| X.147 | 6-methylpyridin-2-yl | CH₃ | CH₂CH₃ | H | N(CH₃)₂ |
| X.148 | 6-methylpyridin-2-yl | CH₃ | cyclopropyl | H | N(CH₃)₂ |
| X.149 | 6-methylpyridin-2-yl | CH₃ | CF₃ | H | N(CH₃)₂ |
| X.150 | 6-methylpyridin-2-yl | CH₃ | Phenyl | H | N(CH₃)₂ |
| X.151 | 6-methylpyridin-2-yl | CH₃ | OH | H | N(CH₃)₂ |
| X.152 | 6-methylpyridin-2-yl | CH₃ | OCH₃ | H | N(CH₃)₂ |
| X.153 | 6-methylpyridin-2-yl | CH₃ | NH(CH₃) | H | N(CH₃)₂ |
| X.154 | 6-methylpyridin-2-yl | CH₃ | N(CH₃)₂ | H | N(CH₃)₂ |
| X.155 | 6-methylpyridin-2-yl | CH₃ | CH₃ | H | CF₃ |
| X.156 | 6-methylpyridin-2-yl | CH₃ | CH₂CH₃ | H | CF₃ |
| X.157 | 6-methylpyridin-2-yl | CH₃ | cyclopropyl | H | CF₃ |
| X.158 | 6-methylpyridin-2-yl | CH₃ | CF₃ | H | CF₃ |
| X.159 | 6-methylpyridin-2-yl | CH₃ | Phenyl | H | CF₃ |
| X.160 | 6-methylpyridin-2-yl | CH₃ | OH | H | CF₃ |
| X.161 | 6-methylpyridin-2-yl | CH₃ | OCH₃ | H | CF₃ |
| X.162 | 6-methylpyridin-2-yl | CH₃ | NH(CH₃) | H | CF₃ |
| X.163 | 6-methylpyridin-2-yl | CH₃ | N(CH₃)₂ | H | CF₃ |
| X.164 | 6-methylpyridin-2-yl | CH₃ | CH₃ | H | Cl |
| X.165 | 6-methylpyridin-2-yl | CH₃ | OCH₃ | H | Cl |
| X.166 | 6-methylpyridin-2-yl | CH₃ | CH₃ | Cl | H |
| X.167 | 4,6-dimethylpyridin-2-yl | CH₃ | H | H | H |
| X.168 | 4,6-dimethylpyridin-2-yl | CH₃ | CH₃ | H | H |
| X.169 | 4,6-dimethylpyridin-2-yl | CH₃ | CH₃ | H | H |
| X.170 | 4,6-dimethylpyridin-2-yl | CH₃ | CH₂CH₃ | H | H |
| X.171 | 4,6-dimethylpyridin-2-yl | CH₃ | CH(CH₃)₂ | H | H |
| X.172 | 4,6-dimethylpyridin-2-yl | CH₃ | cyclopropyl | H | H |
| X.173 | 4,6-dimethylpyridin-2-yl | CH₃ | cyclohexyl | H | H |
| X.174 | 4,6-dimethylpyridin-2-yl | CH₃ | CH₂Cl | H | H |
| X.175 | 4,6-dimethylpyridin-2-yl | CH₃ | CH₂F | H | H |
| X.176 | 4,6-dimethylpyridin-2-yl | CH₃ | CF₃ | H | H |
| X.177 | 4,6-dimethylpyridin-2-yl | CH₃ | phenyl | H | H |
| X.178 | 4,6-dimethylpyridin-2-yl | CH₃ | 3-F-phenyl | H | H |
| X.179 | 4,6-dimethylpyridin-2-yl | CH₃ | 4-Cl-phenyl | H | H |
| X.180 | 4,6-dimethylpyridin-2-yl | CH₃ | 2-(CH₃)-phenyl | H | H |
| X.181 | 4,6-dimethylpyridin-2-yl | CH₃ | 2,4-di-Cl-phenyl | H | H |
| X.182 | 4,6-dimethylpyridin-2-yl | CH₃ | 3,5-di-F-phenyl | H | H |
| X.183 | 4,6-dimethylpyridin-2-yl | CH₃ | CN | H | H |
| X.184 | 4,6-dimethylpyridin-2-yl | CH₃ | Br | H | H |
| X.185 | 4,6-dimethylpyridin-2-yl | CH₃ | OH | H | H |
| X.186 | 4,6-dimethylpyridin-2-yl | CH₃ | OCH₃ | H | H |
| X.187 | 4,6-dimethylpyridin-2-yl | CH₃ | OCH₂CH₃ | H | H |
| X.188 | 4,6-dimethylpyridin-2-yl | CH₃ | OCH(CH₃)₂ | H | H |
| X.189 | 4,6-dimethylpyridin-2-yl | CH₃ | NH₂ | H | H |
| X.190 | 4,6-dimethylpyridin-2-yl | CH₃ | NH(CH₃) | H | H |
| X.191 | 4,6-dimethylpyridin-2-yl | CH₃ | N(CH₃)₂ | H | H |
| X.192 | 4,6-dimethylpyridin-2-yl | CH₃ | NH(CH₂CH═CH₂) | H | H |
| X.193 | 4,6-dimethylpyridin-2-yl | CH₃ | NH(CH₂CH₃) | H | H |
| X.194 | 4,6-dimethylpyridin-2-yl | CH₃ | 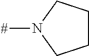 | H | H |
| X.195 | 4,6-dimethylpyridin-2-yl | CH₃ | 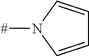 | H | H |
| X.196 | 4,6-dimethylpyridin-2-yl | CH₃ | 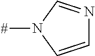 | H | H |
| X.197 | 4,6-dimethylpyridin-2-yl | CH₃ | 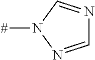 | H | H |
| X.198 | 4,6-dimethylpyridin-2-yl | CH₃ | 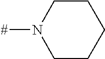 | H | H |
| X.199 | 4,6-dimethylpyridin-2-yl | CH₃ | NH(COCH₃) | H | H |
| X.200 | 4,6-dimethylpyridin-2-yl | CH₃ | NH(COCH(CH₃)₂) | H | H |
| X.201 | 4,6-dimethylpyridin-2-yl | CH₃ | H | H | CH₃ |
| X.202 | 4,6-dimethylpyridin-2-yl | CH₃ | CH₃ | H | CH₃ |
| X.203 | 4,6-dimethylpyridin-2-yl | CH₃ | CH₂CH₃ | H | CH₃ |
| X.204 | 4,6-dimethylpyridin-2-yl | CH₃ | CH(CH₃)₂ | H | CH₃ |
| X.205 | 4,6-dimethylpyridin-2-yl | CH₃ | cyclopropyl | H | CH₃ |
| X.206 | 4,6-dimethylpyridin-2-yl | CH₃ | cyclohexyl | H | CH₃ |
| X.207 | 4,6-dimethylpyridin-2-yl | CH₃ | CH₂Cl | H | CH₃ |
| X.208 | 4,6-dimethylpyridin-2-yl | CH₃ | CH₂F | H | CH₃ |
| X.209 | 4,6-dimethylpyridin-2-yl | CH₃ | CF₃ | H | CH₃ |
| X.210 | 4,6-dimethylpyridin-2-yl | CH₃ | phenyl | H | CH₃ |
| X.211 | 4,6-dimethylpyridin-2-yl | CH₃ | 3-F-phenyl | H | CH₃ |
| X.212 | 4,6-dimethylpyridin-2-yl | CH₃ | 4-Cl-phenyl | H | CH₃ |
| X.213 | 4,6-dimethylpyridin-2-yl | CH₃ | 2-(CH₃)-phenyl | H | CH₃ |
| X.214 | 4,6-dimethylpyridin-2-yl | CH₃ | 2,4-di-Cl-phenyl | H | CH₃ |
| X.215 | 4,6-dimethylpyridin-2-yl | CH₃ | 3,5-di-F-phenyl | H | CH₃ |
| X.216 | 4,6-dimethylpyridin-2-yl | CH₃ | CN | H | CH₃ |
| X.217 | 4,6-dimethylpyridin-2-yl | CH₃ | Br | H | CH₃ |
| X.218 | 4,6-dimethylpyridin-2-yl | CH₃ | OH | H | CH₃ |
| X.219 | 4,6-dimethylpyridin-2-yl | CH₃ | OCH₃ | H | CH₃ |
| X.220 | 4,6-dimethylpyridin-2-yl | CH₃ | OCH₂CH₃ | H | CH₃ |
| X.221 | 4,6-dimethylpyridin-2-yl | CH₃ | OCH(CH₃)₂ | H | CH₃ |
| X.222 | 4,6-dimethylpyridin-2-yl | CH₃ | NH₂ | H | CH₃ |
| X.223 | 4,6-dimethylpyridin-2-yl | CH₃ | NH(CH₃) | H | CH₃ |
| X.224 | 4,6-dimethylpyridin-2-yl | CH₃ | N(CH₃)₂ | H | CH₃ |
| X.225 | 4,6-dimethylpyridin-2-yl | CH₃ | NH(CH₂CH═CH₂) | H | CH₃ |
| X.226 | 4,6-dimethylpyridin-2-yl | CH₃ | NH(CH₂CH₃) | H | CH₃ |
| X.227 | 4,6-dimethylpyridin-2-yl | CH₃ | 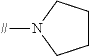 | H | CH₃ |
| X.228 | 4,6-dimethylpyridin-2-yl | CH₃ | 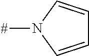 | H | CH₃ |
| X.229 | 4,6-dimethylpyridin-2-yl | CH₃ | 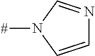 | H | CH₃ |
| X.230 | 4,6-dimethylpyridin-2-yl | CH₃ | 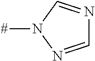 | H | CH₃ |
| X.231 | 4,6-dimethylpyridin-2-yl | CH₃ | 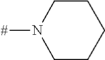 | H | CH₃ |
| X.232 | 4,6-dimethylpyridin-2-yl | CH₃ | NH(COCH₃) | H | CH₃ |
| X.233 | 4,6-dimethylpyridin-2-yl | CH₃ | NH(COCH(CH₃)₂) | H | CH₃ |
| X.234 | 4,6-dimethylpyridin-2-yl | CH₃ | H | CH₃ | H |
| X.235 | 4,6-dimethylpyridin-2-yl | CH₃ | CH₃ | CH₃ | H |
| X.236 | 4,6-dimethylpyridin-2-yl | CH₃ | CH₂CH₃ | CH₃ | H |
| X.237 | 4,6-dimethylpyridin-2-yl | CH₃ | CH(CH₃)₂ | CH₃ | H |
| X.238 | 4,6-dimethylpyridin-2-yl | CH₃ | cyclopropyl | CH₃ | H |
| X.239 | 4,6-dimethylpyridin-2-yl | CH₃ | cyclohexyl | CH₃ | H |
| X.240 | 4,6-dimethylpyridin-2-yl | CH₃ | CH₂Cl | CH₃ | H |
| X.241 | 4,6-dimethylpyridin-2-yl | CH₃ | CH₂F | CH₃ | H |
| X.242 | 4,6-dimethylpyridin-2-yl | CH₃ | CF₃ | CH₃ | H |
| X.243 | 4,6-dimethylpyridin-2-yl | CH₃ | phenyl | CH₃ | H |
| X.244 | 4,6-dimethylpyridin-2-yl | CH₃ | 3-F-phenyl | CH₃ | H |
| X.245 | 4,6-dimethylpyridin-2-yl | CH₃ | 4-Cl-phenyl | CH₃ | H |
| X.246 | 4,6-dimethylpyridin-2-yl | CH₃ | 2-(CH₃)-phenyl | CH₃ | H |
| X.247 | 4,6-dimethylpyridin-2-yl | CH₃ | 2,4-di-Cl-phenyl | CH₃ | H |
| X.248 | 4,6-dimethylpyridin-2-yl | CH₃ | 3,5-di-F-phenyl | CH₃ | H |
| X.249 | 4,6-dimethylpyridin-2-yl | CH₃ | CN | CH₃ | H |
| X.250 | 4,6-dimethylpyridin-2-yl | CH₃ | Br | CH₃ | H |
| X.251 | 4,6-dimethylpyridin-2-yl | CH₃ | OH | CH₃ | H |
| X.252 | 4,6-dimethylpyridin-2-yl | CH₃ | OCH₃ | CH₃ | H |
| X.253 | 4,6-dimethylpyridin-2-yl | CH₃ | OCH₂CH₃ | CH₃ | H |
| X.254 | 4,6-dimethylpyridin-2-yl | CH₃ | OCH(CH₃)₂ | CH₃ | H |
| X.255 | 4,6-dimethylpyridin-2-yl | CH₃ | NH₂ | CH₃ | H |
| X.256 | 4,6-dimethylpyridin-2-yl | CH₃ | NH(CH₃) | CH₃ | H |
| X.257 | 4,6-dimethylpyridin-2-yl | CH₃ | N(CH₃)₂ | CH₃ | H |
| X.258 | 4,6-dimethylpyridin-2-yl | CH₃ | NH(CH₂CH═CH₂) | CH₃ | H |
| X.259 | 4,6-dimethylpyridin-2-yl | CH₃ | NH(CH₂CH₃) | CH₃ | H |
| X.260 | 4,6-dimethylpyridin-2-yl | CH₃ | 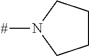 | CH₃ | H |
| X.261 | 4,6-dimethylpyridin-2-yl | CH₃ | 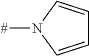 | CH₃ | H |
| X.262 | 4,6-dimethylpyridin-2-yl | CH₃ | 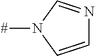 | CH₃ | H |
| X.263 | 4,6-dimethylpyridin-2-yl | CH₃ | 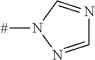 | CH₃ | H |
| X.264 | 4,6-dimethylpyridin-2-yl | CH₃ | 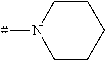 | CH₃ | H |
| X.265 | 4,6-dimethylpyridin-2-yl | CH₃ | NH(COCH₃) | CH₃ | H |
| X.266 | 4,6-dimethylpyridin-2-yl | CH₃ | NH(COCH(CH₃)₂) | CH₃ | H |
| X.267 | 4,6-dimethylpyridin-2-yl | CH₃ | CH₃ | H | OCH₃ |
| X.268 | 4,6-dimethylpyridin-2-yl | CH₃ | CH₂CH₃ | H | OCH₃ |
| X.269 | 4,6-dimethylpyridin-2-yl | CH₃ | cyclopropyl | H | OCH₃ |
| X.270 | 4,6-dimethylpyridin-2-yl | CH₃ | CF₃ | H | OCH₃ |
| X.271 | 4,6-dimethylpyridin-2-yl | CH₃ | phenyl | H | OCH₃ |
| X.272 | 4,6-dimethylpyridin-2-yl | CH₃ | CN | H | OCH₃ |
| X.273 | 4,6-dimethylpyridin-2-yl | CH₃ | Br | H | OCH₃ |
| X.274 | 4,6-dimethylpyridin-2-yl | CH₃ | OH | H | OCH₃ |
| X.275 | 4,6-dimethylpyridin-2-yl | CH₃ | OCH₃ | H | OCH₃ |
| X.276 | 4,6-dimethylpyridin-2-yl | CH₃ | NH₂ | H | OCH₃ |
| X.277 | 4,6-dimethylpyridin-2-yl | CH₃ | NH(CH₃) | H | OCH₃ |
| X.278 | 4,6-dimethylpyridin-2-yl | CH₃ | N(CH₃)₂ | H | OCH₃ |
| X.279 | 4,6-dimethylpyridin-2-yl | CH₃ | 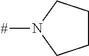 | H | OCH₃ |
| X.280 | 4,6-dimethylpyridin-2-yl | CH₃ | 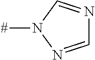 | H | OCH₃ |
| X.281 | 4,6-dimethylpyridin-2-yl | CH₃ | NH(COCH₃) | H | OCH₃ |
| X.282 | 4,6-dimethylpyridin-2-yl | CH₃ | CH₃ | OCH₃ | H |
| X.283 | 4,6-dimethylpyridin-2-yl | CH₃ | CH₂CH₃ | OCH₃ | H |
| X.284 | 4,6-dimethylpyridin-2-yl | CH₃ | cyclopropyl | OCH₃ | H |
| X.285 | 4,6-dimethylpyridin-2-yl | CH₃ | CF₃ | OCH₃ | H |
| X.286 | 4,6-dimethylpyridin-2-yl | CH₃ | phenyl | OCH₃ | H |
| X.287 | 4,6-dimethylpyridin-2-yl | CH₃ | OH | OCH₃ | H |
| X.288 | 4,6-dimethylpyridin-2-yl | CH₃ | OCH₃ | OCH₃ | H |
| X.289 | 4,6-dimethylpyridin-2-yl | CH₃ | NH₂ | OCH₃ | H |
| X.290 | 4,6-dimethylpyridin-2-yl | CH₃ | NH(CH₃) | OCH₃ | H |
| X.291 | 4,6-dimethylpyridin-2-yl | CH₃ | N(CH₃)₂ | OCH₃ | H |
| X.292 | 4,6-dimethylpyridin-2-yl | CH₃ | 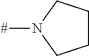 | OCH₃ | H |
| X.293 | 4,6-dimethylpyridin-2-yl | CH₃ | 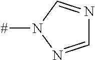 | OCH₃ | H |
| X.294 | 4,6-dimethylpyridin-2-yl | CH₃ | CH₃ | CH₃ | CH₃ |
| X.295 | 4,6-dimethylpyridin-2-yl | CH₃ | CH₂CH₃ | CH₃ | CH₃ |
| X.296 | 4,6-dimethylpyridin-2-yl | CH₃ | cyclopropyl | CH₃ | CH₃ |
| X.297 | 4,6-dimethylpyridin-2-yl | CH₃ | CF₃ | CH₃ | CH₃ |
| X.298 | 4,6-dimethylpyridin-2-yl | CH₃ | Phenyl | CH₃ | CH₃ |
| X.299 | 4,6-dimethylpyridin-2-yl | CH₃ | OH | CH₃ | CH₃ |
| X.300 | 4,6-dimethylpyridin-2-yl | CH₃ | OCH₃ | CH₃ | CH₃ |
| X.301 | 4,6-dimethylpyridin-2-yl | CH₃ | NH(CH₃) | CH₃ | CH₃ |
| X.302 | 4,6-dimethylpyridin-2-yl | CH₃ | N(CH₃)₂ | CH₃ | CH₃ |
| X.303 | 4,6-dimethylpyridin-2-yl | CH₃ | CH₃ | CH₃ | OCH₃ |
| X.304 | 4,6-dimethylpyridin-2-yl | CH₃ | CH₂CH₃ | CH₃ | OCH₃ |
| X.305 | 4,6-dimethylpyridin-2-yl | CH₃ | cyclopropyl | CH₃ | OCH₃ |
| X.306 | 4,6-dimethylpyridin-2-yl | CH₃ | CF₃ | CH₃ | OCH₃ |
| X.307 | 4,6-dimethylpyridin-2-yl | CH₃ | phenyl | CH₃ | OCH₃ |
| X.308 | 4,6-dimethylpyridin-2-yl | CH₃ | OH | CH₃ | OCH₃ |
| X.309 | 4,6-dimethylpyridin-2-yl | CH₃ | OCH₃ | CH₃ | OCH₃ |
| X.310 | 4,6-dimethylpyridin-2-yl | CH₃ | NH(CH₃) | CH₃ | OCH₃ |
| X.311 | 4,6-dimethylpyridin-2-yl | CH₃ | N(CH₃)₂ | CH₃ | OCH₃ |
| X.312 | 4,6-dimethylpyridin-2-yl | CH₃ | CH₃ | H | N(CH₃)₂ |
| X.313 | 4,6-dimethylpyridin-2-yl | CH₃ | CH₂CH₃ | H | N(CH₃)₂ |
| X.314 | 4,6-dimethylpyridin-2-yl | CH₃ | cyclopropyl | H | N(CH₃)₂ |
| X.315 | 4,6-dimethylpyridin-2-yl | CH₃ | CF₃ | H | N(CH₃)₂ |
| X.316 | 4,6-dimethylpyridin-2-yl | CH₃ | phenyl | H | N(CH₃)₂ |
| X.317 | 4,6-dimethylpyridin-2-yl | CH₃ | OH | H | N(CH₃)₂ |
| X.318 | 4,6-dimethylpyridin-2-yl | CH₃ | OCH₃ | H | N(CH₃)₂ |
| X.319 | 4,6-dimethylpyridin-2-yl | CH₃ | NH(CH₃) | H | N(CH₃)₂ |
| X.320 | 4,6-dimethylpyridin-2-yl | CH₃ | N(CH₃)₂ | H | N(CH₃)₂ |
| X.321 | 4,6-dimethylpyridin-2-yl | CH₃ | CH₃ | H | CF₃ |
| X.322 | 4,6-dimethylpyridin-2-yl | CH₃ | CH₂CH₃ | H | CF₃ |
| X.323 | 4,6-dimethylpyridin-2-yl | CH₃ | cyclopropyl | H | CF₃ |
| X.324 | 4,6-dimethylpyridin-2-yl | CH₃ | CF₃ | H | CF₃ |
| X.325 | 4,6-dimethylpyridin-2-yl | CH₃ | phenyl | H | CF₃ |
| X.326 | 4,6-dimethylpyridin-2-yl | CH₃ | OH | H | CF₃ |
| X.327 | 4,6-dimethylpyridin-2-yl | CH₃ | OCH₃ | H | CF₃ |
| X.328 | 4,6-dimethylpyridin-2-yl | CH₃ | NH(CH₃) | H | CF₃ |
| X.329 | 4,6-dimethylpyridin-2-yl | CH₃ | N(CH₃)₂ | H | CF₃ |
| X.330 | 4,6-dimethylpyridin-2-yl | CH₃ | CH₃ | H | Cl |
| X.331 | 4,6-dimethylpyridin-2-yl | CH₃ | OCH₃ | H | Cl |
| X.332 | 4,6-dimethylpyridin-2-yl | CH₃ | CH₃ | Cl | H |
| X.333 | 6-methyl-4- | CH₃ | H | H | H |
| | isopropoxypyridin-2-yl | | | | |
| X.334 | 6-methyl-4- | CH₃ | CH₃ | H | H |
| | isopropoxypyridin-2-yl | | | | |
| X.335 | 6-methyl-4- | CH₃ | CH₂CH₃ | H | H |
| | isopropoxypyridin-2-yl | | | | |
| X.336 | 6-methyl-4- | CH₃ | CH₃ | H | CH₃ |
| | isopropoxypyridin-2-yl | | | | |
| X.337 | 6-methyl-4- | CH₃ | CH₃ | CH₃ | H |
| | isopropoxypyridin-2-yl | | | | |
| X.338 | 6-methyl-4- | CH₃ | CH₃ | CH₃ | CH₃ |
| | isopropoxypyridin-2-yl | | | | |
| X.339 | 6-methyl-4- | CH₃ | CH₃ | H | OCH₃ |
| | isopropoxypyridin-2-yl | | | | |
| X.340 | 6-methyl-4- | CH₃ | CH₃ | H | N(CH₃)₂ |
| | isopropoxypyridin-2-yl | | | | |
| X.341 | 6-methyl-4- | CH₃ | CH₃ | OCH₃ | H |
| | isopropoxypyridin-2-yl | | | | |
| X.342 | 6-methyl-4- | CH₃ | CH₃ | H | Cl |
| | isopropoxypyridin-2-yl | | | | |
| X.343 | 6-methyl-4- | CH₃ | OCH₃ | H | H |
| | isopropoxypyridin-2-yl | | | | |
| X.344 | 6-methyl-4- | CH₃ | OCH₃ | H | CH₃ |
| | isopropoxypyridin-2-yl | | | | |
| X.345 | 6-methyl-4- | CH₃ | OCH₃ | CH₃ | H |
| | isopropoxypyridin-2-yl | | | | |
| X.346 | 6-methyl-4- | CH₃ | OCH₃ | H | OCH₃ |
| | isopropoxypyridin-2-yl | | | | |
| X.347 | 6-methyl-4- | CH₃ | OCH₃ | CH₃ | OCH₃ |
| | isopropoxypyridin-2-yl | | | | |
| X.348 | 6-methyl-4- | CH₃ | CH₃ | H | CF₃ |
| | isopropoxypyridin-2-yl | | | | |
| X.349 | 6-methyl-4- | CH₃ | N(CH₃)₂ | H | H |
| | isopropoxypyridin-2-yl | | | | |
| X.350 | 6-methyl-4- | CH₃ | N(CH₃)₂ | H | CH₃ |
| | isopropoxypyridin-2-yl | | | | |
| X.351 | 6-methyl-4- | CH₃ | N(CH₃)₂ | CH₃ | H |
| | isopropoxypyridin-2-yl | | | | |
| X.352 | 6-methyl-4- | CH₃ | N(CH₃)₂ | H | Cl |
| | isopropoxypyridin-2-yl | | | | |
| X.353 | 6-methyl-4- | CH₃ | H | H | H |
| | cyclopropylpyridin-2-yl | | | | |
| X.354 | 6-methyl-4- | CH₃ | CH₃ | H | H |
| | cyclopropylpyridin-2-yl | | | | |
| X.355 | 6-methyl-4- | CH₃ | CH₂CH₃ | H | H |
| | cyclopropylpyridin-2-yl | | | | |
| X.356 | 6-methyl-4- | CH₃ | CH₃ | H | CH₃ |
| | cyclopropylpyridin-2-yl | | | | |
| X.357 | 6-methyl-4- | CH₃ | CH₃ | CH₃ | H |
| | cyclopropylpyridin-2-yl | | | | |
| X.358 | 6-methyl-4- | CH₃ | CH₃ | CH₃ | CH₃ |
| | cyclopropylpyridin-2-yl | | | | |
| X.359 | 6-methyl-4- | CH₃ | CH₃ | H | OCH₃ |
| | cyclopropylpyridin-2-yl | | | | |
| X.360 | 6-methyl-4- | CH₃ | CH₃ | H | N(CH₃)₂ |
| | cyclopropylpyridin-2-yl | | | | |
| X.361 | 6-methyl-4- | CH₃ | CH₃ | OCH₃ | H |
| | cyclopropylpyridin-2-yl | | | | |
| X.362 | 6-methyl-4- | CH₃ | CH₃ | H | Cl |
| | cyclopropylpyridin-2-yl | | | | |
| X.363 | 6-methyl-4- | CH₃ | OCH₃ | H | H |
| | cyclopropylpyridin-2-yl | | | | |
| X.364 | 6-methyl-4- | CH₃ | OCH₃ | H | CH₃ |
| | cyclopropylpyridin-2-yl | | | | |
| X.365 | 6-methyl-4- | CH₃ | OCH₃ | CH₃ | H |
| | cyclopropylpyridin-2-yl | | | | |
| X.366 | 6-methyl-4- | CH₃ | OCH₃ | H | OCH₃ |
| | cyclopropylpyridin-2-yl | | | | |
| X.367 | 6-methyl-4- | CH₃ | OCH₃ | CH₃ | OCH₃ |
| | cyclopropylpyridin-2-yl | | | | |
| X.368 | 6-methyl-4- | CH₃ | CH₃ | H | CF₃ |
| | cyclopropylpyridin-2-yl | | | | |
| X.369 | 6-methyl-4- | CH₃ | N(CH₃)₂ | H | H |
| | cyclopropylpyridin-2-yl | | | | |
| X.370 | 6-methyl-4- | CH₃ | N(CH₃)₂ | H | CH₃ |
| | cyclopropylpyridin-2-yl | | | | |
| X.371 | 6-methyl-4- | CH₃ | N(CH₃)₂ | CH₃ | H |
| | cyclopropylpyridin-2-yl | | | | |
| X.372 | 6-methyl-4- | CH₃ | N(CH₃)₂ | H | Cl |
| | cyclopropylpyridin-2-yl | | | | |
| X.373 | 6-methoxy-pyridin-2-yl | H | CH₃ | H | H |
| X.374 | 6-methoxypyridin-2-yl | CH₃ | CH₃ | H | H |
| X.375 | 6-methoxypyridin-2-yl | CH₃ | CH₂CH₃ | H | H |
| X.376 | 6-methoxypyridin-2-yl | CH₃ | CH₃ | H | CH₃ |
| X.377 | 6-methoxypyridin-2-yl | CH₃ | CH₃ | CH₃ | H |
| X.378 | 6-methoxypyridin-2-yl | CH₃ | CH₃ | H | OCH₃ |
| X.379 | 6-methoxypyridin-2-yl | CH₃ | OCH₃ | H | CH₃ |
| X.380 | 6-methoxypyridin-2-yl | CH₃ | OCH₃ | CH₃ | H |
| X.381 | 6-methoxypyridin-2-yl | CH₃ | OCH₃ | H | OCH₃ |
| X.382 | 6-methoxypyridin-2-yl | CH₃ | CH₃ | H | N(CH₃)₂ |
| X.383 | 6-methoxy-4- | CH₃ | CH₃ | H | H |
| | methylpyridin-2-yl | | | | |
| X.384 | 6-methoxy-4- | CH₃ | CH₂CH₃ | H | H |
| | methylpyridin-2-yl | | | | |
| X.385 | 6-methoxy-4- | CH₃ | CH₃ | H | CH₃ |
| | methylpyridin-2-yl | | | | |
| X.386 | 6-methoxy-4- | CH₃ | CH₃ | CH₃ | H |
| | methylpyridin-2-yl | | | | |
| X.387 | 6-methoxy-4- | CH₃ | CH₃ | H | OCH₃ |
| | methylpyridin-2-yl | | | | |
| X.388 | 6-methoxy-4- | CH₃ | OCH₃ | H | CH₃ |
| | methylpyridin-2-yl | | | | |
| X.389 | 6-methoxy-4- | CH₃ | OCH₃ | CH₃ | H |
| | methylpyridin-2-yl | | | | |
| X.390 | 6-methoxy-4- | CH₃ | OCH₃ | H | OCH₃ |
| | methylpyridin-2-yl | | | | |
| X.391 | 6-methoxy-4- | CH₃ | CH₃ | H | N(CH₃)₂ |
| | methylpyridin-2-yl | | | | |
| X.392 | 6-methyl-4- | CH₃ | CH₃ | H | H |
| | dimethylaminopyridin-2-yl | | | | |
| X.393 | 6-methyl-4- | CH₃ | CH₂CH₃ | H | H |
| | dimethylaminopyridin-2-yl | | | | |
| X.394 | 6-methyl-4- | CH₃ | CH₃ | H | CH₃ |
| | dimethylaminopyridin-2-yl | | | | |
| X.395 | 6-methyl-4- | CH₃ | CH₃ | CH₃ | H |
| | dimethylaminopyridin-2-yl | | | | |
| X.396 | 6-methyl-4- | CH₃ | CH₃ | H | OCH₃ |
| | dimethylaminopyridin-2-yl | | | | |
| X.397 | 6-methyl-4- | CH₃ | OCH₃ | H | CH₃ |
| | dimethylaminopyridin-2-yl | | | | |
| X.398 | 6-methyl-4- | CH₃ | OCH₃ | CH₃ | H |
| | dimethylaminopyridin-2-yl | | | | |
| X.399 | 6-methyl-4- | CH₃ | OCH₃ | H | OCH₃ |
| | dimethylaminopyridin-2-yl | | | | |
| X.400 | 6-methyl-4- | CH₃ | CH₃ | H | N(CH₃)₂ |
| | dimethylaminopyridin-2-yl | | | | |
| X.401 | 6-methyl-5- | CH₃ | CH₃ | H | H |
| | methoxypyridin-2-yl | | | | |
| X.402 | 6-methyl-5- | CH₃ | CH₂CH₃ | H | H |
| | methoxypyridin-2-yl | | | | |
| X.403 | 6-methyl-5- | CH₃ | CH₃ | H | CH₃ |
| | methoxypyridin-2-yl | | | | |
| X.404 | 6-methyl-5- | CH₃ | CH₃ | CH₃ | H |
| | methoxypyridin-2-yl | | | | |
| X.405 | 6-methyl-5- | CH₃ | CH₃ | H | OCH₃ |
| | methoxypyridin-2-yl | | | | |
| X.406 | 6-methyl-5- | CH₃ | OCH₃ | H | CH₃ |
| | methoxypyridin-2-yl | | | | |
| X.407 | 6-methyl-5- | CH₃ | OCH₃ | CH₃ | H |
| | methoxypyridin-2-yl | | | | |
| X.408 | 6-methyl-5- | CH₃ | OCH₃ | H | OCH₃ |
| | methoxypyridin-2-yl | | | | |
| X.409 | 6-methyl-5- | CH₃ | CH₃ | H | N(CH₃)₂ |
| | methoxypyridin-2-yl | | | | |
| X.410 | 6-methoxy-5- | CH₃ | CH₃ | H | H |
| | methylpyridin-2-yl | | | | |
| X.411 | 6-methoxy-5- | CH₃ | CH₂CH₃ | H | H |
| | methylpyridin-2-yl | | | | |
| X.412 | 6-methoxy-5- | CH₃ | CH₃ | H | CH₃ |
| | methylpyridin-2-yl | | | | |
| X.413 | 6-methoxy-5- | CH₃ | CH₃ | CH₃ | H |
| | methylpyridin-2-yl | | | | |
| X.414 | 6-methoxy-5- | CH₃ | CH₃ | H | OCH₃ |
| | methylpyridin-2-yl | | | | |
| X.415 | 6-methoxy-5- | CH₃ | OCH₃ | H | CH₃ |
| | methylpyridin-2-yl | | | | |
| X.416 | 6-methoxy-5- | CH₃ | OCH₃ | CH₃ | H |
| | methylpyridin-2-yl | | | | |
| X.417 | 6-methoxy-5- | CH₃ | OCH₃ | H | OCH₃ |
| | methylpyridin-2-yl | | | | |
| X.418 | 6-methoxy-5- | CH₃ | CH₃ | H | N(CH₃)₂ |
| | methylpyridin-2-yl | | | | |
| X.419 | 6-methylpyridin-2-yl | CH₂CH₃ | CH₃ | H | H |
| X.420 | 6-methylpyridin-2-yl | CH₂CH₃ | CH₂CH₃ | H | H |
| X.421 | 6-methylpyridin-2-yl | CH₂CH₃ | CH₃ | H | CH₃ |
| X.422 | 6-methylpyridin-2-yl | CH₂CH₃ | CH₃ | CH₃ | H |
| X.423 | 6-methylpyridin-2-yl | CH₂CH₃ | CH₃ | CH₃ | CH₃ |
| X.424 | 6-methylpyridin-2-yl | CH₂CH₃ | CH₃ | H | OCH₃ |
| X.425 | 6-methylpyridin-2-yl | CH₂CH₃ | CH₃ | OCH₃ | H |
| X.426 | 6-methylpyridin-2-yl | CH₂CH₃ | CH₃ | H | N(CH₃)₂ |
| X.427 | 6-methylpyridin-2-yl | CH₂CH₃ | OCH₃ | H | H |
| X.428 | 6-methylpyridin-2-yl | CH₂CH₃ | OCH₃ | H | CH₃ |
| X.429 | 6-methylpyridin-2-yl | CH₂CH₃ | OCH₃ | H | OCH₃ |
| X.430 | 4,6-dimethylpyridin-2-yl | CH₂CH₃ | CH₃ | H | H |
| X.431 | 4,6-dimethylpyridin-2-yl | CH₂CH₃ | CH₂CH₃ | H | H |
| X.432 | 4,6-dimethylpyridin-2-yl | CH₂CH₃ | CH₃ | H | CH₃ |
| X.433 | 4,6-dimethylpyridin-2-yl | CH₂CH₃ | CH₃ | CH₃ | H |
| X.434 | 4,6-dimethylpyridin-2-yl | CH₂CH₃ | CH₃ | CH₃ | CH₃ |
| X.435 | 4,6-dimethylpyridin-2-yl | CH₂CH₃ | CH₃ | H | OCH₃ |
| X.436 | 4,6-dimethylpyridin-2-yl | CH₂CH₃ | CH₃ | OCH₃ | H |
| X.437 | 4,6-dimethylpyridin-2-yl | CH₂CH₃ | CH₃ | H | N(CH₃)₂ |
| X.438 | 4,6-dimethylpyridin-2-yl | CH₂CH₃ | OCH₃ | H | H |
| X.439 | 4,6-dimethylpyridin-2-yl | CH₂CH₃ | OCH₃ | H | CH₃ |
| X.440 | 4,6-dimethylpyridin-2-yl | CH₂CH₃ | OCH₃ | H | OCH₃ |
| X.441 | 6-methyl-4- | CH₂CH₃ | CH₃ | H | H |
| | isopropoxypyridin-2-yl | | | | |
| X.442 | 6-methyl-4- | CH₂CH₃ | CH₂CH₃ | H | H |
| | isopropoxypyridin-2-yl | | | | |
| X.443 | 6-methyl-4- | CH₂CH₃ | CH₃ | H | CH₃ |
| | isopropoxypyridin-2-yl | | | | |
| X.444 | 6-methyl-4- | CH₂CH₃ | CH₃ | CH₃ | H |
| | isopropoxypyridin-2-yl | | | | |
| X.445 | 6-methyl-4- | CH₂CH₃ | CH₃ | CH₃ | CH₃ |
| | isopropoxypyridin-2-yl | | | | |
| X.446 | 6-methyl-4- | CH₂CH₃ | CH₃ | H | OCH₃ |
| | isopropoxypyridin-2-yl | | | | |
| X.447 | 6-methyl-4- | CH₂CH₃ | CH₃ | OCH₃ | H |
| | isopropoxypyridin-2-yl | | | | |
| X.448 | 6-methyl-4- | CH₂CH₃ | CH₃ | H | N(CH₃)₂ |
| | isopropoxypyridin-2-yl | | | | |
| X.449 | 6-methyl-4- | CH₂CH₃ | OCH₃ | H | H |
| | isopropoxypyridin-2-yl | | | | |
| X.450 | 6-methyl-4- | CH₂CH₃ | OCH₃ | H | CH₃ |
| | isopropoxypyridin-2-yl | | | | |
| X.451 | 6-methyl-4- | CH₂CH₃ | OCH₃ | H | OCH₃ |
| | isopropoxypyridin-2-yl | | | | |
| X.452 | 6-methoxy-4- | CH₂CH₃ | CH₃ | H | H |
| | methylpyridin-2-yl | | | | |
| X.453 | 6-methoxy-4- | CH₂CH₃ | CH₂CH₃ | H | H |
| | methylpyridin-2-yl | | | | |
| X.454 | 6-methoxy-4- | CH₂CH₃ | CH₃ | H | CH₃ |
| | methylpyridin-2-yl | | | | |
| X.455 | 6-methoxy-4- | CH₂CH₃ | CH₃ | CH₃ | H |
| | methylpyridin-2-yl | | | | |
| X.456 | 6-methoxy-4- | CH₂CH₃ | CH₃ | CH₃ | CH₃ |
| | methylpyridin-2-yl | | | | |
| X.457 | 6-methoxy-4- | CH₂CH₃ | CH₃ | H | OCH₃ |
| | methylpyridin-2-yl | | | | |
| X.458 | 6-methoxy-4- | CH₂CH₃ | CH₃ | OCH₃ | H |
| | methylpyridin-2-yl | | | | |
| X.459 | 6-methoxy-4- | CH₂CH₃ | CH₃ | H | N(CH₃)₂ |
| | methylpyridin-2-yl | | | | |
| X.460 | 6-methoxy-4- | CH₂CH₃ | OCH₃ | H | H |
| | methylpyridin-2-yl | | | | |
| X.461 | 6-methoxy-4- | CH₂CH₃ | OCH₃ | H | CH₃ |
| | methylpyridin-2-yl | | | | |
| X.462 | 6-methoxy-4- | CH₂CH₃ | OCH₃ | H | OCH₃ |
| | methylpyridin-2-yl | | | | |
| X.463 | 6-methoxypyridin-2-yl | CH₂CH₃ | CH₃ | H | H |
| X.464 | 6-methoxypyridin-2-yl | CH₂CH₃ | CH₂CH₃ | H | H |
| X.465 | 6-methoxypyridin-2-yl | CH₂CH₃ | CH₃ | H | CH₃ |
| X.466 | 6-methoxypyridin-2-yl | CH₂CH₃ | CH₃ | CH₃ | H |
| X.467 | 6-methoxypyridin-2-yl | CH₂CH₃ | CH₃ | CH₃ | CH₃ |
| X.468 | 6-methoxypyridin-2-yl | CH₂CH₃ | CH₃ | H | OCH₃ |
| X.469 | 6-methoxypyridin-2-yl | CH₂CH₃ | CH₃ | OCH₃ | H |
| X.470 | 6-methoxypyridin-2-yl | CH₂CH₃ | CH₃ | H | N(CH₃)₂ |
| X.471 | 6-methoxypyridin-2-yl | CH₂CH₃ | OCH₃ | H | H |
| X.472 | 6-methoxypyridin-2-yl | CH₂CH₃ | OCH₃ | H | CH₃ |
| X.473 | 6-methoxypyridin-2-yl | CH₂CH₃ | OCH₃ | H | OCH₃ |
| X.474 | 6-methylpyridin-2-yl | phenyl | CH₃ | H | H |
| X.475 | 6-methylpyridin-2-yl | phenyl | CH₂CH₃ | H | H |
| X.476 | 6-methylpyridin-2-yl | phenyl | CH₃ | H | CH₃ |
| X.477 | 6-methylpyridin-2-yl | phenyl | CH₃ | CH₃ | H |
| X.478 | 6-methylpyridin-2-yl | phenyl | CH₃ | CH₃ | CH₃ |
| X.479 | 6-methylpyridin-2-yl | phenyl | CH₃ | H | OCH₃ |
| X.480 | 6-methylpyridin-2-yl | phenyl | CH₃ | OCH₃ | H |
| X.481 | 6-methylpyridin-2-yl | phenyl | CH₃ | H | N(CH₃)₂ |
| X.482 | 6-methylpyridin-2-yl | phenyl | OCH₃ | H | H |
| X.483 | 6-methylpyridin-2-yl | phenyl | OCH₃ | H | CH₃ |
| X.484 | 6-methylpyridin-2-yl | phenyl | OCH₃ | H | OCH₃ |
| X.485 | 4,6-dimethylpyridin-2-yl | phenyl | CH₃ | H | H |
| X.486 | 4,6-dimethylpyridin-2-yl | phenyl | CH₂CH₃ | H | H |
| X.487 | 4,6-dimethylpyridin-2-yl | phenyl | CH₃ | H | CH₃ |
| X.488 | 4,6-dimethylpyridin-2-yl | phenyl | CH₃ | CH₃ | H |
| X.489 | 4,6-dimethylpyridin-2-yl | phenyl | CH₃ | CH₃ | CH₃ |
| X.490 | 4,6-dimethylpyridin-2-yl | phenyl | CH₃ | H | OCH₃ |
| X.491 | 4,6-dimethylpyridin-2-yl | phenyl | CH₃ | OCH₃ | H |
| X.492 | 4,6-dimethylpyridin-2-yl | phenyl | CH₃ | H | N(CH₃)₂ |
| X.493 | 4,6-dimethylpyridin-2-yl | phenyl | OCH₃ | H | H |
| X.494 | 4,6-dimethylpyridin-2-yl | phenyl | OCH₃ | H | CH₃ |
| X.495 | 4,6-dimethylpyridin-2-yl | phenyl | OCH₃ | H | OCH₃ |
| X.496 | 6-methylpyridin-2-yl | 6-methyl- | CH₃ | H | H |
| | | pyridin-2-yl | | | |
| X.497 | 6-methylpyridin-2-yl | 6-methyl- | CH₂CH₃ | H | H |
| | | pyridin-2-yl | | | |
| X.498 | 6-methylpyridin-2-yl | 6-methyl- | CH₃ | H | CH₃ |
| | | pyridin-2-yl | | | |
| X.499 | 6-methylpyridin-2-yl | 6-methyl- | CH₃ | CH₃ | H |
| | | pyridin-2-yl | | | |
| X.500 | 6-methylpyridin-2-yl | 6-methyl- | CH₃ | CH₃ | CH₃ |
| | | pyridin-2-yl | | | |
| X.501 | 6-methylpyridin-2-yl | 6-methyl- | CH₃ | H | OCH₃ |
| | | pyridin-2-yl | | | |
| X.502 | 6-methylpyridin-2-yl | 6-methyl- | CH₃ | OCH₃ | H |
| | | pyridin-2-yl | | | |
| X.503 | 6-methylpyridin-2-yl | 6-methyl- | CH₃ | H | N(CH₃)₂ |
| | | pyridin-2-yl | | | |
| X.504 | 6-methylpyridin-2-yl | 6-methyl- | OCH₃ | H | H |
| | | pyridin-2-yl | | | |
| X.505 | 6-methylpyridin-2-yl | 6-methyl- | OCH₃ | H | CH₃ |
| | | pyridin-2-yl | | | |
| X.506 | 6-methylpyridin-2-yl | 6-methyl- | OCH₃ | H | OCH₃ |
| | | pyridin-2-yl | | | |
| X.507 | quinolin-2-yl | H | CH₃ | H | H |
| X.508 | 6-bromopyridin-2-yl | CH₃ | CH₃ | H | H |
| X.509 | 6-fluoro-5-chloropyridin- | CH₃ | CH₃ | H | H |
| | 2-yl | | | | |
| X.510 | pyrid-2-yl | phenyl | CH₃ | H | H |
| X.511 | pyrid-2-yl | pyrid-2-yl | CH₃ | H | H |
| X.512 | quinolin-2-yl | CH₃ | CH₃ | H | H |
| X.513 | 5,6,7,8-tetrahydro- | CH₃ | CH₃ | H | H |
| | quinolin-2-yl | | | | |
| X.514 | 6-phenyl-pyrid-2-yl | CH₃ | CH₃ | H | H |
| X.515 | pyrid-2-yl | CH₂Br | CH₃ | H | H |
| X.516 | pyrid-2-yl | CH₂CH₃ | CH₃ | H | H |
| X.517 | pyrid-2-yl | n-C₆H₁₃ | CH₃ | H | H |
| X.518 | 5-chloro-3-fluoro-pyrid-2- | CH₂CO₂CH₃ | CH₃ | H | H |
| | yl | | | | |
| X.519 | pyrid-2-yl | CH₃ | CH₃ | H | H |
| X.520 | 3-chloro-5- | pyrid-2-yl | CH₃ | H | H |
| | trifluoromethyl-pyrid-2-yl | | | | |
| X.521 | 3-chloro-5- | phenyl | CH₃ | H | H |
| | trifluoromethyl-pyrid-2-yl | | | | |
| X.522 | 5-methylthio-pyrid-2-yl | CHBrCH₃ | CH₃ | H | H |
| X.523 | 5-ethoxycarbonyl-6- | CH₃ | CH₃ | H | H |
| | trifluoromethyl-pyrid-2-yl | | | | |
| X.524 | 5-trifluoromethyl-pyrid-2- | CH₂CH₃ | CH₃ | H | H |
| | yl | | | | |
| X.525 | 5-chloro-3-fluoro-pyrid-2- | CH₂CH₃ | CH₃ | H | H |
| | yl | | | | |
| X.526 | 6-ethoxycarbonyl-3- | CH₃ | CH₃ | H | H |
| | methyl-pyrid-2-yl | | | | |
| X.527 | 4-trifluoromethyl-6- | CH₃ | CH₃ | H | H |
| | (1,1,1-trifluoroethoxy)- | | | | |
| | pyrid-2-yl | | | | |
| X.528 | 6-hydroxymethyl-pyrid-2- | H | CH₃ | H | H |
| | yl | | | | |
| X.529 | 4-carboxyamine-pyrid-2-yl | CH₃ | CH₃ | H | H |
| X.530 | 6-phenoxy-pyrid-2-yl | H | CH₃ | H | H |
| X.531 | 4-cyano-pyrid-2-yl | CH₃ | CH₃ | H | H |
| X.532 | pyrid-2-yl | 4-chloro-phenyl | CH₃ | H | H |
| X.533 | 3-hydroxy-6-methyl-pyrid- | H | CH₃ | H | H |
| | 2-yl | | | | |
| X.534 | pryid-2-yl | CH₂CO₂CH₂CH₃ | CH₃ | H | H |
| X.535 | 5-cyano-6-methylthio- | H | CH₃ | H | H |
| | pyrid-2-yl | | | | |
| X.536 | 6-cyano-pyrid-2-yl | CH₃ | CH₃ | H | H |
| X.537 | pyrid-2-yl | pyrid-4-yl | CH₃ | H | H |
| X.538 | 3-allyl-6-methyl-pyrid-2-yl | H | CH₃ | H | H |
| X.539 | 6-bromo-pyrid-2-yl | 2-methyl- | CH₃ | H | H |
| | | phenyl | | | |
| X.540 | 6-methoxycarbonyl-3- | CH₃ | CH₃ | H | H |
| | trifluoromethyl-pyrid-2-yl | | | | |
| X.541 | 4-^{t}butyl-pyrid-2-yl | CH₃ | CH₃ | H | H |
| X.542 | 3,5-dichloro-pyrid-2-yl | CH(CH₃)CH₃ | CH₃ | H | H |
| X.543 | pyrid-2-yl N-oxide | CH₃ | CH₃ | H | H |
| X.544 | 6-(2′-chlorophenyl)-pryid- | CH₃ | CH₃ | H | H |
| | 2-yl | | | | |
| X.545 | 5-chloro-3-fluoro-pyrid-2- | CHClCH₃ | CH₃ | H | H |
| | yl | | | | |
| X.546 | 3-chloro-5- | CO₂CH₃ | CH₃ | H | H |
| | trifluoromethyl-pyrid-2-yl | | | | |
| X.547 | pyrid-2-yl | 3-methyl-4- | CH₃ | H | H |
| | | pyrrol-1-yl- | | | |
| | | phenyl | | | |
| X.548 | 5-chloro-3-hydroxy-pyrid- | CH₃ | CH₃ | H | H |
| | 2-yl | | | | |
| X.549 | 3,5-dichloro-pyrid-2-yl | CH₂CH₃ | CH₃ | H | H |
| X.550 | pyrid-2-yl | CH₃CN | CH₃ | H | H |
| X.551 | 3-chloro-5- | CH₃ | CH₃ | H | H |
| | trifluoromethyl-pyrid-2-yl | | | | |
| X.552 | 5-chloro-3-fluoro-pyrid-2- | H | CH₃ | H | H |
| | yl | | | | |
| X.553 | 5-benzyloxy-pyrid-2-yl | H | CH₃ | H | H |
| X.554 | pryid-2-yl | n-C₁₀H₂₁ | CH₃ | H | H |
| X.555 | 4,6-dimethyl-pyrid-2-yl | CH₃ | Cl | H | H |
| X.556 | 4,6-dimethyl-pyrid-2-yl | CH₃ | phenoxy | H | H |
| X.557 | 4,6-dimethyl-pyrid-2-yl | 4,6-dimethyl- | CH₃ | H | H |
| | | pyrid-2-yl | | | |
| X.558 | 4,6-dimethyl-pyrid-2-yl | CH₃ | 3-hydroxyprop- | H | H |
| | | | 1-ynyl | | |
| X.559 | 4,6-dimethyl-pyrid-2-yl | CH₃ | 6-methyl-pyrid- | H | H |
| | | | 2-yl | | |
| X.560 | 4,6-dimethyl-pyrid-2-yl | CH₃ | CH₃ | H | phenyl |
| X.561 | 4,6-dimethyl-pyrid-2-yl | CH₃ | —CH═CHCH═CH— | | CH₃ |
| X.562 | 4,6-dimethyl-pyrid-2-yl | CH₃ | CH₃ | H | phenoxy |
| X.563 | 4,6-dimethyl-pyrid-2-yl | CH₃ | CH₃ | H | pyrrolidin- |
| | | | | | 1-yl |
| X.564 | 3,6-dimethyl-pyrid-2-yl | CH₃ | CH₃ | H | H |
| X.565 | 4,6-dimethyl-pyrid-2-yl | CH₃ | CH₃ | H | methyl |
| | | | | | thio |
| X.566 | 4,6-dimethyl-pyrid-2-yl | CH₃ | methoxymethyl | H | methyl |
| X.567 | 4,6-dimethyl-pyrid-2-yl | CH₃ | CH₃ | H | CH₂F |
| X.568 | 6-methyl-pyrid-2-yl | CH₃ | CH₃ | H | CH₂F |
| X.569 | 4,6-dimethyl-pyrid-2-yl | CH₃ | CH₃ | H | SO₂CH₃ |

TABLE 1
This table discloses 569 compounds 1.001 to 1.569 of the formula (I-I)

| | |
|---|---|
| | (I-I) |
| 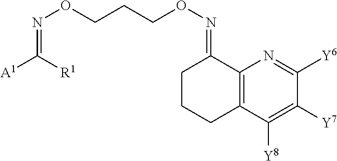 | |

wherein A¹, R¹, Y⁶, Y⁷ and Y⁸ have the specific meanings given in the Table.

TABLE 2
This table discloses 569 compounds 2.001 to 2.569 of the formula (I-II)

| | |
|---|---|
| | (I-II) |
| 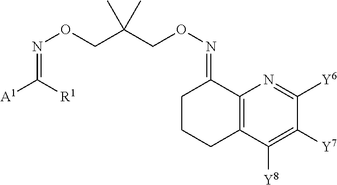 | |

wherein A¹, R¹, Y⁶, Y⁷ and Y⁸ have the specific meanings given in the Table.

TABLE 3
This table discloses 569 compounds 3.001 to 3.569 of the formula (I-III)

| | |
|---|---|
| | (I-III) |
| 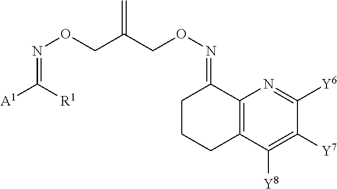 | |

wherein A¹, R¹, Y⁶, Y⁷ and Y⁸ have the specific meanings given in the Table.

TABLE 4
This table discloses 569 compounds 4.001 to 4.569 of the formula (I-IV)

| | |
|---|---|
| | (I-IV) |
| 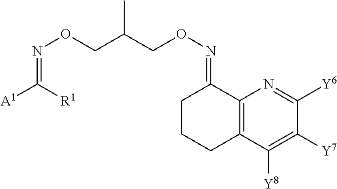 | |

wherein A¹, R¹, Y⁶, Y⁷ and Y⁸ have the specific meanings given in the Table.

TABLE 5
This table discloses 569 compounds 5.001 to 5.569 of the formula (I-V)

| | |
|---|---|
| | (I-V) |
| 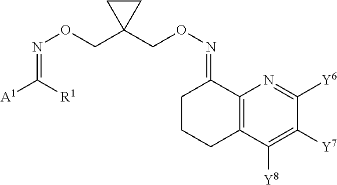 | |

wherein A¹, R¹, Y⁶, Y⁷ and Y⁸ have the specific meanings given in the Table.

TABLE 6
This table discloses 569 compounds 6.001 to 6.569 of the formula (I-VI)

| | |
|---|---|
| | (I-VI) |
| 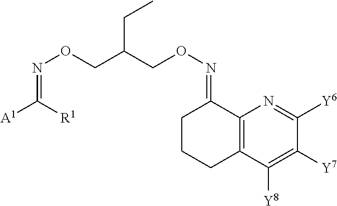 | |

wherein A¹, R¹, Y⁶, Y⁷ and Y⁸ have the specific meanings given in the Table.

TABLE 7
This table discloses 569 compounds 7.001 to 7.569 of the formula (I-VII)

| | |
|---|---|
| | (I-VII) |
| 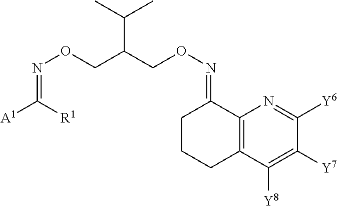 | |

wherein A¹, R¹, Y⁶, Y⁷ and Y⁸ have the specific meanings given in the Table.

TABLE 8
This table discloses 569 compounds 8.001 to 8.569 of the formula (I-VIII)

| | |
|---|---|
| | (I-VIII) |
| 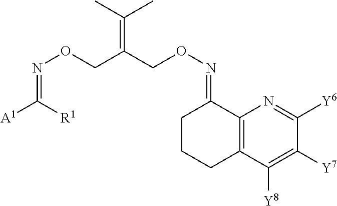 | |

wherein A¹, R¹, Y⁶, Y⁷ and Y⁸ have the specific meanings given in the Table.

TABLE 9
This table discloses 569 compounds 9.001 to 9.569 of the formula (I-IX)

| | |
|---|---|
| | (I-IX) |
| 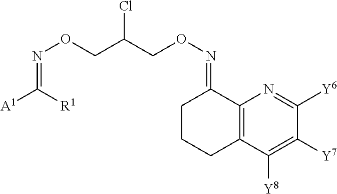 | |

wherein A¹, R¹, Y⁶, Y⁷ and Y⁸ have the specific meanings given in the Table.

TABLE 10
This table discloses 569 compounds 10.001 to 10.569 of the formula (I-X)

| | |
|---|---|
| | (I-X) |
| 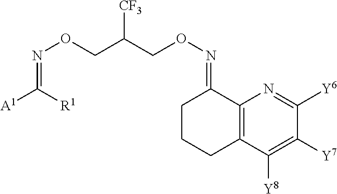 | |

wherein A¹, R¹, Y⁶, Y⁷ and Y⁸ have the specific meanings given in the Table.

TABLE 11
This table discloses 569 compounds 11.001 to 11.569 of the formula (I-XI)

| | |
|---|---|
| | (I-XI) |
| 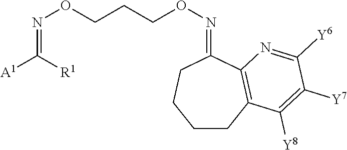 | |

wherein A¹, R¹, Y⁶, Y⁷ and Y⁸ have the specific meanings given in the Table.

TABLE 12
This table discloses 569 compounds 12.001 to 12.569 of the formula (I-XII)

| | |
|---|---|
| | (I-XII) |
| 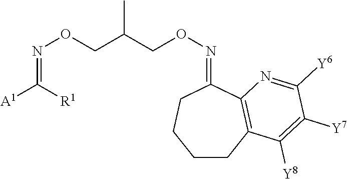 | |

wherein A¹, R¹, Y⁶, Y⁷ and Y⁸ have the specific meanings given in the Table.

TABLE 13
This table discloses 569 compounds 13.001 to 13.569 of the formula (I-XVI)

| | |
|---|---|
| | (I-XIII) |
| 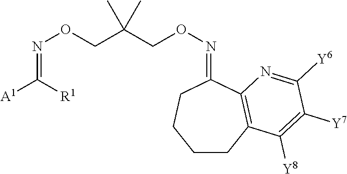 | |

wherein A¹, R¹, Y⁶, Y⁷ and Y⁸ have the specific meanings given in the Table.

TABLE 14
This table discloses 569 compounds 14.001 to 14.569 of the formula (I-XIII)

| | |
|---|---|
| | (I-XIV) |
| 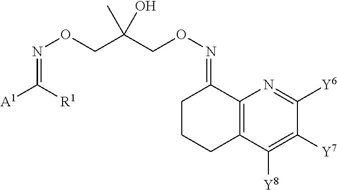 | |

wherein A¹, R¹, Y⁶, Y⁷ and Y⁸ have the specific meanings given in the Table.

TABLE 15
This table discloses 569 compounds 15.001 to 15.569 of the formula (I-XV)

| | |
|---|---|
| | (I-XV) |
| 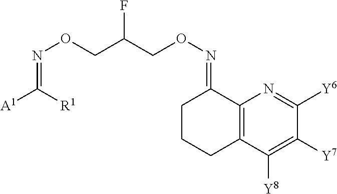 | |

wherein A¹, R¹, Y⁶, Y⁷ and Y⁸ have the specific meanings given in the Table.

The compounds in Tables 1 to 15 include all isomers, tautomers and mixtures thereof, including the cis/trans isomers shown above.

The compounds of the invention may be made by a variety of methods, illustrated in schemes 1-8. The compounds depicted in the schemes also indicate any isomers and tautomers, in particular the geometric isomers arising from the oxime and oxime ether moieties.

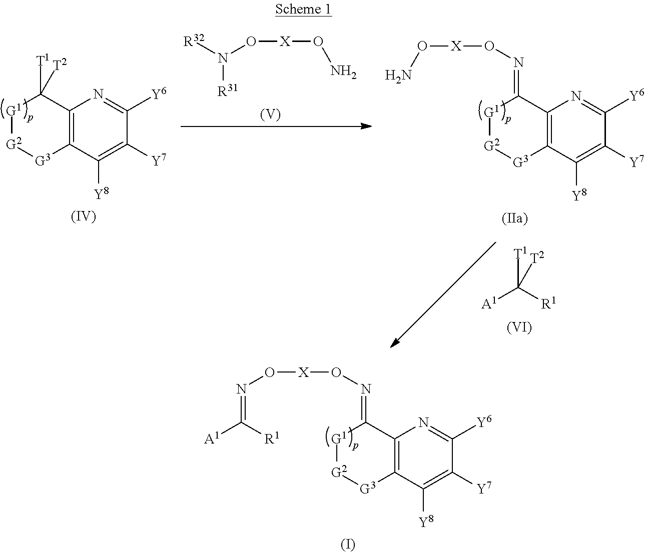

1) Compounds of formula (I) may be prepared by reacting a compound of formula (IIa), wherein X, G¹, G², G³, p, Y⁶, Y² and Y⁸ are as defined herein for compounds of formula (I), with a compound of formula (VI), wherein A¹ and R¹ are as defined herein for compounds of formula (I), and T¹ and T² are C₁-C₈ alkoxy, or T¹ and T² together with the carbon they are attached to form a carbonyl group or an acetal or ketal function of the form C(O—C₁-C₆-alkylidene-O) whereby the alkylidene fragment may optionally be mono- to tetra-substituted by C₁-C₆ alkyl, as seen in scheme 1.

A general description of condensation reactions is given below, and typical reaction conditions for this type of reaction may be found in Journal of Organic Chemistry, 52(22), 4978-84; 1987; Chemical & Pharmaceutical Bulletin, 51(2), 138-151; 2003; Organic Letters, 10(2), 285-288; 2008; Journal of the American Chemical Society, 130(12), 4196-4201; 2008; Chemistry & Biology, 9(1), 113-129; 2002; Organic Preparations and Procedures International, 32(2), 153-159; 2000; Scientia Pharmaceutica, 66(1), 9-21; 1998, Journal of Medicinal Chemistry, 49(17), 5177-5186; 2006, Journal of Agricultural and Food Chemistry, 38(3), 839-44; 1990; Tetrahedron: Asymmetry, 8(2), 253-263; 1997; Journal of Medicinal Chemistry, 44(21), 3339-3342; 2001; Bioorganic & Medicinal Chemistry Letters, 12(3), 341-344; 2002; US 2007032470; WO 07/058,504; Journal of Organic Chemistry, 73(5), 2007-2010; 2008; Bioorganic & Medicinal Chemistry Letters, 19(10), 2683-2687; 2009; and

Bioorganic & Medicinal Chemistry Letters, 19(10), 2654-2660; 2009.

2) Hydroxylamine derivatives of formula (IIa) may be made by reacting compounds of formula (IV), wherein G¹, G², G³, p, Y⁶, Y² and Y⁸ are as defined herein for compounds of formula (I), and T¹ and T² are C₁-C₈ alkoxy, or T¹ and T² together with the carbon they are attached to form a carbonyl group or an acetal or ketal function of the form C(O—C₁-C₆-alkylidene-O) whereby the alkylidene fragment may optionally be mono- to tetra-substituted by C₁-C₆ alkyl, with a bishydroxylamine derivative of formula (V), wherein X is as defined herein for a compound of formula (I) and R³¹ and R³² are either hydrogen or suitable protecting groups such as tert-butyloxycarbonyl (BOC), allyloxycarbonyl, fluorenylmethyloxycarbonyl (FMOC), formyl, acetyl, propionyl, trifluoroacetyl, benzoyl, substituted benzoyl, STABASE, Si(O—C₁-C₈-alkyl)₃, bis-Si(O—C₁-C₈-alkyl)₃, bis-benzyl, substituted bis-benzyl, bis-allyl, substituted bis-allyl, bis C₁-C₈-alkoxy-alkyl, N-phenylmethylene, substituted N-phenylmethylene, trityl, benzhydryl, substituted benzhydryl, or R³¹ and R³² together with the nitrogen atom to which they are attached may form a phthalyl group (scheme 1). General conditions for this type of condensation reaction can be found below.

When R³¹ and R³² are hydrogen, in order to optimize the yield of compound (IIa) an excess of intermediate (V) over intermediate (IV) may preferably be used. If R³¹ or R³² is not hydrogen, the hydroxylamine derivative may be deprotected using techniques well known to the person skilled in the art. Examples can be found in Greene, T. W., Wuts, P. G. N., Protective Groups in Organic Synthesis, John Wiley & Sons, Inc, 2006.

Monoprotection of bis-hydroxylamines has been described in Tetrahedron (1997), 53(15), 5485-5492. It is to be understood that methods used to obtain mono-protected diamines can be used in an analogous way to obtain mono-protected bis-hydroxylamine derivatives. Typical conditions for this type of reaction can be found in Synthetic Communications (2007), 37(5), 737-742; Organic Preparations and Procedures International (2009), 41(4), 301-307; Tetrahedron: Asymmetry (2003), 14(11), 1559-1563; Bulletin of the Korean Chemical Society (1994), 15(12), 1025-7; Synthesis (1990), (4), 366-8; and

Synthesis (1984), (12), 1032-3.

Bishydroxylamine derivatives are known in the literature. A description of their preparation can be found in WO 08/074,418; Inorganic Chemistry Communications (2009), 12(3), 234-236; WO 99/49314; Synthesis (1997), (1), 38-40; and Gazzetta Chimica Italiana (1954), 84 915-20.

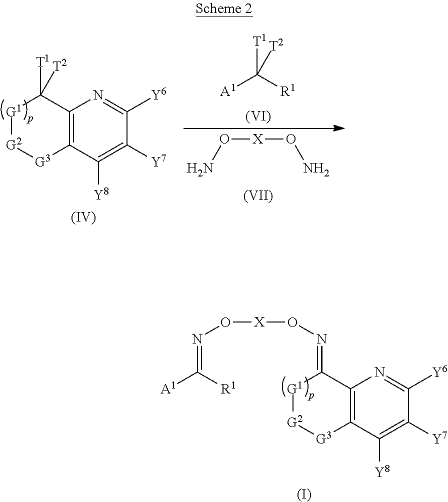

3) Alternatively, as seen in scheme 2, compounds of formula (I) may be prepared by reacting a compound of formula (IV) and a compound of formula (VI) in the presence of a compound of formula (VII), wherein X is as defined herein for compounds of formula (I). Compounds of formula (IV) and (VI) are described under Scheme 1.

Typical reaction conditions for condensation reactions are seen below, and typical conditions for this particular condensation reaction are seen in the following references: Nature Chemical Biology, 5(6), 407-413; 2009; Acta Crystallographica, Section E: Structure Reports Online, E65(7), o1657; 2009; Acta Crystallographica, Section E: Structure Reports Online, E64(8), o1405, o1405/1-o1405/7; 2008; Acta Crystallographica, Section E: Structure Reports Online, E64(7), o1324, o1324/1-o1324/6; 2008; Acta Crystallographica, Section E: Structure Reports Online, E63(10), o4080, So4080/1-So4080/7; 2007; Synthetic Communications, 33(4), 543-546; 2003.

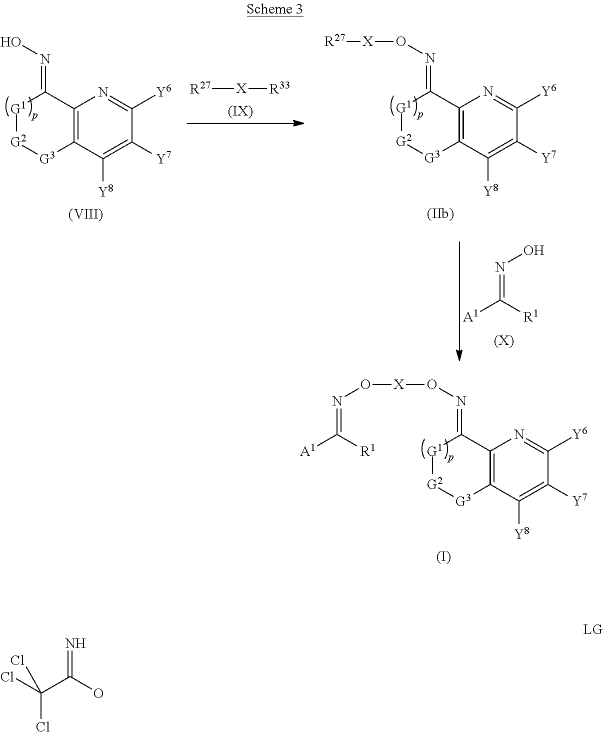

4) Alternatively, compounds of formula (I) can be obtained by reacting a compound of formula (IIb), that is a compound of formula (II) wherein R²⁷ is a halogen, in particular chlorine, bromine or iodine, or a sulfonic acid ester group, such as mesylate, tosylate, triflate, a phenylsulfonic acid ester, a nitro-phenylsulfonic acid ester, or a nonafluorobutylsulfonic acid ester, or LG, and G¹, G², G³, p, X, Y⁶, Y⁷ and Y⁸ are as defined herein for compounds of formula (I), with a compound of formula (X), wherein A¹ and R¹ are as defined herein for compounds of formula (I) (scheme 3).

Typical reaction conditions for alkylation reactions such as this may be found below. These are further illustrated in Chinese Journal of Chemistry, 27(1), 33-42; 2009; WO 09/049,846; Journal of Antibiotics, 61(10), 603-614; 2008; Bioorganic & Medicinal Chemistry Letters, 18(24), 6471-6475; 2008; Journal of Medicinal Chemistry, 51(15), 4601-4608; 2008; WO 06/123145, Archiv der Pharmazie (Weinheim, Germany), 340(4), 202-208; 2007; Synthetic Communications, 37(7), 1155-1165; 2007; Russian Journal of Organic Chemistry, 42(5), 735-738; 2006; Bioinorganic Chemistry and Applications, 1(3-4), 299-308; 2003; Synthetic Communications, 28(14), 2621-2633; 1998; Synthetic Communications, 19(18), 3129-38; 1989.

5) Compounds of formula (IIb) may be obtained by reacting an oxime of formula (VIII) wherein G¹, G², G³, p, Y⁶, Y⁷ and Y⁸ are as defined herein for compounds of formula (I), with a compound of formula (IX), wherein R²⁷ is as defined herein for compounds of formula (IIb) and R³³ is a halogen, in particular chlorine, bromine or iodine, a sulfonic acid ester group, or the group LG (scheme 3). R²⁷ and R³³ may be the same or different. Preferentially, R³³ is a better leaving group under the conditions of the reaction, such as tosylate or bromine when R²⁷ is chlorine. Preferentially, an excess of the compound of formula (IX) relative to the oxime (VIII) would be used in the reaction, especially when R²⁷ and R³³ are the same.

Typical reaction conditions for alkylation reactions such as this can be found below, and are further illustrated in Journal of Agricultural and Food Chemistry (2008), 56(23), 11376-1139, Farmaco (2003), 58(9), 707-714; 1985; Journal of Heterocyclic Chemistry (1979), 16(7), 1459-67; WO 08/074,418; Journal of Medicinal Chemistry (2008), 51(20), 6421-6431; Synthetic Communications (2007), 37(7), 1155-1165; Bioorganic & Medicinal Chemistry (2007), 15(13), 4520-4527; Journal of Medicinal Chemistry (2006), 49(15), 4638-4649; and Synlett (2001), (Spec. Issue), 931-936.

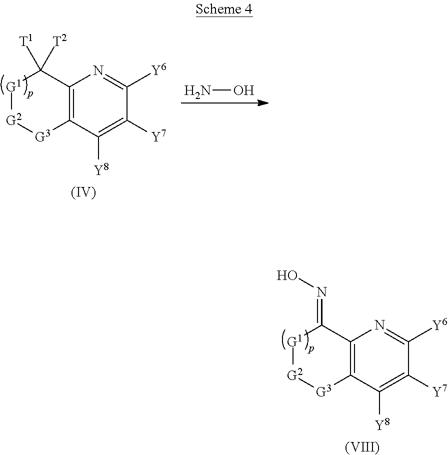

6) Oximes of formula (VIII) may be obtained by a condensation reaction, whereby a compound of formula (IV), wherein G¹, G², G³, p, Y⁶, Y² and Y⁸ are as defined herein for compounds of formula (I) and T¹ and T² are C₁-C₈ alkoxy, or T¹ and T² together with the carbon they are attached to form a carbonyl group or an acetal or ketal function of the form C(O—C₁-C₆-alkylidene-O) whereby the alkylidene fragment may optionally be mono- to tetra-substituted by C₁-C₆-alkyl, is reacted with hydroxylamine, or, alternatively, with a salt of hydroxylamine. A more detailed description of condensation processes is given below.

Related references include the following: Journal of Heterocyclic Chemistry, 46(1), 116-118; 2009; Journal of Medicinal Chemistry, 20(5), 718-21; 1977; Journal of Organic Chemistry, 73(11), 4017-4026; 2008; EJEAFChe, Electronic Journal of Environmental, Agricultural and Food Chemistry, 5(5), 1515-1521; 2006; Advanced Synthesis & Catalysis, 346(13-15), 1798-1811; 2004.

Some compounds of formula (IV) are known and their preparation has been published or they are available commercially. A few typical examples are given in Table 19 together with the corresponding CAS numbers. Analogous protocols to those used to prepare the following compounds can be used to prepare other compounds of formula (IV).

TABLE 16

| | | |
|---|---|---|
| 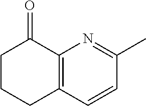 849643-01-2 | 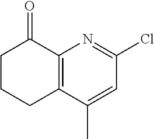 904915-35-1 | 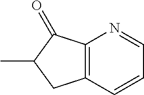 31170-79-3 |
| 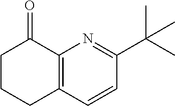 263566-88-7 | 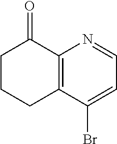 [SMILES: O=C1CCCc2c(Br)ccnc21] 1196155-16-4 | 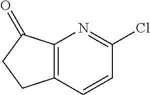 [SMILES: O=C1CCc2ccc(Cl)nc21] 1150617-92-7 |
| 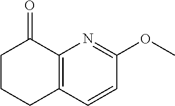 209741-58-2 | 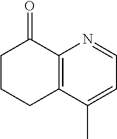 73123-86-1 | 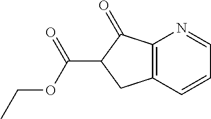 52402-29-6 |
| 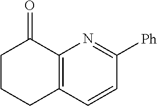 [SMILES: O=C1CCCc2ccc(-c3ccccc3)nc21] 78590-01-9 | 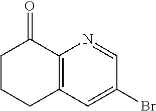 [SMILES: O=C1CCCc2cc(Br)cnc21] 904929-24-4 | 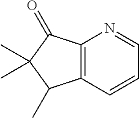 76474-76-5 |
| 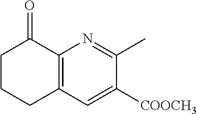 [SMILES: COC(=O)c1cc2c(nc1C)C(=O)CCC2] 212762-37-3 | 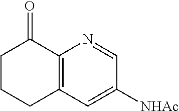 [SMILES: CC(=O)Nc1cnc2c(c1)CCCC2=O] 331759-68-3 | 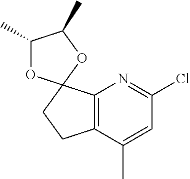 [SMILES: Cc1cc(Cl)nc2c1CCC21O[C@H](C)[C@@H](C)O1] 745075-86-9 |
| 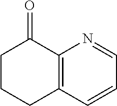 [SMILES: O=C1CCCc2cccnc21] 56826-69-8 | 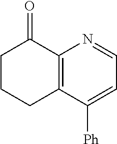 [SMILES: O=C1CCCc2c(-c3ccccc3)ccnc21] 135761-75-0 | 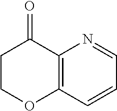 [SMILES: O=C1CCOc2cccnc21] 405174-48-3 |
| 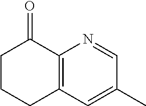 62230-65-3 | 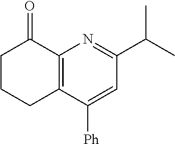 [SMILES: CC(C)c1cc(-c2ccccc2)c2c(n1)C(=O)CCC2] 906668-73-3 | 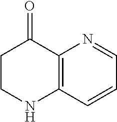 [SMILES: O=C1CCNc2cccnc21] 1211528-89-0 |
| 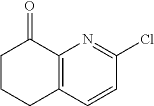 [SMILES: O=C1CCCc2ccc(Cl)nc21] 129337-86-6 | 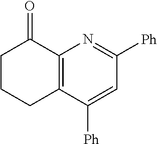 [SMILES: O=C1CCCc2c(-c3ccccc3)cc(-c3ccccc3)nc21] 1033623-16-3 | 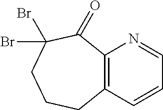 [SMILES: O=C1c2ncccc2CCCC1(Br)Br] 41043-16-7 |
| 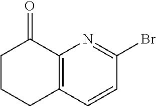 [SMILES: O=C1CCCc2ccc(Br)nc21] 130861-70-010 | 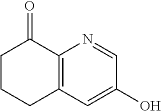 [SMILES: O=C1CCCc2cc(O)cnc21] 1196153-30-6 | 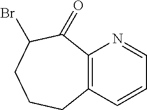 [SMILES: O=C1c2ncccc2CCCC1Br] 41043-14-5 |
| 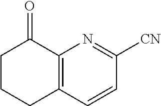 [SMILES: N#Cc1ccc2c(n1)C(=O)CCC2] 399042-43-4 | 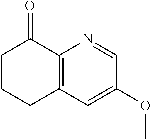 558444-62-5 | 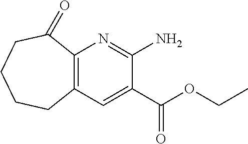 [SMILES: CCOC(=O)c1cc2c(nc1N)C(=O)CCCC2] 908231-09-4 |
| 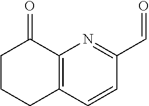 [SMILES: O=Cc1ccc2c(n1)C(=O)CCC2] 238755-38-9 | 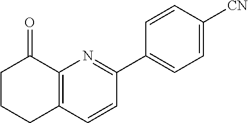 [SMILES: N#Cc1ccc(-c2ccc3c(n2)C(=O)CCC3)cc1] 423116-28-3 | 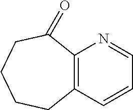 [SMILES: O=C1CCCCc2cccnc21] 41043-13-4 |
| 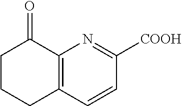 [SMILES: O=C(O)c1ccc2c(n1)C(=O)CCC2] 238755-39-0 | 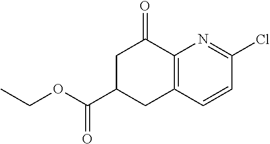 [SMILES: CCOC(=O)C1CC(=O)c2nc(Cl)ccc2C1] 864830-54-6 | 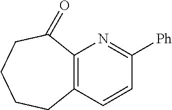 [SMILES: O=C1CCCCc2ccc(-c3ccccc3)nc21] 78509-53-2 |
| 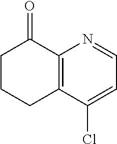 [SMILES: O=C1CCCc2c(Cl)ccnc21] 1196156-61-2 | 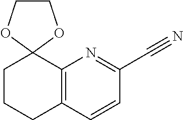 [SMILES: N#Cc1ccc2c(n1)C1(CCC2)OCCO1] 399042-44-5 | 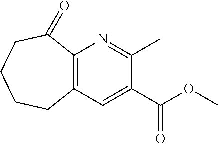 [SMILES: COC(=O)c1cc2c(nc1C)C(=O)CCCC2] 212762-38-4 |
| 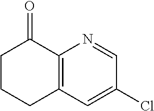 [SMILES: O=C1CCCc2cc(Cl)cnc21] 1196151-83-3 | 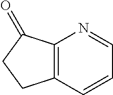 31170-78-2 | 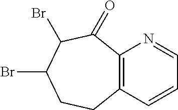 [SMILES: O=C1c2ncccc2CCC(Br)C1Br] 41043-15-6 |
| 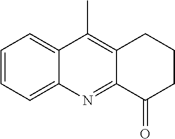 [SMILES: Cc1c2c(nc3ccccc13)C(=O)CCC2] 14428-47-8 | 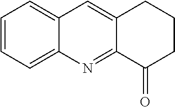 [SMILES: O=C1CCCc2cc3ccccc3nc21] 9568-10-7 | 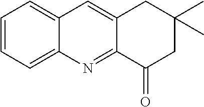 [SMILES: CC1(C)CC(=O)c2nc3ccccc3cc2C1] 01622-35-9 |
| 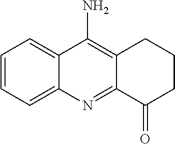 [SMILES: Nc1c2c(nc3ccccc13)C(=O)CCC2] 122910-29-6 | 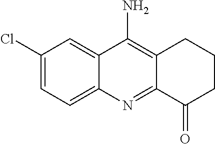 [SMILES: Nc1c2c(nc3ccc(Cl)cc13)C(=O)CCC2] 149194-86-5 | 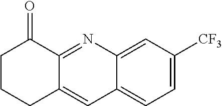 [SMILES: O=C1CCCc2cc3ccc(C(F)(F)F)cc3nc21] 844891-39-0 |
| 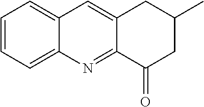 [SMILES: CC1CC(=O)c2nc3ccccc3cc2C1] 857613-10-6 | 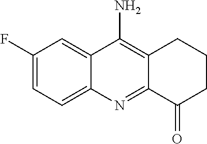 [SMILES: Nc1c2c(nc3ccc(F)cc13)C(=O)CCC2] 149194-90-1 | |

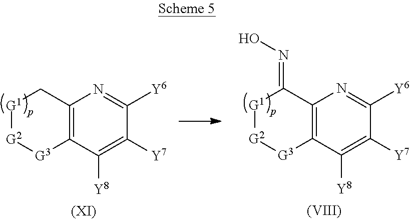

7) Alternatively, oximes of formula (VIII) can be obtained by a nitrosation reaction of compounds of formula (XI), wherein G¹, G², G³, p, Y⁶, Y⁷ and Y⁸ are as defined herein for compounds of formula (I), with base and an alkyl nitrite, as seen in scheme 5. Typical bases include lithium diisopropyl amide (LDA), lithium hexamethyldisilazane, n-butyl lithium, s-butyl lithium, tert-butyl lithium, sodium tert-butylate or potassium tert-butylate. Typical alkyl nitrites include isopentyl nitrite and tert-butyl nitrite. The compound of formula (XI), the alkyl nitrite or the base can be used in different stoichiometric amounts, with each reagent possibly being in excess with respect to the others. Preferentially, such reactions are carried out under non-aqueous conditions in an inert solvent such as hexane, heptanes, cyclohexane, toluene or ethers such as THF or tert-butyl methyl ether. The reaction may be performed at temperatures ranging from −80 to 250° C., preferably between −50 and 120° C.

Such reactions can lead to a mixture of the E- and the Z-oxime (ether) product, or the product may also be exclusively either the E- or the Z-oxime (ether).

A large number of these types of transformations are known in the art. Typical reaction conditions for this type of reaction may be found in Crawford, Jason B.; Chen, Gang; Gauthier, David; Wilson, Trevor; Carpenter, Bryon; Baird, Ian R.; McEachern, Ernie; Kaller, Alan; Harwig, Curtis; Atsma, Bem; Skerlj, Renato T.; Bridger, Gary J., Organic Process Research & Development (2008), 12(5), 823-830, McEachern, E. J.; Yang, W.; Chen, G.; Skerlj, R. T.; Bridger, G. J., Synthetic Communications (2003), 33(20), 3497-350; and Bark, Thomas; Thummel, Randolph P., Inorganic Chemistry (2005), 44(24), 8733-8739.

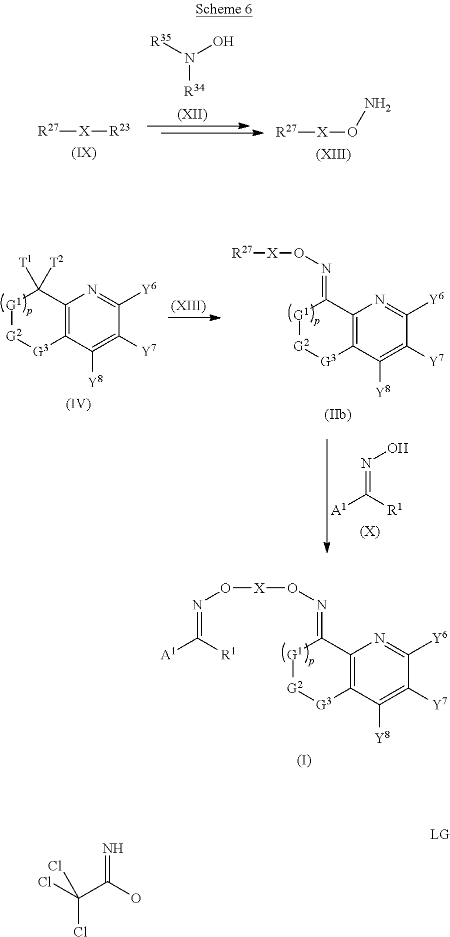

8) An alternative route to compounds of formula (I) is shown in Scheme 6. As in scheme 3, the compound of formula (I) is obtained by the reacting a compound of formula (IIb) with a compound of formula (X) as an alkylation reaction. Typical conditions for this type of reaction are described below.

9) The compounds of (IIb) can be formed by reacting a hydroxylamine derivative of formula (XIII), wherein R²⁷ halogen, with a compound of formula (IV), as seen in scheme 6. Compounds of formula (IV) are described above.

Typical reaction conditions for this type of condensation reaction may be found below, and are further illustrated in Angewandte Chemie, International Edition (2006), 45(32), 5307-5311.

10) Compounds of formula (XIII) can be made by alkylating a hydroxylamine derivative of formula (XII), wherein R³⁴ and R³⁵, either independently of each other, or together with each other and the nitrogen atom to which they are attached, are protecting groups, such as tert-butoxy carbonyl, acetyl, benzyl, or phthalyl, with the alkylating agent (IX), wherein R²⁷ is halogen and R³³ is halogen, in particular chloro, bromo or iodo, a sulfonic acid ester group, or LG (scheme 6). Typical conditions for such an alkylation reaction may be found below. The protecting groups or group can then be removed using techniques well known to a person skilled in the art, examples of which can be found in Greene, T. W., Wuts, P. G. N., Protective Groups in Organic Synthesis, John Wiley & Sons, Inc, 2006.

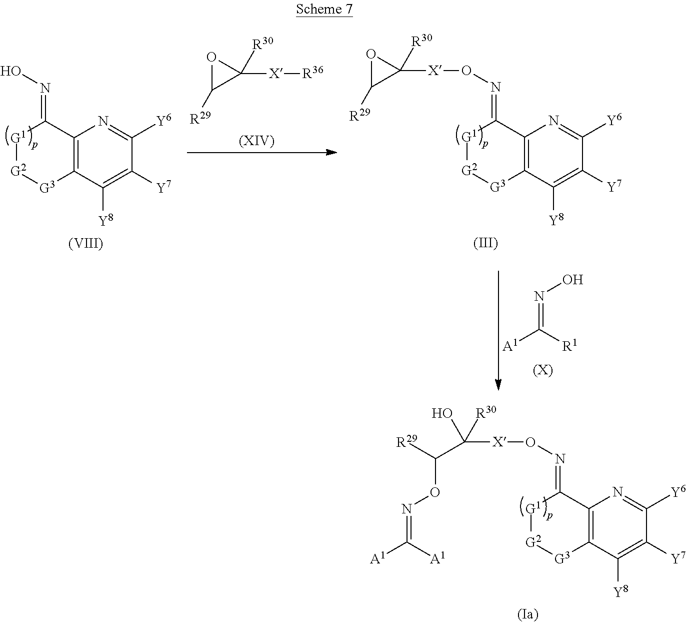

11) Compounds of formula (Ia), that is compounds of formula I wherein A¹, R¹, G¹, G², G³, Y⁶, Y⁷, Y⁸ and p are as defined herein for formula (I), X′ represents X′-1, X′-2 or X′-3

#—Z⁶—# X′-1

#—Z⁹—Z¹⁰—# X′-2

#—Z¹³—Z¹⁴—Z¹⁵—# X′-3

wherein Z⁶, Z⁹, Z¹⁰, Z¹³, Z¹⁴ and Z¹⁵ are as defined herein for compounds of formula (I), and R²⁹ and R³⁰ independently of one another represent hydrogen, halogen, C₁-C₄ alkyl, C₁-C₄ haloalkyl, phenyl or CN, wherein phenyl is optionally substituted by one or more groups, e.g. one to five groups, independently selected from halogen, CN, C₁-C₄ alkyl, C₁-C₄ haloalkyl, C₁-C₄ alkoxy and C₁-C₄ haloalkyoxy, can be made by reacting compounds of formula (III), wherein G¹, G², G³, Y⁶, Y⁷, Y⁸ and p are as defined herein for formula (I) and R²⁹ and R³⁰ independently of one another represent hydrogen, halogen, C₁-C₄ alkyl, C₁-C₄ haloalkyl, phenyl or CN, wherein phenyl is optionally substituted by one or more groups, e.g. one to five groups, independently selected from halogen, CN, C₁-C₄ alkyl, C₁-C₄ haloalkyl, C₁-C₄ alkoxy and C₁-C₄ haloalkyoxy, with compounds of formula (X) wherein A¹ and R¹ are as defined herein for compounds of formula (I) (scheme 7).

Typical conditions for an alkylation such as this are described below, and a further illustrated in Synthesis, (13), 2055-2064; 2008; Russian Journal of Organic Chemistry, 43(2), 181-183; 2007; Russian Journal of Organic Chemistry 43(3), 449-453; 2007; and Journal of Molecular Catalysis B: Enzymatic, 11(4-6), 255-263; 2001.

Compounds of formula (Ia) are especially useful as intermediates to a number of other compounds, wherein the hydroxy group formed by the opening of the epoxide is transformed into other functional groups, for example carbonyl, fluorine or chlorine. Such transformations can be effected using a number of conditions well known to the person skilled in the art.

12) Compounds of formula (III) can be obtained by the alkylation of oximes of formula (VIII) with epoxides of formula (XIV) wherein X′, R²⁹ and R³⁰ are as defined above for compounds of formula (III) and R³⁶ is halogen. Such alkylation processes are described in more detail below.

Relevant references include the following: Synthetic Communications, 37(7), 1155-1165; 2007; Molecules, 10(7), 747-754; 2005; Molecules, 10(11), 1399-1408; 2005; European Journal of Medicinal Chemistry, 40(12), 1351-1358; 2005; Organic Preparations and Procedures International, 30(2), 195-202; 1998; WO 08/074,418; and Journal fuer Praktische Chemie/Chemiker-Zeitung (1993), 335(7), 623-7.

A large number of compounds of formula (XIV) are commercially available or their preparation is to be found in the literature. Commercially available compounds (XIV) include epichlorohydrin, 2(S)-epichlorohydrin, 2(R)-epichlorohydrin, 2-methyl-epichlorohydrin, epibromohydrin.

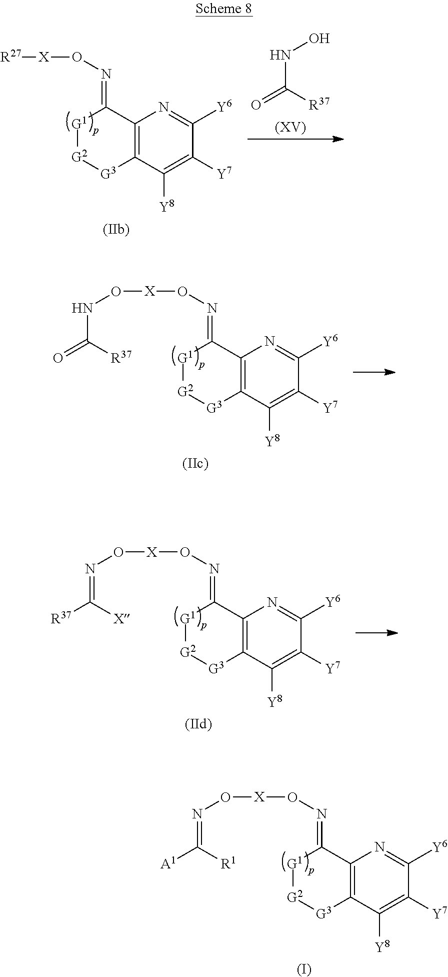

13) Compounds of formula (I) may be formed from compounds of formula (IId) wherein R³⁷ represents either A¹ or R¹ as defined herein for compounds of formula (I), and X, G¹, G², G³, p, Y⁶, Y⁷ and Y⁸ are as defined herein for compounds of formula (I) and X″ is halogen, preferably chlorine or bromine, by displacing the group X″ with a suitable derivative of the group A¹ or R¹, wherein A¹ or R¹ is as defined herein for compounds of formula (I). This can be done using one of several techniques well known to the person skilled in the art, including coupling reactions such as Suzuki (Suzuki-Miyaura) couplings and Stille couplings, (scheme 8).

The Suzuki coupling comprises the reaction between an organoboron compound, such as the boronic acid derivative of A¹ or R¹, or their esters, wherein A¹ or W is as described herein for a compound of formula (I), and a halide of formula (IId) to give compounds of formula (I).

The reaction may be done in the presence of a palladium catalyst such as Pd(PPh₃)₄, Pd(OAc)₂, Pd(dppf)Cl₂ and a base such as Na₂CO₃, Ba(OH)₂, K₃PO₄, Cs₂CO₃, K₂CO₃, KF, NaOH or alkali alcoholates, such as potassium tert-butoxide or sodium ethoxide. Typical catalyst loadings are in the range of 0.01 to 10 mol %. Preferred solvents for such cross coupling reactions include ethers such as THF or dimethoxyethane, acetonitrile, DMF, NMP, benzene or toluene or a mixture of such solvents. Such solvents can also be used together with water. The preferred temperature range for carrying out such reactions is between 0° C. and 180° C.

The Stille coupling comprises the use of an organotin compound, such as the tributylstannane derivative of A¹ or R¹ and a halide of formula (IId) to give compounds of formula (I).

The reaction can be done in the presence of a palladium catalyst such as Pd(PPh₃)₄, Pd₂(dba)₃.CHCl₃ with or with an added ligand such as P(2-furyl)₃ or Pd(OAc)₂, Pd(dppf)Cl₂, Pd(MeCN)₂Cl₂. Typical catalyst loadings are in the range of 0.01 to 10 mol %. Preferred solvents for such cross coupling reactions include ethers such as THF or dimethoxyethane, acetonitrile, DMF, NMP, benzene or toluene. Such solvents can also be used together with water. The preferred temperature range for carrying out such reactions is between 0° C. and 180° C.

Typical reaction conditions for these types of reaction may be found in Bioorganic and Medicinal Chemistry Letters 19(18), 5339-5345; 2009; Canadian Journal of Chemistry, 85(11), 913-922; 2007; Journal of Organic Chemistry, 72(13), 4892-4899; 2007; Tetrahedron Letters, 43(40), 7189-7191; 2002; Synlett, (10), 1557-1558; 2001; EP 792870; WO 95/20569.

14) Compounds of formula (IId) can be formed from compounds of formula (IIc) wherein R³⁷ represents either A¹ or R¹ as defined herein for compounds of formula (I), and, X, G¹, G², G³, p, Y⁶, Y⁷ and Y⁸ are as defined herein for compounds of formula (I), by the action of a halogenating agent.

The reaction can be performed using an excess of either the halogenating agent, equimolar amounts of halogenating agent and the hydroxamic acid ester (XVI), or with an excess of the hydroxamic acid ester (IIc). Preferentially it is carried out with an excess of halogenating agent over the hydroxamic acid ester (IIc).

Typical halogenating agents include CCl₄ or CBr₄ along with and a phosphine such as triphenyl phosphine or tributylphosphine. Other typical halogenating agents include Et₂NSF₃, (MeOCH₂CH₂)₂NSF₃ (Deoxo-Fluor), morpholinotrifluorosulfurane and SF₄, SOCl₂, COCl₂, PCl₅, PCl_{S}, PBr₃ or POCl₃, or a mixture of PCl₅ and POCl₃. Typical conditions include the use of a sub-stoichiometric, equimolar or excess amount of PCl₅ in POCl₃ relative to the compound of formula (IIc), where POCl₃ itself may be present in an equimolar amount or alternatively, be used in a sub-stoichiometric amount or excess relative to the compound of formula (IIc).

The halogenation of hydroxamic acid ester of formula (IIc) can be done without a solvent in certain cases or, preferentially, in the presence of a solvent or mixture of solvents. Any organic solvent that is inert under the specific reaction conditions can be chosen. Preferred solvents include the following, without limiting the selection: aliphatic or aromatic hydrocarbons that may optionally be substituted by one or several halogen atoms such as pentane, hexanes, heptanes, cyclohexane, petroleum ether, benzene, toluene, xylene, chlorobenzene, dichlorobenzenes, dichloromethane, chloroform, 1,2-dichloroethane, carbon tetrachloride, ethers such as diethylether, diisopropyl ether, dibutyl ether, tert-butyl methyl ether, tetrahydrofuran, 1,4-dioxane, dimethoxyethane, triethylene glycol dimethyl ether (methyltriglyme), or acetonitrile, propionitrile, benzonitrile or a substituted benzonitrile.

The use of catalysts to facilitate this type of reaction is established, comprising the use of catalysts such as dimethylformamide, diethylformamide and formylpiperidine. The transformation can also be done without a reaction catalyst.

Depending on the properties of the starting materials the reaction temperature can be varied over a wide range. Typical reaction temperatures vary between −100° C. and 250° C. Preferentially, the temperature range is between 0° C. and 100° C. On some occasions, the reaction may be carried out under reflux.

This transformation can also optionally be carried out under ultrasonication.

The use of organic bases in this type of reaction is well exemplified in the literature. The amount of base can be stoichiometric, sub- or super-stoichiometric. Typically an excess of base is used. Typical bases include the following without limiting the selection: Triethylamine, tripropylamine, tributylamine, di-isopropyl-ethylamine, N,N-dimethyl-cyclohexylamine, N-methyl-dicyclohexylamine, N,N-dimethyl-aniline, N,N-diethyl-aniline, N,N-dimethyl-benzylamine, N,N-diethyl-benzylamine, pyridine, 2-methyl-pyridine, 3-methyl-pyridine, 4-methyl-pyridine, 2,6-dimethyl-pyridine, 2,4,6-trimethyl-pyridine, 4-dimethylamino-pyridine, N-methyl-piperidine, N-ethyl-piperidine, N-methyl-morpholine, N-ethyl-morpholine, N,N′-dimethyl-piperazine. The transformation can also be done in the absence of bases

Typical reaction protocols for this type of reaction may be found in Bioorganic & Medicinal Chemistry Letters (2009), 19(18), 5339-5345; Journal of Medicinal Chemistry, 50(14), 3314-3321; 2007; Journal of Organic Chemistry, 69(8), 2741-2749; 2004; WO 01/025206; Australian Journal of Chemistry, 52(8), 807-811; 1999; Indian Journal of Chemistry, Section B: Organic Chemistry Including Medicinal Chemistry, 23B(8), 728-32; 1984.

15) Compounds of formula (IIc) can be formed by reacting compounds of formula (IIb) with a hydroxamic acid derivative of formula (XV) wherein R³⁷ is as defined herein for compounds of formula (IId) (scheme 8). This transformation is an alkylation of the hydroxamic acid derivative (XV) or its salt generated in situ by the action of a base. Typical conditions for alkylation reaction such as this are seen below, and are also described in the following references: WO 09/036,020; Journal of the Chemical Society, Perkin Transactions 2, (10), 1728-1739; 2002; Journal of Medicinal Chemistry, 42(1), 153-163; 1999; Journal of Medicinal Chemistry, 34(1), 57-65; 1991; Synthetic Communications, 19(3-4), 339-44; 1989; Journal of Organic Chemistry, 54(14), 3394-403; 1989; Tetrahedron, 43(11), 2577-92; 1987; Journal of Organic Chemistry, 51(26), 5047-50; 1986.

Typical Conditions for Condensation Reactions:

This applies to procedures 1, 2, 3, 6 and 9.

Different stoichiometric set-ups may be used for these reactions, depending on the properties of reactants and product. An excess of the electrophile, the nucleophile, or equimolar amounts may be chosen. Preferentially equimolar amounts of electrophilic and nucleophilic compounds are used.

The reaction may be performed in the presence or absence of an inert organic or inorganic solvent, or in the presence of a mixture of such solvents. Preferentially, it is performed in the presence of one or more solvents. Preferred solvents include the following aliphatic or aromatic hydrocarbons, which may optionally be substituted by one or more halogen atoms, such as pentane, hexanes, heptanes, cyclohexane, petroleum ether, benzene, toluene, xylene, chlorobenzene, dichlorobenzenes, dichloromethane, chloroform, 1,2-dichloroethane or carbon tetrachloride, ethers such as diethylether, diisopropyl ether, tert-butyl methyl ether, tetrahydrofuran, 1,4-dioxane, dimethoxyethane or diglycol dimethyl ether, ketones such as acetone, methyl ethyl ketone, methyl isopropyl ketone or methyl isobutyl ketone, acids and ester such as acetic acid, ethyl acetate or methyl acetate, aprotic polar solvents such as acetonitrile, pripionitril, dimethyl formamide, dimethyl acetamide, N-methyl-pyrrolidone, dimethyl sulfoxide, sulfolane, DMPU, or pyridine and picolines. The selection of solvents includes water and alcohols such as methanol, ethanol, propanol, isopropanol, butanol, isobutanol, tert-butanol, pentanol, isopentanol, hexanol, trifluorethanol, ethylene glycol or methoxyethanol.

The reaction may be performed between −20° C. and 250° C., preferentially between 0° C. and 100° C. In some cases the reaction mixture may be heated to reflux.

Where appropriate, compounds can be used in the form of the free compound, or, alternatively, they can be used in the form of a salt such as the acetate, trifluoroacetate, propionate, benzoate, oxalate, methylsolfonate, phenylsulfonate, p-tolylsulfonate, trifluormethylsulfonate, fluoride, chloride, bromide, iodide, sulphate, hydrogensulphate or nitrate, including bis-salts if appropriate.

The reaction can be carried out in the absence of an acid using the free compounds. Alternatively, the reaction may be performed in the presence of an acid in catalytic, stoichiometric or excess amounts. Acids that could be used include acetic acid, propionic acid, oxalic acid, trifluoroacetic acid, hydrochloric acid, hydrobromic acid, hydroiodic acid, methansulfonic acid, para-toluenesulfonic acid, sulphuric acid, sodium hydrogensulphate and phosphoric acid. The reaction can optionally be carried out in a water-free solvent system in the presence of a drying agent, such as sodium or magnesium sulphate, potassium carbonate or molecular sieves.

If the two substituents at the carbon atom of the oxime or oxime ether function are different from each other, the condensation reaction can lead to a mixture of the E- and the Z-oxime (ether) product. The condensation product may also be exclusively either the E- or the Z-oxime (ether).

Condensations can be performed under reduced pressure, normal pressure or increased pressure. Preferentially the reaction is performed under normal pressure.

Typical Conditions for Alkylation Reactions:

This applies to procedures 4, 5, 8, 10, 11, 12 and 15.

Different stoichiometric set-ups may be used for these reactions, depending on the properties of reactants and product. An excess of the electrophile, the nucleophile, or neither may be chosen. Usually, it is preferable that equimolar amounts of electrophilic and nucleophilic compounds are used.

The reaction may be performed in the absence or presence of a solvent or a mixture of solvents. Preferential solvents include the following aliphatic or aromatic hydrocarbons that may optionally be substituted by one or more halogen atoms such as pentane, hexanes, heptanes, cyclohexane, petroleum ether, benzene, toluene, xylene, chlorobenzene, dichlorobenzenes, dichloromethane, chloroform, 1,2-dichloroethanev or carbon tetrachloride, ethers such as diethyl ether, diisopropyl ether, tert-butyl-methyl ether, tetrahydrofuran, 1,4-dioxane, dimethoxyethane or diglycol dimethyl ether, ketones such as acetone, methyl ethyl ketone, methyl isopropyl ketone or methyl isobutyl ketone, acids and ester such as acetic acid, ethyl acetate or methyl acetate, aprotic polar solvents such as acetonitrile, pripionitrile, dimethyl formamide, dimethyl acetamide, N-methyl-pyrrolidone, dimethyl sulfoxide, sulfolane, DMPU, or pyridine and picolines. The selection of solvents includes also water and alcohols such as methanol, ethanol, propanol, isopropanol, butanol, isobutanol, tert-butanol, pentanol, isopentanol, hexanol, trifluorethanol, ethylene glycol or methoxyethanol.

The reaction may be performed in a biphasic system comprising an organic solvent that is not miscible with water, such as toluene, dichloromethane, dichloro-ethylene, and an aqueous solvent, such as water. Such a reaction would be performed in the presence of a phase-transfer catalyst, such as tetra-n-butylammonium bromide (TBAB), Tetradecyldimethylbenzylammonium chloride (TDMBAC), N-Benzyltrimethylammonium hydroxide, along with aqueous sodium or potassium hydroxide in stoichiometric amounts. The biphasic reaction may be performed with or without ultrasonication.

The reaction may be carried out at temperatures varying from −100° C. and 250° C. Preferentially, the temperature range is between 0° C. and 100° C.

Optionally, an organic or inorganic base may be present such as alkali- and earth alkali acetates, amides, carbonates, hydrogencarbonates, hydrides, hydroxides or alcoholates such as sodium, potassium, caesium or calcium acetate, sodium, potassium, caesium or calcium carbonate, sodium, potassium, caesium or calcium hydrogencarbonate, sodium, potassium, caesium or calcium hydride, sodium, potassium, caesium or calcium amide, sodium, potassium, caesium or calcium hydroxide, sodium, potassium, caesium or calcium methanolate, sodium, potassium, caesium or calcium ethanolate, sodium, potassium, caesium or calcium n-, i-, s- or t-butanolate, triethylamine, tripropylamine, tributylamine, di-isopropyl-ethylamine, N,N-dimethyl-cyclohexylamine, N-methyl-dicyclohexylamine, N,N-dimethyl-aniline, N,N-diethyl-aniline, N,N-dimethyl-benzylamine, N,N-diethyl-benzylamine, pyridine, 2-methyl-pyridine, 3-methyl-pyridine, 4-methyl-pyridine, 2,6-dimethyl-pyridine, 2,4,6-trimethyl-pyridine, 4-dimethylamino-pyridine, N-methyl-piperidine, N-ethyl-piperidine, N-methyl-morpholine, N-ethyl-morpholine, N,N′-dimethyl-piperazine, 1,4-Diazabicyclo[2.2.2]octane (DABCO), 1,8-Diaza-7-bicyclo[5.4.0]undecene (DBU), 1,5-Diazabicyclo[4.3.0]non-5-ene (DBN), 1-tert-Butyl-2,2,2-tri(1-pyrrolidinyl)phosphazene (BTPP), 1-tert-Butyl-2,2,2-tris(dimethylamino)phosphazene, sodium hexamethyldisilazane, potassium hexamethyldisilazane, lithium diisopropylamide, ethyl magnesium chloride, isopropylmagnesium chloride.

The alkylation can be performed under reduced pressure, normal pressure or increased pressure. Preferentially the reaction is performed under normal pressure.

The products of steps 1) to 15) may be required to be purified using, for example, chromatography, crystallisation or other purification techniques well known to the person skilled in the art.

The compounds of formula (I) to formula (XV) and, where appropriate, the tautomers thereof, can, if appropriate, also be obtained in the form of hydrates and/or include other solvents, for example those which may have been used for the crystallization of compounds which are present in solid form.

It has now been found that the compounds of formula (I) according to the invention have, for practical purposes, a very advantageous spectrum of activities for protecting useful plants against diseases that are caused by phytopathogenic microorganisams, such as fungi, bacteria or viruses.

The invention therefore also relates to a method of controlling or preventing infestation of useful plants by phytopathogenic microorganisms, wherein a compound of formula (I) is applied as active ingredient to the plants, to parts thereof or the locus thereof. The compounds of formula (I) according to the invention are distinguished by excellent activity at low rates of application, by being well tolerated by plants and by being environmentally safe. They have very useful curative, preventive and systemic properties and are used for protecting numerous useful plants. The compounds of formula (I) can be used to inhibit or destroy the diseases that occur on plants or parts of plants (fruit, blossoms, leaves, stems, tubers, roots) of different crops of useful plants, while at the same time protecting also those parts of the plants that grow later e.g. from phytopathogenic microorganisms.

It is also possible to use compounds of formula (I) as dressing agents for the treatment of plant propagation material, in particular of seeds (fruit, tubers, grains) and plant cuttings (e.g. rice), for the protection against fungal infections as well as against phytopathogenic fungi occurring in the soil.

Furthermore the compounds of formula (I) according to the invention may be used for controlling fungi in related areas, for example in the protection of technical materials, including wood and wood related technical products, in food storage or in hygiene management.

The compounds of formula (I) are, for example, effective against the phytopathogenic fungi of the following classes: Fungi imperfecti (e.g. Botrytis, Pyricularia, Helminthosporium, Fusarium, Septoria, Cercospora and Alternaria) and Basidiomycetes (e.g. Rhizoctonia, Hemileia, Puccinia). Additionally, they are also effective against the Ascomycetes classes (e.g. Venturia and Erysiphe, Podosphaera, Monilinia, Uncinula) and of the Oomycetes classes (e.g. Phytophthora, Pythium, Plasmopara). Within the scope of the invention, useful plants to be protected typically comprise the following species of plants: cereal (wheat, barley, rye, oat, rice, maize, sorghum and related species); beet (sugar beet and fodder beet); pomes, drupes and soft fruit (apples, pears, plums, peaches, almonds, cherries, strawberries, raspberries and blackberries); leguminous plants (beans, lentils, peas, soybeans); oil plants (rape, mustard, poppy, olives, sunflowers, coconut, castor oil plants, cocoa beans, groundnuts); cucumber plants (pumpkins, cucumbers, melons); fibre plants (cotton, flax, hemp, jute); citrus fruit (oranges, lemons, grapefruit, mandarins); vegetables (spinach, lettuce, asparagus, cabbages, carrots, onions, tomatoes, potatoes, paprika); lauraceae (avocado, cinnamomum, camphor) or plants such as tobacco, nuts, coffee, eggplants, sugar cane, tea, pepper, vines, hops, bananas and natural rubber plants, as well as ornamentals.

The term “useful plants” is to be understood as including also useful plants that have been rendered tolerant to herbicides like bromoxynil or classes of herbicides (such as, for example, HPPD inhibitors, ALS inhibitors, for example primisulfuron, prosulfuron and trifloxysulfuron, EPSPS (5-enol-pyrovyl-shikimate-3-phosphate-synthase) inhibitors, GS (glutamine synthetase) inhibitors or PPO (protoporphyrinogen-oxidase) inhibitors) as a result of conventional methods of breeding or genetic engineering. An example of a crop that has been rendered tolerant to imidazolinones, e.g. imazamox, by conventional methods of breeding (mutagenesis) is Clearfield® summer rape (Canola). Examples of crops that have been rendered tolerant to herbicides or classes of herbicides by genetic engineering methods include glyphosate- and glufosinate-resistant maize varieties commercially available under the trade names RoundupReady®, Herculex I® and LibertyLink®.

The term “useful plants” is to be understood as including also useful plants which have been so transformed by the use of recombinant DNA techniques that they are capable of synthesising one or more selectively acting toxins, such as are known, for example, from toxin-producing bacteria, especially those of the genus Bacillus.

Examples of such plants are: YieldGard® (maize variety that expresses a CryIA(b) toxin); YieldGard Rootworm® (maize variety that expresses a CryIIIB(b1) toxin); YieldGard Plus® (maize variety that expresses a CryIA(b) and a CryIIIB(b1) toxin); Starlink® (maize variety that expresses a Cry9(c) toxin); Herculex I® (maize variety that expresses a CryIF(a2) toxin and the enzyme phosphinothricine N-acetyltransferase (PAT) to achieve tolerance to the herbicide glufosinate ammonium); NuCOTN 33B® (cotton variety that expresses a CryIA(c) toxin); Bollgard I® (cotton variety that expresses a CryIA(c) toxin); Bollgard II® (cotton variety that expresses a CryIA(c) and a CryIIA(b) toxin); VIPCOT® (cotton variety that expresses a VIP toxin); NewLeaf® (potato variety that expresses a CryIIIA toxin); NatureGard® Agrisure® GT Advantage (GA21 glyphosate-tolerant trait), Agrisure® CB Advantage (Bt11 corn borer (CB) trait), Agrisure® RW (corn rootworm trait) and Protecta®.

The term “useful plants” is to be understood as including also useful plants which have been so transformed by the use of recombinant DNA techniques that they are capable of synthesising antipathogenic substances having a selective action, such as, for example, the so-called “pathogenesis-related proteins” (PRPs, see e.g. EP-A-0 392 225). Examples of such antipathogenic substances and transgenic plants capable of synthesising such antipathogenic substances are known, for example, from EP-A-0 392 225, WO 95/33818, and EP-A-0 353 191. The methods of producing such transgenic plants are generally known to the person skilled in the art and are described, for example, in the publications mentioned above.

The term “locus” of a useful plant as used herein is intended to embrace the place on which the useful plants are growing, where the plant propagation materials of the useful plants are sown or where the plant propagation materials of the useful plants will be placed into the soil. An example for such a locus is a field, on which crop plants are growing.

The term “plant propagation material” is understood to denote generative parts of the plant, such as seeds, which can be used for the multiplication of the latter, and vegetative material, such as cuttings or tubers, for example potatoes. There may be mentioned for example seeds (in the strict sense), roots, fruits, tubers, bulbs, rhizomes and parts of plants. Germinated plants and young plants which are to be transplanted after germination or after emergence from the soil, may also be mentioned. These young plants may be protected before transplantation by a total or partial treatment by immersion. Preferably “plant propagation material” is understood to denote seeds.

The compounds of formula (I) can be used in unmodified form or, preferably, together with carriers and adjuvants conventionally employed in the art of formulation.

Therefore the invention also relates to compositions for controlling and protecting against phytopathogenic microorganisms, comprising a compound of formula (I) and an inert carrier, and to a method of controlling or preventing infestation of useful plants by phytopathogenic microorganisms, wherein a composition, comprising a compound of formula (I) as active ingredient and an inert carrier, is applied to the plants, to parts thereof or the locus thereof.

To this end compounds of formula (I) and inert carriers are conveniently formulated in known manner to emulsifiable concentrates, coatable pastes, directly sprayable or dilutable solutions, dilute emulsions, wettable powders, soluble powders, dusts, granulates, and also encapsulations e.g. in polymeric substances. As with the type of the compositions, the methods of application, such as spraying, atomising, dusting, scattering, coating or pouring, are chosen in accordance with the intended objectives and the prevailing circumstances. The compositions may also contain further adjuvants such as stabilizers, antifoams, viscosity regulators, binders or tackifiers as well as fertilizers, micronutrient donors or other formulations for obtaining special effects.

Suitable carriers and adjuvants (auxiliaries) can be solid or liquid and are substances useful in formulation technology, e.g. natural or regenerated mineral substances, solvents, dispersants, wetting agents, tackifiers, thickeners, binders or fertilizers. Such carriers are for example described in WO 97/33890.

The compounds of formula (I) or compositions, comprising a compound of formula (I) as active ingredient and an inert carrier, can be applied to the locus of the plant or plant to be treated, simultaneously or in succession with further compounds. These further compounds can be e.g. fertilizers or micronutrient donors or other preparations which influence the growth of plants. They can also be selective herbicides as well as insecticides, fungicides, bactericides, nematicides, molluscicides or mixtures of several of these preparations, if desired together with further carriers, surfactants or application promoting adjuvants customarily employed in the art of formulation.

A preferred method of applying a compound of formula (I), or a composition, comprising a compound of formula (I) as active ingredient and an inert carrier, is foliar application. The frequency of application and the rate of application will depend on the risk of infestation by the corresponding pathogen. However, the compounds of formula (I) may also penetrate the plant through the roots via the soil (systemic action) by drenching the locus of the plant with a liquid formulation, or by applying the compounds in solid form to the soil, e.g. in granular form (soil application). In crops of water rice such granulates can be applied to the flooded rice field. The compounds of formula (I) may also be applied to seeds (coating) by impregnating the seeds or tubers either with a liquid formulation of the fungicide or coating them with a solid formulation.

A formulation, i.e. a composition comprising the compound of formula (I) and, if desired, a solid or liquid adjuvant, is prepared in a known manner, typically by intimately mixing and/or grinding the compound with extenders, for example solvents, solid carriers and, optionally, surface-active compounds (surfactants).

The agrochemical formulations will usually contain from 0.1 to 99% by weight, preferably from 0.1 to 95% by weight, of the compound of formula (I), 99.9 to 1% by weight, preferably 99.8 to 5% by weight, of a solid or liquid adjuvant, and from 0 to 25% by weight, preferably from 0.1 to 25% by weight, of a surfactant.

Whereas it is preferred to formulate commercial products as concentrates, the end user will normally use dilute formulations.

Advantageous rates of application are normally from 5 g to 2 kg of active ingredient (a.i.) per hectare (ha), preferably from 10 g to 1 kg a.i./ha, most preferably from 20 g to 600 g a.i./ha. When used as seed drenching agent, convenient rates of application are from 10 mg to 1 g of active substance per kg of seeds. The rate of application for the desired action can be determined by experiments. It depends for example on the type of action, the developmental stage of the useful plant, and on the application (location, timing, application method) and can, owing to these parameters, vary within wide limits.

The compounds of formula (I), or a pharmaceutical salt thereof, described above may also have an advantageous spectrum of activity for the treatment and/or prevention of microbial infection in an animal. “Animal” can be any animal, for example, insect, mammal, reptile, fish, amphibian, preferably mammal, most preferably human. “Treatment” means the use on an animal which has microbial infection in order to reduce or slow or stop the increase or spread of the infection, or to reduce the infection or to cure the infection. “Prevention” means the use on an animal which has no apparent signs of microbial infection in order to prevent any future infection, or to reduce or slow the increase or spread of any future infection.

According to the present invention there is provided the use of a compound of formula (I) in the manufacture of a medicament for use in the treatment and/or prevention of microbial infection in an animal. There is also provided the use of a compound of formula (I) as a pharmaceutical agent. There is also provided the use of a compound of formula (I) as an antimicrobial agent in the treatment of an animal. According to the present invention there is also provided a pharmaceutical composition comprising as an active ingredient a compound of formula (I), or a pharmaceutically acceptable salt thereof, and a pharmaceutically acceptable diluent or carrier. This composition can be used for the treatment and/or prevention of antimicrobial infection in an animal. This pharmaceutical composition can be in a form suitable for oral administration, such as tablet, lozenges, hard capsules, aqueous suspensions, oily suspensions, emulsions dispersible powders, dispersible granules, syrups and elixirs. Alternatively this pharmaceutical composition can be in a form suitable for topical application, such as a spray, a cream or lotion. Alternatively this pharmaceutical composition can be in a form suitable for parenteral administration, for example injection. Alternatively this pharmaceutical composition can be in inhalable form, such as an aerosol spray.

The compounds of formula (I) may be effective against various microbial species able to cause a microbial infection in an animal. Examples of such microbial species are those causing Aspergillosis such as Aspergillus fumigatus, A. flavus, A. terrus, A. nidulans and A. niger, those causing Blastomycosis such as Blastomyces dermatitidis; those causing Candidiasis such as Candida albicans, C. glabrata, C. tropicalis, C. parapsilosis, C. krusei and C. lusitaniae; those causing Coccidioidomycosis such as Coccidioides immitis; those causing Cryptococcosis such as Cryptococcus neoformans, those causing Histoplasmosis such as Histoplasma capsulatum and those causing Zygomycosis such as Absidia corymbifera, Rhizomucor pusillus and Rhizopus arrhizus. Further examples are Fusarium Spp such as Fusarium oxysporum and Fusarium solani and Scedosporium Spp such as Scedosporium apiospermum and Scedosporium prolificans. Still further examples are Microsporum Spp, Trichophyton Spp, Epidermophyton Spp, Mucor Spp, Sporothorix Spp, Phialophora Spp, Cladosporium Spp, Petriellidium spp, Paracoccidioides Spp and Histoplasma Spp.

The compositions of this invention may contain other compounds having biological activity, for example micronutrients or compounds having fungicidal activity or which possess plant growth regulating, herbicidal, insecticidal, nematicidal or acaricidal activity.

The present invention relates additionally to mixtures comprising at least a compound of formula I and at least a further, other biocidally active ingredient and optionally further ingredients. The further, other biocidally active ingredient are known for example from “The Pesticide Manual” [The Pesticide Manual—A World Compendium; Thirteenth Edition (New edition (2 Nov. 2003)); Editor: C. D. S. Tomlin; The British Crop Protection Council, ISBN-10: 1901396134; ISBN-13: 978-1901396133] or its electronic version “e-Pesticide Manual V4.2” or from the website http://www.alanwood.net/pesticides/ or preferably one of the further pesticides listed below.

The following mixtures of the compounds of TX with a further active ingredient (B) are preferred (the abbreviation “TX” means a compound encompassed by the compounds of formula I, or preferably the term “TX” refers to a compound selected from the Tables 1-15):

(B)

(B1) a strobilurin fungicide+TX,

(B2) an azole fungicide+TX,

(B3) a morpholine fungicide+TX,

(B4) an anilinopyrimidine fungicide+TX,

(B5) a fungicide selected from the group consisting of

Fluconazole+TX, Fluconazole-cis+TX, Fluxapyroxad+TX, Ametoctradin+TX, Flutianil+TX, Isotianil+TX, Valiphenal+TX, Anilazine+TX, arsenates+TX, benalaxyl+TX, benalaxyl-M+TX, benodanil+TX, benomyl+TX, benthiavalicarb+TX, benthiavalicarb-isopropyl+TX, biphenyl+TX, bitertanol+TX, blasticidin-S+TX, bordeaux mixture+TX, boscalid+TX, bupirimate+TX, cadmium chloride+TX, captafol+TX, captan+TX, carbendazim+TX, carbon disulfide+TX, carboxin+TX, carpropamid+TX, cedar leaf oil+TX, chinomethionat+TX, chlorine+TX, chloroneb+TX, chlorothalonil+TX, chlozolinate+TX, cinnamaldehyde+TX, copper+TX, copper ammoniumcarbonate+TX, copper hydroxide+TX, copper octanoate+TX, copper oleate+TX, copper sulphate+TX, cyazofamid+TX, cycloheximide+TX, cymoxanil+TX, dichlofluanid+TX, dichlone+TX, dichloropropene+TX, diclocymet+TX, diclomezine+TX, dicloran+TX, diethofencarb+TX, diflumetorim+TX, dimethirimol+TX, dimethomorph+TX, dinocap+TX, dithianon+TX, dodine+TX, edifenphos+TX, ethaboxam+TX, ethirimol+TX, etridiazole+TX, famoxadone+TX, fenamidone+TX, fenaminosulf+TX, fenamiphos+TX, fenarimol+TX, fenfuram+TX, fenhexamid+TX, fenoxanil+TX, fenpiclonil+TX, fentin acetate+TX, fentin chloride+TX, fentin hydroxide+TX, ferbam+TX, ferimzone+TX, fluazinam+TX, fludioxonil+TX, flusulfamide+TX, flusulfamide+TX, flutolanil+TX, folpet+TX, formaldehyde+TX, fosetyl-aluminium+TX, fthalide+TX, fuberidazole+TX, furalaxyl+TX, furametpyr+TX, flyodin+TX, fuazatine+TX, hexachlorobenzene+TX, hymexazole+TX, iminoctadine+TX, iodocarb+TX, iprobenfos+TX, iprodione+TX, iprovalicarb+TX, isoprothiolane+TX, kasugamycin+TX, mancozeb+TX, maneb+TX, manganous dimethyldithiocarbamate+TX, mefenoxam+TX, mepronil+TX, mercuric chloride+TX, mercury+TX, metalaxyl+TX, methasulfocarb+TX, metiram+TX, metrafenone+TX, nabam+TX, neem oil (hydrophobic extract)+TX, nuarimol+TX, octhilinone+TX, ofurace+TX, oxadixyl+TX, oxine copper+TX, oxolinic acid+TX, oxycarboxin+TX, oxytetracycline+TX, paclobutrazole+TX, paraffin oil+TX, paraformaldehyde+TX, pencycuron+TX, pentachloronitrobenzene+TX, pentachlorophenol+TX, penthiopyrad+TX, perfurazoate+TX, phosphoric acid+TX, polyoxin+TX, polyoxin D zinc salt+TX, potassium bicarbonate+TX, probenazole+TX, procymidone+TX, propamocarb+TX, propineb+TX, proquinazid+TX, prothiocarb+TX, pyrazophos+TX, pyrifenox+TX, pyroquilon+TX, quinoxyfen+TX, quintozene+TX, silthiofam+TX, sodium bicarbonate+TX, sodium diacetate+TX, sodium propionate+TX, streptomycin+TX, sulphur+TX, TCMTB+TX, tecloftalam+TX, tecnazene+TX, thiabendazole+TX, thifluzamide+TX, thiophanate+TX, thiophanate-methyl+TX, thiram+TX, tolclofos-methyl+TX, tolyfluanid+TX, triazoxide+TX, trichoderma harzianum+TX, tricyclazole+TX, triforine+TX, triphenyltin hydroxide+TX, validamycin+TX, vinclozolin+TX, zineb+TX, ziram+TX, zoxamide+TX, 1+TX, 1-bis(4-chlorophenyl)-2-ethoxyethanol+TX, 2+TX, 4-dichlorophenyl benzenesulfonate+TX, 2-fluoro-N-methyl-N-1-naphthylacetamide+TX, 4-chlorophenyl phenyl sulfone+TX,

a compound of formula B-5.1+TX

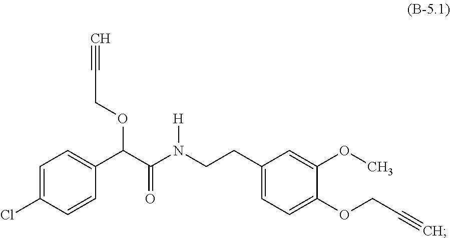 [SMILES: C#CCOc1ccc(CCNC(=O)C(OCC#C)c2ccc(Cl)cc2)cc1OC]

a compound of formula B-5.2+TX

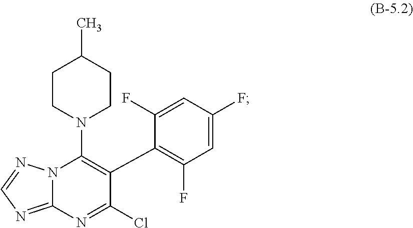 [SMILES: CC1CCN(c2c(-c3c(F)cc(F)cc3F)c(Cl)nc3ncnn23)CC1]

a compound of formula B-5.3+TX

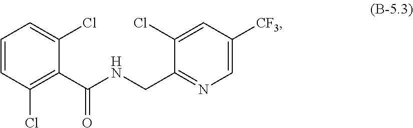 [SMILES: O=C(NCc1ncc(C(F)(F)F)cc1Cl)c1c(Cl)cccc1Cl]

a compound of formula B-5.4+TX

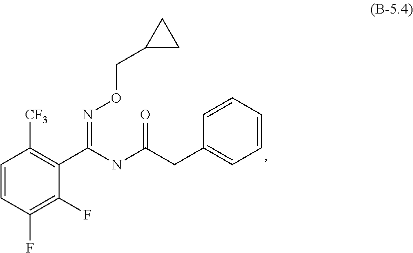 [SMILES: O=C(Cc1ccccc1)N/C(=N\OCC1CC1)c1c(C(F)(F)F)ccc(F)c1F]

a compound of formula B-5.5+TX

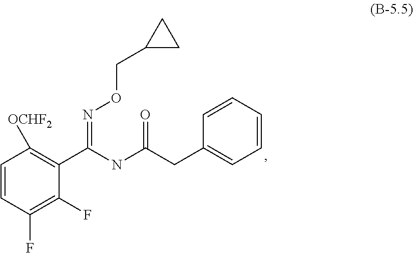 [SMILES: O=C(Cc1ccccc1)N/C(=N\OCC1CC1)c1c(OC(F)F)ccc(F)c1F]

a compound of formula B-5.6+TX

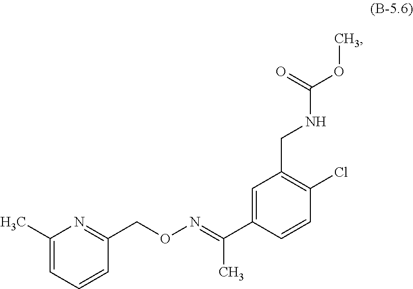 [SMILES: COC(=O)NCc1cc(/C(C)=N/OCc2cccc(C)n2)ccc1Cl]

a compound of formula B-5.7+TX

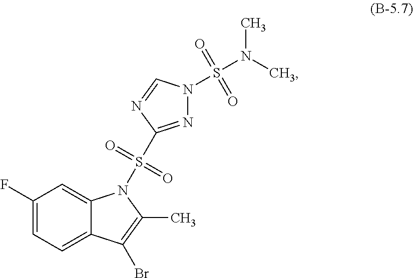 [SMILES: Cc1c(Br)c2ccc(F)cc2n1S(=O)(=O)c1ncn(S(=O)(=O)N(C)C)n1]

3-difluoromethyl-1-methyl-1H-pyrazole-4-carboxylic acid (2-bicyclopropyl-2-yl-phenyl)-amide (compound B-5.8)+TX, 3-difluoromethyl-1-methyl-1H-pyrazole-4-carboxylic acid (9-isopropyp-1,2,3,4-tetrahydro-1,4-methano-naphthalen-5-yl)-amide (compound B-5.9)+TX, 1,3-dimethyl-5-fluoro-1H-pyrazole-4-carboxylic acid [2-(1,3-dimethylbutyl)phenyl]-amide (compound B-5.10)+TX, 3-difluoromethyl-1-methyl-1H-pyrazole-4-carboxylic acid (3′,4′-dichloro-5-fluoro-1,1′-biphenyl-2-yl)-amide (compound B-5.11)+TX, N-{2-[3-chloro-5-(trifluoromethyl)pyridin-2-yl]ethyl}-2-(trifluoromethyl)benzamid (compound B-5.12)+TX, 3-difluoromethyl-1-methyl-1H-pyrazole-4-carboxylic acid N-[2-(1,1,2,2-tetrafluoroethoxy)phenyl]-amide (compound B-5.13)+TX, 3-difluoromethyl-1-methyl-1H-pyrazole-4-carboxylic acid N-[2-(1,1,2,3,3,3-hexafluoropropoxy)phenyl]-amide (compound B-5.14), 3-difluoromethyl-1-methyl-1H-pyrazole-4-carboxylic acid N-[2-(2-chloro-1+TX, 1,2-trifluoroethoxy)phenyl]-amide (compound B-5.15)+TX, 3-difluoromethyl-1-methyl-1H-pyrazole-4-carboxylic acid N-(4′-trifluoromethyl-biphen-2-yl)-amide (compound B-5.16)+TX, 3-difluoromethyl-1-methyl-1H-pyrazole-4-carboxylic acid N-(2′-trifluoromethyl-biphen-2-yl)-amide (compound B-5.17)+TX, 3-difluoromethyl-1-methyl-1H-pyrazole-4-carboxylic acid N-(2′-trifluoromethyl-biphen-2-yl)-amide (compound B-5.18)+TX; 3-difluoromethyl-1-methyl-1H-pyrazole-4-carboxylic acid (4′-methylsulfanyl-biphenyl-2-yl)-amide (compound B-5.19)+TX, 3-difluoromethyl-1-methyl-1H-pyrazole-4-carboxylic acid (2-dichloromethylene-3-ethyl-1-methyl-indan-4-yl)-amide (compound B-5.20)+TX

a compound of formula B-5.21+TX

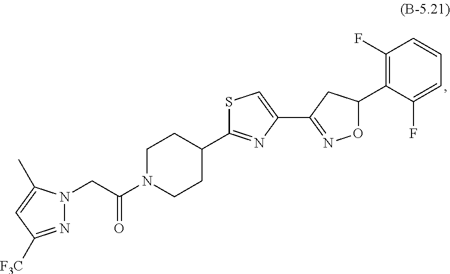 [SMILES: Cc1cc(C(F)(F)F)nn1CC(=O)N1CCC(c2nc(C3=NOC(c4c(F)cccc4F)C3)cs2)CC1]

(B6) a plant-bioregulator selected from the group consisting of

Acibenzolar+TX, 1-methyl-cyclopropene+TX, acibenzolar-S-methyl+TX, chlormequat chloride+TX, ethephon+TX, mepiquat chloride and trinexapc-ethyl;

(B7) an insecticide selected from the group consisting of

abamectin+TX, clothianidin+TX, emamectin benzoate+TX, imidacloprid+TX, tefluthrin+TX, thiamethoxam+TX,

and a compound of formula IV+TX

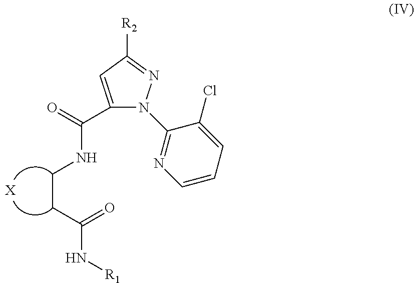

wherein X is a bivalent group selected from

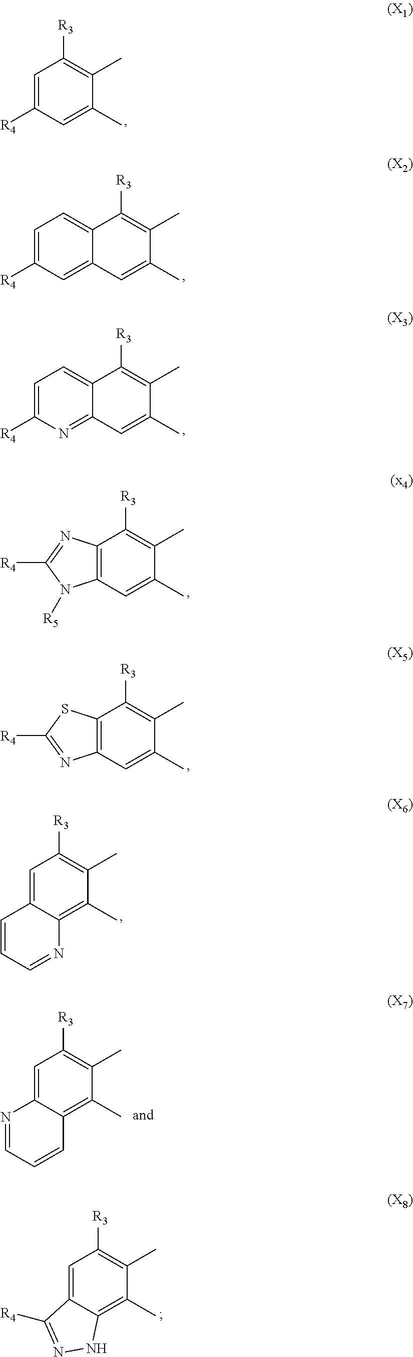

wherein

a) R₁ is cyclopropyl substituted by cyclopropyl at the 1-position, R₂ is bromine, R₃ is methyl, R₄ is CN and X is X₁;

b) R₁ is methyl substituted by cyclopropyl, R₂ is CF₃, R₃ is methyl, R₄ is C₁ and X is X₁;

c) R₁ is cyclopropyl substituted by cyclopropyl at the 1-position, R₂ is CF₃, R₃ is methyl, R₄ is Cland X is X₁;

d) R₁ is cyclopropyl substituted by cyclopropyl at the 1-position, R₂ is CF₃, R₃ is methyl, R₄ is CN and X is X₁;

e) R₁ is cyclopropyl substituted by cyclopropyl at the 1-position, R₂ is OCH₂CF₃, R₃ is methyl, R₄ is CN and X is X₁;

f) R₁ is isopropyl, R₂ is methoxy; R₃ is methyl, R₄ is hydrogen and X is X₈;

g) R₁ is isopropyl, R₂ is trifluoromethyl, R₃ is chlorine, R₄ is hydrogen and X is X₈;

h) R₁ is isopropyl, R₂ is trifluoromethyl, R₃ is methyl, R₄ is hydrogen and X is X₈;

i) R₁ is methyl, R₂ is bromine, R₃ is methyl, R₄ is CN and X is X₁;

j) R₁ is methyl, R₂ is bromine, R₃ is methyl, R₄ is C₁ and X is X₁;

and (B8) glyphosate+TX, glyphosate diammonium+TX, glyphosate dimethylammonium+TX, glyphosate isopropylammonium+TX, glyphosate monoammonium+TX, glyphosate potassium+TX, glyphosate sesquisodium+TX, glyphosate trimesium+TX, (5-chloro-2,4-dimethyl-pyridin-3-yl)-(2,3,4-trimethoxy-6-methyl-phenyl)-methanone+TX, (5-bromo-4-chloro-2-methoxy-pyridin-3-yl)-(2,3,4-trimethoxy-6-methyl-phenyl)-methanone+TX, 2-{2-[(E)-3-(2,6-Dichloro-phenyl)-1-methyl-prop-2-en-(E)-ylideneaminooxymethyl]-phenyl}-2-[(Z)-methoxyimino]-N-methyl-acetamide+TX, 3-[5-(4-Chloro-phenyl)-2,3-dimethyl-isoxazolidin-3-yl]-pyridine+TX

a compound of formula V+TX

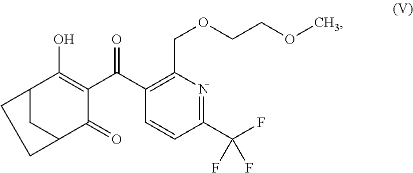 [SMILES: COCCOCc1nc(C(F)(F)F)ccc1C(=O)C1=C(O)C2CCC(C2)C1=O]

fomesafen+TX, Glufosinate and its salts TX, and (B9) Isopyrazam+TX, Sedaxane+TX,

a compound of formula (VI)+TX

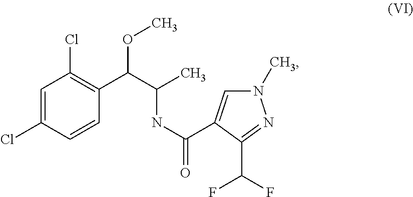 [SMILES: COC(c1ccc(Cl)cc1Cl)C(C)NC(=O)c1cn(C)nc1C(F)F]

a compound of formula (VII)+TX

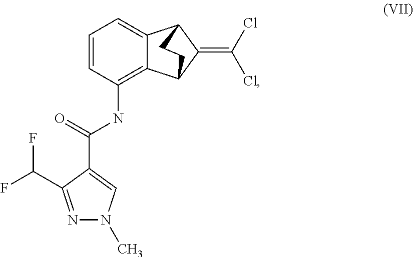 [SMILES: Cn1cc(C(=O)Nc2cccc3c2[C@@H]2CC[C@H]3C2=C(Cl)Cl)c(C(F)F)n1]

Preferred compositions comprising a compound of formula TX and

(B) a compound selected from the group consisting of

(B1) a strobilurin fungicide+TX, (B2) an azole fungicide+TX, (B3) a morpholine fungicide+TX, (B4) an anilinopyrimidine fungicide+TX, (B5) a fungicide selected from the group consisting of

anilazine (878)+TX, arsenates+TX, benalaxyl (56)+TX, benalaxyl-M+TX, benodanil (896)+TX, benomyl (62)+TX, benthiavalicarb+TX, benthiavalicarb-isopropyl (68)+TX, biphenyl (81)+TX, bitertanol (84)+TX, blasticidin-S (85)+TX, bordeaux mixture (87)+TX, boscalid (88)+TX, bupirimate (98)+TX, cadmium chloride+TX, captafol (113)+TX,

captan (114)+TX, carbendazim (116)+TX, carbon disulfide (945)+TX, carboxin (120)+TX, carpropamid (122)+TX, cedar leaf oil+TX, chinomethionat (126)+TX, chlorine+TX, chloroneb (139)+TX, chlorothalonil (142)+TX, chlozolinate (149)+TX, cinnamaldehyde+TX, copper+TX, copper ammoniumcarbonate+TX, copper hydroxide (169)+TX, copper octanoate (170)+TX, copper oleate+TX, copper sulphate (87)+TX, cyazofamid (185)+TX, cycloheximide (1022)+TX, cymoxanil (200)+TX, dichlofluanid (230)+TX, dichlone (1052)+TX, dichloropropene (233)+TX, diclocymet (237)+TX, diclomezine (239)+TX, dicloran (240)+TX, diethofencarb (245)+TX, diflumetorim (253)+TX, dimethirimol (1082)+TX, dimethomorph (263)+TX, dinocap (270)+TX, dithianon (279)+TX, dodine (289)+TX, edifenphos (290)+TX, ethaboxam (304)+TX, ethirimol (1133)+TX, etridiazole (321)+TX, famoxadone (322)+TX, fenamidone (325)+TX, fenaminosulf (1144)+TX, fenamiphos (326)+TX, fenarimol (327)+TX, fenfuram (333)+TX, fenhexamid (334)+TX, fenoxanil (338)+TX, fenpiclonil (341)+TX, fentin acetate (347)+TX, fentin chloride+TX, fentin hydroxide (347)+TX, ferbam (350)+TX, ferimzone (351)+TX, fluazinam (363)+TX, fludioxonil (368)+TX, flusulfamide (394)+TX, flutolanil (396)+TX, folpet (400)+TX, formaldehyde (404)+TX, fosetyl-aluminium (407)+TX, fthalide (643)+TX, fuberidazole (419)+TX, furalaxyl (410)+TX, furametpyr (411)+TX, flyodin (1205)+TX, fuazatine (422)+TX, hexachlorobenzene (434)+TX, hymexazole+TX, iminoctadine (459)+TX, iodocarb (3-Iodo-2-propynyl butyl carbamate)+TX, iprobenfos (IBP) (469)+TX, iprodione (470)+TX, iprovalicarb (471)+TX, isoprothiolane (474)+TX, kasugamycin (483)+TX, mancozeb (496)+TX, maneb (497)+TX, manganous dimethyldithiocarbamate+TX, mefenoxam (Metalaxyl-M) (517)+TX, mepronil (510)+TX, mercuric chloride (511)+TX, mercury+TX, metalaxyl (516)+TX, methasulfocarb (528)+TX, metiram (546)+TX, metrafenone+TX, nabam (566)+TX, neem oil (hydrophobic extract)+TX, nuarimol (587)+TX, octhilinone (590)+TX, ofurace (592)+TX, oxadixyl (601)+TX, oxine copper (605)+TX, oxolinic acid (606)+TX, oxycarboxin (608)+TX, oxytetracycline (611)+TX, paclobutrazole (612)+TX, paraffin oil (628)+TX, paraformaldehyde+TX, pencycuron (620)+TX, pentachloronitrobenzene (716)+TX, pentachlorophenol (623)+TX, penthiopyrad+TX, perfurazoate+TX, phosphoric acid+TX, polyoxin (654)+TX, polyoxin D zinc salt (654)+TX, potassium bicarbonate+TX, probenazole (658)+TX, procymidone (660)+TX, propamocarb (668)+TX, propineb (676)+TX, proquinazid (682)+TX, prothiocarb (1361)+TX, pyrazophos (693)+TX, pyrifenox (703)+TX, pyroquilon (710)+TX, quinoxyfen (715)+TX, quintozene (PCNB) (716)+TX, silthiofam (729)+TX, sodium bicarbonate+TX, sodium diacetate+TX, sodium propionate+TX, streptomycin (744)+TX, sulphur (754)+TX, TCMTB+TX, tecloftalam+TX, tecnazene (TCNB) (767)+TX, thiabendazole (790)+TX, thifluzamide (796)+TX, thiophanate (1435)+TX, thiophanate-methyl (802)+TX, thiram (804)+TX, tolclofos-methyl (808)+TX, tolylfluanid (810)+TX, triazoxide (821)+TX, trichoderma harzianum (825)+TX, tricyclazole (828)+TX, triforine (838)+TX, triphenyltin hydroxide (347)+TX, validamycin (846)+TX, vinclozolin (849)+TX, zineb (855)+TX, ziram (856)+TX, zoxamide (857)+TX, 1,1-bis(4-chlorophenyl)-2-ethoxyethanol (IUPAC-Name) (910)+TX, 2 35+TX, 4-dichlorophenyl benzenesulfonate (IUPAC-/Chemical Abstracts-Name) (1059)+TX, 2-fluoro-N-methyl-N-1-naphthylacetamide (IUPAC-Name) (1295)+TX, 4-chlorophenyl phenyl sulfone (IUPAC-Name) (981)+TX,

a compound of formula B-5.1+TX

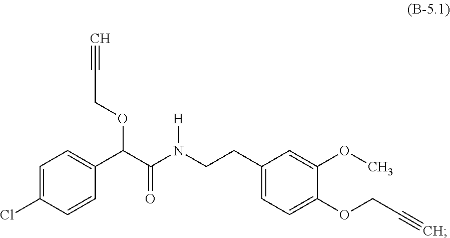 [SMILES: C#CCOc1ccc(CCNC(=O)C(OCC#C)c2ccc(Cl)cc2)cc1OC]

a compound of formula B-5.2+TX

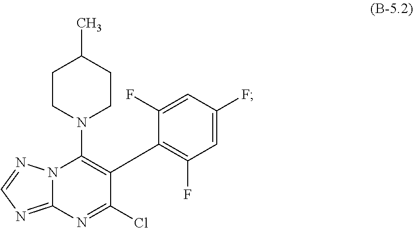 [SMILES: CC1CCN(c2c(-c3c(F)cc(F)cc3F)c(Cl)nc3ncnn23)CC1]

a compound of formula B-5.3+TX

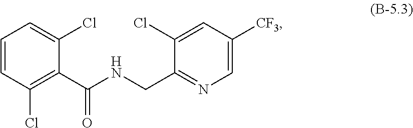 [SMILES: O=C(NCc1ncc(C(F)(F)F)cc1Cl)c1c(Cl)cccc1Cl]

a compound of formula B-5.4+TX

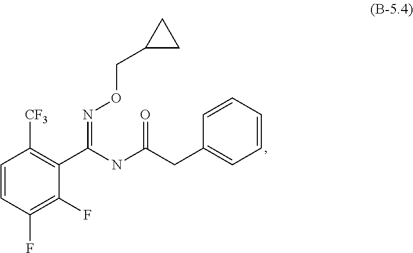 [SMILES: O=C(Cc1ccccc1)N/C(=N\OCC1CC1)c1c(C(F)(F)F)ccc(F)c1F]

a compound of formula B-5.5+TX

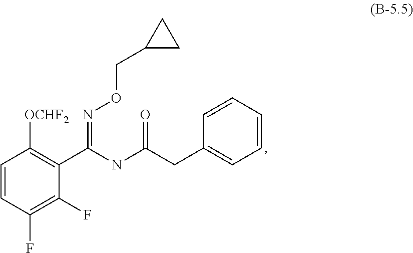 [SMILES: O=C(Cc1ccccc1)N/C(=N\OCC1CC1)c1c(OC(F)F)ccc(F)c1F]

a compound of formula B-5.6+TX

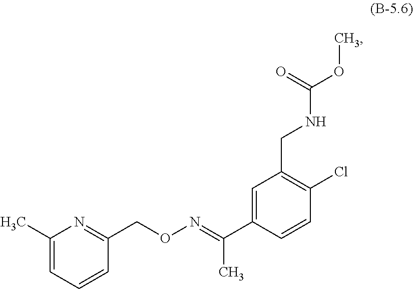 [SMILES: COC(=O)NCc1cc(/C(C)=N/OCc2cccc(C)n2)ccc1Cl]

a compound of formula B-5.7+TX

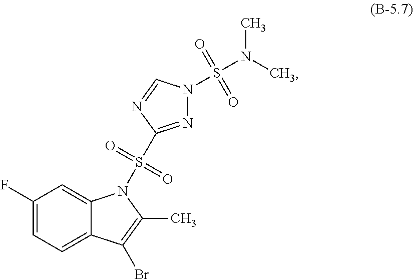 [SMILES: Cc1c(Br)c2ccc(F)cc2n1S(=O)(=O)c1ncn(S(=O)(=O)N(C)C)n1]

3-difluoromethyl-1-methyl-1H-pyrazole-4-carboxylic acid (2-bicyclopropyl-2-yl-phenyl)-amide (compound B-5.8)+TX, 3-difluoromethyl-1-methyl-1H-pyrazole-4-carboxylic acid (9-isopropyp-1,2,3,4-tetrahydro-1,4-methano-naphthalen-5-yl)-amide (compound B-5.9)+TX, 1,3-dimethyl-5-fluoro-1H-pyrazole-4-carboxylic acid [2-(1,3-dimethylbutyl)phenyl]-amide (compound B-5.10)+TX, 3-difluoromethyl-1-methyl-1H-pyrazole-4-carboxylic acid (3′,4′-dichloro-5-fluoro-1,1′-biphenyl-2-yl)-amide (compound B-5.11)+TX, N-{2-[3-chloro-5-(trifluoromethyl)pyridin-2-yl]ethyl}-2-(trifluoromethyl)benzamid (compound B-5.12)+TX, 3-difluoromethyl-1-methyl-1H-pyrazole-4-carboxylic acid N-[2-(1,1,2,2-tetrafluoroethoxy)phenyl]-amide (compound B-5.13)+TX, 3-difluoromethyl-1-methyl-1H-pyrazole-4-carboxylic acid N-[2-(1,1,2,3,3,3-hexafluoropropoxy)phenyl]-amide (compound B-5.14)+TX, 3-difluoromethyl-1-methyl-1H-pyrazole-4-carboxylic acid N-[2-(2-chloro-1,1,2-trifluoroethoxy)phenyl]-amide (compound B-5.15)+TX, 3-difluoromethyl-1-methyl-1H-pyrazole-4-carboxylic acid N-(4′-trifluoromethyl-biphen-2-yl)-amide (compound B-5.16)+TX, 3-difluoromethyl-1-methyl-1H-pyrazole-4-carboxylic acid N-(2′-trifluoromethyl-biphen-2-yl)-amide (compound B-5.17)+TX, and 3-difluoromethyl-1-methyl-1H-pyrazole-4-carboxylic acid N-(2′-trifluoromethyl-biphen-2-yl)-amide (compound B-5.18)+TX

(B6) a plant-bioregulator selected from the group consisting of

acibenzolar-S-methyl (6)+TX, chlormequat chloride (137)+TX, ethephon (307)+TX, mepiquat chloride (509) and trinexapc-ethyl (841);

(B7) an insecticide selected from the group consisting of

abamectin (1)+TX, clothianidin (165)+TX, emamectin benzoate (291)+TX, imidacloprid (458)+TX, tefluthrin (769)+TX, thiamethoxam (792)+TX, a compound of formula B-7.1+TX

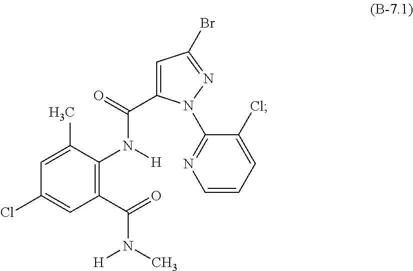 [SMILES: CNC(=O)c1cc(Cl)cc(C)c1NC(=O)c1cc(Br)nn1-c1ncccc1Cl]

and a compound of formula B-7.2+TX;

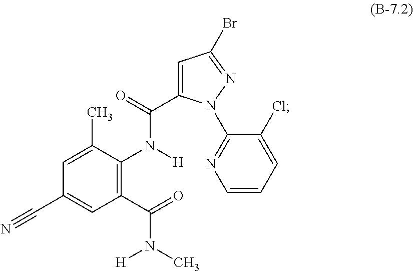 [SMILES: CNC(=O)c1cc(C#N)cc(C)c1NC(=O)c1cc(Br)nn1-c1ncccc1Cl]

and (B8) glyphosate (419)+TX.

Examples of especially suitable mixtures selected from the following group P

Group P: Especially Suitable Mixtures According to the Invention:

a strobilurin fungicide selected from azoxystrobin (47)+TX, dimoxystrobin (226)+TX, fluoxastrobin (382)+TX, kresoxim-methyl (485)+TX, metominostrobin (551)+TX, orysastrobin+TX, picoxystrobin (647)+TX, pyraclostrobin (690); trifloxystrobin (832)+TX, a compound of formula B-1.1+TX

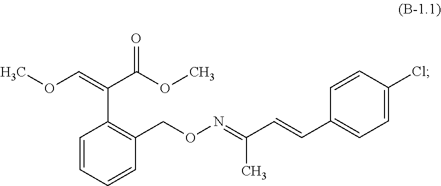 [SMILES: CO/C=C(/C(=O)OC)c1ccccc1CO/N=C(C)/C=C/c1ccc(Cl)cc1]

an azole fungicide selected from azaconazole (40)+TX, bromuconazole (96)+TX, cyproconazole (207)+TX, difenoconazole (247)+TX, diniconazole (267)+TX, diniconazole-M (267)+TX, epoxiconazole (298)+TX, fenbuconazole (329)+TX, fluquinconazole (385)+TX, flusilazole (393)+TX, flutriafol (397)+TX, hexaconazole (435)+TX, imazalil (449)+TX, imibenconazole (457)+TX, ipconazole (468)+TX, metconazole (525)+TX, myclobutanil (564)+TX, oxpoconazole (607)+TX, pefurazoate (618)+TX, penconazole (619)+TX, prochloraz (659)+TX, propiconazole (675)+TX, prothioconazole (685)+TX, simeconazole (731)+TX, tebuconazole (761)+TX, tetraconazole (778)+TX, triadimefon (814)+TX, triadimenol (815)+TX, triflumizole (834)+TX, triticonazole (842)+TX, diclobutrazol (1068)+TX, etaconazole (1129)+TX, furconazole (1198)+TX, furconazole-cis (1199) and quinconazole (1378);

a morpholine fungicide mixture selected from aldimorph+TX, dodemorph (288)+TX, fenpropimorph (344)+TX, tridemorph (830)+TX, fenpropidin (343)+TX, spiroxamine (740)+TX, piperalin (648) and a compound of formula B-3.1+TX

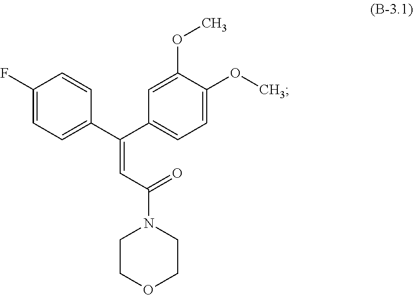 [SMILES: COc1ccc(/C(=C\C(=O)N2CCOCC2)c2ccc(F)cc2)cc1OC]

an anilino-pyrimidine fungicide selected from cyprodinil (208)+TX, mepanipyrim (508) and pyrimethanil (705);

a fungicide mixture selected from the group consisting of

anilazine (878)+TX, arsenates+TX, benalaxyl (56)+TX, benalaxyl-M+TX, benodanil (896)+TX, benomyl (62)+TX, benthiavalicarb+TX, benthiavalicarb-isopropyl (68)+TX, biphenyl (81)+TX, bitertanol (84)+TX, blasticidin-S (85)+TX, bordeaux mixture (87)+TX, boscalid (88)+TX, bupirimate (98)+TX, cadmium chloride+TX, captafol (113)+TX,

captan (114)+TX, carbendazim (116)+TX, carbon disulfide (945)+TX, carboxin (120)+TX, carpropamid (122)+TX, cedar leaf oil+TX, chinomethionat (126)+TX, chlorine+TX, chloroneb (139)+TX, chlorothalonil (142)+TX, chlozolinate (149)+TX, cinnamaldehyde+TX, copper+TX, copper ammoniumcarbonate+TX, copper hydroxide (169)+TX, copper octanoate (170)+TX, copper oleate+TX, copper sulphate (87)+TX, cyazofamid (185)+TX, cycloheximide (1022)+TX, cymoxanil (200)+TX, dichlofluanid (230)+TX, dichlone (1052)+TX, dichloropropene (233)+TX, diclocymet (237)+TX, diclomezine (239)+TX, dicloran (240)+TX, diethofencarb (245)+TX, diflumetorim (253)+TX, dimethirimol (1082)+TX, dimethomorph (263)+TX, dinocap (270)+TX, dithianon (279)+TX, dodine (289)+TX, edifenphos (290)+TX, ethaboxam (304)+TX, ethirimol (1133)+TX, etridiazole (321)+TX, famoxadone (322)+TX, fenamidone (325)+TX, fenaminosulf (1144)+TX, fenamiphos (326)+TX, fenarimol (327)+TX, fenfuram (333)+TX, fenhexamid (334)+TX, fenoxanil (338)+TX, fenpiclonil (341)+TX, fentin acetate (347)+TX, fentin chloride+TX, fentin hydroxide (347)+TX, ferbam (350)+TX, ferimzone (351)+TX, fluazinam (363)+TX, fludioxonil (368)+TX, flusulfamide (394)+TX, flutolanil (396)+TX, folpet (400)+TX, formaldehyde (404)+TX, fosetyl-aluminium (407)+TX, fthalide (643)+TX, fuberidazole (419)+TX, furalaxyl (410)+TX, furametpyr (411)+TX, flyodin (1205)+TX, fuazatine (422)+TX, hexachlorobenzene (434)+TX, hymexazole+TX, iminoctadine (459)+TX, iodocarb (3-Iodo-2-propynyl butyl carbamate)+TX, iprobenfos (IBP) (469)+TX, iprodione (470)+TX, iprovalicarb (471)+TX, isoprothiolane (474)+TX, kasugamycin (483)+TX, mancozeb (496)+TX, maneb (497)+TX, manganous dimethyldithiocarbamate+TX, mefenoxam (Metalaxyl-M) (517)+TX, mepronil (510)+TX, mercuric chloride (511)+TX, mercury+TX, metalaxyl (516)+TX, methasulfocarb (528)+TX, metiram (546)+TX, metrafenone+TX, nabam (566)+TX, neem oil (hydrophobic extract)+TX, nuarimol (587)+TX, octhilinone (590)+TX, ofurace (592)+TX, oxadixyl (601)+TX, oxine copper (605)+TX, oxolinic acid (606)+TX, oxycarboxin (608)+TX, oxytetracycline (611)+TX, paclobutrazole (612)+TX, paraffin oil (628)+TX, paraformaldehyde+TX, pencycuron (620)+TX, pentachloronitrobenzene (716)+TX, pentachlorophenol (623)+TX, penthiopyrad+TX, perfurazoate+TX, phosphoric acid+TX, polyoxin (654)+TX, polyoxin D zinc salt (654)+TX, potassium bicarbonate+TX, probenazole (658)+TX, procymidone (660)+TX, propamocarb (668)+TX, propineb (676)+TX, proquinazid (682)+TX, prothiocarb (1361)+TX, pyrazophos (693)+TX, pyrifenox (703)+TX, pyroquilon (710)+TX, quinoxyfen (715)+TX, quintozene (PCNB) (716)+TX, silthiofam (729)+TX, sodium bicarbonate+TX, sodium diacetate+TX, sodium propionate+TX, streptomycin (744)+TX, sulphur (754)+TX, TCMTB+TX, tecloftalam+TX, tecnazene (TCNB) (767)+TX, thiabendazole (790)+TX, thifluzamide (796)+TX, thiophanate (1435)+TX, thiophanate-methyl (802)+TX, thiram (804)+TX, tolclofos-methyl (808)+TX, tolylfluanid (810)+TX, triazoxide (821)+TX, trichoderma harzianum (825)+TX, tricyclazole (828)+TX, triforine (838)+TX, triphenyltin hydroxide (347)+TX, validamycin (846)+TX, vinclozolin (849)+TX, zineb (855)+TX, ziram (856)+TX, zoxamide (857)+TX, 1+TX, 1-bis(4-chlorophenyl)-2-ethoxyethanol (IUPAC-Name) (910)+TX, 2+TX, 4-dichlorophenyl benzenesulfonate (IUPAC-/Chemical Abstracts-Name) (1059)+TX, 2-fluoro-N-methyl-N-1-naphthylacetamide (IUPAC-Name) (1295)+TX, 4-chlorophenyl phenyl sulfone (IUPAC-Name) (981)+TX,

a compound of formula B-5.1+TX, a compound of formula B-5.2+TX, a compound of formula B-5.3+TX, a compound of formula B-5.4+TX, a compound of formula B-5.5+TX,

a compound of formula B-5.6+TX, a compound of formula B-5.7+TX, compound B-5.8+TX, compound B-5.9+TX, compound B-5.10+TX, compound B-5.11+TX, compound B-5.12+TX, compound B-5.13+TX, compound B-5.14+TX, compound B-5.15+TX, compound B-5.16+TX, compound B-5.17 and compound B-5.18;

a plant-bioregulator selected from the group consisting of

acibenzolar-S-methyl (6)+TX, chlormequat chloride (137)+TX, ethephon (307)+TX, mepiquat chloride (509) and trinexapc-ethyl (841);

an insecticide selected from the group consisting of

abamectin (1)+TX, clothianidin (165)+TX, emamectin benzoate (291)+TX, imidacloprid (458)+TX, tefluthrin (769)+TX, thiamethoxam (792)+TX, and glyphosate (419)+TX, a compound of formula V)+TX

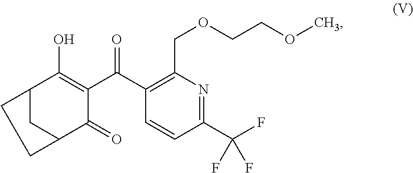 [SMILES: COCCOCc1nc(C(F)(F)F)ccc1C(=O)C1=C(O)C2CCC(C2)C1=O]

fomesafen+TX, and (B9) Isopyrazam+TX, Sedaxane+TX,

a compound of formula (VI)+TX

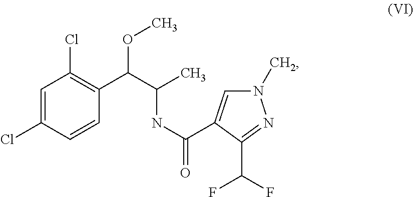

a compound of formula (VII)+TX

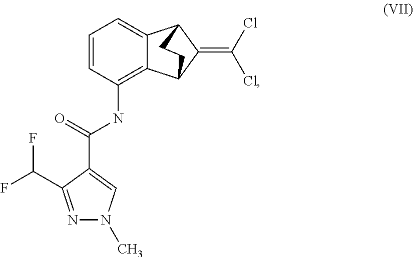 [SMILES: Cn1cc(C(=O)Nc2cccc3c2[C@@H]2CC[C@H]3C2=C(Cl)Cl)c(C(F)F)n1]

Further examples of especially suitable mixtures selected from the following group Q:

Group Q: Especially Suitable Compositions According to the Invention:

a strobilurin fungicide selected from the group consisting of azoxystrobin+TX, dimoxystrobin+TX, fluoxastrobin+TX, kresoxim-methyl+TX, metominostrobin+TX, orysastrobin+TX, picoxystrobin+TX, pyraclostrobin; trifloxystrobin and a compound of formula B-1.1;

an azole fungicide selected from the group consisting of azaconazole+TX, bromuconazole+TX, cyproconazole+TX, difenoconazole+TX, diniconazole+TX, diniconazole-M+TX, epoxiconazole+TX, fenbuconazole+TX, fluquinconazole+TX, flusilazole+TX, flutriafol+TX, hexaconazole+TX, imazalil+TX, imibenconazole+TX, ipconazole+TX, metconazole+TX, myclobutanil+TX, oxpoconazole+TX, pefurazoate+TX, penconazole+TX, prochloraz+TX, propiconazole+TX, prothioconazole+TX, simeconazole+TX, tebuconazole+TX, tetraconazole+TX, triadimefon+TX, triadimenol+TX, triflumizole+TX, triticonazole+TX, diclobutrazol+TX, etaconazole+TX, furconazole+TX, furconazole-cis+TX and quinconazole+TX;

a morpholine fungicide selected from the group consisting of aldimorph+TX, dodemorph+TX, fenpropimorph+TX, tridemorph+TX, fenpropidin+TX, spiroxamine+TX, piperalin and a compound of formula B-3.1;

an anilino-pyrimidine fungicide selected from the group consisting of cyprodinil+TX, mepanipyrim and pyrimethanil;

a fungicide selected from the group consisting of benalaxyl+TX, benalaxyl-M+TX, benomyl+TX, bitertanol+TX, boscalid+TX, captan+TX, carboxin+TX, carpropamid+TX, chlorothalonil+TX, copper+TX, cyazofamid+TX, cymoxanil+TX, diethofencarb+TX, dithianon+TX, famoxadone+TX, fenamidone+TX, fenhexamide+TX, fenoxycarb+TX, fenpiclonil+TX, fluazinam+TX, fludioxonil+TX, flutolanil+TX, folpet+TX, guazatine+TX, hymexazole+TX, iprodione+TX, lufenuron+TX, mancozeb+TX, metalaxyl+TX, mefenoxam+TX, metrafenone+TX, nuarimol+TX, paclobutrazol+TX, pencycuron+TX, penthiopyrad+TX, procymidone+TX, proquinazid+TX, pyroquilon+TX, quinoxyfen+TX, silthiofam+TX, sulfur+TX, thiabendazole+TX, thiram+TX, triazoxide+TX, tricyclazole+TX, a compound of formula B-5.1+TX, a compound of formula B-5.2+TX, a compound of formula B-5.3+TX, a compound of formula B-5.4+TX, a compound of formula B-5.5+TX, a compound of formula B-5.6+TX, a compound of formula B-5.7+TX, a compound of formula B-5.8+TX, a compound of formula B-5.9+TX, a compound of formula B-5.10 and a compound of formula B-5.12;

a plant-bioregulator selected from acibenzolar-S-methyl+TX, chlormequat chloride+TX, ethephon+TX, mepiquat chloride and trinexapc-ethyl;

an insecticide selected from abamectin+TX, emamectin benzoate+TX, tefluthrin+TX, thiamethoxam+TX, and glyphosate+TX, a compound of formula V

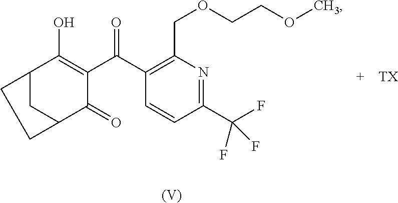

fomesafen+TX, and (B9) Isopyrazam+TX, Sedaxane+TX,

a compound of formula (VI)+TX

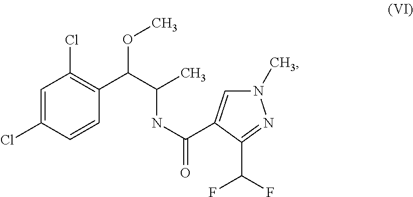 [SMILES: COC(c1ccc(Cl)cc1Cl)C(C)NC(=O)c1cn(C)nc1C(F)F]

a compound of formula (VII)+TX

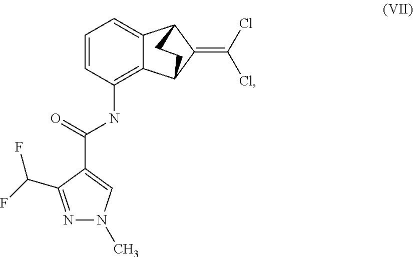 [SMILES: Cn1cc(C(=O)Nc2cccc3c2[C@@H]2CC[C@H]3C2=C(Cl)Cl)c(C(F)F)n1]

It has been found that the use of component (B) in combination with component TX surprisingly and substantially may enhance the effectiveness of the latter against fungi, and vice versa. Additionally, the method of the invention is effective against a wider spectrum of such fungi that can be combated with the active ingredients of this method, when used solely.

The active ingredient mixture of component TX to component (B) comprises compounds of formula I and a further, other biocidally active ingredients or compositions or if desired, a solid or liquid adjuvant preferably in a mixing ratio of from 100:1 to 1:6000, especially from 50:1 to 1:50, more especially in a ratio of from 20:1 to 1:20, even more especially from 10:1 to 1:10, very especially from 5:1 and 1:5, special preference being given to a ratio of from 2:1 to 1:2, and a ratio of from 4:1 to 2:1 being likewise preferred, above all in a ratio of 1:1, or 5:1, or 5:2, or 5:3, or 5:4, or 4:1, or 4:2, or 4:3, or 3:1, or 3:2, or 2:1, or 1:5, or 2:5, or 3:5, or 4:5, or 1:4, or 2:4, or 3:4, or 1:3, or 2:3, or 1:2, or 1:600, or 1:300, or 1:150, or 1:35, or 2:35, or 4:35, or 1:75, or 2:75, or 4:75, or 1:6000, or 1:3000, or 1:1500, or 1:350, or 2:350, or 4:350, or 1:750, or 2:750, or 4:750. Those mixing ratios are understood to include, on the one hand, ratios by weight and also, on other hand, molar ratios.

It has been found, surprisingly, that certain weight ratios of component TX to component (B) are able to give rise to synergistic activity. Therefore, a further aspect of the invention are compositions, wherein component TX and component (B) are present in the composition in amounts producing a synergistic effect. This synergistic activity is apparent from the fact that the fungicidal activity of the composition comprising component TX and component (B) is greater than the sum of the fungicidal activities of component TX and of component (B). This synergistic activity extends the range of action of component TX and component (B) in two ways. Firstly, the rates of application of component TX and component (B) are lowered whilst the action remains equally good, meaning that the active ingredient mixture still achieves a high degree of phytopathogen control even where the two individual components have become totally ineffective in such a low application rate range. Secondly, there is a substantial broadening of the spectrum of phytopathogens that can be controlled.

A synergistic effect exists whenever the action of an active ingredient combination is greater than the sum of the actions of the individual components. The action to be expected E for a given active ingredient combination obeys the so-called COLBY formula and can be calculated as follows (COLBY, S. R. “Calculating synergistic and antagonistic responses of herbicide combination”. Weeds, Vol. 15, pages 20-22; 1967):

ppm=milligrams of active ingredient (=a.i.) per liter of spray mixture

X=% action by active ingredient A) using p ppm of active ingredient

Y=% action by active ingredient B) using q ppm of active ingredient.

According to COLBY, the expected (additive) action of active ingredients A)+B) using p+q ppm of active ingredient is

$$E = X + Y - \frac{X \cdot Y}{100}$$

If the action actually observed (O) is greater than the expected action (E), then the action of the combination is super-additive, i.e. there is a synergistic effect. In mathematical terms, synergism corresponds to a positive value for the difference of (O-E). In the case of purely complementary addition of activities (expected activity), said difference (O-E) is zero. A negative value of said difference (O-E) signals a loss of activity compared to the expected activity.

However, besides the actual synergistic action with respect to fungicidal activity, the compositions according to the invention can also have further surprising advantageous properties. Examples of such advantageous properties that may be mentioned are: more advantageuos degradability; improved toxicological and/or ecotoxicological behaviour; or improved characteristics of the useful plants including: emergence, crop yields, more developed root system, tillering increase, increase in plant height, bigger leaf blade, less dead basal leaves, stronger tillers, greener leaf colour, less fertilizers needed, less seeds needed, more productive tillers, earlier flowering, early grain maturity, less plant verse (lodging), increased shoot growth, improved plant vigor, and early germination.

Some compositions according to the invention have a systemic action and can be used as foliar, soil and seed treatment fungicides.

With the compositions according to the invention it is possible to inhibit or destroy the phytopathogenic microorganisms which occur in plants or in parts of plants (fruit, blossoms, leaves, stems, tubers, roots) in different useful plants, while at the same time the parts of plants which grow later are also protected from attack by phytopathogenic microorganisms.

The compositions according to the invention can be applied to the phytopathogenic microorganisms, the useful plants, the locus thereof, the propagation material thereof, storage goods or technical materials threatened by microorganism attack.

The compositions according to the invention may be applied before or after infection of the useful plants, the propagation material thereof, storage goods or technical materials by the microorganisms.

A further aspect of the present invention is a method of controlling diseases on useful plants or on propagation material thereof caused by phytopathogens, which comprises applying to the useful plants, the locus thereof or propagation material thereof a composition according to the invention. Preferred is a method, which comprises applying to the useful plants or to the locus thereof a composition according to the invention, more preferably to the useful plants. Further preferred is a method, which comprises applying to the propagation material of the useful plants a composition according to the invention.

The components (B) are known. Where the components (B) are included in “The Pesticide Manual” [The Pesticide Manual—A World Compendium; Thirteenth Edition; Editor: C. D. S. Tomlin; The British Crop Protection Council], they are described therein under the entry number given in round brackets hereinabove for the particular component (B); for example, the compound “abamectin” is described under entry number (1). Most of the components (B) are referred to hereinabove by a so-called “common name”, the relevant “ISO common name” or another “common name” being used in individual cases. If the designation is not a “common name”, the nature of the designation used instead is given in round brackets for the particular component (B); in that case, the IUPAC name, the IUPAC/Chemical Abstracts name, a “chemical name”, a “traditional name”, a “compound name” or a “development code” is used or, if neither one of those designations nor a “common name” is used, an “alternative name” is employed.

The following components B) are registered under a CAS-Reg. No.

Fluconazole (86386-73-4), Fluconazole-cis (112839-32-4), Fluxapyroxad (907204-31-3), Ametoctradin (865318-97-4), Flutianil (958647-10-4), Isotianil (224049-04-1), Valiphenal (283159-90-0), Acibenzolar (126448-41-7), 1-methyl-cyclopropene (3100-04-7), glyphosate diammonium (69254-40-6), glyphosate dimethylammonium (34494-04-7), glyphosate isopropylammonium (38641-94-0), glyphosate monoammonium (40465-66-5), glyphosate potassium (70901-20-1), glyphosate sesquisodium (70393-85-0), glyphosate trimesium (81591-81-3), Glufosinate and its salts (51276-47-2, 35597-44-5 (S-isomer)), aldimorph (CAS 91315-15-0); arsenates (CAS 1327-53-3); benalaxyl-M (CAS 98243-83-5); benthiavalicarb (CAS 413615-35-7); cadmium chloride (CAS 10108-64-2); cedar leaf oil (CAS 8007-20-3); chlorine (CAS 7782-50-5); cinnamaldehyde (CAS: 104-55-2); copper ammoniumcarbonate (CAS 33113-08-5); copper oleate (CAS 1120-44-1); iodocarb (3-Iodo-2-propynyl butyl carbamate) (CAS 55406-53-6); hymexazole (CAS 10004-44-1); manganous dimethyldithiocarbamate (CAS 15339-36-3); mercury (CAS 7487-94-7; 21908-53-2; 7546-30-7); metrafenone (CAS 220899-03-6); neem oil (hydrophobic extract) (CAS 8002-65-1); orysastrobin CAS 248593-16-0); paraformaldehyde (CAS 30525-89-4); penthiopyrad (CAS 183675-82-3); phosphoric acid (CAS 7664-38-2); potassium bicarbonate (CAS 298-14-6); sodium bicarbonate (CAS 144-55-8); sodium diacetate (CAS 127-09-3); sodium propionate (CAS 137-40-6); TCMTB (CAS 21564-17-0); and tolyfluanid (CAS 731-27-1).

Compound B-1.1 (“enestrobin”) is described in EP-O-936-213; compound B-3.1 (“flumorph”) in U.S. Pat. No. 6,020,332, CN-1-167-568, CN-1-155-977 and in EP-O-860-438; compound B-5.1 (“mandipropamid”) in WO 01/87822; compound B-5.2 in WO 98/46607; compound B-5.3 (“fluopicolide”) in WO 99/42447; compound B-5.4 (“cyflufenamid”) in WO 96/19442; compound B-5.5 in WO 99/14187; compound B-5.6 (“pyribencarb”) is registered under CAS-Reg. No. 325156-49-8; compound B-5.7 (“amisulbrom” or “ambromdole”) is registered under CAS-Reg. No. 348635-87-0; compound B-5.8 (3-difluoromethyl-1-methyl-1H-pyrazole-4-carboxylic acid (2-bicyclopropyl-2-yl-phenyl)-amide) is described in WO 03/74491; compound B-5.9 (3-difluoromethyl-1-methyl-1H-pyrazole-4-carboxylic acid (9-isopropyp-1,2,3,4-tetrahydro-1,4-methano-naphthalen-5-yl)-amide) is described in WO 04/35589 and in WO 06/37632; compound B-5.10 (1,3-dimethyl-5-fluoro-1H-pyrazole-4-carboxylic acid [2-(1,3-dimethylbutyl)phenyl]-amide) is described in WO 03/10149; compound B-5.11 (3-difluoromethyl-1-methyl-1H-pyrazole-4-carboxylic acid (3′,4′-dichloro-5-fluoro-1,1′-biphenyl-2-yl)-amide; “bixafen”) is registered under CAS-Reg. No.: 581809-46-3 and described in WO 03/70705; compound B-5.12 (N-{2-[3-Chloro-5-(trifluoromethyl)pyridin-2-yl]ethyl}-2-(trifluoromethyl)benzamid; “fluopyram”) is registered under CAS-Reg. No: 658066-35-4 and described in WO 04/16088; compounds B-5.13, B-5.14 and B-5.15 are described in WO 2007/17450; compounds B-5.16, B-5.17 and B-5.18 are described in WO 2006/120219; The compounds of formula IV are for example described in WO 04/067528, WO 2005/085234, WO 2006/111341, WO 03/015519, WO 2007/020050, WO 2006/040113, and WO 2007/093402; The compound of formula V is described in WO 2001/094339; compound B-21 is described in WO 2010/123791. Isopyrazam (3-(difluoromethyl)-1-methyl-N-[1,2,3,4-tetrahydro-9-(1-methylethyl)-1,4-methanonaphthalen-5-yl]-1H-pyrazole-4-carboxamide) is described in WO 2004/035589, in WO 2006/037632 and in EP1556385B1 and is registered under the CAS-Reg. 881685-58-1. Sedaxane (N-[2-[1,1′-bicyclopropyl]-2-ylphenyl]-3-(difluoromethyl)-1-methyl-1H-pyrazole-4-carboxamide) is described in WO 2003/074491 and is registered under the CAS-Reg. 874967-67-6; The compound of formula (VI) is described in WO 2008/014870; and the compounds of formula (VII) is described in WO 2007/048556. Fomesafen is registered under the CAS-Reg. No. 72178-02-0.

3-Difluoromethyl-1-methyl-1H-pyrazole-4-carboxylic acid (4′-methylsulfanyl-biphenyl-2-yl)-amide (compound B-5.19) is registered under CAS number 1021864-46-9,3-difluoromethyl-1-methyl-1H-pyrazole-4-carboxylic acid (2-dichloromethylene-3-ethyl-1-methyl-indan-4-yl)-amide (compound B-5.20) is registered under CAS number

The compositions according to the invention may also comprise more than one of the active components (B), if, for example, a broadening of the spectrum of disease control is desired. For instance, it may be advantageous in the agricultural practice to combine two or three components (B) with component TX. An example is a composition comprising a compound of formula (I), azoxystrobin and cyproconazole.

In the above different lists of active ingredients to be mixed with a TX, the compound of the formula I is preferably a compound of Tables 1-15; and more preferably, a compound selected from

E/E-2.168, E/E-2.002, 2.557, E/E-2.474, 5.561, 2.561, E/E-2.508, 2.513, 2.559, 2.556, 2.555, 2.374, 2.544, 2.537, 2.530, 2.525 (fraction B), 2.519, 2.516, 2.514, E/E-5.167, E/E-3.002, 2.512, E/E-2.507, E/E-2.208, 2.552, E/E-14.002, E/E-2.507, P.57, 2.558, 2.541, 2.539, 2.536, 2.535, 2.533, 2.526, 2.510, 2.528, Z/E-2.474, E/E-2.509, 2.553, 2.551 (fraction A), 2.548, 2.545, 2.542, 2.540, 2.532, 2.531, 2.525 (fraction A), 2.524, 2.520, 2.518, 2.517, 2.551 (Fraction B) 2.511, 2.549, 2.523, 2.521

In the above-mentioned mixtures of compounds of formula I, in particular a compound selected from said Tables 1-15, with other insecticides, fungicides, herbicides, safeners, adjuvants and the like, the mixing ratios can vary over a large range and are, preferably 100:1 to 1:6000, especially 50:1 to 1:50, more especially 20:1 to 1:20, even more especially 10:1 to 1:10. Those mixing ratios are understood to include, on the one hand, ratios by weight and also, on other hand, molar ratios.

The mixtures can advantageously be used in the above-mentioned formulations (in which case “active ingredient” relates to the respective mixture of TX with the mixing partner).

Some mixtures may comprise active ingredients which have significantly different physical, chemical or biological properties such that they do not easily lend themselves to the same conventional formulation type. In these circumstances other formulation types may be prepared. For example, where one active ingredient is a water insoluble solid and the other a water insoluble liquid, it may nevertheless be possible to disperse each active ingredient in the same continuous aqueous phase by dispersing the solid active ingredient as a suspension (using a preparation analogous to that of an SC) but dispersing the liquid active ingredient as an emulsion (using a preparation analogous to that of an EW). The resultant composition is a suspoemulsion (SE) formulation.

The mixtures comprising a TX selected from Tables 1-15 and one or more active ingredients as described above can be applied, for example, in a single “ready-mix” form, in a combined spray mixture composed from separate formulations of the single active ingredient components, such as a “tank-mix”, and in a combined use of the single active ingredients when applied in a sequential manner, i.e. one after the other with a reasonably short period, such as a few hours or days. The order of applying the compounds of formula I selected from Tables 1-18 and the active ingredients as described above is not essential for working the present invention.

The following non-limiting Examples illustrate the above-described invention in greater detail without limiting it. Those skilled in the art will promptly recognize appropriate variations from the procedures both as to reactants and as to reaction conditions and techniques. All references mentioned herein are incorporated by reference in their entirety.

### PREPARATORY EXAMPLES

### Preparation of Compound E/E-14.002

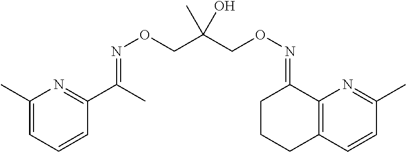

Step A) To a 100 mL single-necked round-bottomed flask, kept under an atmosphere of argon, was charged a solution of E-1-(6-methyl-2-pyridinyl)-ethanone oxime (6.00 g) in absolute acetone (35 mL). Under stirring, finely powdered NaOH (3.20 g) was added portionwise. Stirring was continued at room temperature for 4.5 hours, giving a light orange suspension. A solution of α-methylepichlorohydrin (6.08 g) dissolved in absolute acetone (5.00 mL) was then added to the flask slowly, using a syringe. The resulting mixture was then heated to reflux under stirring for 2.5 hours, when TLC indicated that the starting materials had been consumed. The suspension was cooled to room temperature and then filtered. The filter cake was washed with absolute acetone. Water (50 mL) was added to the filtrate, giving a pH in the range of 7-8. Extraction was carried out using ethyl acetate (2×100 ml). The combined organic phases were dried over sodium sulphate, filtered and the solvent removed in vacuo to give an orange oil (12.0 g). The crude material was purified by chromatography on silica gel (eluent: heptane/ethyl acetate 95:5 (v:v)). This gave 1-(6-methyl-pyridin-2-yl)-ethanone-O-(2-methyl-oxiranylmethyl)-oxime (7.35 g) in the form of a yellow oil.

LC-MS (Method ZCQ): UV Detection: 220 nm; Rₜ=1.21 min. MS: (M⁺+1)=221, (M⁺+23)=243.

TLC: Plates: Merck DC-Plates, silica gel F₂₅₄, saturated atmosphere in developing tank, UV detection, eluent: heptane/ethyl acetate 1:2 (v:v); R_{f} of title compound=0.37, R_{f} of oxime starting material=0.29.

Step B) A solution of 2-methyl-6,7-dihydro-5H-quinolin-8-one oxime (176 mg) in absolute dimethylsulfoxide (2.00 mL) was charged to a 10 mL single-necked round-bottomed flask. Under stirring NaOH (48 mg) was added to the flask. The reaction mixture was stirred at room temperature for 45 minutes. Then a solution of 1-(6-methyl-pyridin-2-yl)-ethanone O-(2-methyl-oxiranylmethyl)-oxime (220 mg) in absolute dimethylsulfoxide (0.50 mL) was slowly added to the reaction mixture. The resulting orange.brown solution was stirred at room temperature for 19 hours. The course of the reaction was followed by TLC, which indicated that substantial amounts of starting materials were consumed at this point in time. The reaction was quenched by the addition of water (10 mL), whereupon a pH in the range of 7-8 of the aqueous phase was observed. The solution was extracted using ethyl acetate (2×15 mL). The combined organic phases were dried over sodium sulphate, filtered and the solvent removed in vacuo to give an orange oil (280 mg). The crude material was partially purified by chromatography on silica gel (eluent: heptane/ethyl acetate 4:1 (v:v) with 1% v/v of triethylamine), giving a light orange gum (135 mg). In order to remove a remaining amount of the oxime starting material, a solution of the partially purified product in diethyl ether was extracted with an excess of 2 M aqueous NaOH solution. The ether phase was then dried over sodium sulphate, filtered and the solvent removed in vacuo to give of the title compound (88.5 mg) in the form of a light orange gum.

LC-MS (Method ZCQ): UV Detection: 220 nm; Rₜ=1.16 min. MS: (M⁺+1)=397, (M⁺+23)=419.

TLC: Plates: Merck DC-Plates, silica gel F₂₅₄, saturated atmosphere in developing tank, UV detection, eluent: heptane/ethyl acetate 1:2 (v:v); R_{f} of title compound=0.14, R_{f} of oxime starting material=0.13, R_{f} of epoxide starting material=0.51.

### Preparation of Compound E/E-2.507

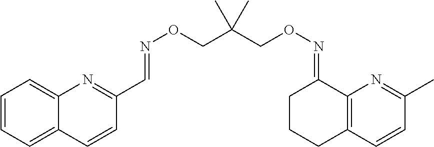 [SMILES: Cc1ccc2c(n1)/C(=N/OCC(C)(C)CO/N=C/c1ccc3ccccc3n1)CCC2]

Step A) A 500 mL reaction vessel was set under argon and then charged with a solution of hydroxylamine-O-[3-(aminooxy)-2,2-dimethylpropyl]hydrochloride (1:2) (21.7 g) in absolute ethanol (300 mL). Under stirring p-toluenesulphonic acid (1.2 g) was added, followed by the dropwise addition of a solution of 2-methyl-6,7-dihydro-5H-quinolin-8-one (6.77 g) in absolute ethanol (30 mL). The resulting yellow solution was stirred at room temperature for 1.5 hours, after which time TLC of the reaction mixture indicated that no starting materials were left. The ethanol was removed in vacuo, and aqueous sodium bicarbonate solution (150 mL) was added to the residue. The resulting solution was extracted using ethyl acetate (2×100 mL). The organic layer was dried over sodium sulphate, filtered and the solvent was removed in vacuo to give a beige oil (10.9 g). This crude material was purified by chromatography on silica gel (eluent: heptane/ethyl acetate 2:1 (v:v) with 1% v/v triethylamine). This was followed by RP-HPLC chromatography (Method A). E-2-methyl-6,7-dihydro-5H-quinolin-8-one-O-(3-aminooxy-2,2-dimethyl-propyl)-oxime (6.00 g) was obtained in the form of a light yellow gum.

LC-MS (Method ZMD): UV Detection: 220 nm; Rₜ=0.83 min.

TLC: Plates: Merck DC-Plates, silica gel F₂₅₄, saturated atmosphere in developing tank, UV detection, eluent: heptane/ethyl acetate 1:2 (v:v); R_{f} of title compound=0.11.

Step B) A solution of E-2-methyl-6,7-dihydro-5H-quinolin-8-one-O-(3-aminooxy-2,2-dimethyl-propyl)-oxime (80 mg) in absolute ethanol (2.00 mL) was charged to a 10 mL single-necked round-bottomed flask. Under stirring, p-toluenesulphonic acid (3.3 mg) was added, followed by the addition of 2-quinoline-carboxaldehyde (45.3 mg). The resulting yellow solution was stirred at room temperature for 2 hours, after which time TLC indicated that no starting materials were left. The ethanol was removed in vacuo, and water (2.00 mL) was added to the residue. The pH was adjusted to pH 8-9 using aqueous 2 M NaOH solution. The resulting solution was extracted carried out using ethyl acetate (2×10 mL). The combined organic phases were dried over sodium sulphate, filtered and the solvent was removed in vacuo to give 119.4 mg of a yellow gum. This crude material was purified by chromatography on silica gel (eluent: heptane/ethyl acetate 2:1 (v:v) with 1% v/v triethylamine). This gave the title compound (95.2 mg) as a light yellow gum.

LC-MS (Method ZCQ): UV Detection: 220 nm; Rₜ=1.61 min. MS: (M⁺+1)=417, (M²⁺+2)=209.

TLC: Plates: Merck DC-Plates, silica gel F₂₅₄, saturated atmosphere in developing tank, UV detection, eluent: heptane/ethyl acetate 1:2 (v:v); R_{f} of title compound=0.34, R_{f} of aldehyde starting material=0.54.

### Preparation of Compound E/E-2.002

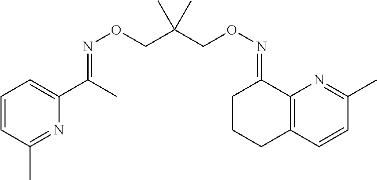 [SMILES: C/C(=N\OCC(C)(C)CO/N=C1\CCCc2ccc(C)nc21)c1cccc(C)n1]

Step A) Hydroxylamine-O—[3-(aminooxy)-2,2-dimethylpropyl]-hydrochloride (1:2) (750 mg) was charged to a 25 mL single-necked round-bottomed flask containing absolute ethanol (15.0 mL), forming a suspension. Under stirring, p-toluenesulphonic acid (19 mg) was added to the reaction mixture, followed by the dropwise addition of a solution of 2-acetyl-6-methylpyridine (227 mg) in absolute ethanol (2.00 mL). The resulting mixture was stirred at room temperature for 1.5 hours to give a light-yellow solution, at which time analysis of the reaction mixture by TLC indicated that starting materials were consumed. The ethanol was removed in vacuo, then water (2.00 mL) was added to the residue. The pH was adjusted to pH 7-8 using a small amount of triethylamine. The resulting solution was extracted using dichloromethane (2×30 ml). The organic layer was dried over sodium sulphate, filtered and the solvent removed in vacuo to give an oil (665 mg). The crude material was purified by chromatography on silica gel (eluent: heptane/ethyl acetate gradient from 98:2 to 95:5 (v:v)). This gave (1E)-1-(6-methyl-pyridin-2-yl)-ethanone-O-(3-aminooxy-2,2-dimethyl-propyl)-oxime (300 mg) as a colourless oil.

LC-MS (ZMD): UV Detection: 220 nm; Rₜ=1.05 min. MS: (M⁺+1) 252

TLC: Plates: Merck DC-Plates, silica gel F₂₅₄, saturated atmosphere in developing tank, UV detection, eluent: heptane/ethyl acetate 4:1 (v:v); R_{f} of title compound=0.29, R_{f} of ketone starting material=0.54.

Step B) A solution of E-1-(6-methyl-pyridin-2-yl)-ethanone-O-(3-aminooxy-2,2-dimethyl-propyl)-oxime (40 mg) in absolute ethanol (2.00 mL) was charged to a 5 mL single-necked round-bottomed flask. Under stirring, p-toluenesulphonic acid (1.8 mg) was added to the reaction vessel, followed by the dropwise addition of a solution of 2-methyl-6,7-dihydro-5H-quinolin-8-one (26 mg) in absolute ethanol (2.00 mL). The resulting light-yellow solution was stirred at room temperature for 1.5 hours, after which time TLC indicated that no starting materials were remaining. The ethanol was removed in vacuo and water (2.00 mL) was added to the residue. The pH was adjusted to pH 7-8 using a trace of triethylamine. The resulting solution was extracted using dichloromethane (2×30 mL). The organic layer was dried over sodium sulphate, filtered and the solvent was removed in vacuo to give a beige oil (53 mg). The crude material was purified by chromatography on silica gel (eluent: heptane/ethyl acetate gradient from 98:2 to 95:5 (v:v)). This gave the title compound (25 mg) as a colourless oil.

LC-MS (ZCQ): UV Detection: 220 nm; Rₜ=1.53 min. MS: (M⁺+1)=395, (M²⁺+2)=198.

TLC: Plates: Merck DC-Plates, silica gel F₂₅₄, saturated atmosphere in developing tank, UV detection, eluent: heptane/ethyl acetate 4:1 (v:v); R_{f} of title compound=0.29, R_{f} of ketone starting material=0.55.

### Preparation of Compound 2.569

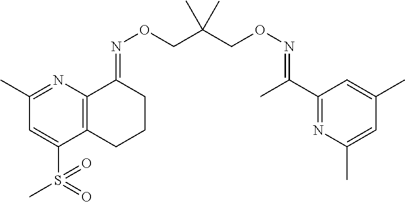

Step A) 2-Methyl-4-methylsulfanyl-5,6,7,8-tetrahydro-quinolin-8-ol:

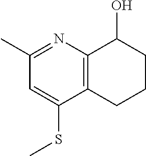

A flask equipped with a condenser was charged with a mixture of 4-chloro-2-methyl-5,6,7,8-tetrahydro-quinolin-8-ol (0.9 g; 4.55 mmol) in DMF (27 mL). Under stirring sodium methanethiolate (1.6 g; 22.77 mmol) was added and the resulting mixture was stirred under heating to reflux for 3 hours. The resulting solution was cooled to room temperature and diluted with water and 2M aqueous NaOH. The reaction mixture was extracted with diethyl ether. The combined organic phases were dried over sodium sulphate, filtered and the solvent was removed in vacuo to give a yellow gum (600 mg) which was used as such for the next step.

LC-MS (ZCQ) UV Detection: 220 nm; Rₜ=0.23, MS: (M⁺+1)=210.

Step B) 2-Methyl-4-methylsulfanyl-6,7-dihydro-5H-quinolin-8-one:

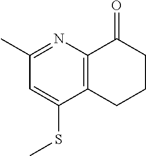

A flask, equipped with a condenser was charged with a mixture of 2-methyl-4-methylsulfanyl-5,6,7,8-tetrahydro-quinolin-8-ol (500 mg; 2.4 mmol) in chloroform (10 mL). Under stirring, manganese (IV) oxide (830 mg) was added and the resulting black suspension was stirred under heating to reflux for 18 hours, after which time TLC indicated that no starting material remained. The resulting black material was allowed to return to ambient temperature and filtered over hyflo before purification by chromatography on silica gel (eluent: heptane/ethyl acetate). This gave the title compound (420 mg) as an orange solid. LC-MS (ZMD): UV Detection: 220 nm; Rₜ=0.2 min. MS: (M⁺+1)=208.

Step C) 4-Methanesulfonyl-2-methyl-6,7-dihydro-5H-quinolin-8-one:

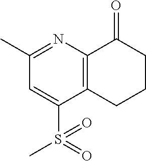 [SMILES: Cc1cc(S(C)(=O)=O)c2c(n1)C(=O)CCC2]

To a solution of 2-methyl-4-methylsulfanyl-6,7-dihydro-5H-quinolin-8-one (110 mg; 0.53 mmol) in dichloromethane (10 mL) was added a solution of sodium bicarbonate (267 mg; 3.18 mmol) in water (3.5 mL) at 0° C. After 1 h, a solution of 3-chloroperbenzoic acid (183 mg, 1.06 mmol) in dichloromethane (35 mL) was slowly added over 1 h at 0° C. The resulting solution was stirred at 0° C. for 30 min then ambient temperature for 12 h. Following the course of the reaction by TLC indicated that starting material was not completely consumed by this time, so the same quantity of 3-chloroperbenzoic acid was further added and the mixture was stirred for a few hours more. The phases of the reaction mixture were separated and the aqueous layer was extracted with dichloromethane. The combined organic phases were dried over sodium sulphate, filtered and the solvent was removed in vacuo. The resulting material was purified by chromatography on silica gel (eluent: heptane/ethyl acetate 1:1 (v:v)). This gave the title compound (90 mg) as a yellow solid. LC-MS (ZMD): UV Detection: 220 nm; Rₜ=1.06 min. MS: (M⁺+1)=240.

Step D) 4-Methanesulfonyl-2-methyl-6,7-dihydro-5H-quinolin-8-one O-{3-[1-(4,6-dimethyl-pyridin-2-yl)-eth-(E)-ylideneaminooxy]-2,2-dimethyl-propyl}-oxime: To a solution of 1-(4,6-dimethyl-pyridin-2-yl)-ethanone O-(3-aminooxy-2,2-dimethyl-propyl)-oxime (56 mg; 0.21 mmol) in ethanol (0.2 mL) was added 4-methanesulfonyl-2-methyl-6,7-dihydro-5H-quinolin-8-one (50 mg; 0.21 mmol). After stirring at ambient temperature for 3 h, water was added to the reaction mixture and the pH was adjusted to 14 by the addition of 2M aqueous NaOH. Extraction was carried out using ethyl acetate. The combined organic layers were washed with brine and then dried over sodium sulphate, filtered and the solvent was removed in vacuo. The resulting material was purified by chromatography on silica gel (eluent: heptane/ethyl acetate). This gave the title compound (102 mg) as a colorless gum. LC-MS (ZMD): UV Detection: 220 nm; Rₜ=1.7 min. MS: (M⁺+1)=487.

¹H NMR (200.131 MHz, CDCl₃) δ₍ₚₚₘ₎: 7.7 (s, 1H), 7.45 (s, 1H), 6.9 (s, 1H), 4.25 (s, 2H), 4.1 (s, 2H), 3.15 (t, 2H), 3.1 (s, 3H), 2.9 (t, 2H), 2.65 (s, 3H), 2.5 (s, 3H), 2.35 (s, 3H), 2. (s, 3H), 1.9 (q, 2H), 1.55 (s, 3H), 1.25 (s, 3H).

The following examples provide useful intermediates:

### 2-Methyl-6,7-Dihydro-5H-quinolin-8-one-(E)-oxime

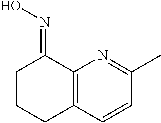

A 250 mL single-necked round-bottomed flask, equipped with a condenser, was charged with a solution of 2-methyl-6,7-dihydro-5H-quinolin-8-one (7.00 g) (CA Registry Number: 849643-01-2) in absolute ethanol (70 mL). Under stirring, first hydroxylamine-hydrochloride (4.50 g) was added and then a solution of NaOH (8.70 g) dissolved in water (14.00 mL) was added in portions. Stirring was continued under heating to reflux for 6.0 hours. Following the course of the reaction by TLC indicated that starting materials were consumed by this time. The suspension was cooled to room temperature. Under stirring and cooling with an ice-water cooling bath, 10 mL of water was added and the pH was adjusted to 6 by the addition of 6 M aqueous HCl. Extraction was carried out using ethyl acetate (2×100 mL). The combined organic phases were washed with brine and then dried over sodium sulphate, filtered and the solvent was removed in vacuo to give a yellow solid (7.65 g).

Analytical data for the title compound:

LC-MS (Method ZMD) UV Detection: 220 nm; Rₜ=0.20, MS: (M⁺+1)=177, (M⁺+23)=179; melting point=177-181° C.

TLC: Plates: Merck DC-Plates, silica gel F₂₅₄, saturated atmosphere in developing tank, UV detection, eluent: ethyl acetate/triethylamine 10:10 (v:v); R_{f} of title compound=0.26, R_{f} of the ketone starting material=0.46.

The preparation of the following starting materials is described in the literature

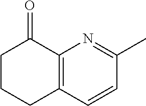

CA Registry Number: 849643-01-2

U.S. Pat. Appl. Publ. (2005), 75 pp., Cont.-in-part of U.S. Ser. No. 437,807. CODEN: USXXCO US 2005075366 A1 20050407

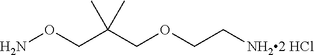

CA Registry Number: 1034433-68-5

PCT Int. Appl. (2008), 187 pp. CODEN: PIXXD2 WO 2008074418 A2 20080626

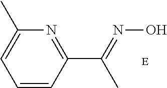

CA Registry Number: 23089-39-6

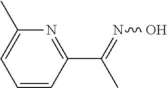

CA Registry Number: 18103-88-3

Talanta (1969), 16(3), 448-52; DE 2447258 (19760408); Journal of Heterocyclic Chemistry (1968), 5(2), 161-4.

### Preparation of 4-chloro-2-methyl-6,7-dihydro-8(5H)-quinolinone

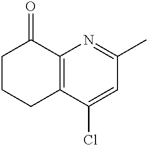

Step A) 4-Hydroxy-2-methylquinoline (10.0 g) (CA Registry Number: 607-67-0) was charged to a reactor containing absolute ethanol (90.0 mL) under nitrogen atmosphere. Under stirring, a suspension of Raney nickel (2.0 g) in absolute ethanol (10.0 mL) was added to the reaction mixture. The nitrogen atmosphere was then replaced by hydrogen. The reaction mixture was stirred at 75° C. for 22 hours under a 100 bar hydrogen atmosphere, at which time analysis of the reaction mixture by TLC indicated that the starting material was consumed. The catalyst was filtered off and the solvent was removed in vacuo to give a white solid (8.35 g). The compound was used as such for the next step.

LC-MS (ZMD): UV Detection: 220 nm; Rₜ=0.40 min. MS: (M⁺+1) 164; melting point=237-240° C.

TLC: Plates: Merck DC-Plates, silica gel F₂₅₄, saturated atmosphere in developing tank, UV detection, eluent: dichloromethane/methanol 9:1 (v:v); R_{f} of title compound=0.22, R_{f} of quinoline starting material=0.34.

Step B) A 50 mL single-necked round-bottom flask, equipped with a condenser, was charged with a solution of 2-methyl-5,6,7,8-tetrahydro-quinolin-4-ol (4.00 g) in phosphorus oxide chloride (18.3 mL) under an argon atmosphere. The resulting colorless solution was stirred at 100° C. for 3.5 hours, after which time TLC indicated that no starting material was remaining. The solvent was removed in vacuo and hot water (40-50° C.) was added carefully and slowly to the residue to hydrolyse the remaining phosphorus oxide chloride. Under cooling with an ice-water cooling bath, the pH was adjusted to 12 by the addition of 4 M aqueous NaOH. The resulting solution was extracted using chloroform (2×50 mL). The combined organic layers were washed with brine (25 mL) and then dried over sodium sulphate, filtered and the solvent was removed in vacuo to give a light yellow oil (4.21 g).

The compound was used as such for the next step.

LC-MS (ZMD): UV Detection: 220 nm; Rₜ=0.87 min. MS: (M⁺+1)=182.

TLC: Plates: Merck DC-Plates, silica gel F₂₅₄, saturated atmosphere in developing tank, UV detection, eluent: heptane/ethyl acetate 1:4 (v:v); R_{f} of title compound=0.40, R_{f} of quinolinol starting material=0.

Step C) A 25 mL single-necked round-bottom flask, equipped with a condenser, was charged with a solution of 4-chloro-2-methyl-5,6,7,8-tetrahydroquinoline (560 mg) in acetic anhydride (0.49 mL). Under stirring, benzaldehyde (0.34 mL) was added and the resulting yellow solution was stirred under heating to reflux for 3.5 hours. Following the course of the reaction by TLC indicated that the starting material was consumed by this time. The resulting brown solution was cooled to room temperature. Crushed ice was added and the pH was adjusted to 10 using a small amount of 2M aqueous NaOH. Extraction was carried out using ethyl acetate (2×20 mL). The combined organic phases were dried over sodium sulphate, filtered and the solvent was removed in vacuo to give a brown gum (750 mg). The crude material was purified by chromatography on silica gel (eluent: heptane/ethyl acetate 98:2 (v:v)). This gave the desired compound (263 mg) as a yellow oil.

LC-MS (Method ZMD) UV Detection: 220 nm; Rₜ=2.23, MS: (M⁺+1)=270. TLC: Plates: Merck DC-Plates, silica gel F₂₅₄, saturated atmosphere in developing tank, UV detection, eluent: heptanes/ethyl acetate 9:1 (v:v); R_{f} of title compound=0.44, R_{f} of the chloroquinoline starting material=0.09.

Step D) A 25 mL single-necked round-bottom flask was charged with a solution of 8-benzylidene-4-chloro-2-methyl-5,6,7,8-tetrahydro-quinoline (263 mg) in dichloromethane/methanol (2.0:3.8 mL). Under stirring and cooling to −78° C. with a dry ice-acetone cooling bath, ozone was passed through the reaction mixture for 3 minutes until a light blue color was observed. Then dimethyl sulfide (2.0 mL) was added at −78° C. The reaction mixture was then allowed to reach room temperature and stirred for 4 hours. Solvents were removed in vacuo, then the resulting orange gum was taken up in diethyl ether and aqueous HCl (1M; 5 mL) was added. Extraction of the acidic by-products was carried out using diethyl ether (2×20 mL). Crushed ice was added to the aqueous layer and the pH was adjusted to 10 by the addition of 2M aqueous NaOH. The resulting solution was extracted using chloroform (2×20 mL). The combined organic layers were dried over sodium sulphate, filtered and the solvent was removed in vacuo to give a yellow solid (96 mg).

Analytical data for the title compound:

LC-MS (Method ZMD) UV Detection: 220 nm; Rₜ=1.28, MS: (M⁺+1)=196.

TLC: Plates: Merck DC-Plates, silica gel F₂₅₄, saturated atmosphere in developing tank, UV detection, eluent: heptanes/ethyl acetate 4:1 (v:v); R_{f} of title compound=0.04, R_{f} of the benzylidene starting material=0.59.

### Preparation of 2,4-dimethyl-6,7-dihydro-8(5H)-quinolinone

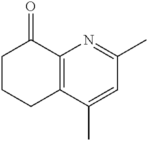

Step A) A 5 mL microwave tube was charged with a solution of 4-chloro-2-methyl-5,6,7,8-tetrahydroquinoline (500 mg) in 1,2-dichloroethane (2.50 mL). Under stirring, trimethylboroxine (380 mg), potassium carbonate (647 mg) and dichloro[1,1′-bis(diphenylphosphino)ferrocene]palladium(II) dichloromethane adduct (101 mg) were added and the resulting red suspension was degassed under argon for 5 minutes. The reaction mixture was subjected to microwave irradiation at 120° C. for 0.5 hour. After addition of new portions of trimethylboroxine (2×380 mg) and catalyst (101 mg), the reaction mixture was again subjected to microwave irradiation at 120° C. for 2×0.5 hour. Following the course of the reaction by TLC indicated that the starting material was consumed by this time. The resulting brown material was purified by chromatography on silica gel (eluent: heptane/ethyl acetate 4:1 (v:v)). This gave the desired compound (345 mg) as a light brown oil.

LC-MS (Method ZMD) UV Detection: 220 nm; Rₜ=0.75, MS: (M⁺+1)=162.

TLC: Plates: Merck DC-Plates, silica gel F₂₅₄, saturated atmosphere in developing tank, UV detection, eluent: heptane/ethyl acetate 1:2 (v:v); R_{f} of title compound=0.28, R_{f} of the chloroquinoline starting material=0.40.

Step B) 2,4-Dimethyl-5,6,7,8-tetrahydro-quinoline (150 mg) was charged to a 10 mL single-necked round-bottom flask containing chloroform (1.50 mL). Under stirring and cooling with an ice-water cooling bath, 3-chloroperbenzoic acid (344 mg) was added portion wise. The resulting orange solution was stirred at room temperature for 5 hours, at which time analysis of the reaction mixture by TLC indicated that the starting material was consumed. Under cooling with an ice-water cooling bath, the pH was adjusted to pH 12 by the addition of aqueous NaOH (4M; 2.0 mL). The resulting solution was extracted using chloroform (3×10 mL). The combined organic layers were washed with brine (10 mL) and then dried over sodium sulphate, filtered and the solvent was removed in vacuo to give a light orange gum (180 mg). This intermediate was used as such for the following step.

LC-MS (ZMD): UV Detection: 220 nm; Rₜ=1.26 min. MS: (M⁺+1) 178.

TLC: Plates: Merck DC-Plates, silica gel F₂₅₄, saturated atmosphere in developing tank, UV detection, eluent: heptane/ethyl acetate 1:2 (v:v); R_{f} of title compound=0, R_{f} of quinoline starting material=0.28.

Step C) A 25 mL single-necked round-bottom flask, equipped with a condenser, was charged with a solution of 2,4-dimethyl-5,6,7,8-tetrahydro-quinoline-1-oxide (334 mg) in dichloromethane (2.00 mL) under an argon atmosphere. Under stirring and cooling with an ice-water cooling bath, trifluoroacetic anhydride (2.66 mL) was added dropwise and the resulting orange solution was stirred under heating to reflux for 22 hours. Following the course of the reaction by TLC indicated that starting material was consumed by this time. The resulting brown solution was cooled to room temperature. Crushed ice was added and the pH was adjusted to 12 using aqueous NaOH (2M; 5 mL). Extraction was carried out using dichloromethane (3×10 mL). The combined organic phases were dried over sodium sulphate, filtered and the solvent was removed in vacuo to give a dark brown gum (226 mg). This intermediate was used without further purification in the next step.

LC-MS (Method ZMD) UV Detection: 220 nm; Rₜ=0.25, MS: (M⁺+1)=178, (M⁺−18)=160.

TLC: Plates: Merck DC-Plates, silica gel F₂₅₄, saturated atmosphere in developing tank, UV detection, eluent: heptanes/ethyl acetate 1:4 (v:v); R_{f} of title compound=0.08, R_{f} of the quinoline-oxide starting material=0.

Step D) A 25 mL single-necked round-bottom flask, equipped with a condenser, was charged with a solution of 2,4-dimethyl-5,6,7,8-tetrahydro-quinolin-8-ol (226 mg) in chloroform (2.00 mL). Under stirring, manganese(IV) oxide (443 mg) was added and the resulting black suspension was stirred under heating to reflux for 18 hours, after which time TLC indicated that no starting material remained. The resulting black material was purified by chromatography on silica gel (eluent: heptane/ethyl acetate gradient from 1:1 to 1:2 (v:v)). This gave the tittle compound (78 mg) as an orange gum.

Analytical data for the title compound:

LC-MS (ZMD): UV Detection: 220 nm; Rₜ=0.34 min. MS: (M⁺+1)=176.

TLC: Plates: Merck DC-Plates, silica gel F₂₅₄, saturated atmosphere in developing tank, UV detection, eluent: heptane/ethyl acetate 1:4 (v:v); R_{f} of title compound=0.11, R_{f} of quinolinol starting material=0.08.

### Preparation of 9-methyl-2,3-dihydro-1H-acridin-4-one

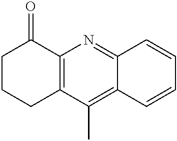 [SMILES: Cc1c2c(nc3ccccc13)C(=O)CCC2]

Step A) 9-Methyl-1,2,3,4-tetrahydroacridine: In a round-bottom flask, cyclohexanone (6.1 mL, 58 mmol) was heated to 90° C., and 2-aminoacetophenone hydrochloride (10 g; 58 mmol) was added in small fractions. The flask was then equipped with a condenser, and the crude heterogeneous mixture was further heated overnight at 110° C. After cooling to room temperature, the red-orange solid was dissolved in ethanol/HCl (12N) [95/5 v/v]. The solution was then neutralized with aqueous NaOH solution. The ethanol was evaporated, and the product extracted with diethyl ether (2×100 mL). The combined organic layers were washed with water (2×100 mL), dried over magnesium sulphate and filtered, and the solvent was removed under reduced pressure. The desired product was finally obtained as a brown-yellow solid (10.2 g, 89%). ¹H NMR (200.131 MHz; CDCl₃) δ₍ₚₚₘ₎: 7.94 (dd, ³J=8.3 Hz and ⁴J=1.1 Hz, 1H), 7.87 (dd, ³J=8.3 Hz and ⁴J=1.3 Hz, 1H), 7.55 (ddd, ³J=8.3 Hz, ³J=8.3 Hz and ⁴J=1.3 Hz, 1H), 7.38 (ddd, ³J=8.3 Hz, ³J=8.3 Hz and ⁴J=1.1 Hz, 1H), 3.07 (t br, ³J=6.7 Hz, 2H), 2.79 (t br, ³J=6.1 Hz, 2H), 2.43 (s, 3H), 1.86 (m, 2×2H). ¹³C NMR (50.332 MHz, CDCl₃) δ₍ₚₚₘ₎: 157.8, 145.5, 140.6, 128.0, 126.4, 128.6, 127.6, 124.8, 122.9, 34.2, 26.5, 22.8, 22.4, 12.9. HRMS (EI) m/z, calcd for [M]⁺ (found): 197.1204 (197.1198). Anal. Calcd for C₁₄H₁₅N (found): C, 85.24 (85.25); H, 7.66 (7.72); N, 7.10 (6.78).

Step B) N-Oxide-9-methyl-1,2,3,4-tetrahydroacridine: A solution of 3-chloroperbenzoic acid (26 g, 105 mmol) in dichloromethane (300 mL) was slowly added to a solution of 9-methyl-1,2,3,4-tetrahydroacridine (10.2 g, 52 mmol) in dichloromethane (100 mL) at 0° C. The mixture was stirred for 4 h at room temperature and quenched with an aqueous NaOH solution. The organic layers were further washed with water (5×100 mL) and dried over magnesium sulphate, and the solvent was removed under reduced pressure giving desired product as a brownish solid. (10.83 g, 98%). ¹H NMR (200.131 MHz, CDCl₃) δ₍ₚₚₘ₎: 8.77 (dd, ³J=8.5 Hz and ⁴J=1.2 Hz, 1H), 7.97 (dd, ³J=8.5 Hz and ⁴J=0.9 Hz, 1H), 7.70-7.50 (m, 2×1H), 3.19 (t, ³J=6.1 Hz, 2H), 2.85 (t, ³J=6.2 Hz, 2H), 2.51 (s, 3H), 1.88 (m, 2×2H).

¹³C NMR (50.332 MHz, CDCl₃) δ₍ₚₚₘ₎: 146.7, 139.1, 131.6, 129.9, 127.7, 129.0, 127.3, 123.9, 119.6, 27.1, 26.6, 22.0, 21.4, 13.4. HRMS (EI) calcd for [M]⁺ (found): 213.1154 (213.1159).

Step C) 9-Methyl-1,2,3,4-tetrahydroacridin-4-ol: In a two-neck round-bottom flask equipped with a reflux condenser, N-oxide-9-methyl-1,2,3,4-tetrahydroacridine (11.2 g, 52 mmol) was dissolved in dichloromethane (250 mL). Trifluoroacetic anhydride (17 mL, 120 mmol) was slowly added at room temperature (the reaction is exothermic). The solution was stirred for 5 h, and the solvent was evaporated. The crude solid was dissolved in methanol (50 mL) and saponified by an aqueous K₂CO₃ solution (2M; 150 mL); a brown solid precipitated. The methanol was removed under reduced pressure, and the product was extracted with dichloromethane (2×150 mL). The combined organic layers were washed with brine (2×50 mL), dried over magnesium sulphate, and evaporated to dryness. The desired product was recovered as a brown solid (9.4 g, 84%). ¹H NMR (200.131 MHz, CDCl₃) δ₍ₚₚₘ₎: 7.96 (d, ³J=8.3 Hz, 1H), 7.91 (d, ³J=8.4 Hz, 1H), 7.58 (dd, ³J=8.3 Hz and ³J=8.1 Hz, 1H), 7.45 (dd, ³J=8.1 Hz and ³J=8.4 Hz, 1H), 4.95 (s br, 1H), 4.76 (dd, ³J=10.3 Hz and ³J=10.0 Hz, 1H), 2.89 (m, 2H), 2.54 (s, 3H), 2.40-1.92 (2×m, 2×1H), 1.82 (m, 2H). ¹³C NMR (50.332 MHz, CDCl₃) δ₍ₚₚₘ₎: 159.2, 145.3, 142.0, 127.7, 127.3, 129.2, 128.5, 126.0, 123.5, 70.2, 30.3, 26.7, 19.6, 13.8. HRMS (EI) calcd for [M]⁺ (found): 213.1153 (213.1154).

Step D) 9-Methyl-2,3-dihydro-1H-acridin-4-one: To a dichloromethane solution (300 mL) of 9-methyl-1,2,3,4-tetrahydroacridin-4-ol (9.4 g, 44 mmol) was added manganese(IV) oxide (23 g, 264 mmol) at room temperature, and the heterogeneous solution was allowed to stir for 2 days. After filtration over Celite, the solvent was evaporated. The crude dark solid was purified by column chromatography (neutral alumina, dichloromethane as eluant). After evaporation of the solvent, the title compound was recovered as a brownish solid (5.41 g, 58%). ¹H NMR (200.131 MHz, CDCl₃) δ₍ₚₚₘ₎: 8.31 (dd, ³J=8.1 Hz and ⁴J=0.8 Hz, 1H), 7.94 (dd, ³J=8.0 Hz and ⁴J=1.4 Hz, 1H), 7.67-7.51 (m, 2H), 3.08 (t, ³J=6.1 Hz, 2H), 2.82 (t, ³J=6.4 Hz, 2H), 2.60 (s, 3H), 2.22 (m, 2H). ¹³C NMR (50.332 MHz, CDCl₃) δ₍ₚₚₘ₎: 198.2, 148.4, 146.9, 143.6, 134.1, 129.4, 132.4, 129.6, 128.9, 123.8, 40.2, 27.2, 22.4, 14.5. HRMS (EI) m/z calcd for [M]⁺ (found): 211.0997 (211.0989). Anal. Calcd for C₁₄H₁₃NO (found): C, 79.59 (79.72); H, 6.20 (6.28); N, 6.63 (6.10).

### Preparation of 2-methyl-4-phenoxy-6,7-dihydro-5H-quinolin-8-one

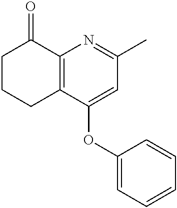 [SMILES: Cc1cc(Oc2ccccc2)c2c(n1)C(=O)CCC2]

Step A) 4-Chloro-2-methyl-5,6,7,8-tetrahydro-quinoline 1-oxide: In a round-bottom flask, 4-chloro-2-methyl-5,6,7,8-tetrahydro-quinoline (3.0 g, 17 mmol) was stirred in chloroform (17 mL) at room temperature to give a light brown solution. The solution was cooled to 0° C. using an ice bath. At this temperature, 3-chloroperbenzoic acid (6.1 g, 25 mmol) was added portionwise over 5 minutes to give a yellow suspension. The reaction mixture was stirred at 0° C. for 10 minutes and the ice bath was then removed. The reaction mixture was allowed to warm to room temperature and further stirred at that temperature for 5 hours, giving a yellow suspension. The reaction was then cooled using an ice bath. Water and aqueous sodium hydroxide solution (4N; 25 mL) were added to the reaction mixture to give a reaction mixture of pH 14. The reaction mixture was extracted twice with chloroform (30 mL). The organic fractions were dried using sodium sulphate and concentrated under reduced pressure to give a light yellow solid (3.36 g). LC-MS (Method ZMD) UV Detection: 220 nm; Rₜ=1.39, MS: (M⁺+1)=198

Step B) 4-Chloro-2-methyl-5,6,7,8-tetrahydro-quinolin-8-ol: In a round-bottom flask, 4-chloro-2-methyl-5,6,7,8-tetrahydro-quinoline 1-oxide (3.1 g, 16 mmol) was stirred in dichloromethane (16 mL) at room temperature to give a yellow solution. The solution was cooled to 0° C. using an ice bath. At this temperature, trifluoroacetic anhydride (17.7 mL, 125 mmol) was added via a syringe over 10 minutes. The reaction mixture was stirred at 0° C. for 15 minutes and the ice bath was then removed. The reaction mixture was allowed to warm to room temperature and further stirred at that temperature for 5 hours, giving a yellow solution. The reaction was then cooled using an ice bath and aqueous sodium hydroxide solution (8N; 35 mL) was added to the reaction mixture over 20 minutes to give an orange suspension, which was stirred at room temperature for a further 4 hours. The reaction mixture was extracted twice with dichloromethane (50 mL). The organic fractions were dried using sodium sulphate and concentrated under reduced pressure to give a light yellow solid (2.75 g). This was used without further purification. mp=87-90° C.

Step C) 2-Methyl-4-phenoxy-5,6,7,8-tetrahydro-quinolin-8-ol: In a 5 mL closed Supelco vessel, phenol (3.1 g, 16 mmol) was stirred in 1-methyl-pyrrolidone (1.0 mL) at room temperature to give a colourless solution. Sodium bis(trimethylsilyl) amide (0.232 g, 1.265 mmol) was added to this solution to give a light yellow suspension. This was stirred at room temperature for 40 minutes resulting in a beige solution. A solution of 4-chloro-2-methyl-5,6,7,8-tetrahydro-quinolin-8-ol (2.5 g, 1.265 mmol) in 1-methyl-pyrrolidone (0.5 mL) was slowly added to the reaction mixture via syringe, giving a yellow suspension. The reaction mixture was stirred at 60° C. for 1 hour, giving a dark green solution. It was then stirred at 120° C. for 90 minutes to give a red-brown solution, followed by stirring for a further 2 hours at 160° C. resulting in a brown solution. At this time, the reaction mixture was transferred to a 10 mL Tiny Clave and stirred at 175° C. for 16 hours, giving a dark brown solution. The reaction mixture was allowed to cool to room temperature and then water and aqueous sodium hydroxide solution (2N; 30 mL) was added. The reaction mixture was extracted twice with diethyl ether (20 mL) and then the combined organic layers were washed twice with water (20 mL). The organic layer was dried over sodium sulphate and concentrated under reduced pressure to give a yellow gum. This gum was further purified by flash chromatography over silica (eluent: heptanes:ethyl acetate 2:1). This gave a yellow gum (0.09 g; 52% pure). This was used without further purification. This was used without further purification.

LC-MS (Method ZMD) UV Detection: 220 nm; Rₜ=1.00, MS: (M⁺+1)=256

Step D) 2-Methyl-4-phenoxy-6,7-dihydro-5H-quinolin-8-one: In a round-bottom flask equipped with a condenser, 2-methyl-4-phenoxy-5,6,7,8-tetrahydro-quinolin-8-ol (0.09 g, 0.35 mmol) was stirred in chloroform (2 mL) at room temperature to give a yellow solution. To the reaction mixture was added manganese (IV) oxide (0.12 g, 1.4 mmol) to give a black suspension. This was stirred at reflux for 74 hours resulting in a black suspension. At this time the reaction mixture was allowed to return to room temperature. The reaction mixture was filtered, and the filter cake was twice washed with chloroform (10 mL). This gave a dark brown gum (0.1 g) which was purified by flash chromatography over silica (heptanes:ethyl acetate 1:1). This gave a yellow gum (0.0154 g).

LC-MS (Method ZMD) UV Detection: 220 nm; Rₜ=1.18, MS: (M⁺+1)=254

### Preparation of 2-methyl-4-pyrrolidin-1-yl-6,7-dihydro-5H-quinolin-8-one

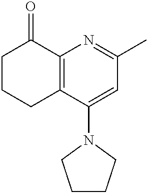 [SMILES: Cc1cc(N2CCCC2)c2c(n1)C(=O)CCC2]

Step A) 4-Chloro-2-methyl-5,6,7,8-tetrahydro-quinoline 1-oxide: In a round-bottom flask, 4-chloro-2-methyl-5,6,7,8-tetrahydro-quinoline (3.0 g, 17 mmol) was stirred in chloroform (17 mL) at room temperature to give a light brown solution. The solution was cooled to 0° C. using an ice bath. At this temperature, 3-chloroperbenzoic acid (6.1 g, 25 mmol) was added portionwise over 5 minutes to give a yellow suspension. The reaction mixture was stirred at 0° C. for 10 minutes and the ice bath was then removed. The reaction mixture was allowed to warm to room temperature and further stirred at that temperature for 5 hours, giving a yellow suspension. The reaction was then cooled using an ice bath. Water and aqueous sodium hydroxide solution (4N; 25 mL) were added to the reaction mixture to give a reaction mixture of pH 14. The reaction mixture was extracted twice with chloroform (30 mL). The organic fractions were dried using sodium sulphate and concentrated under reduced pressure to give a light yellow solid (3.36 g).

LC-MS (Method ZMD) UV Detection: 220 nm; Rₜ=1.39, MS: (M⁺+1)=198

Step B) 4-Chloro-2-methyl-5,6,7,8-tetrahydro-quinolin-8-ol: In a round-bottom flask, 4-chloro-2-methyl-5,6,7,8-tetrahydro-quinoline 1-oxide (3.1 g, 16 mmol) was stirred in dichloromethane (16 mL) at room temperature to give a yellow solution. The solution was cooled to 0° C. using an ice bath. At this temperature, trifluoroacetic anhydride (17.7 mL, 125 mmol) was added via a syringe over 10 minutes. The reaction mixture was stirred at 0° C. for 15 minutes and the ice bath was then removed. The reaction mixture was allowed to warm to room temperature and further stirred at that temperature for 5 hours, giving a yellow solution. The reaction was then cooled using an ice bath and aqueous sodium hydroxide solution (8N; 35 mL) was added to the reaction mixture over 20 minutes to give an orange suspension, which was stirred at room temperature for a further 4 hours. The reaction mixture was extracted twice with dichloromethane (50 mL). The organic fractions were dried using sodium sulphate and concentrated under reduced pressure to give a light yellow solid (2.75 g; mp=87-90° C.). This was used without further purification. mp=87-90° C.

Step C) 2-Methyl-4-pyrrolidin-1-yl-5,6,7,8-tetrahydro-quinolin-8-ol: In a 10 mL Tiny Clave, 4-chloro-2-methyl-5,6,7,8-tetrahydro-quinolin-8-ol (0.4 g, 2.0 mmol) was stirred in pyrrolidone (1.7 mL, 20 mmol) at room temperature to give a yellow solution. The reaction mixture was then stirred at 150° C. for 8 hours to give a brown solution. The reaction mixture was then allowed to return to room temperature. Water and aqueous sodium hydroxide solution (2N; 5 mL) were then added. The reaction mixture was extracted twice with diethyl ether (15 mL) and then the combined organic layers were washed with brine (10 mL). The organic layer was dried over sodium sulphate and concentrated under reduced pressure to give a brown gum (0.5 g). This gum was filtered over silica. The filter cake was washed with a mixture of 95:5 chloroform:methanol. The filtrate was concentrated under reduced pressure to give a yellow solid (0.4 g). mp=97-98° C.

Step D) 2-Methyl-4-pyrrolidin-1-yl-6,7-dihydro-5H-quinolin-8-one: In a round-bottom flask equipped with a condenser, 2-methyl-4-pyrrolidin-1-yl-5,6,7,8-tetrahydro-quinolin-8-ol (0.36 g, 1.55 mmol) was stirred in chloroform (2 mL) at room temperature to give a yellow-orange solution. To the reaction mixture was added manganese (IV) oxide (0.54 g, 6.198 mmol) to give a black suspension. This was stirred at reflux for 74 hours resulting in a black suspension. At this time the reaction mixture was allowed to return to room temperature. The reaction mixture was filtered, and the filter cake was twice washed with chloroform (15 mL). This gave a dark brown gum (0.38 g) which was purified by flash chromatography over silica (eluent: chloroform/methanol 95:5). This gave a yellow gum (0.0154 g).

LC-MS (Method ZMD) UV Detection: 220 nm; Rₜ=0.18, MS: (M⁺+1)=231

### Preparation of 2-Methoxymethyl-4-methyl-6,7-dihydro-5H-quinolin-8-one

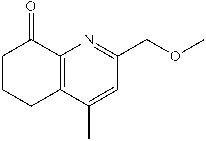

Step A) (4-Methyl-5,6,7,8-tetrahydro-quinolin-2-yl)-methanol:

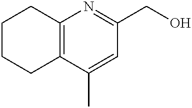

The reactor was charged with a solution of (4-methyl-quinolin-2-yl)-methanol (9 g; 52 mmol) in trifluoroacetic acid (90 mL) and a suspension of platinum (IV) oxide hydrate in trifluoroacetic acid. After 2 h at 22° C./4 bar/H₂ uptake 99%, a NMR control (¹H NMR, CDCl₃ after a basic work-up of the sample with aq. NH₃) indicated complete and clean conversion. The catalyst was filtered off, and the solvent was removed in vacuo to give a dark brown oil. Under ice cooling, this oil was diluted with water (35 mL) and the pH was adjusted to pH 14 by careful addition of 8M aqueous NaOH. Extraction was carried out using ethyl acetate (3×100 mL). The combined organic layers were dried over sodium sulphate, filtered and the solvent was removed in vacuo to give a brown gum. The resulting material was purified by chromatography on silica gel to give the title compound (4.4 g) as a beige solid.

LC-MS (ZCQ) UV Detection: 220 nm; Rₜ=0.23, MS: (M⁺+1)=178

TLC: Plates: Merck DC-Plates, silica gel F₂₅₄, saturated atmosphere in developing tank, UV detection, eluent: ethyl acetate; R_{f} of title compound=0.11, R_{f} of the starting material=0.26.

Step B) 2-Methoxymethyl-4-methyl-5,6,7,8-tetrahydro-quinoline:

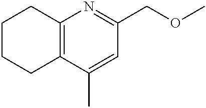

A 25 mL dried single-necked round-bottom flask, under nitrogen, was charged with a solution of (4-methyl-5,6,7,8-tetrahydro-quinolin-2-yl)-methanol (0.5 g; 2.8 mmol) in tetrahydrofuran (3 mL). Sodium hydride (0.123 g; 2.8 mmol) was added portionwise over 2 min. The resulting suspension was stirred at ambient temperature for 45 min. Iodomethane (0.176 mL; 2.8 mmol) was added dropwise. The solution was stirred at ambient temperature for 3 h more. The resulting solution was quenched with water (5 mL) and extraction was carried out using ethyl acetate (2×10 mL). The combined organic layers were dried over sodium sulphate, filtered and the solvent was removed in vacuo to give a yellow oil (0.52 g). This intermediate was used without further purification in the next step.

LC-MS (ZMD): UV Detection: 220 nm; Rₜ=0.48 min. MS: (M⁺+1)=192.

TLC: Plates: Merck DC-Plates, silica gel F₂₅₄, saturated atmosphere in developing tank, UV detection, eluent: heptane/ethyl acetate 1:4 (v:v); R_{f} of title compound=0.28, R_{f} of the starting material=0.13.

Step C) 2-Methoxymethyl-4-methyl-5,6,7,8-tetrahydro-quinoline 1-oxide:

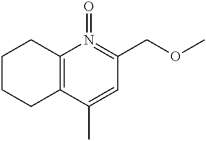

A 25 mL single-necked round-bottom flask, was charged with a solution of 2-methoxymethyl-4-methyl-5,6,7,8-tetrahydro-quinoline (0.58 g; 3.03 mmol) in chloroform (3 mL). Under stirring and cooling with an ice-water cooling bath, 3-chloroperbenzoic acid

(1.12 g; 4.54 mmol) was added portionwise over 2 min. The resulting yellow suspension was stirred at 0° C. for 10 min then at ambient temperature for 16 h. Under ice cooling, the suspension was quenched with water and the pH was adjusted to 14 by the addition of aqueous NaOH (4M; 5 mL). Extraction was carried out using chloroform (2×10 mL). The combined organic layers were dried over sodium sulphate, filtered and the solvent was removed in vacuo to give a light yellow solid (0.65 g). This intermediate was used without further purification in the next step.

LC-MS (ZMD): UV Detection: 220 nm; Rₜ=1.31 min. MS: (M⁺+1)=208.

TLC: Plates: Merck DC-Plates, silica gel F₂₅₄, saturated atmosphere in developing tank, UV detection, eluent: heptane/ethyl acetate 1:4 (v:v); R_{f} of title compound=0.02, R_{f} of the starting material=0.28.

Step D) 2-Methoxymethyl-4-methyl-5,6,7,8-tetrahydro-quinolin-8-ol:

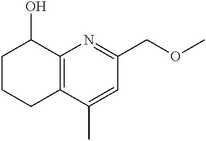

A 25 mL single-necked round-bottom flask, was charged with a solution of 2-methoxymethyl-4-methyl-5,6,7,8-tetrahydro-quinoline 1-oxide (0.65 g; 3.135 mmol) in dichloromethane (3.5 mL). Under stirring and cooling with an ice-water cooling bath trifluoroacetic anhydride (3.54 mL; 25.076 mmol) was added slowly via syringe over 3 min. The resulting yellow solution was stirred at 0° C. for 15 min then ambient temperature for 66 h. Under ice cooling, the pH was adjusted to 14 by the addition of aqueous NaOH solution (8N; 5 mL) over 5 min. The biphasic solution was stirred vigorously at ambient temperature for 4 h. Extraction was carried out using dichloromethane (2×5 mL). The combined organic layers were dried over sodium sulphate, filtered and the solvent was removed in vacuo to give a brown gum (0.53 g). This intermediate was used without further purification in the next step.

LC-MS (ZMD): UV Detection: 220 nm; Rₜ=0.18 min. MS: (M⁺+1)=208.

TLC: Plates: Merck DC-Plates, silica gel F₂₅₄, saturated atmosphere in developing tank, UV detection, eluent: heptane/ethyl acetate 1:4 (v:v); R_{f} of title compound=0.16, R_{f} of the starting material=0.02.

Step E) 2-Methoxymethyl-4-methyl-6,7-dihydro-5H-quinolin-8-one: A 25 mL single-necked round-bottom flask, equipped with a condenser, was charged with a solution of 2-methoxymethyl-4-methyl-5,6,7,8-tetrahydro-quinolin-8-ol (0.36 g; 1.737 mmol) in chloroform (2 mL). Under stirring, manganese (IV) oxide (0.604 g; 6.947 mmol) was added and the resulting black suspension was stirred under heating to reflux for 18 hours, after which time TLC indicated that no starting material was remaining. The resulting black suspension was allowed to return to ambient temperature and filtered over hyflo before purification by chromatography on silica gel (eluent: heptane/ethyl acetate 1:2). This gave the title compound (0.168 g) as a light yellow solid.

LC-MS (ZMD): UV Detection: 220 nm; Rₜ=1.12 min. MS: (M⁺+1)=206.

TLC: Plates: Merck DC-Plates, silica gel F₂₅₄, saturated atmosphere in developing tank, UV detection, eluent: heptane/ethyl acetate 1:4 (v:v); R_{f} of title compound=0.09, R_{f} of the starting material=0.16.

¹H NMR (200.131 MHz, CDCl₃) δ₍ₚₚₘ₎: 7.46 (s, 1H), 4.65 (s, 2H), 3.47 (s, 3H), 2.9 (t, 2H), 2.8 (t, 2H), 2.35 (s, 3H), 2.2 (q, 2H).

### Preparation of 4-Ethoxy-6,7-dihydro-5H-quinolin-8-one

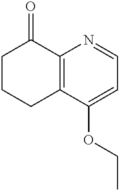

Step A) 4-Ethoxy-5,6,7,8-tetrahydro-quinoline:

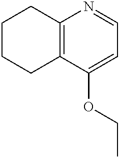

The reactor was charged with a solution of 4-ethoxy-quinoline (1.86 g) in trifluoroacetic acid (17 mL) and a suspension of platinum(IV) oxide hydrate (1.08 g) in trifluoroacetic acid. After 7 h at 22° C./4 bar/H₂ uptake 85%, NMR control (¹H NMR, CDCl₃ after a basic work-up of the sample with aq. NH₃) indicated complete and clean conversion. The catalyst was filtered off and the filtrate was concentrated under reduced pressure. Under ice cooling, 8N aqueous

NaOH solution was added to the resulting oil (10 mL, pH=14). Extraction was carried out using dichloromethane (3×30 mL). The combined organic layers were dried over sodium sulphate, filtered and the solvent was removed in vacuo to give a yellow oil (1.52 g). This intermediate was used without further purification in the next step.

LC-MS (ZMD): UV Detection: 220 nm; Rₜ=0.76 min. MS: (M⁺+1)=178.

TLC: Plates: Merck DC-Plates, silica gel F₂₅₄, saturated atmosphere in developing tank, UV detection, eluent: heptane/ethyl acetate 1:4 (v:v); R_{f} of title compound=0.08, R_{f} of the starting material=0.16.

Step B) 4-Ethoxy-5,6,7,8-tetrahydro-quinoline 1-oxide:

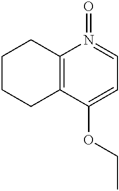

A 50 mL single-necked round-bottom flask, was charged with a solution of 4-ethoxy-5,6,7,8-tetrahydro-quinoline (1.45 g; 8.18 mmol) in chloroform (8 mL). Under stirring and cooling with an ice-water cooling bath, 3-chloroperbenzoic acid (3.03 g; 12.27 mmol) was added portionwise over 2 min. The resulting yellow suspension was stirred at 0° C. for 10 min then at ambient temperature for 19 h. Under ice cooling, the suspension was quenched with water and the pH was adjusted to 14 by the addition of aqueous NaOH (4M; 12 mL). Extraction was carried out using chloroform (2×25 mL). The combined organic layers were dried over sodium sulphate, filtered and the solvent was removed in vacuo to give a yellow oil (1.45 g). This intermediate was used without further purification in the next step.

LC-MS (ZMD): UV Detection: 220 nm; Rₜ=1.24 min. MS: (M⁺+1)=194.

TLC: Plates: Merck DC-Plates, silica gel F₂₅₄, saturated atmosphere in developing tank, UV detection, eluent: dichloromethane/methanol 9:1 (v:v); R_{f} of title compound=0.28, R_{f} of the starting material=0.35.

Step C) 4-Ethoxy-5,6,7,8-tetrahydro-quinolin-8-ol:

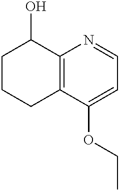

A 5 mL single-necked round-bottom flask was charged with a solution of 4-ethoxy-5,6,7,8-tetrahydro-quinoline 1-oxide (0.1 g; 0.517 mmol) in trifluoroacetic anhydride (0.88 mL). The resulting yellow solution was stirred at reflux for 15 h. The solution was allowed to return to ambient temperature. Under ice cooling, the pH was adjusted to 14 by the addition of aqueous NaOH solution (8N; 2 mL) over 5 min and dichloromethane was then added (2 mL). The biphasic solution was stirred vigorously at ambient temperature for 5 h. As the intermediate product was still observed, methanol was added (3 drops) and the vigorously stirring was continued for 16 h. Extraction was carried out using dichloromethane (2×10 mL). The combined organic layers were dried over sodium sulphate, filtered and the solvent was removed in vacuo to give a yellow solid (61 mg). This intermediate was used without further purification in the next step.

LC-MS (ZMD): UV Detection: 220 nm; Rₜ=0.95 min. MS: (M⁺+1)=194.

TLC: Plates: Merck DC-Plates, silica gel F₂₅₄, saturated atmosphere in developing tank, UV detection, eluent: heptane/ethyl acetate 1:4 (v:v); R_{f} of title compound=0.32, R_{f} of the starting material=0.28.

Step D) 4-Ethoxy-6,7-dihydro-5H-quinolin-8-one: A 25 mL single-necked round-bottom flask, equipped with a condenser, was charged with a solution of 4-ethoxy-5,6,7,8-tetrahydro-quinolin-8-ol (0.193 g; 0.99 mmol) in chloroform (2 mL). Under stirring, manganese (IV) oxide (0.347 g; 3.99 mmol) was added and the resulting black suspension was stirred under heating to reflux for 5 h, after which time TLC indicated that no starting material was remaining. The resulting black suspension was allowed to return to ambient temperature and filtered over hyflo before purification by chromatography on silica gel (eluent: heptane/ethyl acetate 1:2). This gave the title compound (94.9 mg) as a yellow gum.

LC-MS (ZMD): UV Detection: 220 nm; Rₜ=0.41 min. MS: (M⁺+1)=192.

TLC: Plates: Merck DC-Plates, silica gel F₂₅₄, saturated atmosphere in developing tank, UV detection, eluent: heptane/ethyl acetate 1:4 (v:v); R_{f} of title compound=0.11, R_{f} of the starting material=0.02.

¹H NMR (200.131 MHz, CDCl₃) δ₍ₚₚₘ₎: 8.55 (t, 1H), 6.7 (d, 1H), 4.1 (dd, 2H), 2.9 (m, 2H), 2.75 (m, 2H), 2.15 (t, 2H), 1.4 (t, 3H).

### Preparation of 2-Methyl-4-phenyl-6,7-dihydro-5H-quinolin-8-one

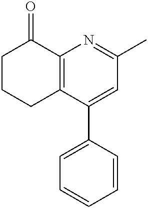 [SMILES: Cc1cc(-c2ccccc2)c2c(n1)C(=O)CCC2]

Step A) 2-Methyl-4-phenyl-5,6,7,8-tetrahydro-quinoline 1-oxide:

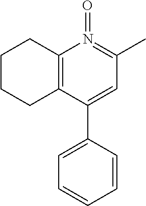 [SMILES: Cc1cc(-c2ccccc2)c2c([n+]1[O-])CCCC2]

A 25 mL single-necked round-bottom flask, was charged with a solution of 2-methyl-4-phenyl-5,6,7,8-tetrahydro-quinoline (0.39 g; 1.76 mmol) in chloroform (2 mL). Under stirring and cooling with an ice-water cooling bath, 3-chloroperbenzoic acid (0.65 g; 2.65 mmol) was added. The resulting light brown suspension was stirred at ambient temperature for 2.5 h.

Under ice cooling, the suspension was quenched with water and the pH was adjusted to 14 by the addition of aqueous NaOH (4M; 2 mL). Extraction was carried out using chloroform (3×10 mL). The combined organic layers were washed with brine (8 mL), dried over sodium sulphate, filtered and the solvent was removed in vacuo to give a light yellow oil (0.36 g). This intermediate was used without further purification in the next step.

LC-MS (ZMD): UV Detection: 220 nm; Rₜ=1.56 min. MS: (M⁺+1)=240.

TLC: Plates: Merck DC-Plates, silica gel F₂₅₄, saturated atmosphere in developing tank, UV detection, eluent: heptane/ethyl acetate 1:2 (v:v); R_{f} of title compound=0, R_{f} of the starting material=0.32.

Step B) 2-Methyl-4-phenyl-5,6,7,8-tetrahydro-quinolin-8-ol:

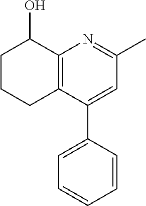 [SMILES: Cc1cc(-c2ccccc2)c2c(n1)C(O)CCC2]

A 25 mL single-necked round-bottom flask was charged with a solution of 2-methyl-4-phenyl-5,6,7,8-tetrahydro-quinoline 1-oxide (0.44 g; 1.85 mmol) in dichloromethane (2 mL). Under stirring and cooling with an ice-water cooling bath, trifluoroacetic anhydride (3.88 mL; 18.51 mmol) was added slowly via a syringe over 2 min. The resulting dark yellow solution was stirred at 0° C. for 15 min then ambient temperature for 2.5 h. Crushed ice was added and the pH was adjusted to 14 using aqueous NaOH (4M; 5 mL). The biphasic solution was stirred vigorously at ambient temperature for 2.5 h. Extraction was carried out using dichloromethane (2×10 mL). The combined organic layers were dried over sodium sulphate, filtered and the solvent was removed in vacuo to give a yellow solid (0.34 g). This intermediate was used without further purification in the next step.

LC-MS (ZMD): UV Detection: 220 nm; Rₜ=0.96 min. MS: (M⁺+1)=240.

TLC: Plates: Merck DC-Plates, silica gel F₂₅₄, saturated atmosphere in developing tank, UV detection, eluent: heptane/ethyl acetate 1:2 (v:v); R_{f} of title compound=0.25, R_{f} of the starting material=0.

Step C) 2-Methyl-4-phenyl-6,7-dihydro-5H-quinolin-8-one: A 50 mL single-necked round-bottom flask, equipped with a condenser, was charged with a solution of 2-methyl-4-phenyl-5,6,7,8-tetrahydro-quinolin-8-ol (0.34 g; 1.41 mmol) in chloroform (2 mL). Under stirring, manganese (IV) oxide (0.49 g; 5.65 mmol) was added and the resulting black suspension was stirred under heating to reflux for 5 h, after which time TLC indicated that no starting material remained. The resulting black suspension was allowed to return to ambient temperature and filtered over hyflo before purification by chromatography on silica gel (eluent: heptane/ethyl acetate 1:1). This gave the title compound (155 mg) as a yellow-orange gum.

LC-MS (ZMD): UV Detection: 220 nm; Rₜ=1.30 min. MS: (M⁺+1)=238.

TLC: Plates: Merck DC-Plates, silica gel F₂₅₄, saturated atmosphere in developing tank, UV detection, eluent: heptane/ethyl acetate 1:2 (v:v); R_{f} of title compound=0.19, R_{f} of the starting material=0.25.

¹H NMR (200.131 MHz, CDCl₃) δ₍ₚₚₘ₎: 7.45 (m, 3H), 7.3 (d, 2H), 7.2 (s, 1H), 2.85 (m, 2H), 2.8 (m, 2H), 2.65 (s, 3H), 2.05 (t, 2H).

### Preparation of Acetic acid 4-ethoxy-5,6,7,8-tetrahydro-quinolin-8-yl ester

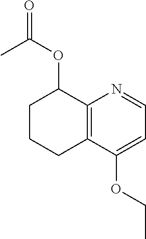 [SMILES: CCOc1ccnc2c1CCCC2OC(C)=O]

A 100 mL single-necked round-bottom flask was charged with a solution of 4-ethoxy-5,6,7,8-tetrahydro-quinoline 1-oxide (1.37 g; 7.089 mmol) in acetic anhydride (12 mL). The resulting yellow solution was stirred at 100° C. for 16 h. Then the solution was allowed to return to ambient temperature. Under ice cooling, the pH was adjusted to 7 by the careful addition of saturated aqueous Na₂CO₃ solution (20 mL). Extraction was carried out using dichloromethane (3×20 mL). The combined organic layers were dried over sodium sulphate, filtered and the solvent was removed in vacuo to give a yellow oil (1.18 g). Purification by flash chromatography over a silica gel cartridge (60 g, 150 mL, 50 mL fractions) of this crude with CH₂Cl₂/MeOH (98:2) gave 0.62 g of the title compound in the form of a yellow oil (85% pure).

LC-MS (ZMD): UV Detection: 220 nm; Rₜ=0.99 min. MS: (M⁺+1)=236, (M⁺+23)=258.

TABLE 17
Physical data:

| | | RT | | |
|---|---|---|---|---|
| | | (mins) | Molecular | mp |
| | Structure | (method) | ion | (° C.) |
| E/E- 2.002 | 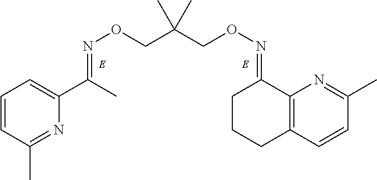 [SMILES: C/C(=N\OCC(C)(C)CO/N=C1\CCCc2ccc(C)nc21)c1cccc(C)n1] | 1.53 (ZMD) | 395 ([M + 1]⁺) 198 ([M + 2]²⁺) | |
| E/E- 2.474 | 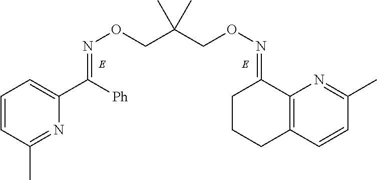 [SMILES: Cc1cccc(/C(=N/OCC(C)(C)CO/N=C2\CCCc3ccc(C)nc32)c2ccccc2)n1] | 1.62 (ZCQ) | 457 ([M + 1]⁺) 229 ([M + 2]²⁺) | |
| Z/E- 2.474 | 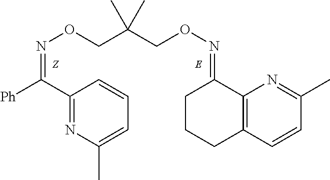 [SMILES: Cc1cccc(/C(=N\OCC(C)(C)CO/N=C2\CCCc3ccc(C)nc32)c2ccccc2)n1] | 1.60 (ZCQ) | 457 ([M + 1]⁺) | |
| 2.510 | 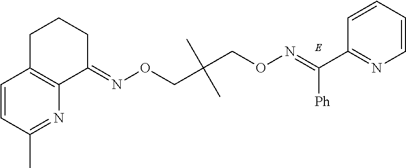 [SMILES: Cc1ccc2c(n1)/C(=N/OCC(C)(C)CO/N=C(\c1ccccc1)c1ccccn1)CCC2] 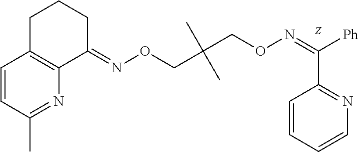 [SMILES: Cc1ccc2c(n1)/C(=N/OCC(C)(C)CO/N=C(/c1ccccc1)c1ccccn1)CCC2] | 1.36 (U) | | |
| E/E- 2.508 | 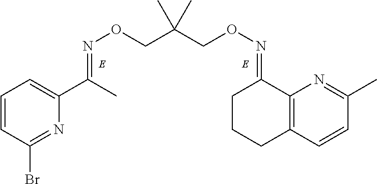 [SMILES: C/C(=N\OCC(C)(C)CO/N=C1\CCCc2ccc(C)nc21)c1cccc(Br)n1] | 1.77 (ZCQ) | 459 ([M + 1]⁺) | |
| E/E- 2.509 | 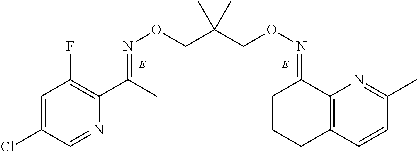 [SMILES: C/C(=N\OCC(C)(C)CO/N=C1\CCCc2ccc(C)nc21)c1ncc(Cl)cc1F] | 1.70 (ZCQ) | 433 ([M + 1]⁺) | |
| E/E- 2.168 | 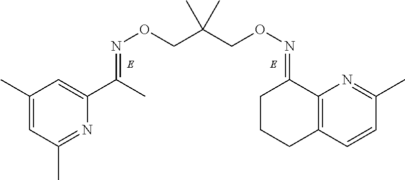 | 1.40 e | 409 ([M + 1]⁺) e | |
| E/E- 2.507 | 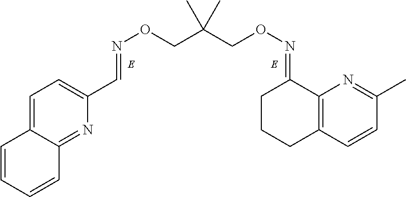 [SMILES: Cc1ccc2c(n1)/C(=N/OCC(C)(C)CO/N=C/c1ccc3ccccc3n1)CCC2] | 1.61 (ZCQ) | 417 ([M + 1]⁺) 209 ([M + 2]²⁺) | |
| E/E- 14.002 | 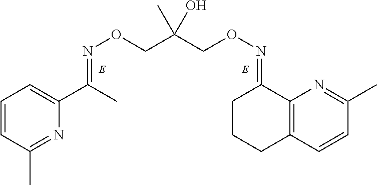 [SMILES: C/C(=N\OCC(C)(O)CO/N=C1\CCCc2ccc(C)nc21)c1cccc(C)n1] racemic form | 1.17 (ZCQ) | 397 ([M + 1]⁺) 199 ([M + 2]²⁺) 419 ([M + 23]⁺) | |
| E/E- 3.002 | 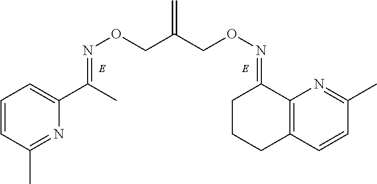 [SMILES: C=C(CO/N=C(\C)c1cccc(C)n1)CO/N=C1\CCCc2ccc(C)nc21] | 1.34 (ZCQ) | 379 ([M + 1]⁺) 401 ([M + 23]⁺) | |
| E/E- 5.168 | 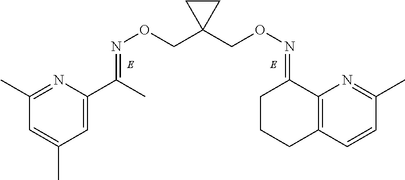 | 1.76 (ZCQ) | 407 ([M + 1]⁺) 429 ([M + 23]⁺) | |
| E/E- 2.208 | 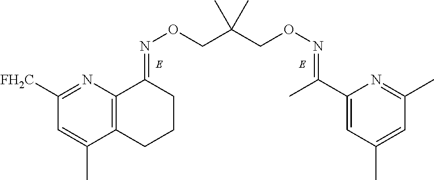 | 1.80 (ZCQ) | 441 ([M + 1]⁺) | |
| E-2.511 | 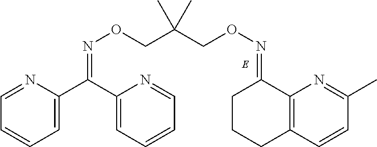 [SMILES: Cc1ccc2c(n1)/C(=N/OCC(C)(C)CON=C(c1ccccn1)c1ccccn1)CCC2] | 1.34 (ZCQ) | 444 ([M + 1]⁺) 466 ([M + 23]⁺) | |
| E/E- 2.512 | 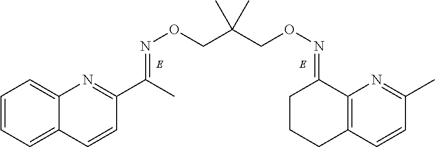 [SMILES: C/C(=N\OCC(C)(C)CO/N=C1\CCCc2ccc(C)nc21)c1ccc2ccccc2n1] | 1.76 (ZCQ) | 431 ([M + 1]⁺) 453 ([M + 23]⁺) | |
| E/E- 2.513 | 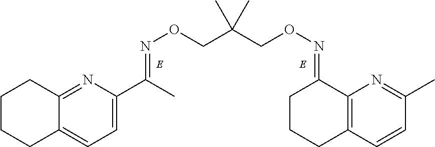 [SMILES: C/C(=N\OCC(C)(C)CO/N=C1\CCCc2ccc(C)nc21)c1ccc2c(n1)CCCC2] | 1.56 (ZMD) | 435 ([M + 1]+) | |
| E/E- 2.514 | 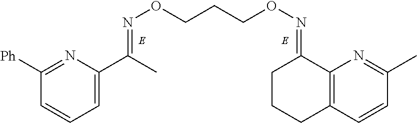 [SMILES: C/C(=N\OCCCO/N=C1\CCCc2ccc(C)nc21)c1cccc(-c2ccccc2)n1] | 1.82 (ZMD) | 429 ([M + 1]⁺) 451 ([M + 23]⁺) | |
| 2.515 | 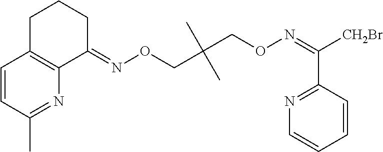 | 1.44 (U) | | |
| 2.516 | 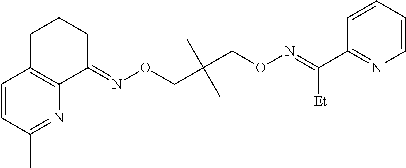 [SMILES: CC/C(=N\OCC(C)(C)CO/N=C1\CCCc2ccc(C)nc21)c1ccccn1] | 1.36 (U) | | |
| 2.517 | 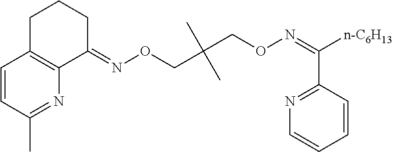 | 1.83 (U) | | |
| 2.518 | 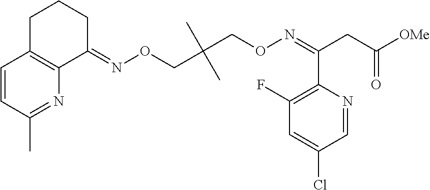 [SMILES: COC(=O)C/C(=N/OCC(C)(C)CO/N=C1\CCCc2ccc(C)nc21)c1ncc(Cl)cc1F] | 1.44 (U) | | |
| 2.519 | 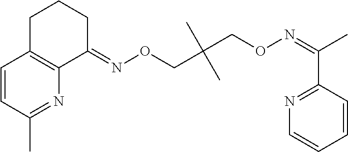 [SMILES: C/C(=N/OCC(C)(C)CO/N=C1\CCCc2ccc(C)nc21)c1ccccn1] | 1.23 (U) | | |
| 2.520 | 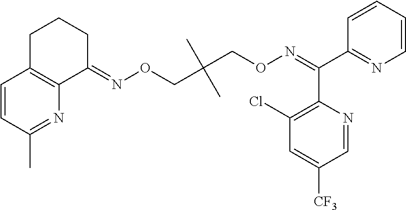 [SMILES: Cc1ccc2c(n1)/C(=N/OCC(C)(C)CO/N=C(/c1ccccn1)c1ncc(C(F)(F)F)cc1Cl)CCC2] | 1.51 (U) | | |
| 2.521 | 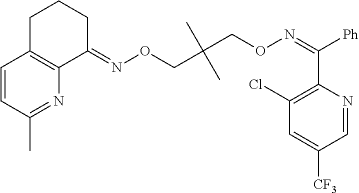 [SMILES: Cc1ccc2c(n1)/C(=N/OCC(C)(C)CO/N=C(/c1ccccc1)c1ncc(C(F)(F)F)cc1Cl)CCC2] | 1.72 (U) | | |
| 2.522 | 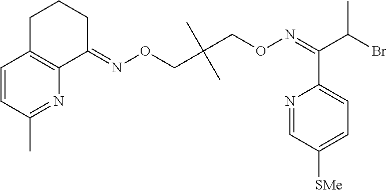 [SMILES: CSc1ccc(/C(=N\OCC(C)(C)CO/N=C2\CCCc3ccc(C)nc32)C(C)Br)nc1] | 1.68 (U) | | |
| 2.523 | 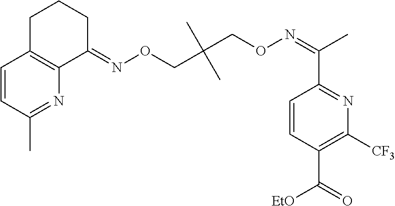 [SMILES: CCOC(=O)c1ccc(/C(C)=N\OCC(C)(C)CO/N=C2\CCCc3ccc(C)nc32)nc1C(F)(F)F] | 1.80 (U) | | |
| 2.524 | 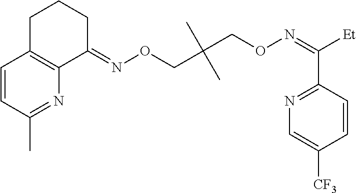 [SMILES: CC/C(=N/OCC(C)(C)CO/N=C1\CCCc2ccc(C)nc21)c1ccc(C(F)(F)F)cn1] | 1.78 (U) | | |
| 2.525 Fraction A | 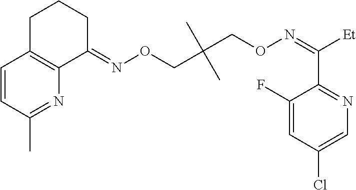 [SMILES: CC/C(=N/OCC(C)(C)CO/N=C1\CCCc2ccc(C)nc21)c1ncc(Cl)cc1F] | 1.53 (U) | | |
| 2.525 Fraction B | 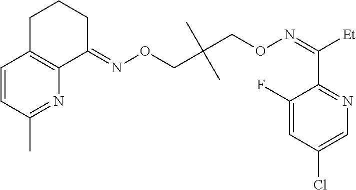 [SMILES: CC/C(=N/OCC(C)(C)CO/N=C1\CCCc2ccc(C)nc21)c1ncc(Cl)cc1F] | 1.61 (U) e | | |
| 2.526 | 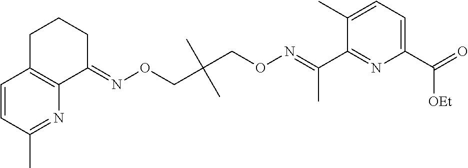 [SMILES: CCOC(=O)c1ccc(C)c(/C(C)=N/OCC(C)(C)CO/N=C2\CCCc3ccc(C)nc32)n1] | 1.57 (U) | | |
| 2.527 | 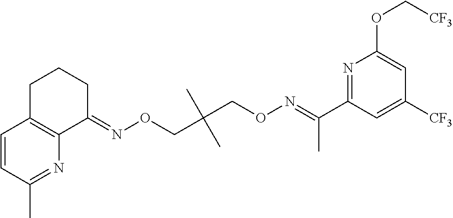 [SMILES: C/C(=N\OCC(C)(C)CO/N=C1\CCCc2ccc(C)nc21)c1cc(C(F)(F)F)cc(OCC(F)(F)F)n1] | 1.99 (U) | | |
| 2.528 | 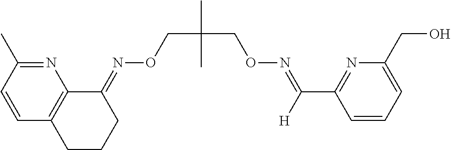 [SMILES: Cc1ccc2c(n1)/C(=N/OCC(C)(C)CO/N=C/c1cccc(CO)n1)CCC2] | 1.05 (U) | | |
| 2.529 | 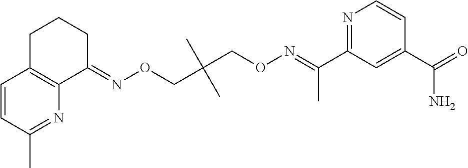 [SMILES: C/C(=N\OCC(C)(C)CO/N=C1\CCCc2ccc(C)nc21)c1cc(C(N)=O)ccn1] | 1.03 (U) | | |
| 2.530 | 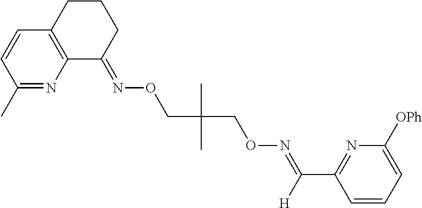 [SMILES: Cc1ccc2c(n1)/C(=N/OCC(C)(C)CO/N=C/c1cccc(Oc3ccccc3)n1)CCC2] | 1.58 (U) | | |
| 2.001 | 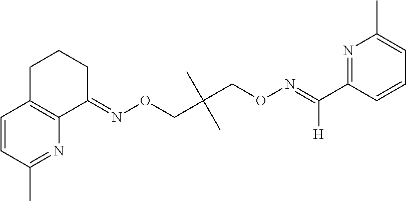 [SMILES: Cc1cccc(/C=N/OCC(C)(C)CO/N=C2\CCCc3ccc(C)nc32)n1] | 1.18 (U) | | |
| 2.531 | 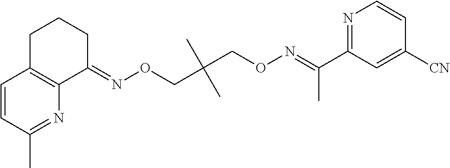 [SMILES: C/C(=N\OCC(C)(C)CO/N=C1\CCCc2ccc(C)nc21)c1cc(C#N)ccn1] | 1.36 (U) | | |
| 2.532 | 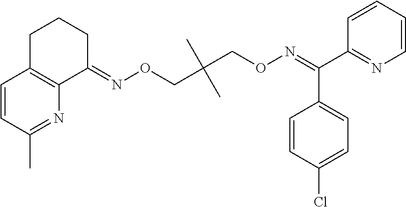 [SMILES: Cc1ccc2c(n1)/C(=N/OCC(C)(C)CO/N=C(\c1ccc(Cl)cc1)c1ccccn1)CCC2] | 1.52 (U) | | |
| 2.533 | 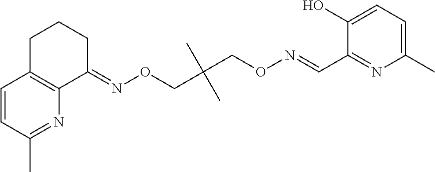 [SMILES: Cc1ccc(O)c(/C=N/OCC(C)(C)CO/N=C2\CCCc3ccc(C)nc32)n1] | 1.21 (U) | | |
| 2.534 | 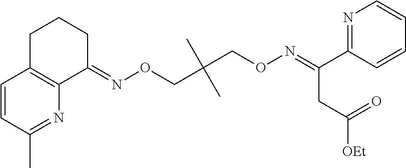 [SMILES: CCOC(=O)C/C(=N\OCC(C)(C)CO/N=C1\CCCc2ccc(C)nc21)c1ccccn1] | 1.37 (U) | | |
| 2.535 | 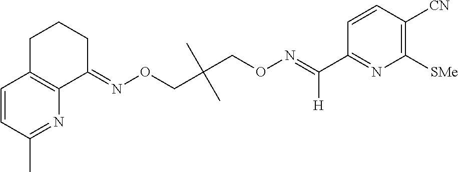 [SMILES: CSc1nc(/C=N/OCC(C)(C)CO/N=C2\CCCc3ccc(C)nc32)ccc1C#N] | 1.53 (U) | | |
| 2.536 | 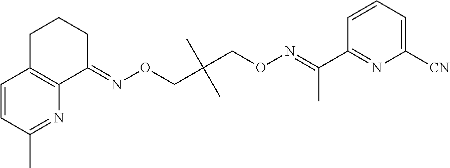 [SMILES: C/C(=N\OCC(C)(C)CO/N=C1\CCCc2ccc(C)nc21)c1cccc(C#N)n1] | 1.40 (U) | | |
| 2.537 | 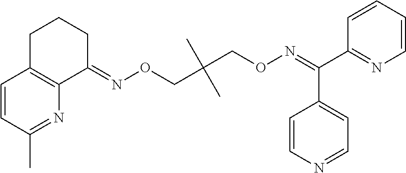 [SMILES: Cc1ccc2c(n1)/C(=N/OCC(C)(C)CO/N=C(\c1ccncc1)c1ccccn1)CCC2] | 1.08 (U) | | |
| 2.538 | 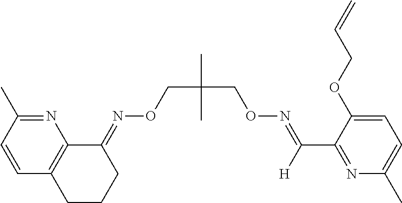 [SMILES: C=CCOc1ccc(C)nc1/C=N/OCC(C)(C)CO/N=C1\CCCc2ccc(C)nc21] | 1.30 (U) | | |
| 2.539 | 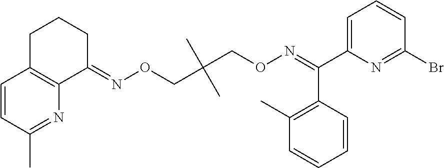 [SMILES: Cc1ccc2c(n1)/C(=N/OCC(C)(C)CO/N=C(/c1cccc(Br)n1)c1ccccc1C)CCC2] | 1.72 (U) | | |
| 2.540 | 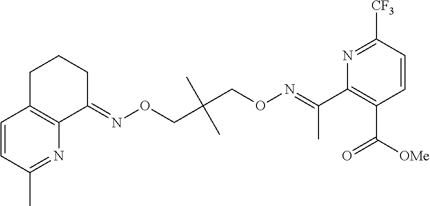 [SMILES: COC(=O)c1ccc(C(F)(F)F)nc1/C(C)=N/OCC(C)(C)CO/N=C1\CCCc2ccc(C)nc21] | 1.56 (U) | | |
| 2.541 | 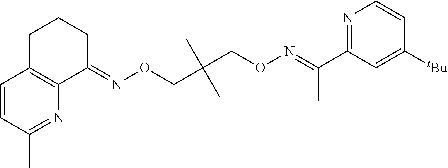 [SMILES: C/C(=N\OCC(C)(C)CO/N=C1\CCCc2ccc(C)nc21)c1cc(C(C)(C)C)ccn1] | 1.58 (U) | | |
| 2.542 | 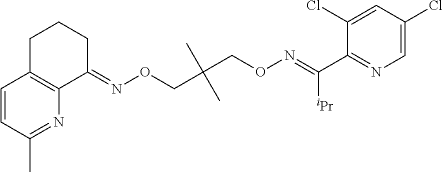 [SMILES: Cc1ccc2c(n1)/C(=N/OCC(C)(C)CO/N=C(/c1ncc(Cl)cc1Cl)C(C)C)CCC2] | 1.78 (U) | | |
| 2.543 | 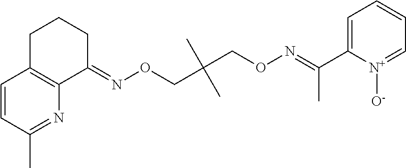 [SMILES: C/C(=N\OCC(C)(C)CO/N=C1\CCCc2ccc(C)nc21)c1cccc[n+]1[O-]] | 0.98 (U) | | |
| 2.544 | 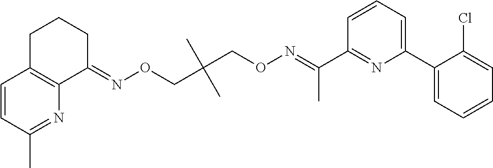 [SMILES: C/C(=N\OCC(C)(C)CO/N=C1\CCCc2ccc(C)nc21)c1cccc(-c2ccccc2Cl)n1] | 1.83 (U) | | |
| 2.545 | 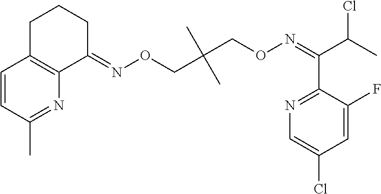 [SMILES: Cc1ccc2c(n1)/C(=N/OCC(C)(C)CO/N=C(\c1ncc(Cl)cc1F)C(C)Cl)CCC2] | 1.64 (U) | | |
| 2.546 | 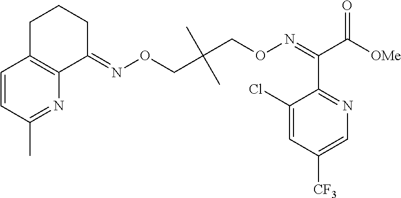 [SMILES: COC(=O)/C(=N/OCC(C)(C)CO/N=C1\CCCc2ccc(C)nc21)c1ncc(C(F)(F)F)cc1Cl] | 1.46 (U) | | |
| 2.547 | 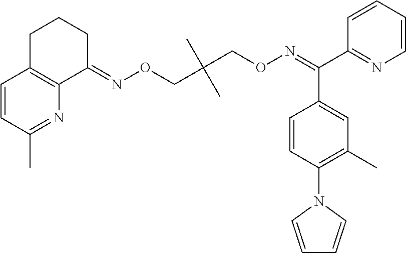 [SMILES: Cc1ccc2c(n1)/C(=N/OCC(C)(C)CO/N=C(\c1ccc(-n3cccc3)c(C)c1)c1ccccn1)CCC2] | 1.65 (U) | | |
| 2.548 | 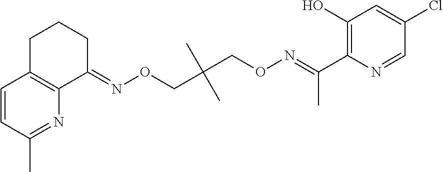 [SMILES: C/C(=N\OCC(C)(C)CO/N=C1\CCCc2ccc(C)nc21)c1ncc(Cl)cc1O] | 1.61 (U) | | |
| 2.549 | 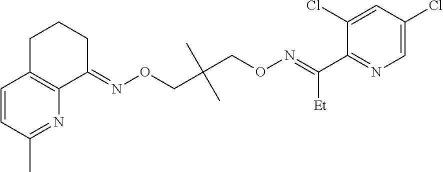 [SMILES: CC/C(=N\OCC(C)(C)CO/N=C1\CCCc2ccc(C)nc21)c1ncc(Cl)cc1Cl] | 1.67 (U) | | |
| 2.550 | 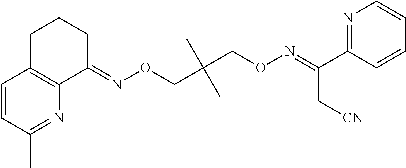 [SMILES: Cc1ccc2c(n1)/C(=N/OCC(C)(C)CO/N=C(\CC#N)c1ccccn1)CCC2] | 1.26 (U) | | |
| 2.551 Fraction A | 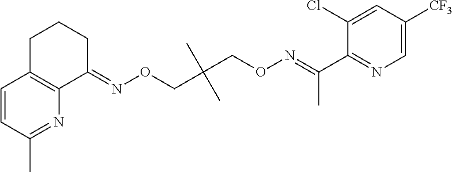 [SMILES: C/C(=N\OCC(C)(C)CO/N=C1\CCCc2ccc(C)nc21)c1ncc(C(F)(F)F)cc1Cl] | 1.64 (U) | | |
| 2.551 Fraction B | 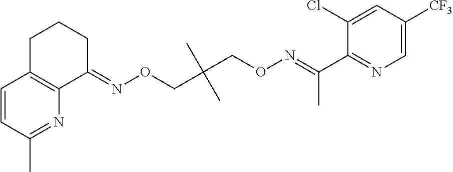 [SMILES: C/C(=N\OCC(C)(C)CO/N=C1\CCCc2ccc(C)nc21)c1ncc(C(F)(F)F)cc1Cl] | 1.65 (U) | | |
| 2.552 | 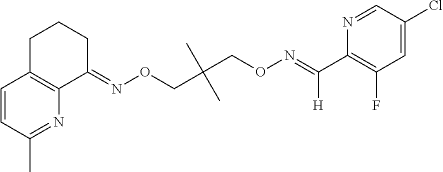 [SMILES: Cc1ccc2c(n1)/C(=N/OCC(C)(C)CO/N=C/c1ncc(Cl)cc1F)CCC2] | 1.36 (U) | | |
| 2.553 | 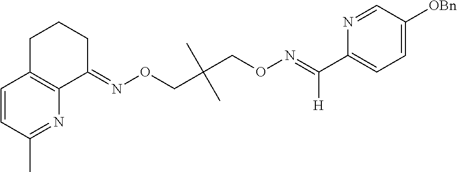 [SMILES: Cc1ccc2c(n1)/C(=N/OCC(C)(C)CO/N=C/c1ccc(OCc3ccccc3)cn1)CCC2] | 1.53 (U) | | |
| 2.554 | 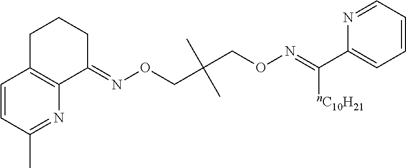 | 2.42 (U) | | |
| 2.374 | 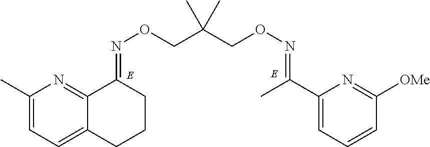 [SMILES: COc1cccc(/C(C)=N/OCC(C)(C)CO/N=C2\CCCc3ccc(C)nc32)n1] | 1.75 (ZMD) | | |
| E/E- 2.555 | 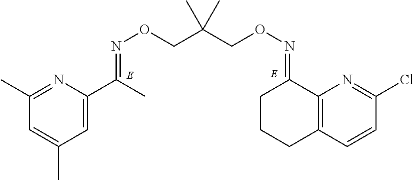 [SMILES: C/C(=N\OCC(C)(C)CO/N=C1\CCCc2ccc(Cl)nc21)c1cc(C)cc(C)n1] | 1.98 (ZMD) | 429 ([M + 1]⁺) 451 ([M + 23]⁺) | |
| E/E- 2.556 | 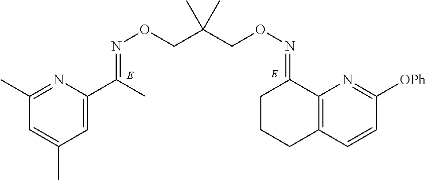 [SMILES: C/C(=N\OCC(C)(C)CO/N=C1\CCCc2ccc(Oc3ccccc3)nc21)c1cc(C)cc(C)n1] | 2.18 (ZMD) | 487 ([M + 1]⁺) 509 ([M + 23]⁺) | |
| E-2.557 | 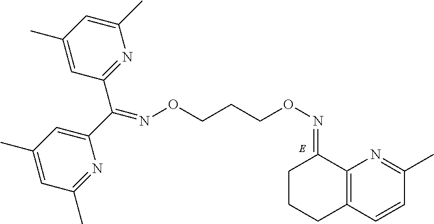 | 1.29 (ZMD) | 472 ([M + 1]⁺) 494 ([M + 23]⁺) | |
| E/E- 2.558 | 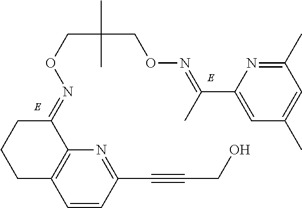 [SMILES: C/C(=N\OCC(C)(C)CO/N=C1\CCCc2ccc(C#CCO)nc21)c1cc(C)cc(C)n1] | 1.74 (ZMD) | 449 ([M + 1]⁺) | |
| E/E- 2.559 | 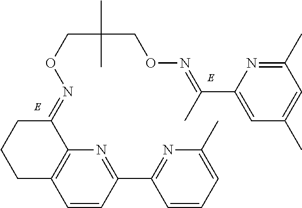 [SMILES: C/C(=N\OCC(C)(C)CO/N=C1\CCCc2ccc(-c3cccc(C)n3)nc21)c1cc(C)cc(C)n1] | 1.56 (ZMD) | 486 ([M + 1]⁺) 508 ([M + 23]⁺) | |
| E/E- 2.560 | 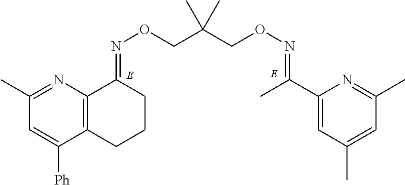 | 1.68 (ZMD) | 485 ([M + 1]⁺) | |
| E/E- 2.561 | 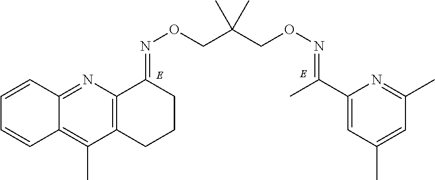 [SMILES: C/C(=N\OCC(C)(C)CO/N=C1\CCCc2c1nc1ccccc1c2C)c1cc(C)cc(C)n1] | 1.59 (ZMD) | | |
| E/E- 5.561 | 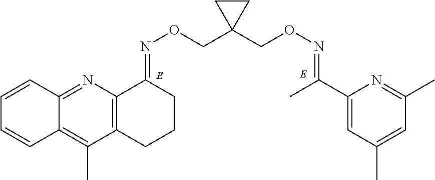 [SMILES: C/C(=N\OCC1(CO/N=C2\CCCc3c2nc2ccccc2c3C)CC1)c1cc(C)cc(C)n1] | 1.51 (ZMD) | | |
| E/E- 2.562 | 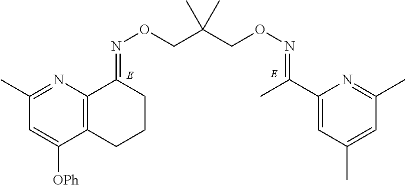 | 1.55 (ZMD) | 501 ([M + 1]⁺) | |
| E/E- 2.563 | 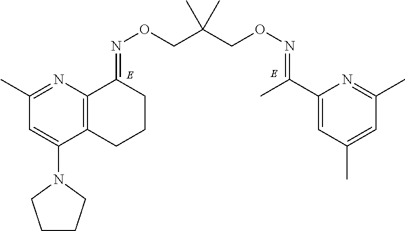 | 1.37 (ZMD) | 478 ([M + 1]⁺) | |
| E/E- 2.564 | 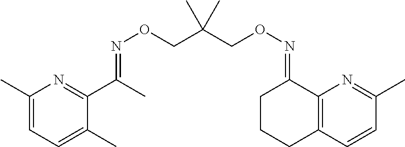 | 1.39 (ZCQ) | 409 ([M + 1]⁺) | |
| 2.565 | 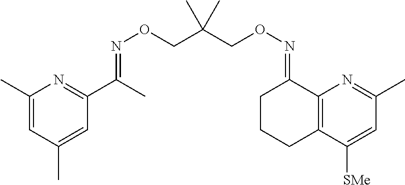 | 1.89 (ZMD) | 455 ([M + 1]⁺) | |
| 2.566 | 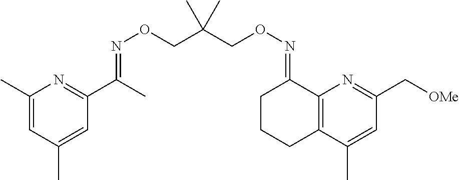 [SMILES: COCc1cc(C)c2c(n1)/C(=N/OCC(C)(C)CO/N=C(\C)c1cc(C)cc(C)n1)CCC2] | 1.59 (ZMD) | 453 ([M + 1]⁺) | |
| 2.567 | 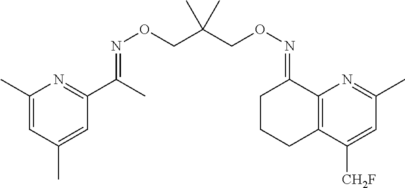 | 1.47 (ZCQ) | 441 ([M + 1]⁺) | |
| E/E- 2.010 | 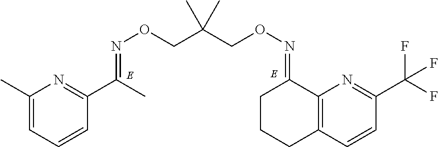 [SMILES: C/C(=N\OCC(C)(C)CO/N=C1\CCCc2ccc(C(F)(F)F)nc21)c1cccc(C)n1] | 2.13 (ZCQ) | 449 ([M + 1]⁺) 471 ([M + 23]⁺) | |
| E/E- 2.568 | 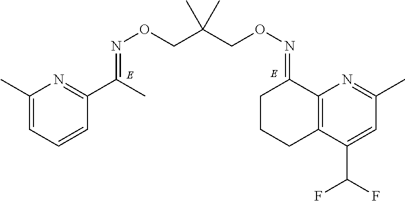 | 1.99 (ZCQ) | 445 ([M + 1]⁺) 467 ([M + 23]⁺) | |
| 2.569 | 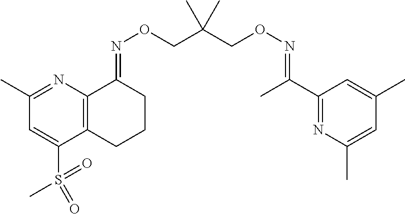 | 1.7 (ZMD) | 487 ([M + 1]⁺) | |
| E-P.01 | 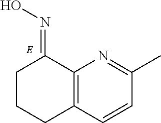 | 0.20 (ZMD) | 177 ([M + 1]⁺) 199 ([M + 23]⁺) | 177- 181 |
| E-P.02 | 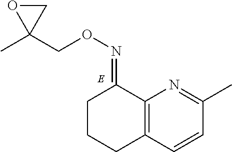 | 1.21 (ZCQ) | 221 ([M + 1]⁺) 243 ([M + 23]⁺) | |
| E-P.03 | 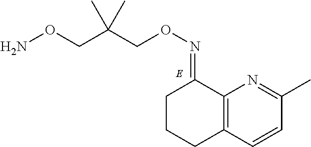 [SMILES: Cc1ccc2c(n1)/C(=N/OCC(C)(C)CON)CCC2] | 0.83 (ZCQ) | 278 ([M + 1]⁺) 300 ([M + 23]⁺) | |
| P.04 | 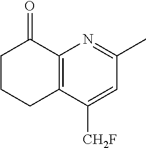 | 1.22 (ZCQ) | 212 ([M + 1]⁺) 234 ([M + 23]⁺) | |
| P.05 | 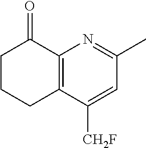 | 0.82 (ZCQ) | 194 ([M + 1]⁺) 216 ([M + 23]⁺) | |
| P.06 | 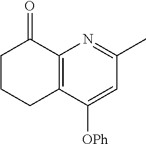 [SMILES: Cc1cc(Oc2ccccc2)c2c(n1)C(=O)CCC2] | 1.18 (ZMD) | 254 ([M + 1]⁺) | |
| P.07 | 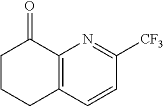 [SMILES: O=C1CCCc2ccc(C(F)(F)F)nc21] | 1.26 (ZCQ) | 216 ([M + 1]⁺) 238 ([M + 23]⁺) | |
| P.08 | 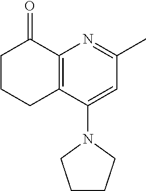 [SMILES: Cc1cc(N2CCCC2)c2c(n1)C(=O)CCC2] | 0.18 (ZMD) | 231 ([M + 1]⁺) | |
| P.09 | 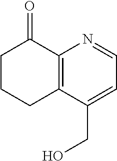 [SMILES: O=C1CCCc2c(CO)ccnc21] | 0.22 (ZCQ) | 178 ([M + 1]⁺) | |
| P.10 | 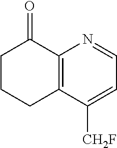 | 0.88 (ZCQ) | 180 ([M + 1]⁺) | |
| P.11 | 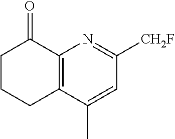 | 1.16 (ZCQ) | 194 ([M + 1]⁺) 216 ([M + 23]⁺) | |
| P.12 | 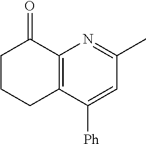 [SMILES: Cc1cc(-c2ccccc2)c2c(n1)C(=O)CCC2] | 1.30 (ZMD) | 238 ([M + 1]⁺) | |
| P.13 | 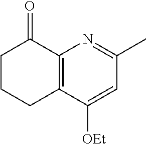 | 0.61 (ZMD) | 206 ([M + 1]⁺) 228 ([M + 23]⁺) | |
| P.14 | 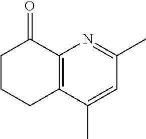 | 0.34 (ZMD) | 176 ([M + 1]⁺) | |
| P.15 | 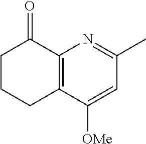 | 0.20 (ZMD) | 208 ([M + 1]⁺) | |
| P.16 | 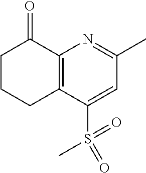 [SMILES: Cc1cc(S(C)(=O)=O)c2c(n1)C(=O)CCC2] | 1.06 (ZMD) | 240 ([M + 1]⁺) | |
| P.17 | 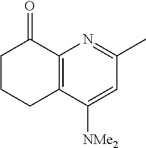 | 0.20 (ZMD) | 205 ([M + 1]⁺) | |
| P.18 | 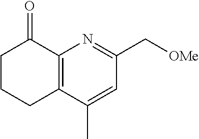 | 1.12 (ZMD) | 206 ([M + 1]⁺) 228 ([M + 23]⁺) | |
| P.18 | 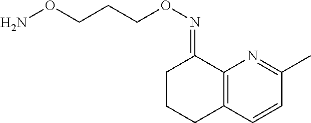 [SMILES: Cc1ccc2c(n1)/C(=N/OCCCON)CCC2] | 0.38 (ZMD) | 250 ([M + 1]⁺) | |
| P.19 | 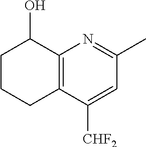 | 0.83 (ZCQ) | 214 ([M + 1]⁺) | 101- 104 |
| P.20 | 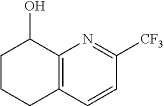 [SMILES: OC1CCCc2ccc(C(F)(F)F)nc21] | 1.41 (ZCQ) | 217 ([M + 1]⁺) | |
| P.21 | 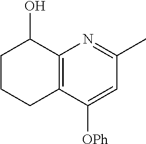 [SMILES: Cc1cc(Oc2ccccc2)c2c(n1)C(O)CCC2] | 1.00 (ZMD) | 256 ([M + 1]⁺) | |
| P.22 | 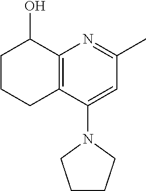 [SMILES: Cc1cc(N2CCCC2)c2c(n1)C(O)CCC2] | 0.87 (ZMD) | 233 ([M + 1]⁺) | |
| P.23 | 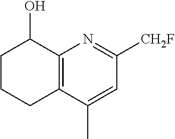 | | | mp 60-62 |
| P.24 | 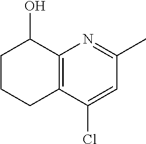 | 0.93 (ZMD) | 198 ([M + 1]⁺) | |
| P.25 | 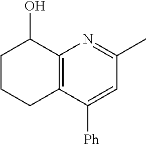 [SMILES: Cc1cc(-c2ccccc2)c2c(n1)C(O)CCC2] | 0.96 (ZMD) | 240 ([M + 1]⁺) | |
| P.26 | 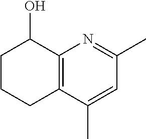 | 0.22 (ZCQ) | 178 ([M + 1]⁺) | |
| P.27 | 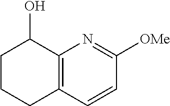 [SMILES: COc1ccc2c(n1)C(O)CCC2] | 1.14 (ZMD) | 180 ([M + 1]⁺) | |

In the table above, where two compounds have the same structure, they may be referred to as ‘Fraction A’ or ‘Fraction B’. Such fractions either come directly from the reaction and work-up or they arise from purification procedures.

This arises due to the different stereochemical isomers of the oxime or oxime ether group. When the stereodescriptors ‘E’ or ‘Z’ are not given, then the corresponding oxime or oxime ether group stereochemistry is not known. With regard to a particular oxime or oxime ether group, the actual stereochemical situation may correspond to either the ‘E-form’ or, alternatively, the ‘Z-form’, or there may be a mixture of both the ‘E’ and the ‘Z-form’.

LC-Methods Used

### Method A

Autopurification System from Waters: 2767 sample Manager, 2489 UV/Visible Detector,

2545 Quaternary Gradient Module.

Column: Phenomenex Synergi C18 Reversed Phase, 4 μm particle size, 80 Å, 75×30.00 mm,
100 mg of product dissolve in DMF injected
DAD Wavelength (nm): 220 and 254
Solvent Gradient:
A=water (Fluka Analytical)
B=Acetonitrile for praep. HPLC (Fluka Analytical)

| Time | A % | B % | Flow (mL/min) |
|---|---|---|---|
| 0.00 | 90.0 | 10.0 | 50.00 |
| 0.01 | 90.0 | 10.0 | 50.00 |
| 6.00 | 60.0 | 40.0 | 50.00 |
| 7.90 | 60.0 | 40.0 | 50.00 |
| 8.00 | 0.0 | 100.0 | 50.00 |
| 8.90 | 0.0 | 100.0 | 50.00 |
| 9.00 | 90.0 | 10.0 | 50.00 |
| 9.50 | 90.0 | 10.0 | 50.00 |
| 9.55 | 90.0 | 10.0 | 50.00 |

Method U

ACQUITY SQD Mass Spectrometer from Waters (Single quadrupole mass spectrometer)

Ionisation method: Electrospray

Polarity: positive ions

Capillary (kV) 3.80, Cone (V) 20.00, Extractor (V) 3.00, Source Temperature (° C.) 150, Desolvation Temperature (° C.) 400, Cone Gas Flow (L/Hr) 60, Desolvation Gas Flow (L/Hr) 700

Mass range: 100 to 800 Da

Column: Waters ACQUITY UPLC HSS T3; Column length: 30 mm; Internal diameter of column: 2.1 mm; Particle Size: 1.8 micron; Temperature: 60° C.

DAD Wavelength range (nm): 210 to 400

Solvent Gradient:

A=water/methanol 9:1, 0.1% HCOOH

B=Acetonitrile+0.1% HCOOH

| Time | A % | B % | Flow (mL/min) |
|---|---|---|---|
| 0 | 100.0 | 0.0 | 0.75 |
| 2.5 | 0.0 | 100.0 | 0.75 |
| 2.8 | 0.0 | 100.0 | 0.75 |
| 3.00 | 100.0 | 0.0 | 0.75 |

LC-Ms Methods Used

Method ZMD

ZMD Mass Spectrometer from Waters (Single quadrupole mass spectrometer)

Instrument Parameter:

Ionisation method: Electrospray

Polarity: positive ions

Capillary (kV) 3.80, Cone (V), Extractor (V) 3.00, Source Temperature (° C.) 150, Desolvation Temperature (° C.) 350, Cone Gas Flow (L/Hr) OFF, Desolvation Gas Flow (L/Hr) 600

Mass range: 100 to 900 Da

HP 1100 HPLC from Agilent: solvent degasser, binary pump, heated column compartment and diode-array detector.

Column: Phenomenex Gemini C18, 3 mm particle size, 110 Å 30×3 mm,

Temp: 60° C.

DAD Wavelength range (nm): 200 to 500

Solvent Gradient:

A=water+0.05% HCOOH

B=Acetonitril/Methanol (4:1, v:v)+0.04% HCOOH

| Time | A % | B % | Flow (mL/min) |
|---|---|---|---|
| 0.00 | 95.0 | 5.0 | 1.700 |
| 2.00 | 0.0 | 100.0 | 1.700 |
| 2.80 | 0.0 | 100.0 | 1.700 |
| 2.90 | 95.0 | 5.0 | 1.700 |
| 3.00 | 95.0 | 5.0 | 1.700 |

Method ZCQ

ZQ Mass Spectrometer from Waters (Single quadrupole mass spectrometer)

Instrument Parameter:

Ionisation method: Electrospray

Polarity: positive ions

Capillary (kV) 3.00, Cone (V) 30.00, Extractor (V) 2.00, Source Temperature (° C.) 100, Desolvation Temperature (° C.) 250, Cone Gas Flow (L/Hr) 50, Desolvation Gas Flow (L/Hr) 400

Mass range: 100 to 900 Da

HP 1100 HPLC from Agilent: solvent degasser, quaternary pump (ZCQ)/binary pump (ZDQ), heated column compartment and diode-array detector.

Column: Phenomenex Gemini C18, 3 mm particle size, 110 Å, 30×3 mm,

Temp: 60° C.

DAD Wavelength range (nm): 200 to 500

Solvent Gradient:

A=water+0.05% HCOOH

B=Acetonitril/Methanol (4:1, v:v)+0.04% HCOOH

| Time | A % | B % | Flow (ml/min) |
|---|---|---|---|
| 0.00 | 95.0 | 5.0 | 1.700 |
| 2.00 | 0.0 | 100.0 | 1.700 |
| 2.80 | 0.0 | 100.0 | 1.700 |
| 2.90 | 95.0 | 5.0 | 1.700 |
| 3.00 | 95.0 | 5.0 | 1.700 |

### Biological Examples

Phytophthora Infestans/Tomato/Leaf Disc Preventative (Late Blight):

Tomato leaf disks were placed on water agar in 24-well plates and sprayed with formulated test compound diluted in water at an application rate of 200 ppm. The leaf disks were inoculated with a spore suspension of the fungus 1 day after application. The inoculated leaf disks were incubated at 16° C. and 75% relative humidity under a light regime of 24 h darkness followed by 12/12 h (light/dark) darkness in a climate cabinet and the activity of a compound was assessed as percent disease control compared to untreated when an appropriate level of disease damage appears in untreated check leaf disks (5-7 days after application). The following compounds gave at least 80% control of Phytophthora infestans: E/E-2.168, E/E-2.002; 2.557

Plasmopara Viticola/Grape/Leaf Disc Preventative (Late Blight):

Grape vine leaf disks were placed on water agar in 24-well plates and sprayed with formulated test compound diluted in water at an application rate of 200 ppm. The leaf disks were inoculated with a spore suspension of the fungus 1 day after application. The inoculated leaf disks were incubated at 19° C. and 80% relative humidity under a light regime of 12/12 h (light/dark) in a climate cabinet and the activity of a compound was assessed as percent disease control compared to untreated when an appropriate level of disease damage appears in untreated check leaf disks (6-8 days after application). The following compounds gave at least 80% control of Plasmopara viticola:E/E-2.474; 5.561; 2.561

Puccinia recondita f. sp. Tritici/Wheat/Leaf Disc Preventative (Brown Rust):

Wheat leaf segments cultivated variety (cv) Kanzler were placed on agar in 24-well plates and sprayed with formulated test compound diluted in water at an application rate of 200 ppm. The leaf disks were inoculated with a spore suspension of the fungus 1 day after application. The inoculated leaf segments were incubated at 19° C. and 75% relative humidity under a light regime of 12/12 h (light/dark) in a climate cabinet and the activity of a compound was assessed as percent disease control compared to untreated when an appropriate level of disease damage appears in untreated check leaf segments (7-9 days after application). The following compounds gave at least 80% control of Puccinia recondita f. sp. tritici: E/E-2.168, E/E-2.474, E/E-2.002, E/E-2.508; 5.561; 2.561; 2.513; 2.559; 2.556; 2.555; 2.374; 2.544; 2.537; 2.530; 2.525 (fraction B); 2.519; 2.516; 2.514; E/E-5.167; E/E-3.002; 2.512; E/E-2.507

Puccinia recondita f. sp. tritici/Wheat/Leaf Disc Curative (Brown Rust):

Wheat leaf segments cv Kanzler were placed on agar in 24-well plates. The leaf segments were inoculated with a spore suspension of the fungus. The plates were stored in darkness at 19° C. and 75% relative humidity. The formulated test compound diluted in water was applied at an application rate of 200 ppm 1 day after inoculation. The leaf segments were incubated at 19° C. and 75% relative humidity under a light regime of 12/12 h (light/dark) in a climate cabinet and the activity of a compound is assessed as percent disease control compared to untreated when an appropriate level of disease damage appears in untreated check leaf segments (6-8 days after application). The following compounds gave at least 80% control of Puccinia recondita f. sp. tritici: E/E-2.168, E/E-2.474, E/E-2.002, E/E-2.508; 5.561; 2.561; E/E-2.208; 2.513; 2.559; 2.556; 2.555; 2.374; 2.552; 2.537; 2.519; 2.516; 2.514; E/E-5.167; E/E-3.002; E/E-14.002; 2.512; E/E-2.507

Phaeosphaeria nodorum (Septoria nodorum)/Wheat/Leaf Disc Preventative (Glume Blotch):

Wheat leaf segments cv Kanzler were placed on agar in a 24-well plate and sprayed with formulated test compound diluted in water at an application rate of 200 ppm. The leaf disks are inoculated with a spore suspension of the fungus 2 days after application. The inoculated test leaf disks are incubated at 20° C. and 75% relative humidity under a light regime of 12/12 h (light/dark) in a climate cabinet and the activity of a compound is assessed as percent disease control compared to untreated when an appropriate level of disease damage appears in untreated check leaf disks (5-7 days after application). The following compounds gave at least 80% control of Phaeosphaeria nodorum: E/E-2.168, E/E-2.474, E/E-2.002, E/E-2.507, E/E-2.508; 5.561; 2.561; E/E-2.208; P.57; 2.513; 2.559; 2.558; 2.557; 2.556; 2.555; 2.374; 2.544; 2.541; 2.539; 2.537; 2.536; 2.535; 2.533; 2.530; 2.526; 2.519; 2.510; 2.516; 2.514; E/E-5.167; E/E-3.002; 2.512

Pyrenophora teres/Barley/Leaf Disc Preventative (Net Blotch):

Barley leaf segments cv Hasso are placed on agar in a 24-well plate and sprayed with formulated test compound diluted in water at an application rate of 200 ppm. The leaf segments are inoculated with a spore suspension of the fungus two days after application of the test solution. The inoculated leaf segments are incubated at 20° C. and 65% relative humidity under a light regime of 12/12 h (light/dark) in a climate cabinet and the activity of a compound is assessed as disease control compared to untreated when an appropriate level of disease damage appears in untreated check leaf segments (5-7 days after application). The following compounds gave at least 80% control of Pyrenophora teres: E/E-2.168, E/E-2.474, E/E-2.002, E/E-2.507; 5.561; 2.561; E/E-2.208; P.57; 2.513; 2.559; 2.558; 2.556; 2.555; 2.374; 2.552; 2.544; 2.537; 2.533; 2.530; 2.528; 2.519; 2.510; 2.516; 2.514; E/E-5.167; E/E-3.002; 2.512

Alternaria solani/Tomato/Leaf Disc (Early Blight):

Tomato leaf disks cv Baby are placed on agar in 24-well plates (24-well format) and sprayed with the formulated test compound diluted in water at an application rate of 200 ppm. The leaf disks are inoculated with a spore suspension of the fungus 2 days after application. The inoculated leaf disks are incubated at 23° C./21° C. (day/night) and 80% relative humidity under a light regime of 12/12 h (light/dark) in a climate cabinet and the activity of a compound is assessed as percent disease control compared to untreated when an appropriate level of disease damage appears on untreated check disk leaf disks (5-7 days after application). The following compounds gave at least 80% control of Alternaria solani: E/E-2.168, E/E-2.474, E/E-2.002; 5.561; E/E-5.167

Pythium ultimum/Liquid Culture (Seedling Damping Off):

Mycelia fragments and oospores of a newly grown liquid culture of the fungus were directly mixed into nutrient broth (PDB potato dextrose broth). After placing a DMSO solution of test compound into a 96 well microtiter plate, the nutrient broth containing the fungal mycelia/spore mixture was added. The test plates were incubated at 24° C. and the inhibition of growth was determined photometrically 2-3 days after application. The following compounds gave at least 80% control of Pythium ultimum at ≦200 ppm: E/E-2.168, E/E-2.002; 2.558; 2.528; E/E-5.167

Botryotinia fuckeliana (Botrytis cinerea)/Liquid Culture (Gray Mould):

Conidia of the fungus from cryogenic storage were directly mixed into nutrient broth (Vogels broth). After placing a DMSO solution of test compound into a 96-well microtiter plate, the nutrient broth containing the fungal spores was added. The test plates were incubated at 24° C. and the inhibition of growth was determined photometrically 3-4 days after application. The following compounds gave at least 80% control of Botryotinia fuckeliana at ≦200 ppm: E/E-2.168, E/E-2.474, Z/E-2.474, E/E-2.002, E/E-2.507, E/E-2.508, E/E-2.509 (200 ppm); 5.561; 2.561; P.57; 2.513; 2.559; 2.558; 2.557; 2.556; 2.374; 2.553; 2.552; 2.551 (fraction A)'; 2.548; 2.545; 2.544; 2.542; 2.541; 2.540; 2.539; 2.537; 2.536; 2.535; 2.533; 2.532; 2.531; 2.530; 2.528; 2.526; 2.525 (fraction B); 2.525 (fraction A); 2.524; 2.520; 2.519; 2.510; 2.518; 2.517; 2.516; 2.514; E/E-5.167; E/E-3.002; E/E-14.002; 2.512; 2.514

Glomerella lagenarium (Colletotfichum lagenarium)/Liquid Culture (Anthracnose):

Conidia of the fungus from cryogenic storage were directly mixed into nutrient broth (PDB potato dextrose broth). After placing a DMSO solution of test compound into a 96-well microtiter, the nutrient broth containing the fungal spores was added. The test plates were incubated at 24° C. and the inhibition of growth is measured photometrically 3-4 days after application. The following compounds gave at least 80% control of Glomerella lagenafium at ≦200 ppm: E/E-2.168, E/E-2.474, E/E-2.002, E/E-2.507, E/E-2.508, E/E-2.509; 5.561; 2.561; E/E-2.208; P.57; 2.513; 2.559; 2.556; 2.555; 2.374; 2.552; 2.548; 2.544; 2.541; 2.540; 2.539; 2.537; 2.536; 2.533; 2.531; 2.530; 2.528; 2.526; 2.519; 2.510; 2.516; 2.514; E/E-5.167; E/E-3.002; 2.512; 2.514; Z/E-2.474

Mycosphaerella arachidis (Cercospora arachidicola)/Liquid Culture (Early Leaf Spot):

Conidia of the fungus from cryogenic storage were directly mixed into nutrient broth (PDB potato dextrose broth). After placing a DMSO solution of test compound into a 96-well microtiter plate, the nutrient broth containing the fungal spores was added. The test plates are incubated at 24° C. and the inhibition of growth was determined photometrically 4-5 days after application. The following compounds gave at least 80% control of Mycosphaerella arachidis at ≦200 ppm: E/E-2.168, E/E-2.474, Z/E-2.474, E/E-2.002, E/E-2.507, E/E-2.508, E/E-2.509; 5.561; 2.561; P.57; 2.513; 2.559; 2.558; 2.557; 2.556; 2.555; 2.374; 2.553; 2.552; 2.551 (fraction B); 2.551 (fraction A); 2.544; 2.541; 2.539; 2.537; 2.536; 2.533; 2.532; 2.531; 2.530; 2.528; 2.526; 2.525 (fraction B); 2.525 (fraction A); 2.524; 2.520; 2.519; 2.510; 2.518; 2.517; 2.516; 2.514; E/E-5.167; E/E-3.002; E/E-14.002; 2.512; 2.511; 2.514

Mycosphaerella graminicola (Septoria tritici)/Liquid Culture Septoria Blotch):

Conidia of the fungus from cryogenic storage were directly mixed into nutrient broth (PDB potato dextrose broth). After placing a DMSO solution of test compound into a 96-well microtiter plate, the nutrient broth containing the fungal spores was added. The test plates were incubated at 24° C. and the inhibition of growth was determined photometrically 4-5 days after application. The following compounds gave at least 80% control of Mycosphaerella graminicola at ≦200 ppm: E/E-2.168, E/E-2.474, E/E-2.002, E/E-2.507, E/E-2.508, E/E-2.509; 5.561; 2.561; P.57; 2.513; 2.559; 2.558; 2.556; 2.555; 2.374; 2.553; 2.552; 2.551 (fraction B); 2.551 (fraction A); 2.549; 2.548; 2.545; 2.544; 2.542; 2.541; 2.540; 2.539; 2.537; 2.536; 2.535; 2.533; 2.532; 2.531; 2.530; 2.528; 2.526; 2.525 (fraction B); 2.525 (fraction A); 2.519; 2.510; 2.517; 2.516; 2.514; E/E-5.167; E/E-3.002; 2.512; 2.514

Gaeumannomyces graminis/Liquid Culture (Take-All of Cereals):

Mycelial fragments of the fungus from cryogenic storage were directly mixed into nutrient broth (PDB potato dextrose broth). After placing a DMSO solution of test compound into a 96-well microtiter plate, the nutrient broth containing the fungal spores is added. The test plates were incubated at 24° C. and the inhibition of growth was determined photometrically 4-5 days after application. The following compounds gave at least 80% control of Gaeumannomyces graminis at ≦200 ppm: E/E-2.168, E/E-2.474, E/E-2.002, E/E-2.507, E/E-2.509; 5.561; 2.561; P.57; 2.513; 2.559; 2.558; 2.557; 2.556; 2.555; 2.374; 2.553; 2.552; 2.551 (fraction B); 2.551 (fraction A); 2.539; 2.533; 2.528; 2.526; 2.525 (fraction B); 2.525 (fraction A); 2.524; 2.523; 2.521; 2.520; 2.519; 2.510; 2.518; 2.516; 2.514; E/E-5.167; E/E-3.002; 2.512; E/E-2.508

Thanatephorus cucumeris (Rhizoctonia solani)/Liquid Culture (Foot Rot, Damping-Off

Mycelia fragments of a newly grown liquid culture of the fungus are directly mixed into nutrient broth (PDB potato dextrose broth). After placing a DMSO solution of the test compounds into a 96-well microtiter, the nutrient broth containing the fungal material was added. The test plates were incubated at 24° C. and the inhibition of growth was determined photometrically 3-4 days after application. The following compounds gave at least 80% control of Thanatephorus cucumeris at ≦200 ppm: E/E-2.168, E/E-2.474, E/E-2.002, E/E-2.507, E/E-2.508 5.561; 2.561; P.57; 2.513; 2.559; 2.558; 2.553; 2.533; 2.530; 2.526; 2.519; 2.517; 2.516; 2.514; 2.512; 2.514

Monographella nivalis (Microdochium nivale)/Liquid Culture (Foot Rot Cereals):

Conidia of the fungus from cryogenic storage were directly mixed into nutrient broth (PDB potato dextrose broth). After placing a DMSO solution of test compound into a 96-well microtiter plate, the nutrient broth containing the fungal spores was added. The test plates were incubated at 24° C. and the inhibition of growth was determined photometrically 4-5 days after application. The following compounds gave at least 80% control of Monographella nivalis at ≦200 ppm: E/E-2.168, E/E-2.474, E/E-2.002, E/E-2.507, E/E-2.508, E/E-2.509; P.57; 2.513; 2.559; 2.558; 2.556; 2.555; 2.374; 2.553; 2.551 (fraction B); 2.551 (fraction A); 2.548; 2.544; 2.542; 2.541; 2.539; 2.536; 2.535; 2.533; 2.530; 2.528; 2.526; 2.525 (fraction B); 2.525 (fraction A); 2.524; 2.519; 2.518; 2.516; 2.514; E/E-5.167; 2.512; 2.514

Blumeria graminis f. sp. tritici (Erysiphe graminis f. sp. tritici)/Wheat/Leaf Disc Preventative (Powdery Mildew on Wheat):

Wheat leaf segments cv. Kanzler were placed on agar in a 24-well plate and sprayed with the formulated test compound diluted in water at an application rate of 200 ppm. The leaf disks were inoculated by shaking powdery mildew infected plants above the test plates 1 day after application. The inoculated leaf disks were incubated at 20° C. and 60% relative humidity under a light regime of 24 h darkness followed by 12 h/12 h (dark/light) in a climate chamber and the activity of a compound was assessed as percent disease control compared to untreated when an appropriate level of disease damage appears on untreated check leaf segments (6-8 days after application). The following compounds gave at least 80% control of Blumeria graminis: E/E-2.168, E/E-2.474, E/E-2.002; 5.561; 2.561; E/E-2.208; 2.513; 2.559; 2.557; 2.556; 2.555; 2.374; 2.552; 2.537; 2.533; 2.530; 2.528; 2.526; 2.519; 2.510; 2.516; 2.514; E/E-5.167; E/E-3.002; 2.512; 2.511

Magnaporthe grisea (Pyriculana oryzae)/Rice/Leaf Disc Preventative (Rice Blast):

Rice leaf segments cv. Ballila were placed on agar in a multiwell plate (24-well format) and sprayed with the formulated test compound diluted in water. The leaf segments were inoculated with a spore suspension of the fungus 2 days after application. The inoculated leaf segments were incubated at 22° C. and 80% rh under a light regime of 24 h darkness followed by 12 h/12 h (dark/light) in a climate cabinet and the activity of a compound was assessed as percent disease control compared to untreated when an appropriate level of disease damage appears in untreated check leaf segments (5-7 days after application). The following compounds gave at least 80% control of Magnaporthe grisea: 5.561; 2.561; E/E-2.208; 2.513; 2.558

1. A compound of formula (I) 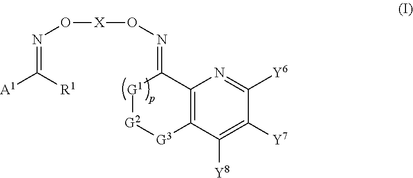
wherein
R¹ represents hydrogen, halogen, CN, OH, SH, C₁-C₈ alkylthio, C₁-C₈ alkylsulphinyl, C₁-C₈ alkylsulphonyl, NH₂, C₁-C₁₀ alkyl, C₃-C₈ cycloalkyl, C₂-C₈ alkenyl, C₂-C₈ alkynyl, (C₁-C₄ alkyloxycarbonyl) C₁-C₄-alkyl, (C₁-C₄ alkyl)O₂C, phenyl or pyridyl, wherein the alkyl, cycloalkyl, alkenyl, alkynyl, phenyl and pyridyl are optionally substituted by one or more groups independently selected from halogen, CN, NH₂, NO₂, OH, C₁-C₄ alkyl, C₁-C₄ haloalkyl, C₁-C₄ alkoxy, C₁-C₄ haloalkoxy, C₃-C₆ cycloalkyl and a 5- or 6-membered heterocycle containing one to three heteroatoms independently selected from O, S and N, providing that the heterocycle does not contain adjacent oxygen atoms, adjacent sulphur atoms, or adjacent sulphur and oxygen atoms;
A¹ represents cycle A-2, A-4, or A-5: 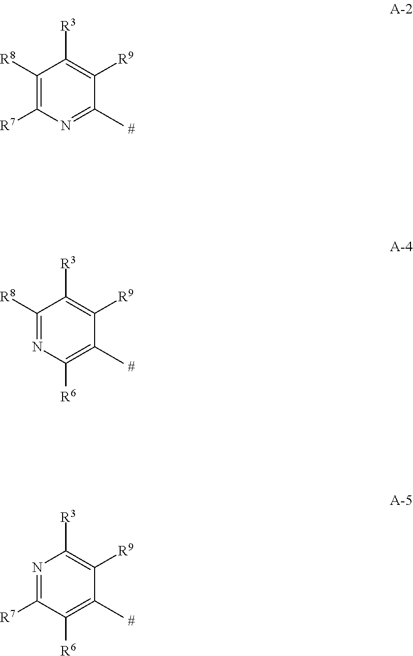
R³, R⁶, R⁷, R⁸ and R⁹ independently of one another represent hydrogen, halogen, CN, NO₂, C₁-C₈ alkyl, C₁-C₄-alkoxy-C₁-C₄-alkyl, C₁-C₄-alkoxy-C₁-C₄-alkoxy-C₁-C₄-alkyl, C₃-C₈ cycloalkyl, C₂-C₈ alkenyl, C₂-C₈ alkynyl, phenyl, a 5- or 6-membered heterocycle containing one to three heteroatoms independently selected from O, S and N, providing that the heterocycle does not contain adjacent oxygen atoms, adjacent sulphur atoms, or adjacent sulphur and oxygen atoms, COR¹³, OR¹¹, SH, C₁-C₈-alkylthio, C₁-C₈-alkylsulphinyl, C₁-C₈-alkylsulphonyl, N(R¹²)₂, CO₂R¹¹, O(CO)R¹³, CON(R¹²)₂, NR¹²COR¹³ or CR¹³N—OR¹¹, wherein the alkyl, alkoxy, cycloalkyl, alkenyl, alkynyl, phenyl and heterocycle are optionally substituted by one or more groups independently selected from halogen, CN, NH₂, NO₂, OH, C₁-C₄ alkyl, C₁-C₄ haloalkyl, C₁-C₄ alkoxy and C₁-C₄ haloalkoxy;
or R⁶ and R⁷, R⁷ and R⁸, R³ and R⁸, or R³ and R⁹ together with the fragment of the pyridyl ring to which they are attached may form a partially or fully unsaturated 5- to 7-membered carbocyclic ring or a 5- to 7-membered heterocyclic ring containing one to three heteroatoms independently selected from O, S, N and N(R¹²), providing that the heterocycle does not contain adjacent oxygen atoms, adjacent sulphur atoms, or adjacent sulphur and oxygen atoms, and wherein the ring formed by R⁶ and R⁷, R⁷ and R⁸, R³ and R⁸, or R³ and R⁹ is optionally substituted by one or more groups independently selected from halogen, CN, NH₂, NO₂, OH, C₁-C₄ alkyl, C₁-C₄ haloalkyl, C₁-C₄ alkoxy and C₁-C₄ haloalkoxy;
X represents X-2, X-3, X-4 or X-5:
#—Z²—Z³—# X-2
#—Z⁴—Z⁵—Z⁶—# X-3
#—Z⁷—Z⁸—Z⁹—Z¹⁰—# X-4
#—Z¹¹—Z¹²—Z¹³—Z¹⁴—Z¹⁵—# X-5
Z², Z³, Z⁴, Z⁶, Z⁷, Z⁸, Z⁹, Z¹⁰, Z¹¹, Z¹², Z¹⁴ and Z¹⁵ independently of one another represent CR¹⁴R¹⁵, C═O or C═CR¹⁹R²⁰;
Z⁵ and Z¹³ independently of one another represent CR^{14′}R^{15′}, SiR¹⁶R¹⁷, C═O or C═CR¹⁹R²⁰;
each R¹⁴ and R¹⁵ independently of one another represent hydrogen, halogen, OH, C₁-C₄ alkyl, C₁-C₄ haloalkyl, phenyl or CN, wherein the phenyl is optionally substituted by one or more groups independently selected from halogen, CN, C₁-C₄ alkyl, C₁-C₄ haloalkyl, C₁-C₄ alkoxy and C₁-C₄ haloalkyoxy;
or R¹⁴ and R¹⁵ together with the carbon atom to which they are attached may form a C₃-C₆ cycloalkyl group or a C₃-C₆ halocycloalkyl group;
each R¹⁹ and R²⁰ independently of one another represent hydrogen, halogen, C₁-C₄ alkyl or C₁-C₄ haloalkyl;
each R^{14′}, R^{15′}, R¹⁶ and R¹⁷ independently of one another represent hydrogen, halogen, OH, C₁-C₄ alkyl, C₁-C₄ haloalkyl, phenyl or CN, wherein phenyl is optionally substituted by one or more groups independently selected from halogen, CN, C₁-C₄ alkyl, C₁-C₄ haloalkyl, C₁-C₄ alkoxy and C₁-C₄ haloalkyoxy;
or R^{14′} and R^{15′} together with the carbon atom to which they are attached may form a C₃-C₆ cycloalkyl group or a C₃-C₆ halocycloalkyl group;
wherein the groupings X-2, X-3, X-4 and X-5 contain at most one ring which contains either only one of the radicals Z² to Z¹⁵ or two radicals Z² to Z¹⁵ or three radicals Z² to Z¹⁵ or four radicals Z² to Z¹⁵ as ring members; and wherein radicals Z², Z³, Z⁴, Z⁶, Z⁷, Z¹⁰, Z¹¹ and Z¹⁵ are not ubstituted by OH;
Y⁶, Y⁷ and Y⁸ independently of one another represent hydrogen, halogen, CN, NO₂, C₁-C₈ alkyl, C₁-C₄-alkoxy-C₁-C₄-alkyl, C₁-C₄-alkoxy-C₁-C₄-alkoxy-C₁-C₄-alkyl, C₃-C₈ cycloalkyl, C₂-C₈ alkenyl, C₂-C₈ alkynyl, phenyl, pyridyl, COR¹³, OR²², SH, C₁-C₈ alkylthio, C₁-C₈ alkylsulphinyl, C₁-C₈ alkylsulphonyl, N(R²³)₂, CO₂R²², O(CO)R¹³, CON(R²³)₂, NR²³COR¹³ or CR¹³N—OR²², wherein the alkyl, alkoxy, cycloalkyl, alkenyl, alkynyl, phenyl and pyridyl are optionally substituted by one or more groups independently selected from halogen, CN, NH₂, NO₂, OH, C₁-C₄ alkyl, C₁-C₄ haloalkyl, C₁-C₄ alkoxy and C₁-C₄ haloalkoxy;
or Y⁶ and Y⁷ or Y⁷ and Y⁸ together with the fragment of the pyridyl ring to which they are attached may form a partially or fully unsaturated 5- to 7-membered carbocyclic ring or a 5- to 7-membered heterocyclic ring containing one to three heteroatoms independently selected from O, S, N and N(R¹²), providing that the heterocycle does not contain adjacent oxygen atoms, adjacent sulphur atoms, or adjacent sulphur and oxygen atoms, and wherein the ring formed by Y⁶ and Y⁷ or Y⁷ and Y⁸ is optionally substituted by one or more groups independently selected from halogen, CN, NH₂, NO₂, OH, C₁-C₄ alkyl, C₁-C₄ haloalkyl, C₁-C₄ alkoxy and C₁-C₄ haloalkoxy;
each R¹¹ and R²² independently of one another represent hydrogen, C₁-C₈ alkyl, C₃-C₈ cycloalkyl, C₃-C₈ alkenyl, C₃-C₈ alkynyl, benzyl, phenyl or pyridyl, wherein the alkyl, cycloalkyl, alkenyl, alkynyl, phenyl, benzyl and pyridyl are optionally substituted by one or more groups independently selected from halogen, CN, NH₂, NO₂, OH, C₁-C₄ alkyl, C₁-C₄-haloalkyl, C₁-C₄ alkoxy, C₁-C₄ haloalkoxy and C₁-C₄-alkoxy-C₁-C₄-alkyl;
each R¹² and R²³ independently of one another represent hydrogen, OH, C₁-C₈ alkyl, C₁-C₈ alkoxy, C₁-C₈-alkoxy-C₁-C₄-alkyl, C₃-C₈ alkenyl, C₃-C₈ alkynyl, or COR¹³, wherein the alkyl, alkoxy, alkenyl and alkynyl are optionally substituted by one or more halogen;
wherein when two radicals R¹² or two radicals R²³ are attached to the same nitrogen atom, these radicals can be identical or different;
wherein when two radicals R¹² or two radicals R²³ are attached to the same nitrogen atom, both of these radicals cannot be OH, C₁-C₄ alkoxy or C₁-C₄ haloalkoxy;
and wherein when two radicals R¹² or two radicals R²³ are attached to the same nitrogen atom, these two radicals together with the nitrogen atom to which they are attached may form a cycle B-1, B-2, B-3, B-4, B-5, B-6, B-7 or B-8: 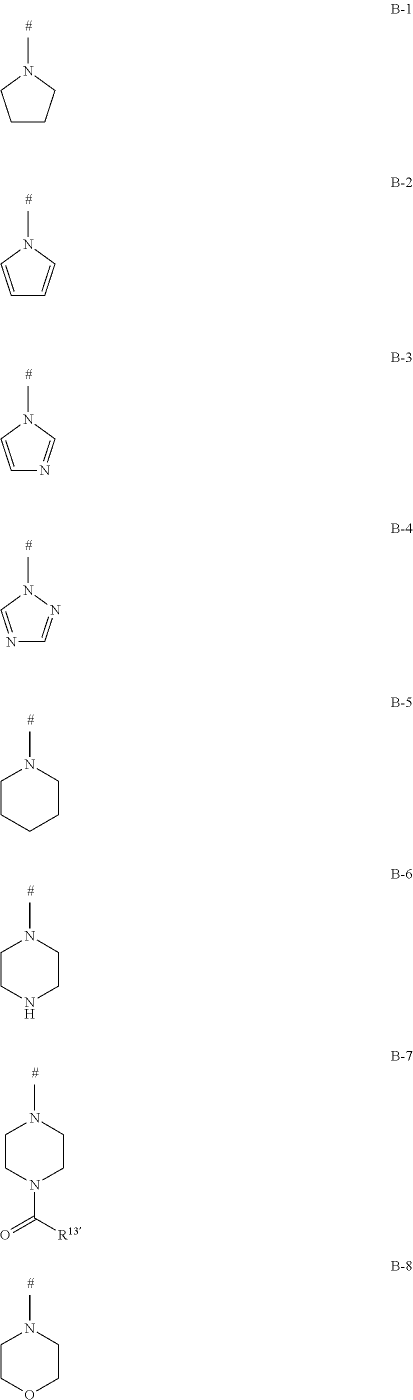
wherein the cycle formed is optionally substituted by one or more groups independently selected from halogen, CN, NH₂, NO₂, OH, C₁-C₄ alkyl, C₁-C₄ haloalkyl, C₁-C₄ alkoxy and C₁-C₄ haloalkoxy;
each R¹³ and R^{13′} independently of one another represent hydrogen, C₁-C₈ alkyl, C₃-C₈ cycloalkyl, C₂-C₈ alkenyl, C₂-C₈ alkynyl, benzyl, phenyl or pyridyl, wherein the alkyl, cycloalkyl, alkenyl, alkynyl, phenyl, benzyl and pyridyl are optionally substituted by one or more groups independently selected from halogen, CN, NH₂, NO₂, OH, C₁-C₄ alkyl, C₁-C₄ haloalkyl, C₁-C₄ alkoxy and C₁-C₄ haloalkoxy;
G¹ and G² independently of one another represent —C(R²⁴R²⁵)—;
G³ represents —C(R²⁴R²⁵)—, O, N(R²⁶) or S;
or G¹ and G², or G² and G³, or G¹ and G¹ together represent —CR²⁴═CR²⁵—;
each R²⁴ and R²⁵ independently of one another represent hydrogen, halogen, C₁-C₄ alkyl, C₁-C₄ haloalkyl, C₁-C₄ alkoxy or C₁-C₄ haloalkoxy;
R²⁶ represents hydrogen, OH, C₁-C₄ alkyl, C₁-C₄ alkoxy, C₁-C₈ alkylcarbonyl or C₁-C₈ haloalkylcarbonyl; and
p is 1 or 2;
or a salt or an N-oxide thereof.

2. A compound according to claim 1 wherein R¹ represents hydrogen, (C₁-C₄ alkyl)O₂C, C₁-C₁₀ alkyl, phenyl or pyridyl, wherein the alkyl, phenyl and pyridyl are optionally substituted by one or more groups independently selected from halogen, CN, C₁-C₄ alkyl, C₁-C₄ haloalkyl, C₃-C₆ cycloalkyl and a 5- or 6-membered heterocycle containing one to three nitrogen atoms;
A¹ represents cycle A-2, A-4, or A-5;
R³, R⁶, R⁷, R⁸ and R⁹ independently of one another represent hydrogen, halogen, CN, C₁-C₈ alkyl, C₃-C₈ cycloalkyl, C₂-C₈ alkenyl, C₂-C₈ alkynyl, phenyl, a 5- or 6-membered heterocycle containing one to three nitrogen atoms, OR¹¹, SH, C₁-C₈-alkylthio, C₁-C₈-alkylsulphinyl, C₁-C₈-alkylsulphonyl, CO₂R¹¹, CON(R¹²)₂, or wherein the alkyl, cycloalkyl, alkenyl, alkynyl, phenyl and heterocycle are optionally substituted by one or more groups independently selected from halogen, CN, NH₂, NO₂, OH, C₁-C₄ alkyl, C₁-C₄ haloalkyl, C₁-C₄ alkoxy and C₁-C₄ haloalkoxy;
or R⁶ and R⁷, R⁷ and R⁸, R³ and R⁸, or R³ and R⁹ together with the fragment of the pyridyl ring to which they are attached may form a partially or fully unsaturated 5- to 7-membered carbocyclic ring, and wherein the ring formed by R⁶ and R⁷, R⁷ and R⁸, R³ and R⁸, or R³ and R⁹ is optionally substituted by one or more groups independently selected from halogen, CN, NH₂, NO₂, OH, C₁-C₄ alkyl, C₁-C₄ haloalkyl, C₁-C₄ alkoxy and C₁-C₄ haloalkoxy;
each R¹¹ independently of one another represent hydrogen, C₁-C₄ alkyl, C₃-C₈ alkenyl, C₃-C₈ alkynyl, benzyl, or phenyl, wherein the alkyl, cycloalkyl, alkenyl, alkynyl, phenyl and benzyl are optionally substituted by one or more groups independently selected from halogen, CN, NH₂, NO₂, OH, C₁-C₄ alkyl, C₁-C₄-haloalkyl, C₁-C₄ alkoxy and C₁-C₄ haloalkoxy;
each R¹² independently of one another represent hydrogen or C₁-C₈ alkyl.

3. A compound according to claim 1 wherein A¹ represents cycle A-2, A-4, or A-5;
R³, R⁶, R⁷, R⁸ and R⁹ independently of one another represent hydrogen, halogen, CN, C₁-C₄ alkyl, C₂-C₆ alkenyl, C₂-C₆ alkynyl, phenyl, OR¹¹, SH, C₁-C₄-alkylthio, C₁-C₄-alkylsulphinyl, C₁-C₄-alkylsulphonyl, wherein the alkyl, alkoxy, alkenyl, alkynyl and phenyl are optionally substituted by one or more groups independently selected from halogen, CN, NH₂, NO₂, OH, C₁-C₄ alkyl, C₁-C₄ haloalkyl, C₁-C₄ alkoxy and C₁-C₄ haloalkoxy;
or R⁶ and R⁷, R⁷ and R⁸, R³ and R⁸, or R³ and R⁹ together with the fragment of the pyridyl ring to which they are attached may form a partially or fully unsaturated 5- to 7-membered carbocyclic ring;
each R¹¹ independently of one another represent hydrogen, C₁-C₄ alkyl, C₃-C₆ alkenyl, C₃-C₆ alkynyl or phenyl;
each R¹² independently of one another represent hydrogen or C₁-C₈ alkyl.

4. A compound according to claim 1, wherein G¹, G² and G³ represent methylene.

5. A compound according to claim 1, wherein p is 1.

6. A compound according to claim 1, wherein X represents X-3.

7. A compound according to claim 1, wherein
X represents X-3;
Z⁴ and Z⁶ represent methylene;
Z⁵ represents CR^{14′}R^{15′} or C═CR¹⁹R²⁰;
each R^{14′} and R^{15′} independently of one another represent hydrogen, halogen, C₁-C₄ alkyl, C₁-C₄ haloalkyl or phenyl, wherein the phenyl is optionally substituted by one or more groups independently selected from halogen, CN, methyl, halomethyl, methoxy and halomethoxy;
or R^{14′} and R^{15′} together with the carbon atom to which they are attached may form a C₃-C₆ cycloalkyl group optionally substituted by halogen; and
each R¹⁹ and R²⁰ independently of one another represent hydrogen, halogen, methyl or halomethyl.

8. A compound according to claim 1 wherein Y⁶, Y⁷ and Y⁸ independently of one another represent hydrogen, halogen, N(R²³)₂CN, NO₂, C₁-C₈ alkyl, C₁-C₆-alkoxy-C₁-C₄-alkyl, C₃-C₈ cycloalkyl, C₂-C₆ alkenyl, C₂-C₆ alkynyl, phenyl, pyridyl, OR²², SH, C₁-C₈ alkylthio, C₁-C₈ alkylsulphinyl or C₁-C₈ alkylsulphonyl, wherein the alkyl, alkoxy, cycloalkyl, alkenyl, alkynyl, phenyl and pyridyl are optionally substituted by one or more groups independently selected from halogen, CN, NH₂, NO₂, OH, C₁-C₄ alkyl, C₁-C₄ haloalkyl, C₁-C₄ alkoxy and C₁-C₄ haloalkoxy;
or Y⁶ and Y⁷ or Y⁷ and Y⁸ together with the fragment of the pyridyl ring to which they are attached may form a partially or fully unsaturated 5- to 7-membered carbocyclic ring, wherein the ring formed by Y⁶ and Y⁷ or Y⁷ and Y⁸ is optionally substituted by one or more groups independently selected from halogen, CN, NH₂, NO₂, OH, C₁-C₄ alkyl, C₁-C₄ haloalkyl, C₁-C₄ alkoxy and C₁-C₄ haloalkoxy;
each R²² independently of one another represent hydrogen, C₁-C₄ alkyl, C₃-C₆ cycloalkyl, C₃-C₆ alkenyl, C₃-C₆ alkynyl, benzyl, phenyl or pyridyl, wherein the alkyl, cycloalkyl, alkenyl, alkynyl, phenyl, benzyl and pyridyl are optionally substituted by one or more groups independently selected from halogen, CN, NH₂, NO₂, OH, C₁-C₄ alkyl, C₁-C₄-haloalkyl, C₁-C₄ alkoxy, C₁-C₄ haloalkoxy and C₁-C₄-alkoxy-C₁-C₄-alkyl;
each R²³ independently of one another represent hydrogen or C₁-C₈ alkyl, wherein the alkyl is optionally substituted by one or more halogen;
wherein when two radicals R²³ are attached to the same nitrogen atom, these radicals can be identical or different;
and wherein when two radicals R²³ are attached to the same nitrogen atom, these two radicals together with the nitrogen atom to which they are attached may form a cycle B-1, B-2, B-3, B-4 or B-5 wherein the cycle formed is optionally substituted by one or more groups independently selected from halogen, methyl and halomethyl.

9. A compound according to claim 1 wherein Y⁶, Y⁷ and Y⁸ independently of one another represent hydrogen, halogen, N(R²³)₂CN, NO₂, C₁-C₆ alkyl, C₁-C₄-alkoxy-C₁-C₄-alkyl, C₃-C₆ cycloalkyl, C₂-C₆ alkenyl, C₂-C₆ alkynyl, phenyl, pyridyl, C₁-C₄-alkoxy, C₁-C₄-alkenoxy, C₁-C₄-alkynoxy, phenoxy, SH, C₁-C₈ alkylthio, C₁-C₈ alkylsulphinyl or C₁-C₈ alkylsulphonyl, wherein the alkyl, alkoxy, alkenoxy, alkynoxy, phenoxy cycloalkyl, alkenyl, alkynyl, phenyl and pyridyl are optionally substituted by one or more groups independently selected from halogen, CN, NH₂, NO₂, OH, methyl and halomethyl;
or Y⁶ and Y⁷ or Y⁷ and Y⁸ together with the fragment of the pyridyl ring to which they are attached may form a partially or fully unsaturated 5- to 7-membered carbocyclic ring, wherein the ring formed by Y⁶ and Y⁷ or Y⁷ and Y⁸ is optionally substituted by one or more groups independently selected from halogen, CN, NH₂, NO₂, OH, methyl and halomethyl;
each R²³ independently of one another represent hydrogen or C₁-C₈ alkyl, wherein the alkyl is optionally substituted by one or more halogen;
wherein when two radicals R²³ are attached to the same nitrogen atom, these radicals can be identical or different;
and wherein when or two radicals R²³ are attached to the same nitrogen atom, these two radicals together with the nitrogen atom to which they are attached may form a cycle B-1, B-2, B-3, B-4 or B-5 wherein the cycle formed is optionally substituted by one or more groups independently selected from halogen, methyl and halomethyl.

10. A compound according to claim 1, wherein R³, R⁶, R⁷, R⁸ and R⁹ independently of one another represent hydrogen, C₁-C₄ alkyl, CN or C₁-C₄ alkoxy, wherein the alkyl and alkoxy are optionally substituted by one or more groups independently selected from halogen, CN, C₁-C₄ alkoxy and C₁-C₄ haloalkoxy.

11. A compound according to claim 1, wherein A¹ represents cycle A-2.

12. A compound according to claim 1 wherein R¹ represents hydrogen, halogen, CN, OH, SH, C₁-C₈ alkylthio, C₁-C₈ alkylsulphinyl, C₁-C₈ alkylsulphonyl, NH₂, C₁-C₁₀ alkyl, C₃-C₈ cycloalkyl, C₂-C₈ alkenyl, C₂-C₈ alkynyl, (C₁-C₄ alkyloxycarbonyl) C₁-C₄-alkyl, (C₁-C₄ alkyl)O₂C, phenyl or pyridyl, wherein the alkyl, cycloalkyl, alkenyl, alkynyl, phenyl and pyridyl are optionally substituted by one or more groups independently selected from halogen, CN, NH₂, NO₂, OH, C₁-C₄ alkyl, C₁-C₄ haloalkyl, C₁-C₄ alkoxy, C₁-C₄ haloalkoxy, C₃-C₆ cycloalkyl and a 5- or 6-membered heterocycle containing one to three heteroatoms independently selected from O, S and N, providing that the heterocycle does not contain adjacent oxygen atoms, adjacent sulphur atoms, or adjacent sulphur and oxygen atoms;
A¹ represents cycle A-2;
R³, R⁷, R⁸ and R⁹ independently of one another represent hydrogen, halogen, CN, C₁-C₈ alkyl, C₃-C₈ cycloalkyl, C₂-C₈ alkenyl, C₂-C₈ alkynyl, phenyl, a 5- or 6-membered heterocycle containing one to three nitrogen atoms, OR¹¹, SH, C₁-C₈-alkylthio, C₁-C₈-alkylsulphinyl, C₁-C₈-alkylsulphonyl, CO₂R¹¹ or CON(R¹²)₂, wherein the alkyl, cycloalkyl, alkenyl, alkynyl, phenyl and heterocycle are optionally substituted by one or more groups independently selected from halogen, CN, NH₂, NO₂, OH, C₁-C₄ alkyl, C₁-C₄ haloalkyl, C₁-C₄ alkoxy and C₁-C₄ haloalkoxy;
or R⁷ and R⁸, R³ and R⁸, or R³ and R⁹ together with the fragment of the pyridyl ring to which they are attached may form a partially or fully unsaturated 5- to 7-membered carbocyclic ring, and wherein the ring formed by R⁷ and R⁸, R³ and R⁸, or R³ and R⁹ is optionally substituted by one or more groups independently selected from halogen, CN, NH₂, NO₂, OH, C₁-C₄ alkyl, C₁-C₄ haloalkyl, C₁-C₄ alkoxy and C₁-C₄ haloalkoxy;
each R¹¹ independently of one another represent hydrogen, C₁-C₄ alkyl, C₃-C₈ alkenyl, C₃-C₈ alkynyl, benzyl, or phenyl, wherein the alkyl, cycloalkyl, alkenyl, alkynyl, phenyl and benzyl are optionally substituted by one or more groups independently selected from halogen, CN, NH₂, NO₂, OH, C₁-C₄ alkyl, C₁-C₄-haloalkyl, C₁-C₄ alkoxy and C₁-C₄ haloalkoxy;
each R¹² independently of one another represent hydrogen or C₁-C₈ alkyl;
X represents X-3;
Z⁴ and Z⁶ represent methylene;
Z⁵ represents CR^{14′}R^{15′} or C═CR¹⁹R²⁰;
each R^{14′} and R^{15′} independently of one another represent hydrogen, halogen, C₁-C₄ alkyl, C₁-C₄ haloalkyl or phenyl, wherein the phenyl is optionally substituted by one or more groups independently selected from halogen, CN, methyl, halomethyl, methoxy and halomethoxy;
or R^{14′} and R^{15′} together with the carbon atom to which they are attached may form a C₃-C₆ cycloalkyl group optionally substituted by halogen; and
each R¹⁹ and R²⁰ independently of one another represent hydrogen, halogen, methyl or halomethyl;
Y⁶, Y⁷ and Y⁸ independently of one another represent hydrogen, halogen, N(R²³)₂CN, NO₂, C₁-C₈ alkyl, C₁-C₆-alkoxy-C₁-C₄-alkyl, C₃-C₈ cycloalkyl, C₂-C₆ alkenyl, C₂-C₆ alkynyl, phenyl, pyridyl, OR²², SH, C₁-C₈ alkylthio, C₁-C₈ alkylsulphinyl or C₁-C₈ alkylsulphonyl, wherein the alkyl, alkoxy, cycloalkyl, alkenyl, alkynyl, phenyl and pyridyl are optionally substituted by one or more groups independently selected from halogen, CN, NH₂, NO₂, OH, C₁-C₄ alkyl, C₁-C₄ haloalkyl, C₁-C₄ alkoxy and C₁-C₄ haloalkoxy;
or Y⁶ and Y⁷ or Y⁷ and Y⁸ together with the fragment of the pyridyl ring to which they are attached may form a partially or fully unsaturated 5- to 7-membered carbocyclic ring, wherein the ring formed by Y⁶ and Y⁷ or Y⁷ and Y⁸ is optionally substituted by one or more groups independently selected from halogen, CN, NH₂, NO₂, OH, C₁-C₄ alkyl, C₁-C₄ haloalkyl, C₁-C₄ alkoxy and C₁-C₄ haloalkoxy;
each R²² independently of one another represent hydrogen, C₁-C₄ alkyl, C₃-C₄ cycloalkyl, C₃-C₆ alkenyl, C₃-C₆ alkynyl, benzyl, phenyl or pyridyl, wherein the alkyl, cycloalkyl, alkenyl, alkynyl, phenyl, benzyl and pyridyl are optionally substituted by one or more groups independently selected from halogen, CN, NH₂, NO₂, OH, C₁-C₄ alkyl, C₁-C₄-haloalkyl, C₁-C₄ alkoxy, C₁-C₄ haloalkoxy and C₁-C₄-alkoxy-C₁-C₄-alkyl;
each R²³ independently of one another represent hydrogen or C₁-C₈ alkyl, wherein the alkyl is optionally substituted by one or more halogen;
wherein when two radicals R²³ are attached to the same nitrogen atom, these radicals can be identical or different;
and wherein when two radicals R²³ are attached to the same nitrogen atom, these two radicals together with the nitrogen atom to which they are attached may form a cycle B-1, B-2, B-3, B-4 or B-5 wherein the cycle formed is optionally substituted by one or more groups independently selected from halogen, methyl and halomethyl;
G¹, G² and G³ represent methylene; and
p is 1 or 2.

13. A compound according to claim 1 wherein R¹ represents hydrogen, (C₁-C₄ alkyl)O₂C, C₁-C₁₀ alkyl, phenyl or pyridyl, wherein the alkyl, phenyl and pyridyl are optionally substituted by one or more groups independently selected from halogen, CN, C₁-C₄ alkyl, C₁-C₄ haloalkyl, C₃-C₆ cycloalkyl and a 5- or 6-membered heterocycle containing one to three nitrogen atoms;
A¹ represents cycle A-2;
R³, R⁷, R⁸ and R⁹ independently of one another represent hydrogen, halogen, CN, C₁-C₄ alkyl, C₂-C₆ alkenyl, C₂-C₆ alkynyl, phenyl, OR¹¹, SH, C₁-C₄-alkylthio, C₁-C₄-alkylsulphinyl, C₁-C₄-alkylsulphonyl, wherein the alkyl, alkoxy, cycloalkyl, alkenyl, alkynyl and phenyl are optionally substituted by one or more groups independently selected from halogen, CN, NH₂, NO₂, OH, C₁-C₄ alkyl, C₁-C₄ haloalkyl, C₁-C₄ alkoxy and C₁-C₄ haloalkoxy;
or R⁷ and R⁸, R³ and R⁸, or R³ and R⁹ together with the fragment of the pyridyl ring to which they are attached may form a partially or fully unsaturated 5- to 7-membered carbocyclic ring;
each R¹¹ independently of one another represent hydrogen, C₁-C₄ alkyl, C₃-C₆ alkenyl, C₃-C₆ alkynyl or phenyl;
each R¹² independently of one another represent hydrogen or C₁-C₈ alkyl;
X represents X-3;
Z⁴ and Z⁶ represent methylene;
Z⁵ represents CR^{14′}R^{15′} or C═CR¹⁹R²⁰;
each R^{14′} and R^{15′} independently of one another represent hydrogen, halogen, C₁-C₄ alkyl, C₁-C₄ haloalkyl or phenyl, wherein the phenyl is optionally substituted by one or more groups independently selected from halogen, CN, methyl, halomethyl, methoxy and halomethoxy;
or R^{14′} and R^{15′} together with the carbon atom to which they are attached may form a C₃-C₆ cycloalkyl group optionally substituted by halogen; and
each R¹⁹ and R²⁰ independently of one another represent hydrogen, halogen, methyl or halomethyl;
Y⁶, Y⁷ and Y⁸ independently of one another represent hydrogen, halogen, N(R²³)₂CN, NO₂, C₁-C₆ alkyl, C₁-C₄-alkoxy-C₁-C₄-alkyl, C₃-C₆ cycloalkyl, C₂-C₆ alkenyl, C₂-C₆ alkynyl, phenyl, pyridyl, C₁-C₄-alkoxy, C₁-C₄-alkenoxy, C₁-C₄-alkynoxy, phenoxy, SH, C₁-C₈ alkylthio, C₁-C₈ alkylsulphinyl or C₁-C₈ alkylsulphonyl, wherein the alkyl, alkoxy, alkenoxy, alkynoxy, phenoxy, cycloalkyl, alkenyl, alkynyl, phenyl and pyridyl are optionally substituted by one or more groups independently selected from halogen, CN, NH₂, NO₂, OH, methyl and halomethyl;
or Y⁶ and Y⁷ or Y⁷ and Y⁸ together with the fragment of the pyridyl ring to which they are attached may form a partially or fully unsaturated 5- to 7-membered carbocyclic ring, wherein the ring formed by Y⁶ and Y⁷ or Y⁷ and Y⁸ is optionally substituted by one or more groups independently selected from halogen, CN, NH₂, NO₂, OH, methyl and halomethyl;
each R²³ independently of one another represent hydrogen or C₁-C₈ alkyl, wherein the alkyl, is optionally substituted by one or more halogen;
wherein when two radicals R²³ are attached to the same nitrogen atom, these radicals can be identical or different;
and wherein when or two radicals R²³ are attached to the same nitrogen atom, these two radicals together with the nitrogen atom to which they are attached may form a cycle B-1, B-2, B-3, B-4 or B-5 wherein the cycle formed is optionally substituted by one or more groups independently selected from halogen, methyl and halomethyl;
G¹, G² and G³ represent methylene; and
p is 1.

14. A compound of formula (II) 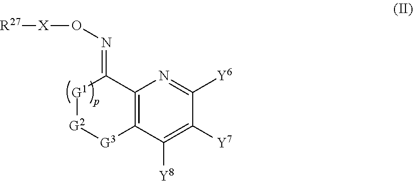
wherein R²⁷ represents —ONH₂, halogen, —O—SO₂—R²⁸ or one of the groups LG, C-1, C-2A or C-2B: 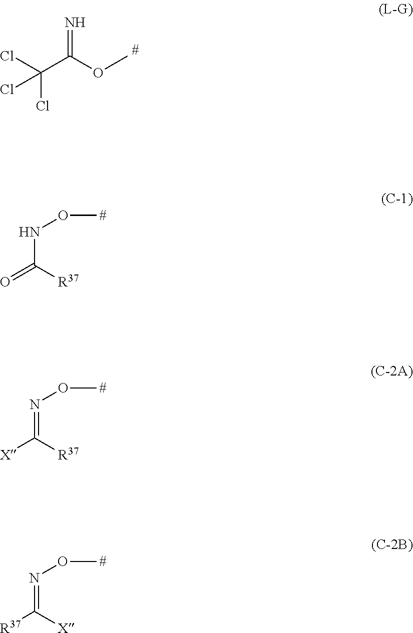
R²⁸ represents C₁-C₄ alkyl, C₁-C₄ haloalkyl or phenyl, wherein the phenyl is optionally substituted by one or two substituents independently selected from methyl, trihalomethyl, NO₂, CN, C₁-C₇-alkoxycarbonyl;
X″ represents halogen;
R³⁷ represents either A¹ or R¹ as defined for the compound of formula (I) in claim 1; and
X, G¹, G², G³, Y⁶, Y⁷, Y⁸ and p are as defined for the compound of formula (I) in claim 1;
or a salt or N-oxide thereof, or;
a compound of formula (III) 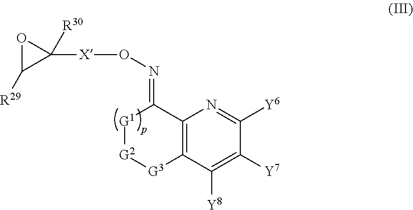
wherein X′ represents one of the groupings X′-1, X′-2 or X′-3:
#—Z⁶—# X′-1
#—Z⁹—Z¹⁰—# X′-2
#—Z¹³—Z¹⁴—Z¹⁵—# X′-3
Z⁶, Z⁹, Z¹⁰, Z¹³, Z¹⁴ and Z¹⁵ are as defined for the compound of formula (I) in claim 1;
R²⁹ and R³⁰ independently of one another represent hydrogen, halogen, C₁-C₄ alkyl, C₁-C₄ haloalkyl, phenyl or CN, wherein phenyl is optionally substituted by one or more groups independently selected from halogen, CN, C₁-C₄ alkyl, C₁-C₄ haloalkyl, C₁-C₄ alkoxy and C₁-C₄ haloalkoxy; and
G¹, G², G³, Y⁶, Y⁷, Y⁸ and p are as defined for the compound of formula (I) in claim 1;
or a salt or N-oxide thereof, or;
a compound of formula (VIII) 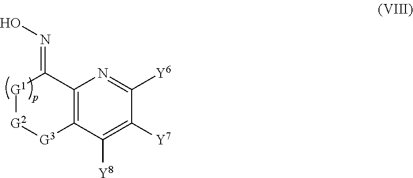
wherein G¹, G², G³, Y⁶, Y⁷, Y⁸ and p are as defined for the compound of formula (I) in claim 1, providing that:
when p is 1 and G¹, G², G³ are —CH₂—, then Y⁶, Y⁷ and Y⁸ are not all H;
when p is 1, G¹, G², G³ are —CH₂—, and Y⁷ and Y⁸ are H, then Y⁶ is not methoxy;
when p is 1, G¹, G², G³ are —CH₂—, and Y⁶ and Y⁸ are H, then Y⁷ is not methyl;
when p is 1, G¹, G² are —CH₂—, and Y⁶, Y⁷ and Y⁸ are H, then G³ is not O;
when p is 1, G¹ and G² together form CH═CH, Y⁶, Y⁷ and Y⁸ are H, then G³ is not C(CHCl₂)(CH₃);
when p is 2 and G¹, G², G³ are —CH₂—, then Y⁶, Y⁷ and Y⁸ are not all H;
or a salt or N-oxide thereof.

15. A compound of formula (II) according to claim 14, wherein
R²⁷ represents —ONH₂, —O—SO₂—R²⁸ or one of the groups LG, C-1, C-2A or C-2B: 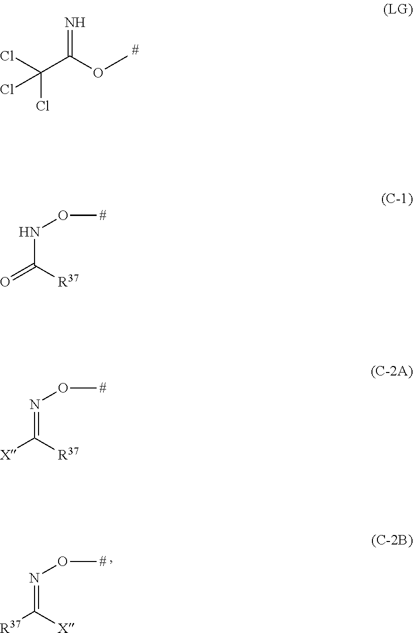
or;
a compound of formula (VIII) according to claim 14, wherein
Y⁶ represents halogen, CN, NO₂, C₁-C₈-alkyl, C₁-C₄-alkoxy-C₁-C₄-alkyl, C₁-C₄-alkoxy-C₁-C₄-alkoxy-C₁-C₄-alkyl, C₃-C₈-cycloalkyl, C₂-C₈-alkenyl, C₂-C₈-alkynyl, phenyl, pyridyl, COR¹³, OR^{22′}, SH, C₁-C₈-alkylthio, C₁-C₈-alkylsulphinyl, C₁-C₈-alkylsulphonyl, N(R²³)₂, CO₂R²², O(CO)R¹³, CON(R²³)₂, NR²³COR¹³ or CR¹³N—OR²², wherein the alkyl, alkoxy, cycloalkyl, alkenyl, alkynyl, phenyl and pyridyl are optionally substituted by one or more groups independently selected from halogen, CN, NH₂, NO₂, OH, C₁-C₄-alkyl, C₁-C₄-haloalkyl, C₁-C₄-alkoxy and C₁-C₄-haloalkoxy;
R^{22′} represents hydrogen, C₂-C₈-alkyl, C₃-C₈-cycloalkyl, C₃-C₈-alkenyl, C₃-C₈-alkynyl, benzyl, phenyl or pyridyl, wherein the alkyl, cycloalkyl, alkenyl, alkynyl, phenyl and pyridyl are optionally substituted by one or more groups independently selected from halogen, CN, NH₂, NO₂, OH, C₁-C₄-alkyl, C₁-C₄-haloalkyl, C₁-C₄-alkoxy, C₁-C₄-haloalkoxy and C₁-C₄-alkoxy-C₁-C₄-alkyl;
R¹³, R²², R²³, G¹, G², G³, Y⁷, Y⁸ and p are as defined for the compound of formula (I) in claim 1.

16. A process for the production of a compound of formula (I) as defined in claim 1 comprising reacting a compound of formula (IIb) with a compound of formula (X) as shown in scheme X
Scheme X 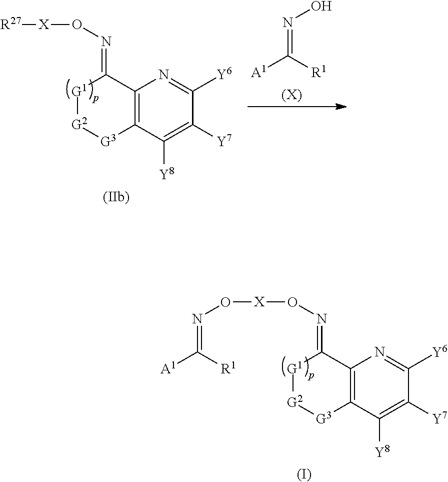
wherein R²⁷ represents halogen, —O—SO₂—R²⁸ or group LG: 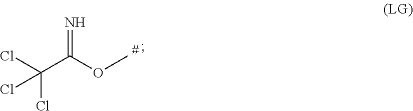
R²⁸ represents C₁-C₄-alkyl, C₁-C₄-haloalkyl or phenyl, wherein the phenyl is optionally substituted by one or two substituents independently selected from methyl, trihalomethyl, NO₂, CN, C₁-C₇-alkoxycarbonyl; and
X, G¹, G², G³, Y⁶, Y⁷, Y⁸, p, A¹ and R¹ are as defined for the compound of formula (I) in claim 1.

17. A fungicidal composition comprising a fungicidally effective amount of a compound of formula (I) as defined in claim 1, optionally comprising an additional active ingredient.

18. A method of controlling phytopathogenic diseases on useful plants or on propagation material thereof, which comprises applying to the useful plants, the locus thereof or propagation material thereof a fungicidally effective amount of a compound of formula (I) as defined in claim 1.

19. A compound of formula IVa 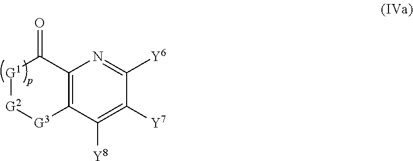
wherein G¹, G², G³, p, Y⁶, Y⁷ and Y⁸ are as defined for a compound of formula I in claim 1, or a salt or N-oxide thereof, wherein the compound is not one of the compounds below:

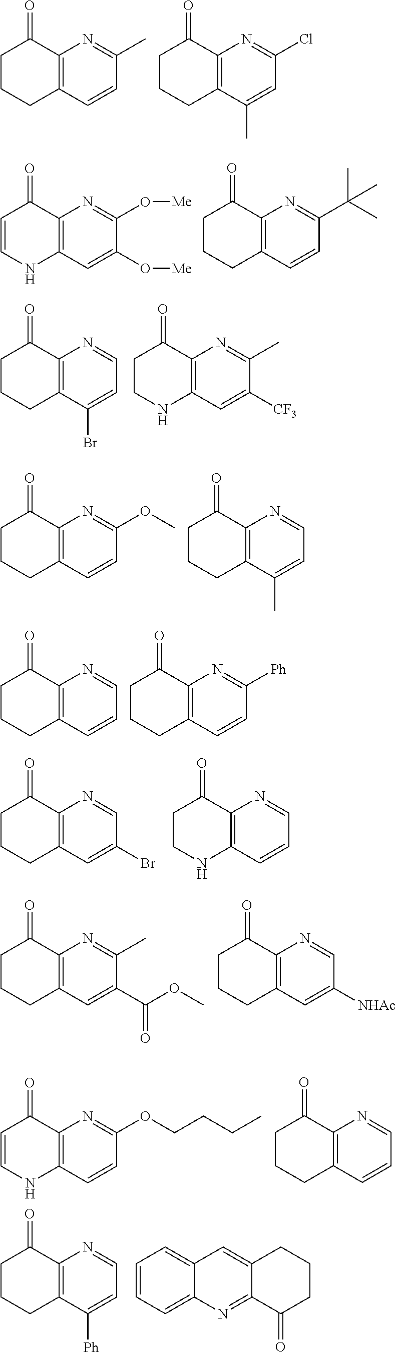

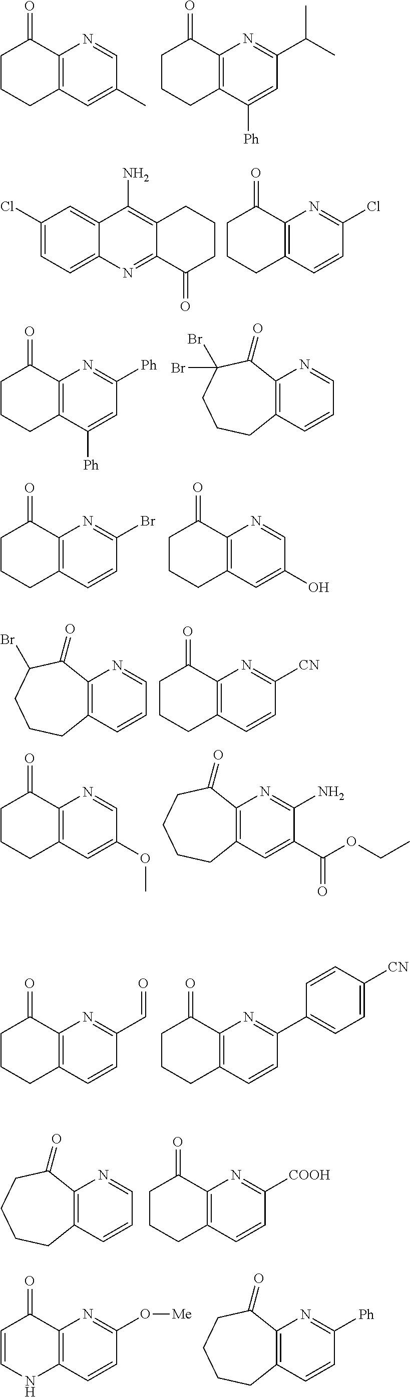

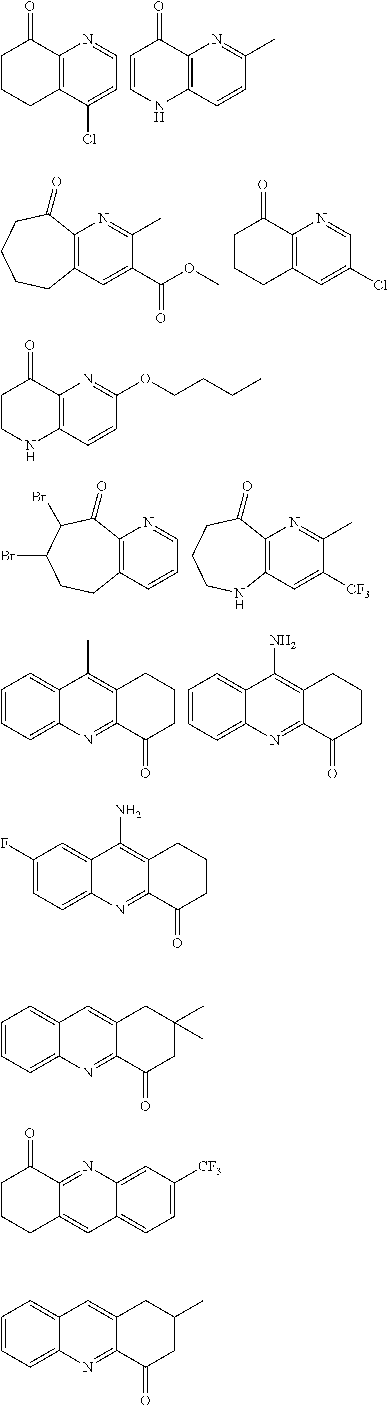

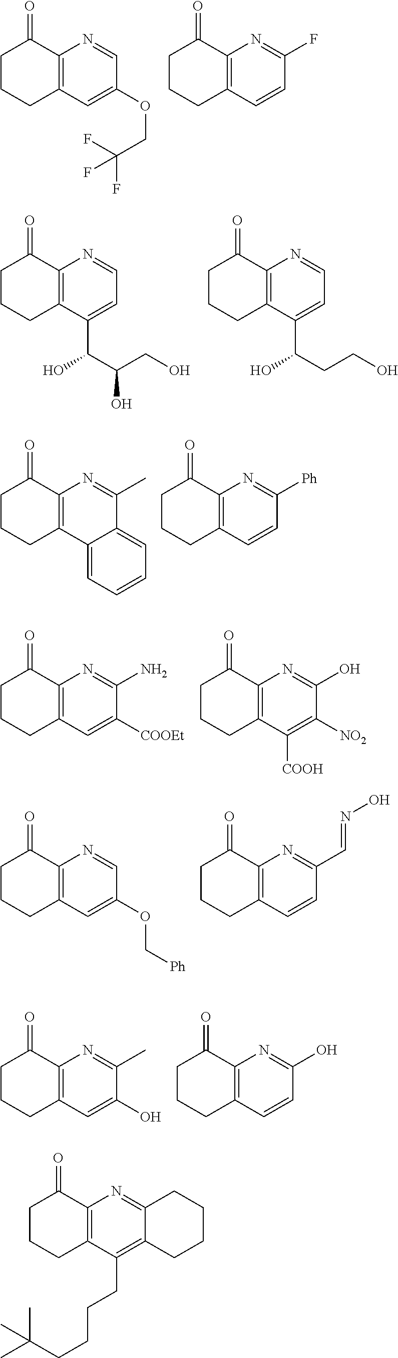

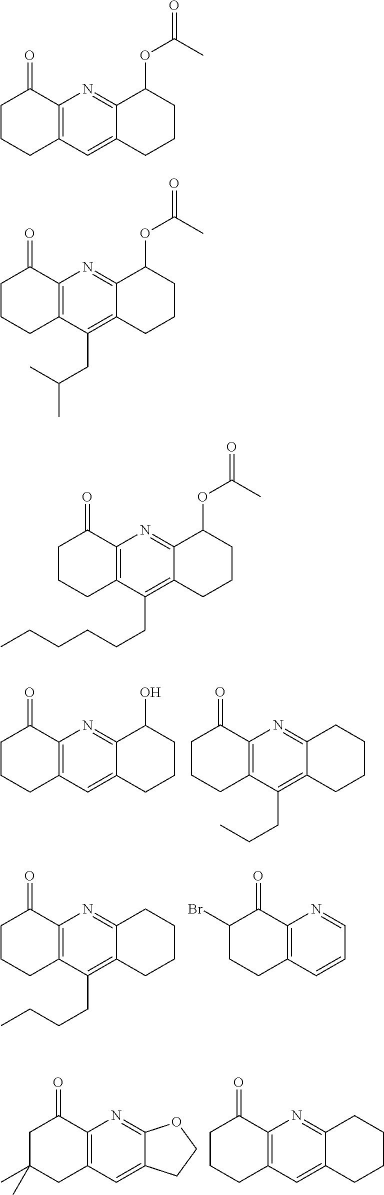

20. A compound according to claim 19, wherein
Y⁶ is C₁-C₆ alkyl, halogen, NH₂, C₁-C₆ haloalkyl, C₁-C₆ alkoxy, or C₁-C₆ haloalkoxy;
Y⁷ and Y⁸ independently of one another represent hydrogen, halogen, CN, NO₂, C₁-C₈ alkyl, C₁-C₄-alkoxy-C₁-C₄-alkyl, C₁-C₄-alkoxy-C₁-C₄-alkoxy-C₁-C₄-alkyl, C₃-C₈ cycloalkyl, C₂-C₈ alkenyl, C₂-C₈ alkynyl, phenyl, pyridyl, COR¹³, OR²², SH, C₁-C₈ alkylthio, C₁-C₈ alkylsulphinyl, C₁-C₈ alkylsulphonyl, N(R²³)₂, CO₂R²², O(CO)R¹³, CON(R²³)₂, NR²³COR¹³ or CR¹³N—OR²², wherein the alkyl, alkoxy, cycloalkyl, alkenyl, alkynyl, phenyl and pyridyl are optionally substituted by one or more groups independently selected from halogen, CN, NH₂, NO₂, OH, C₁-C₄ alkyl, C₁-C₄ haloalkyl, C₁-C₄ alkoxy and C₁-C₄ haloalkoxy;
G¹ and G² independently represent —C(R²⁴)(R²⁵)—;
G³ represents —C(R²⁴)(R²⁵)—, O, N(R²⁶) or S;
each R²⁴ and R²⁵ independently of one another represent hydrogen, halogen, C₁-C₄ alkyl, C₁-C₄ haloalkyl, C₁-C₄ alkoxy, or C₁-C₄ haloalkyl;
R²⁶ is hydrogen, OH, C₁-C₄ alkyl or C₁-C₄ alkoxy;
p is 1 or 2.

21. A compound according to claim 19, wherein Y⁶ is hydrogen, methyl or halomethyl.

22. A compound according to claim 19, wherein the compound is one of the compounds below:

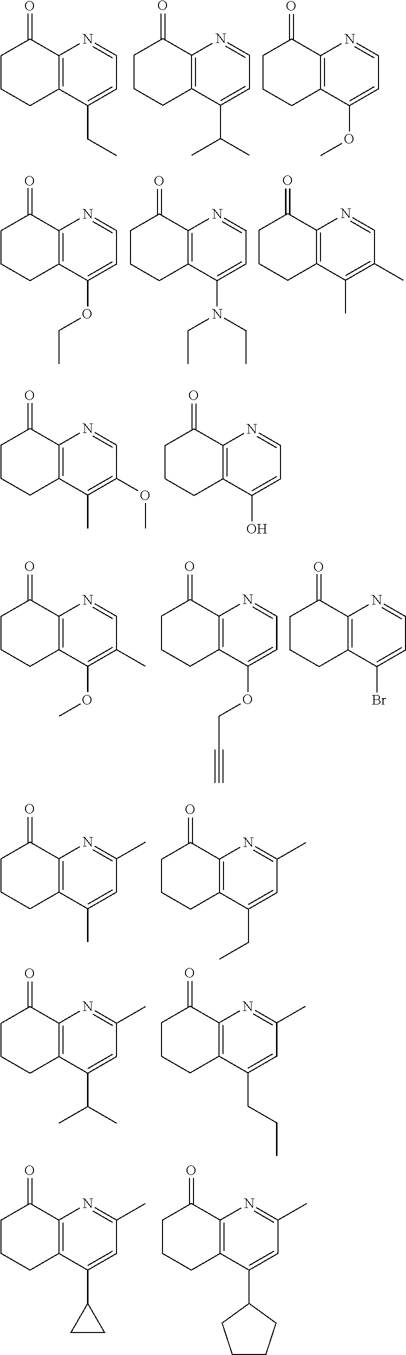

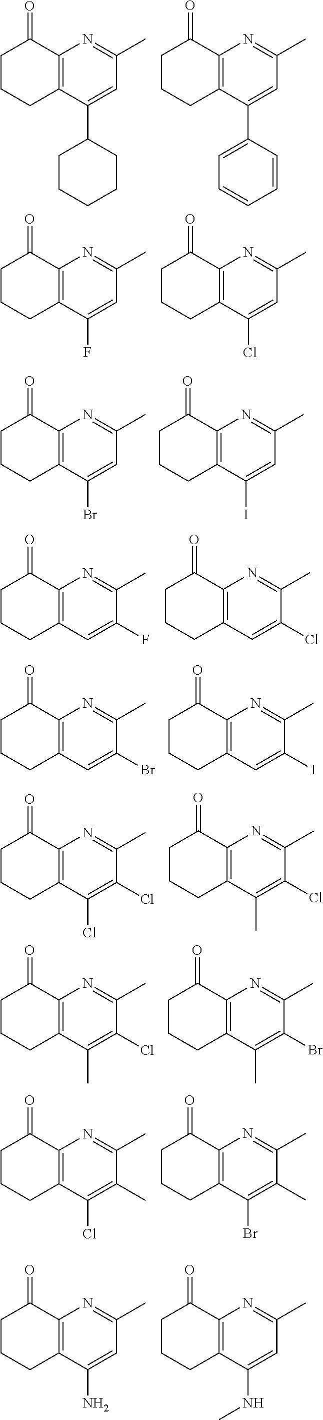

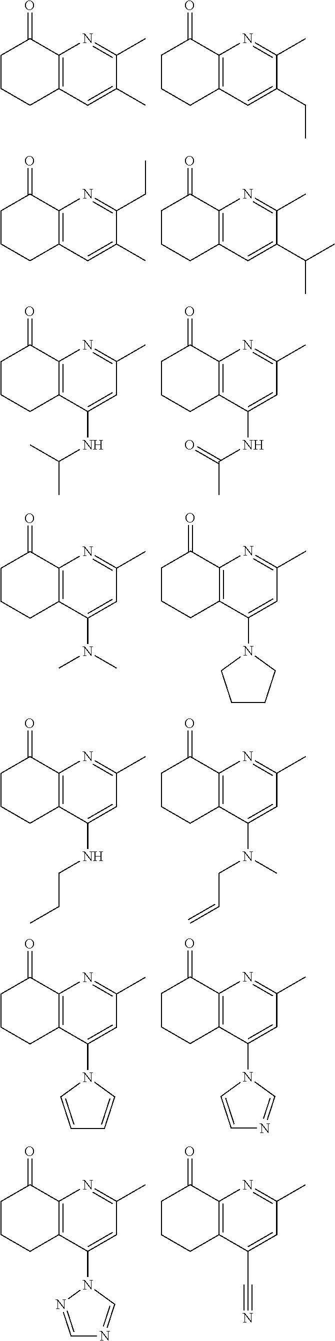

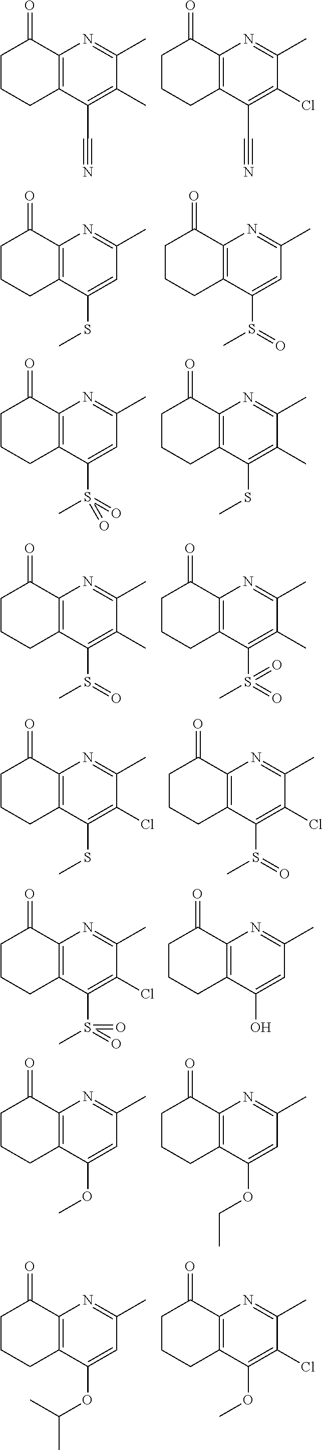

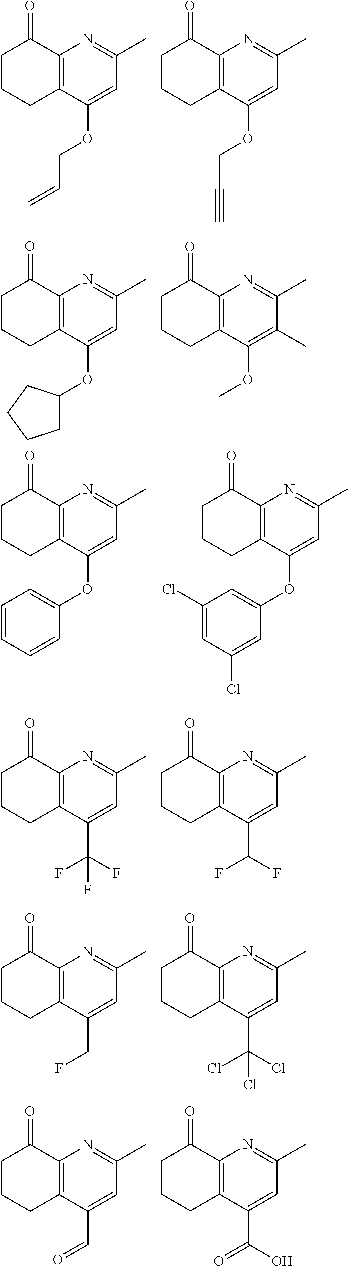

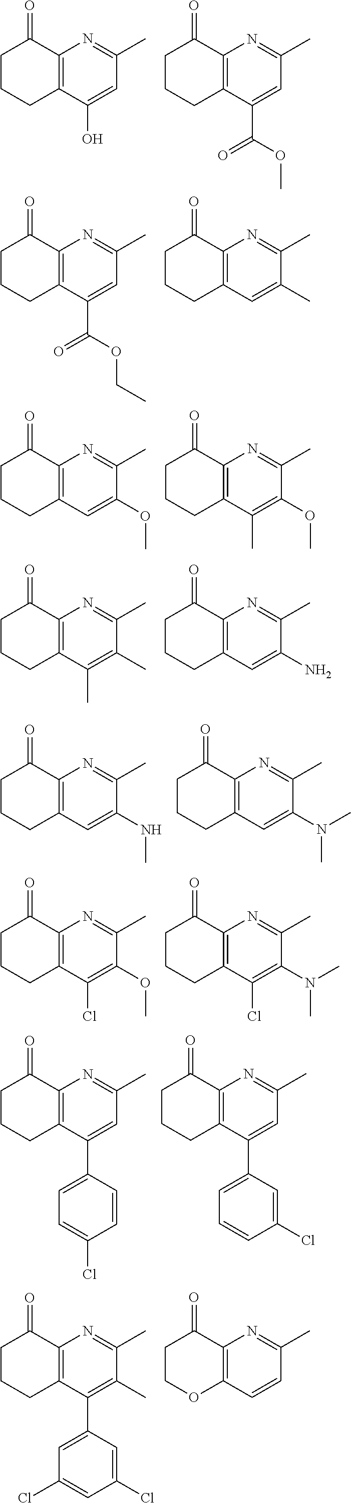

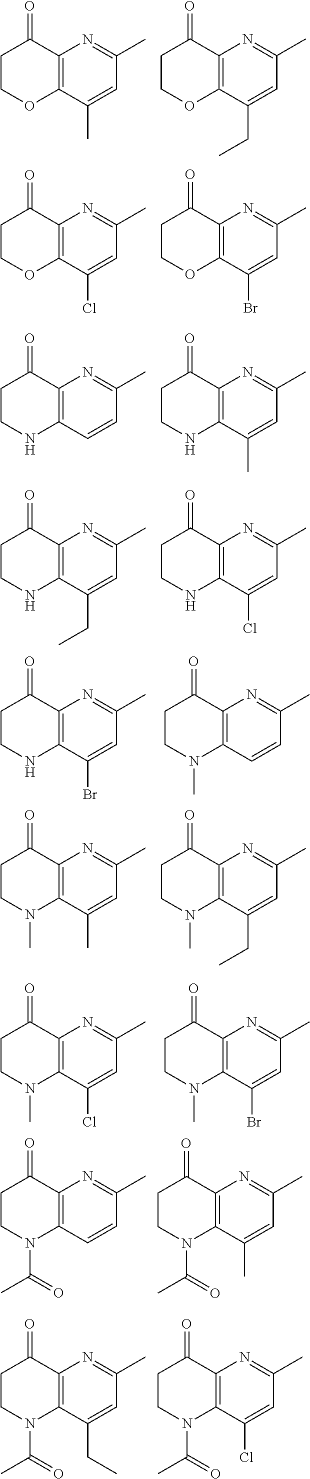

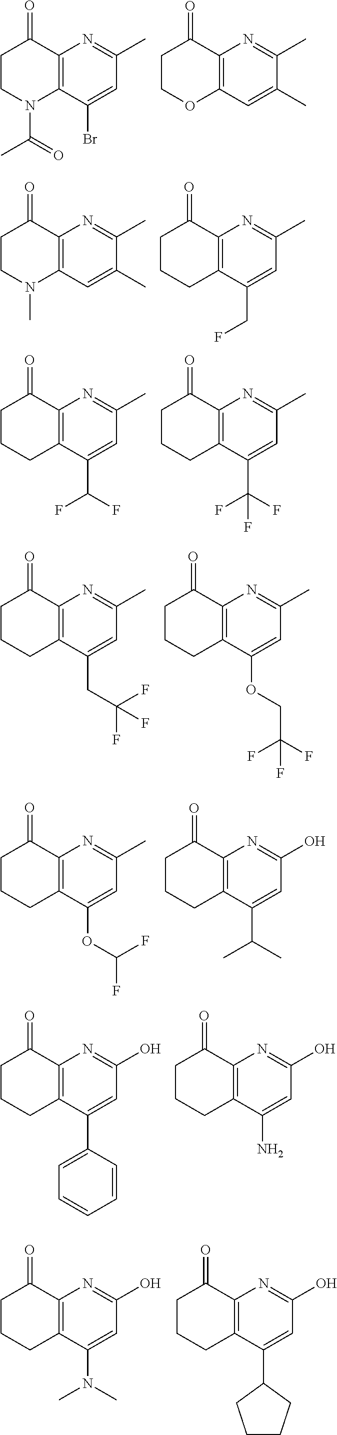

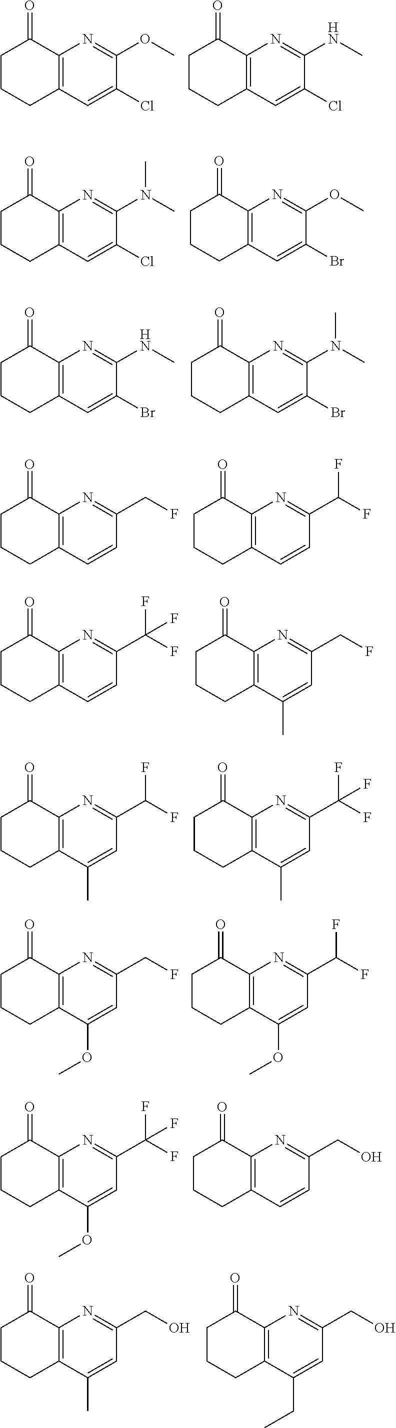

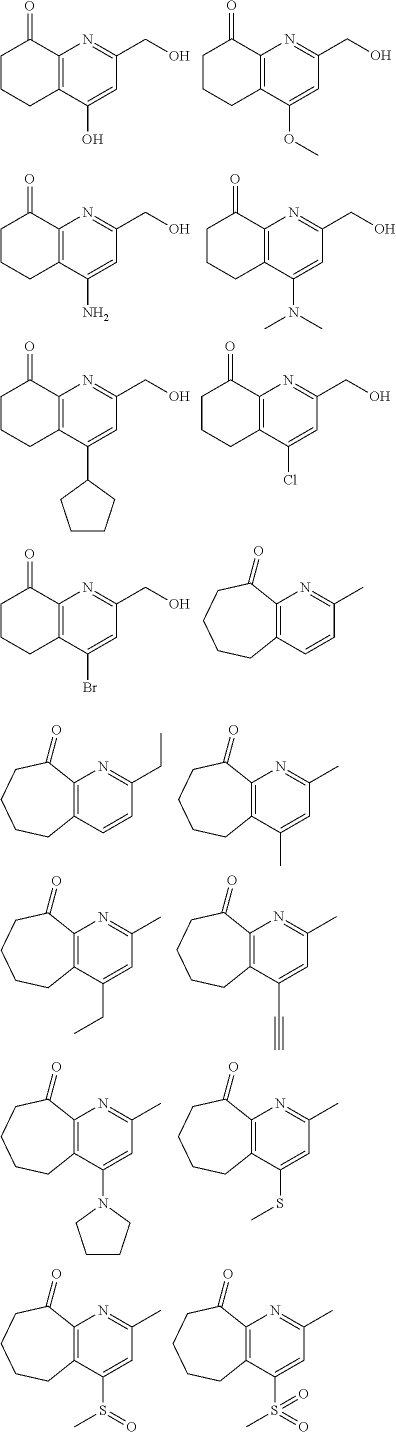

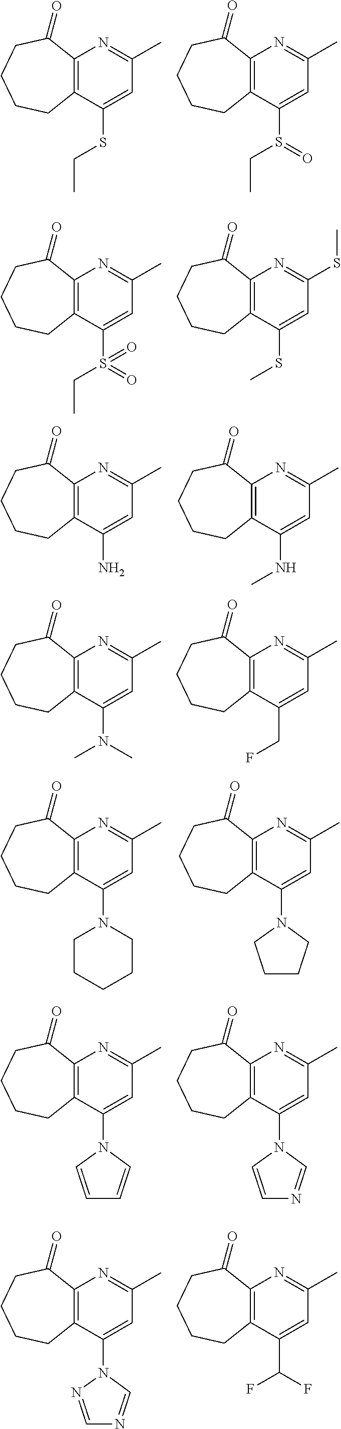

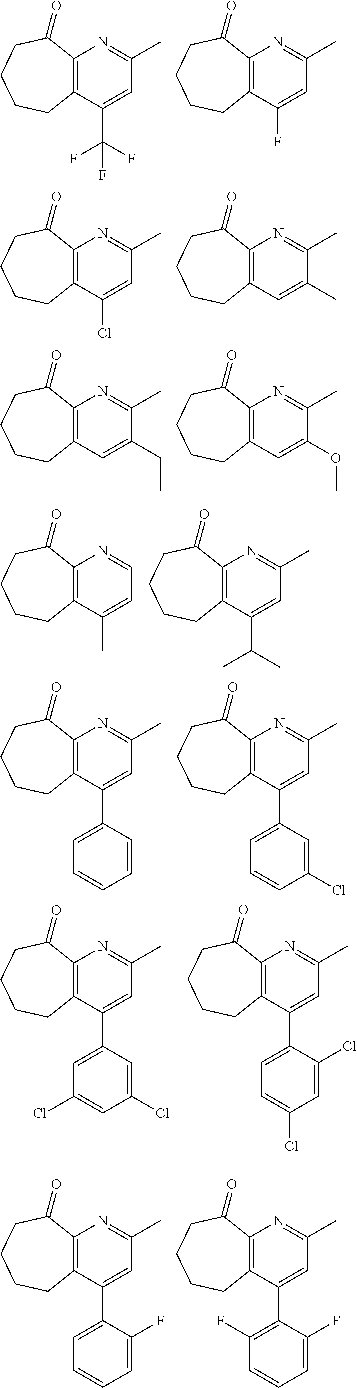

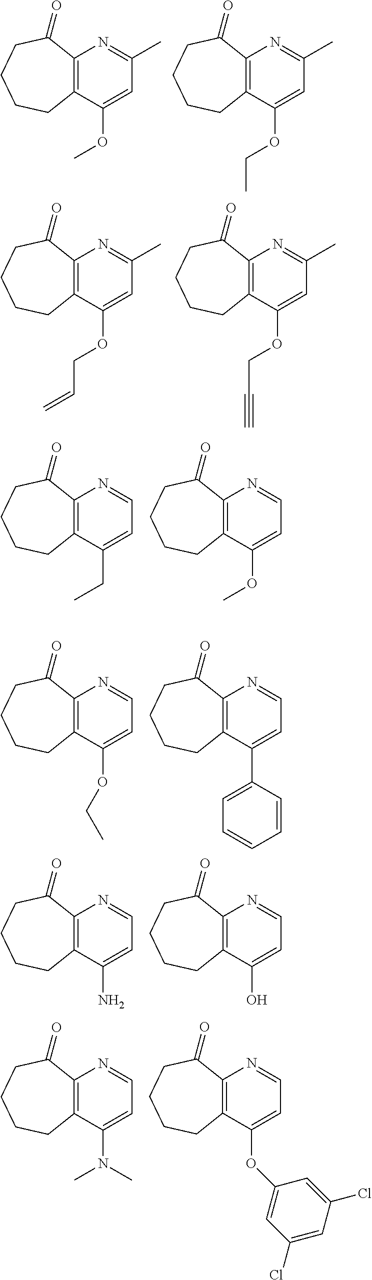

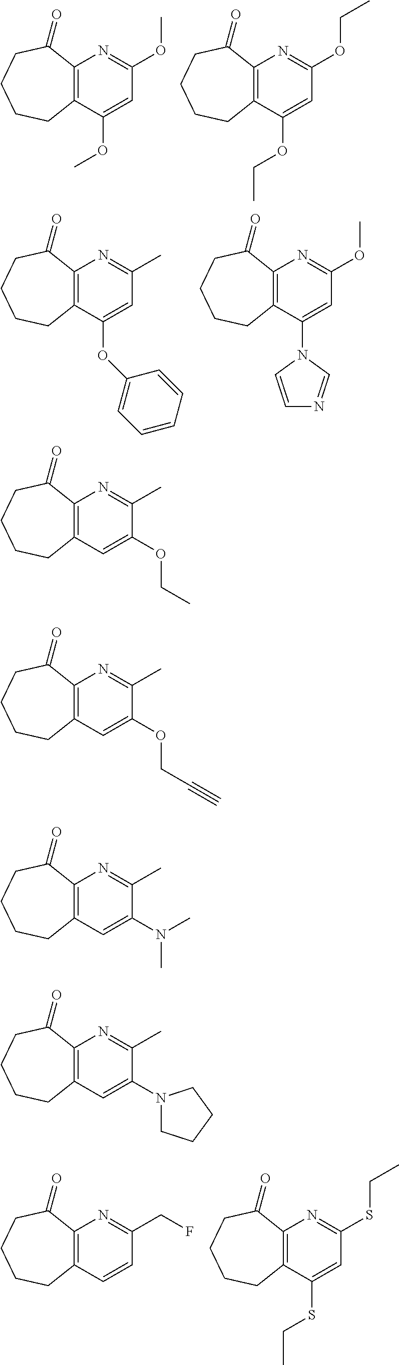

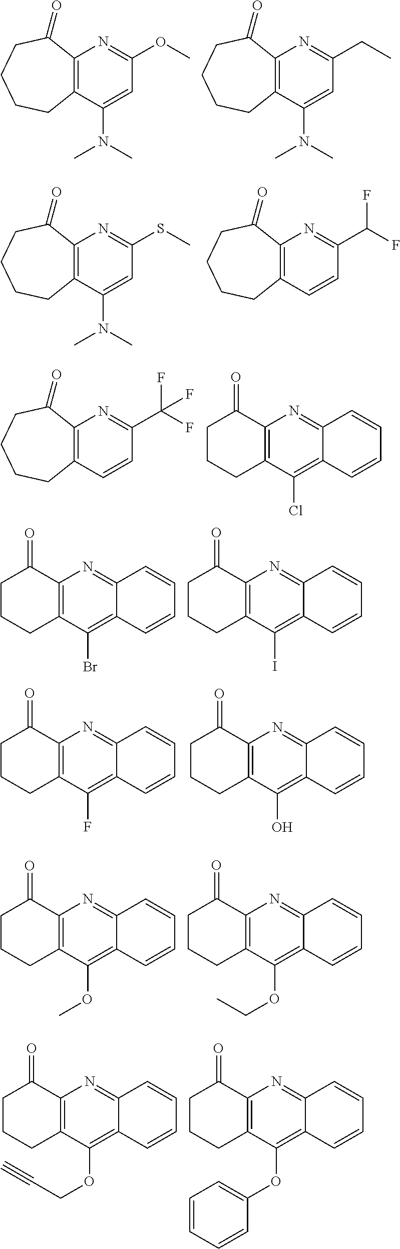

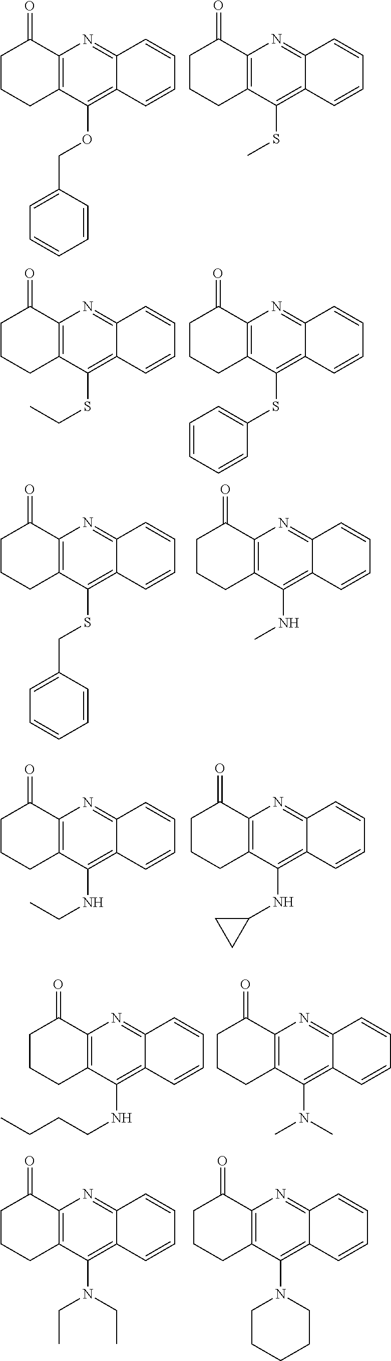

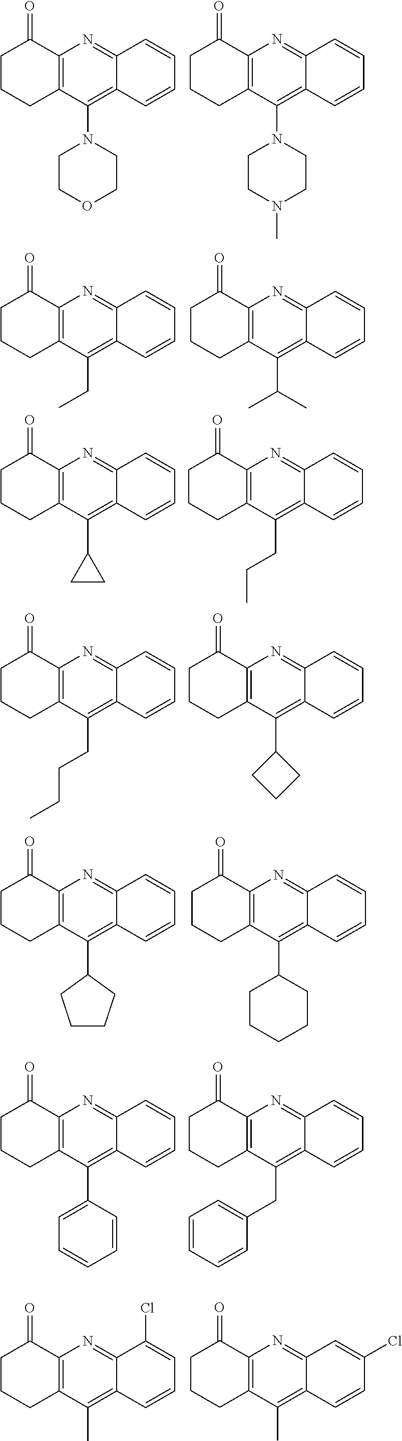

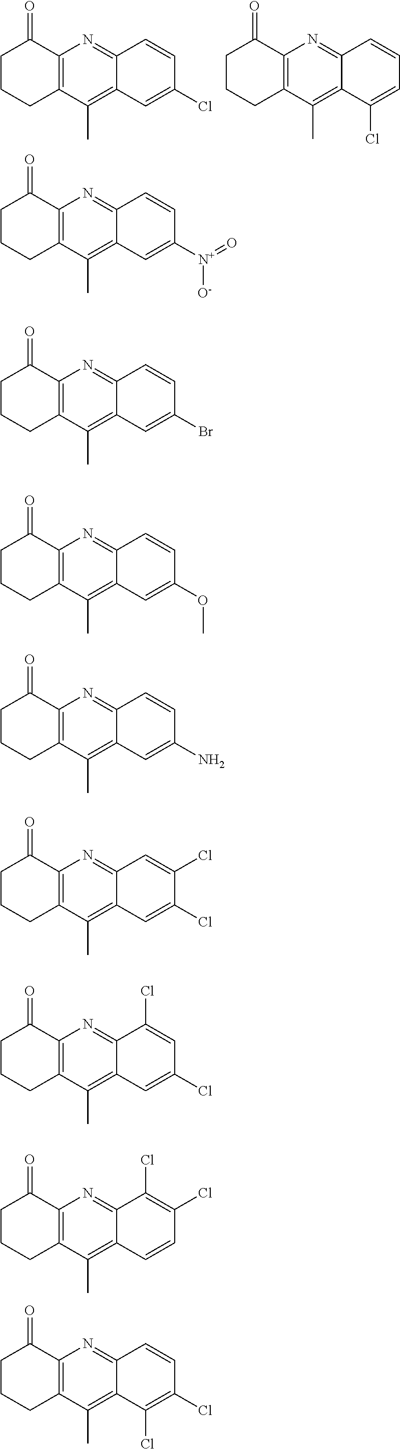

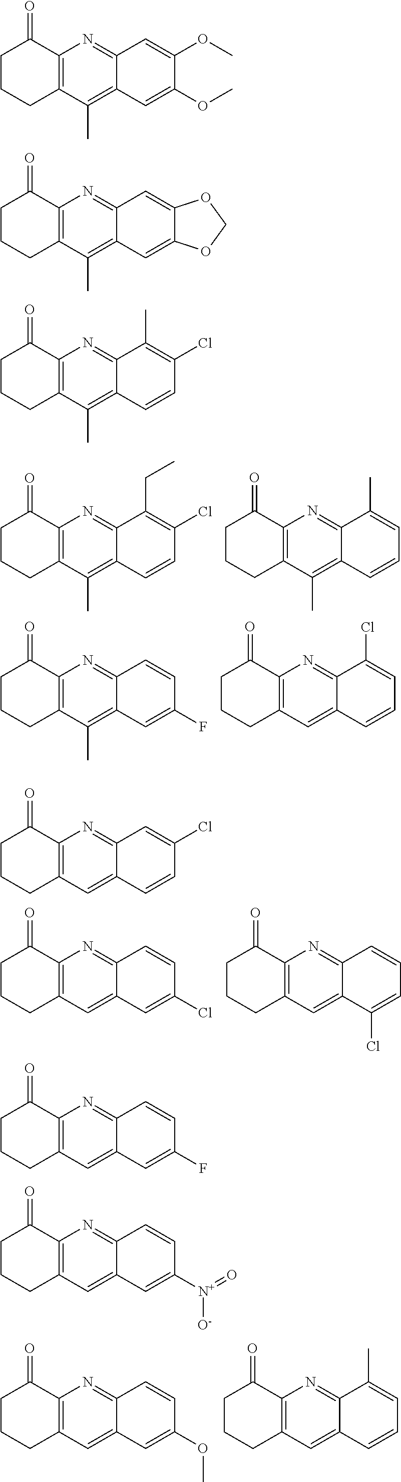

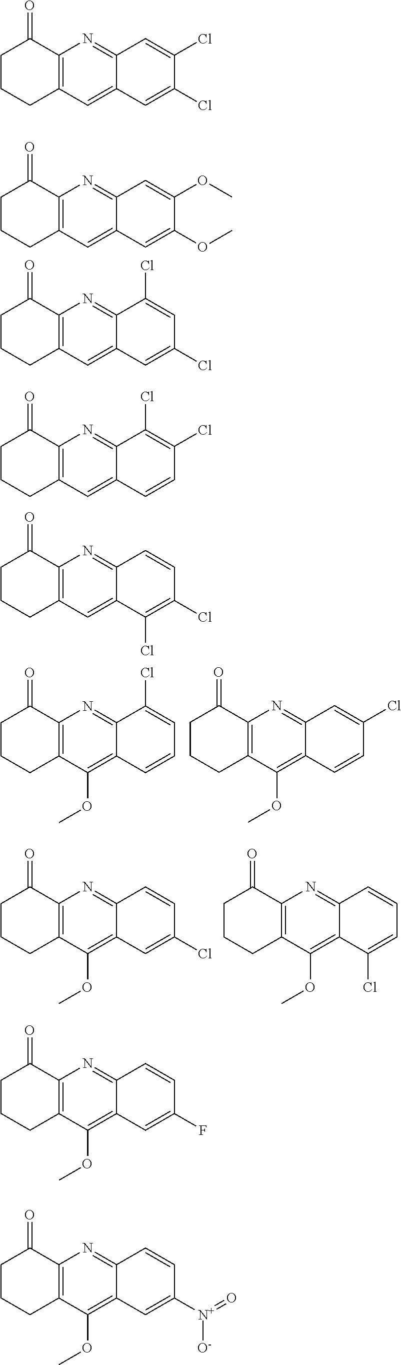

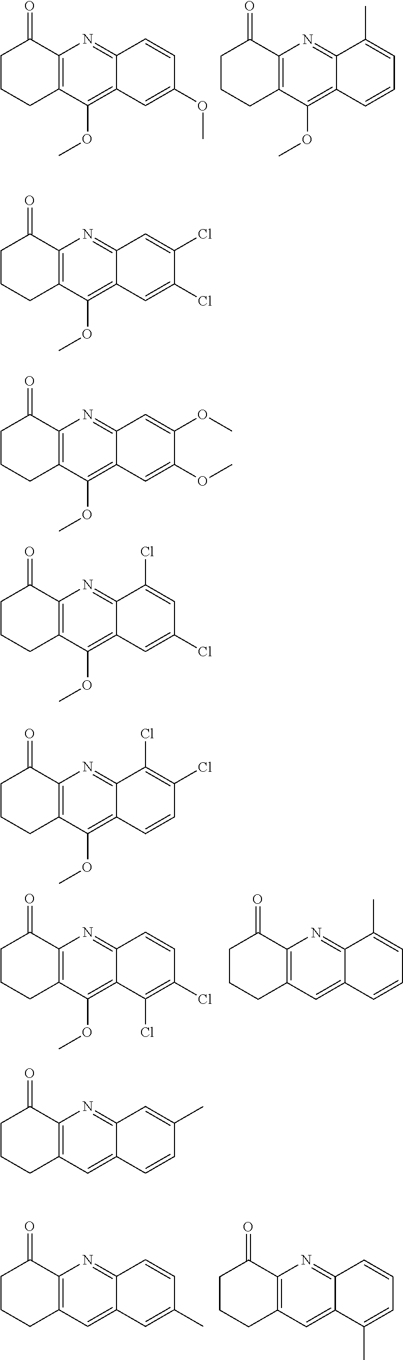

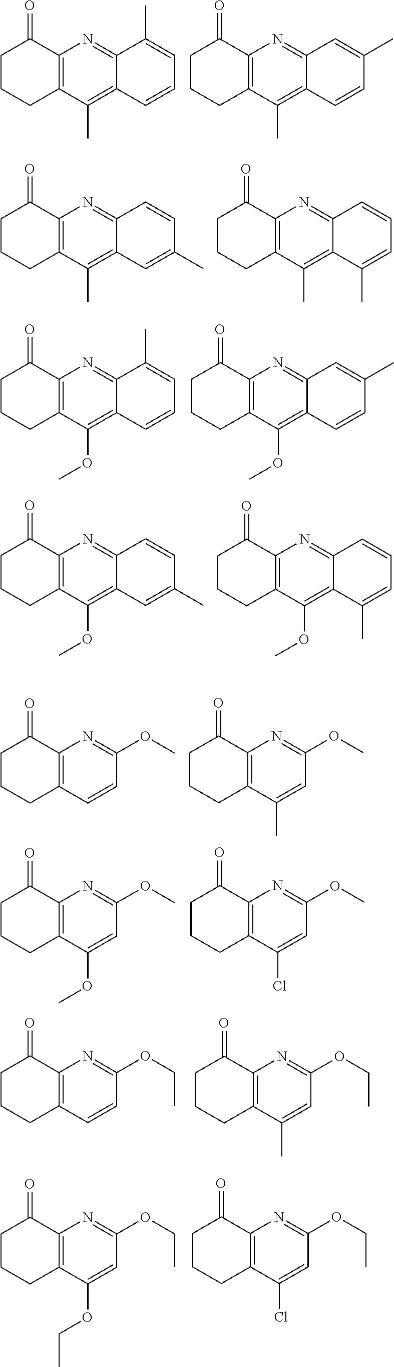

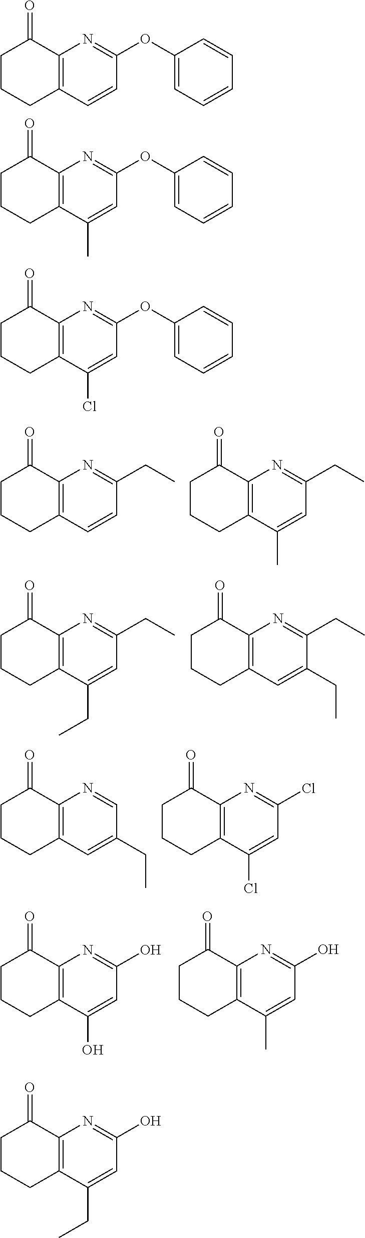

23. A compound of formula XVI 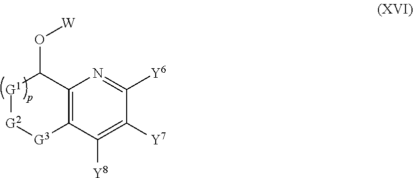
wherein G1, G2, G3, p, Y6, Y7 and Y8 are as defined for a compound of formula I in claim 1, and wherein W is hydrogen, CO—CH3, CO—CH2CH3, CO—CH2CH2CH3, CO—CH(CH3)2, CO—CF3, CO—CF2CF3, or a salt or N-oxide thereof, wherein the compound is not one of the compounds below:

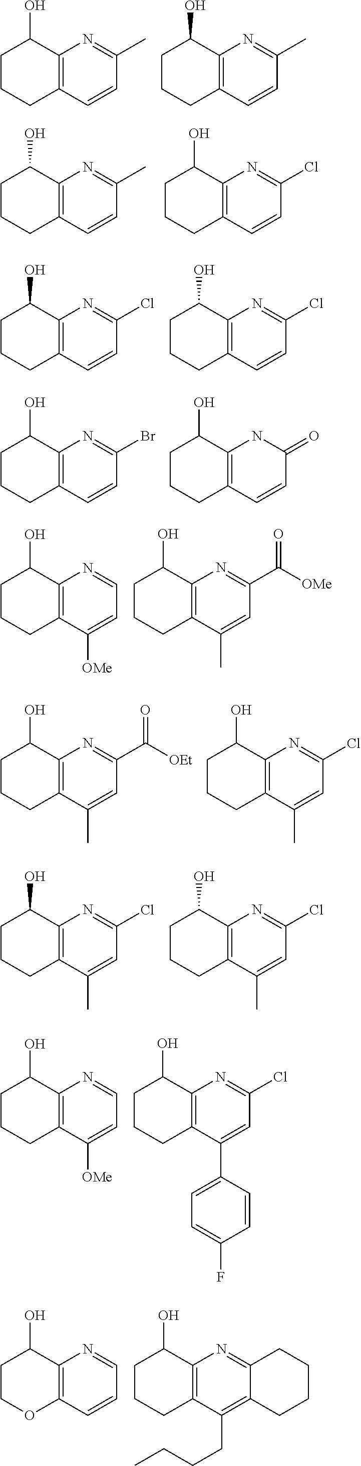

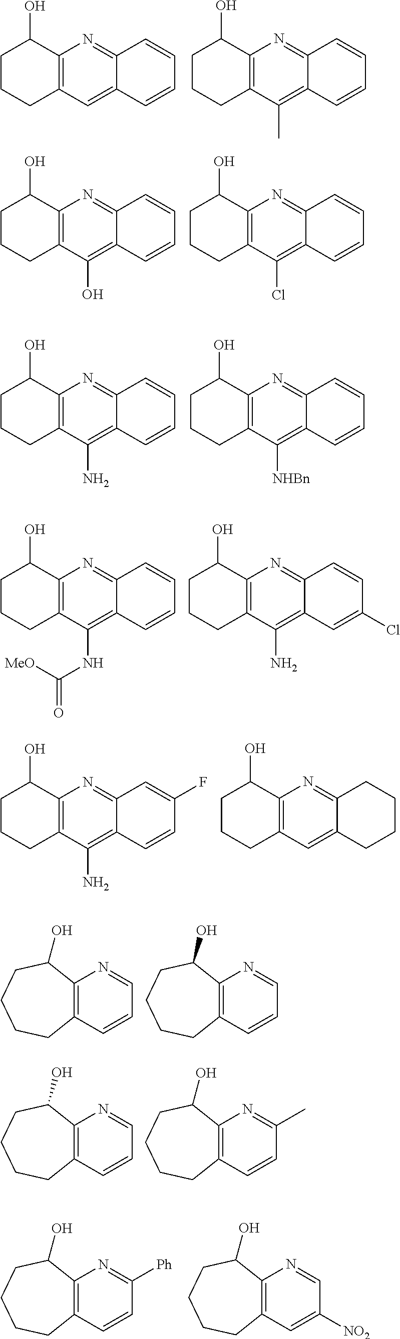

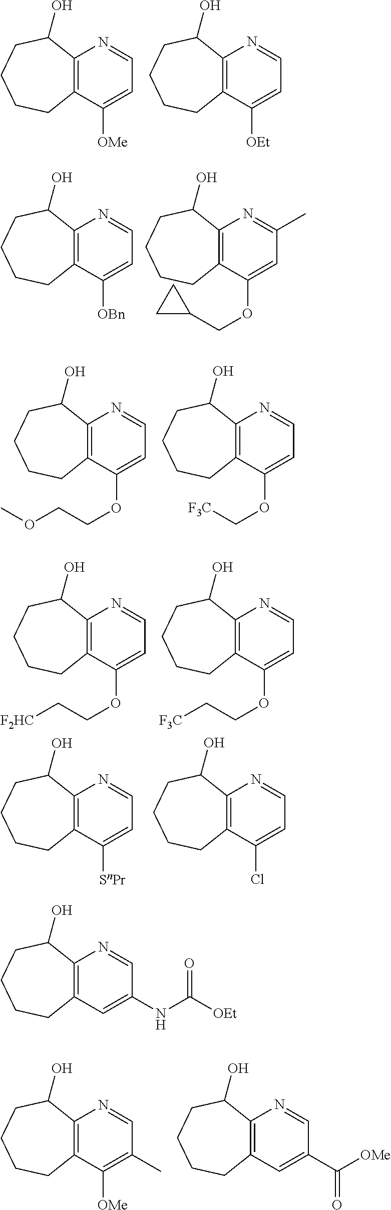

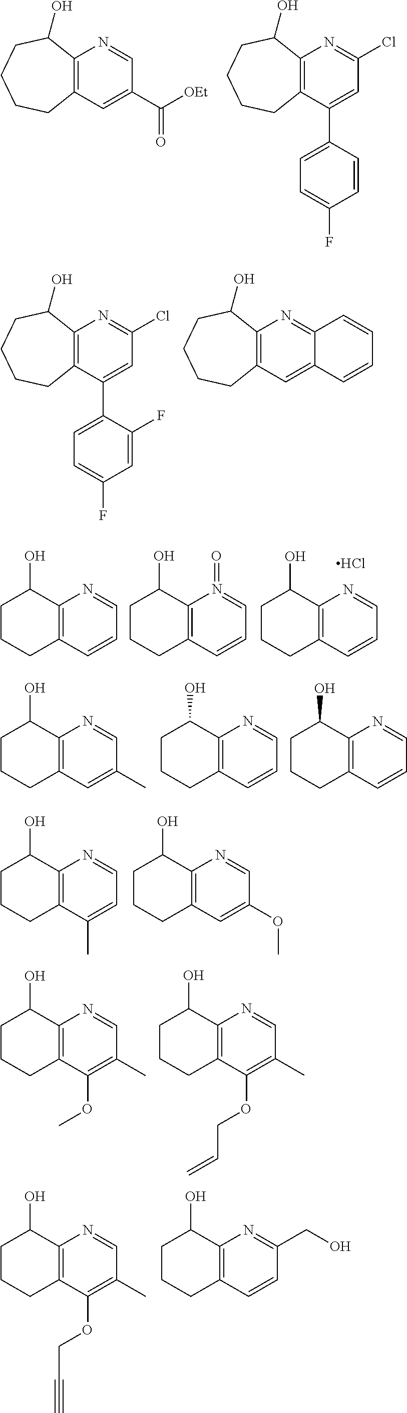

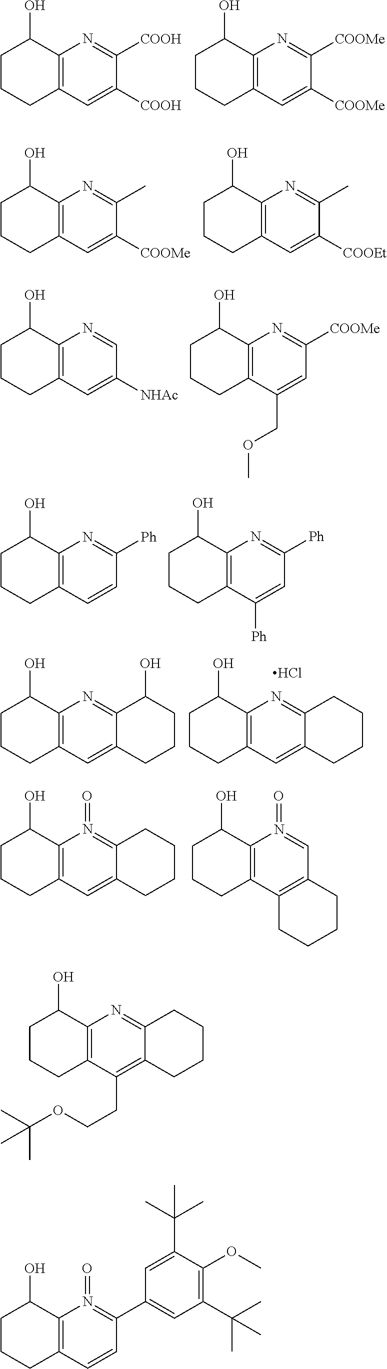

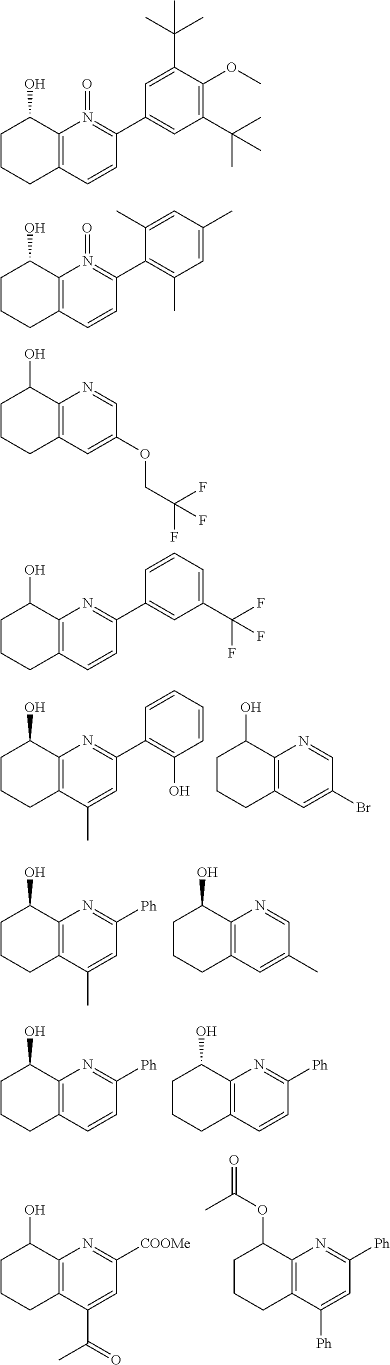

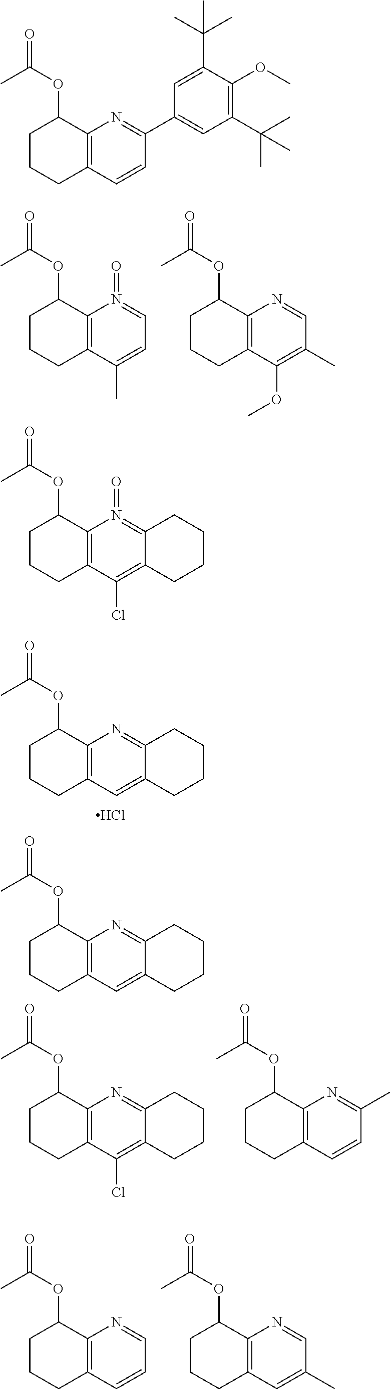

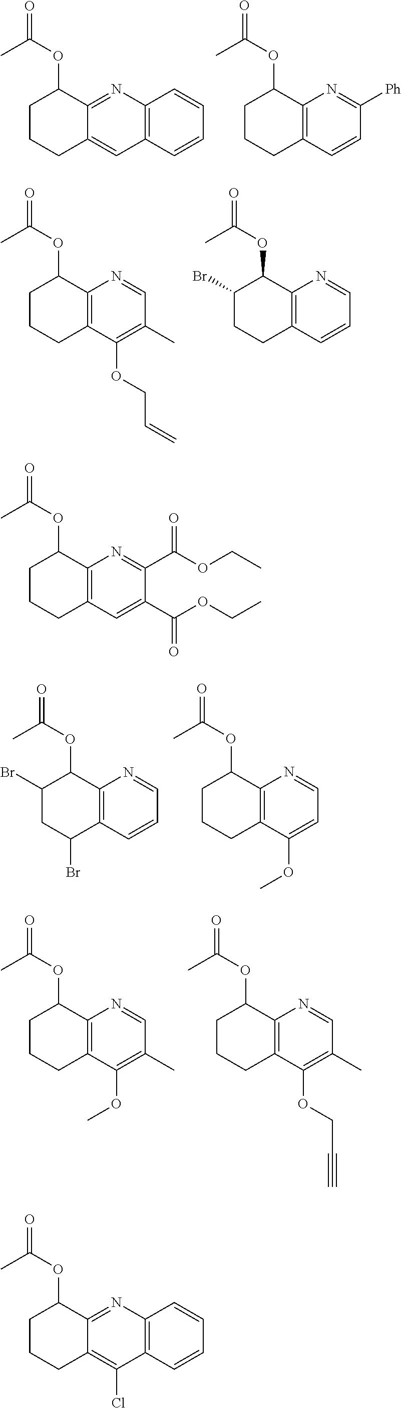

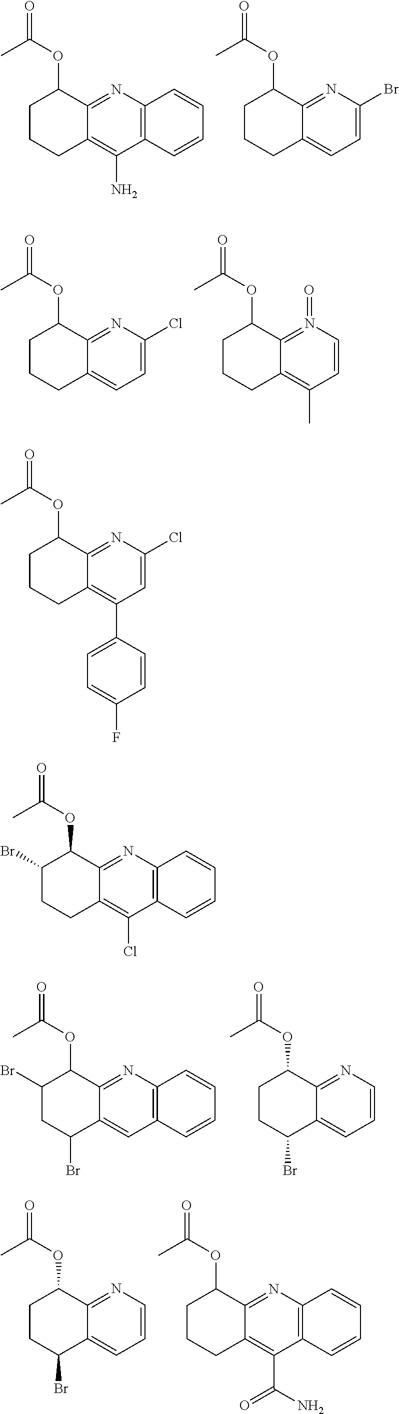

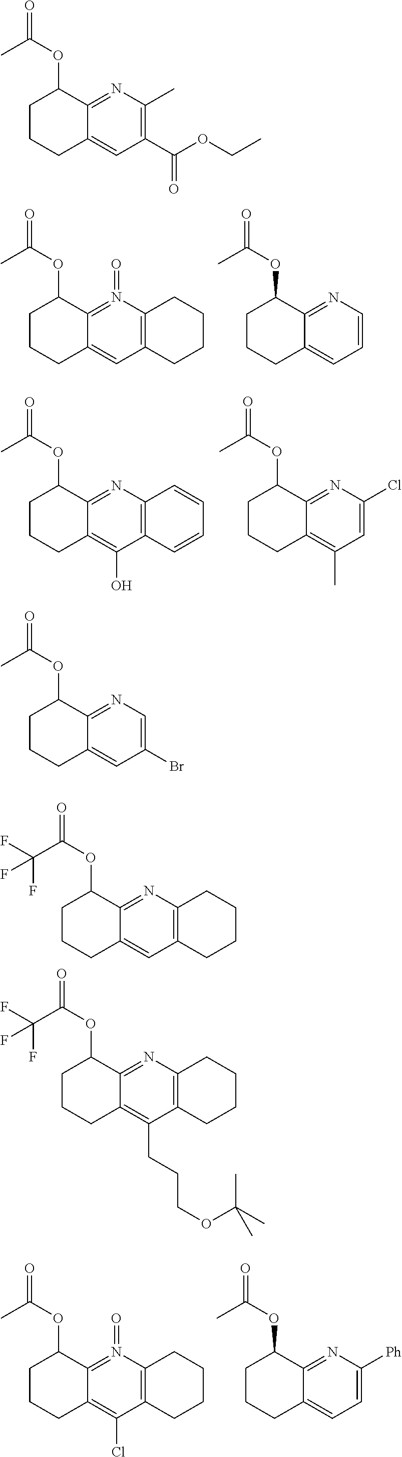

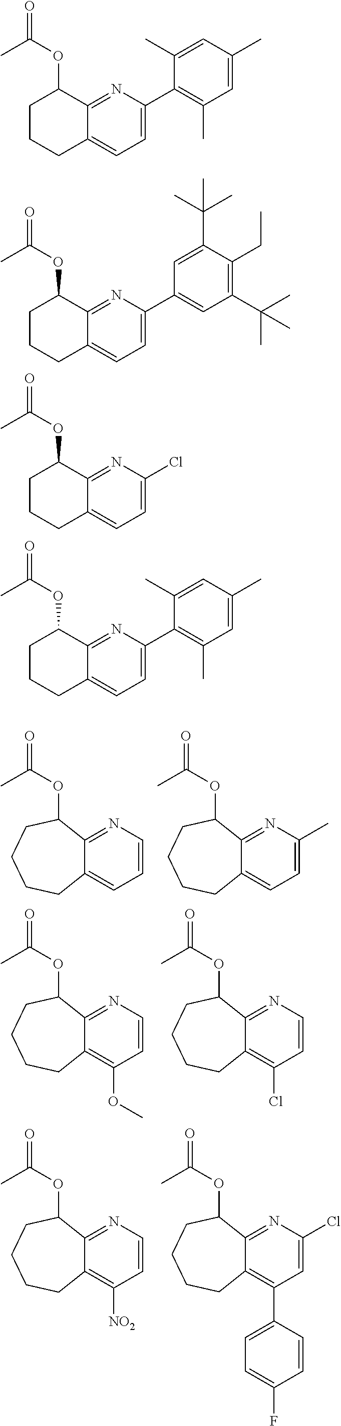

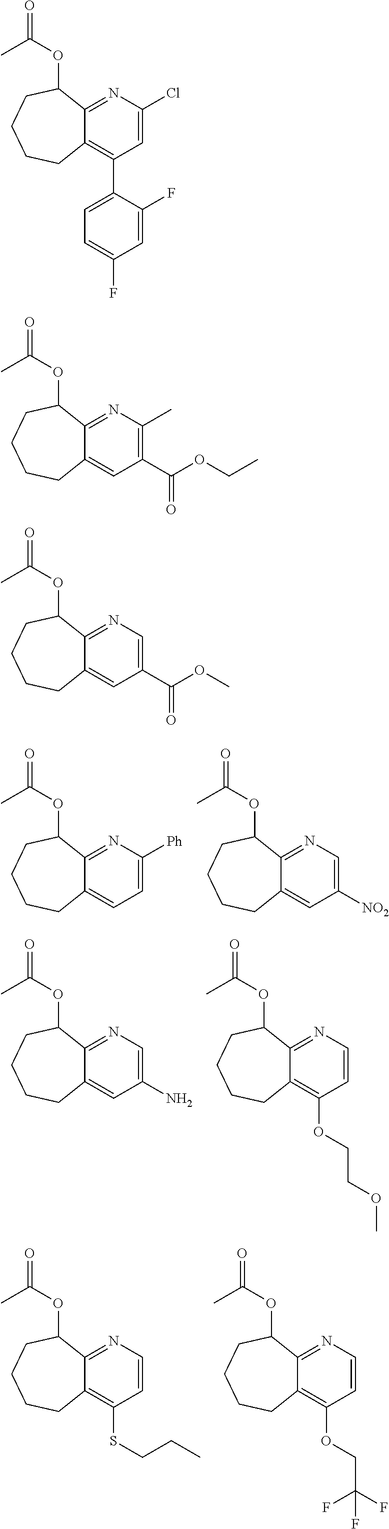

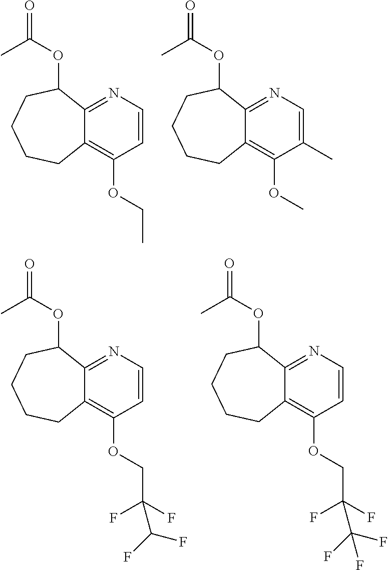

24. A compound according to claim 23 wherein the compound is one of the compounds below: 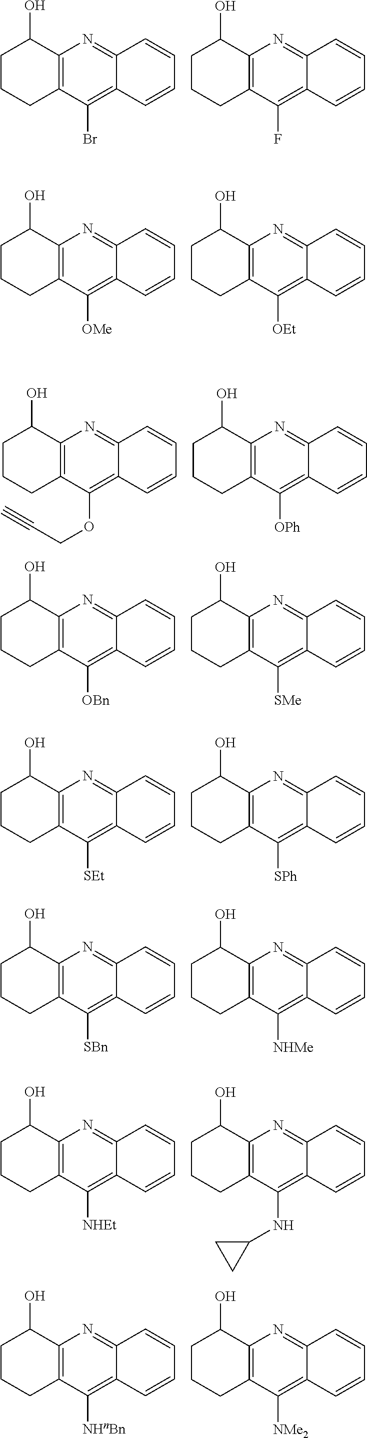 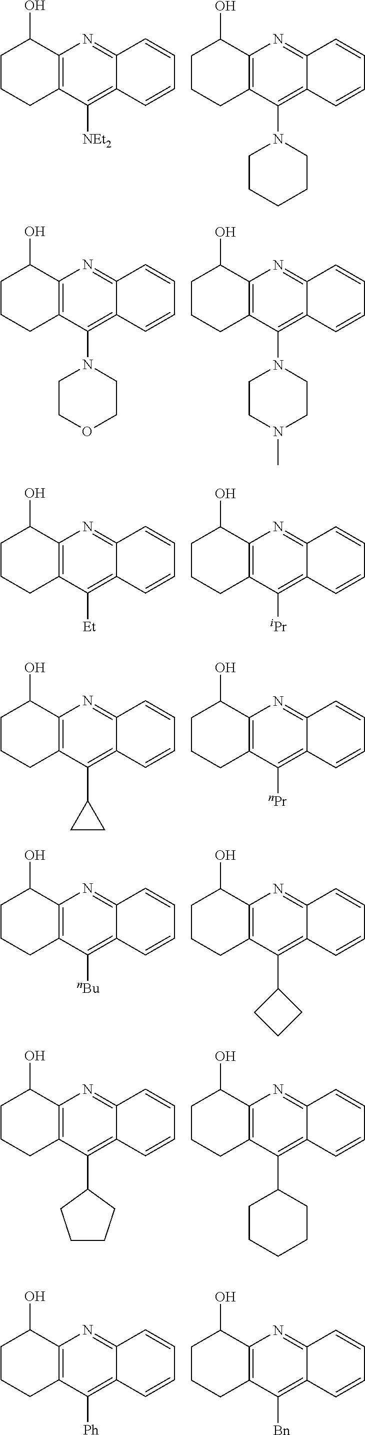 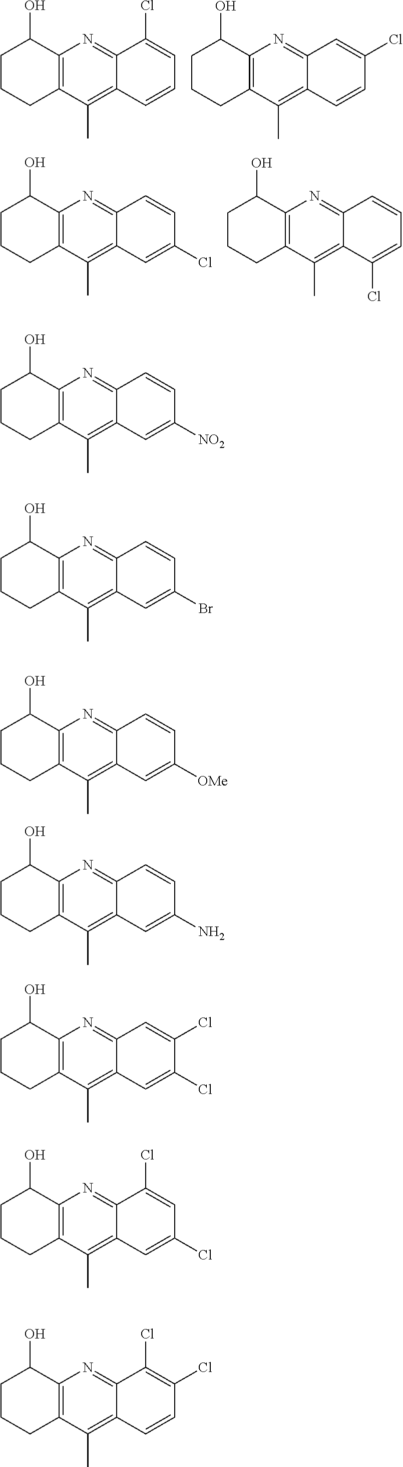 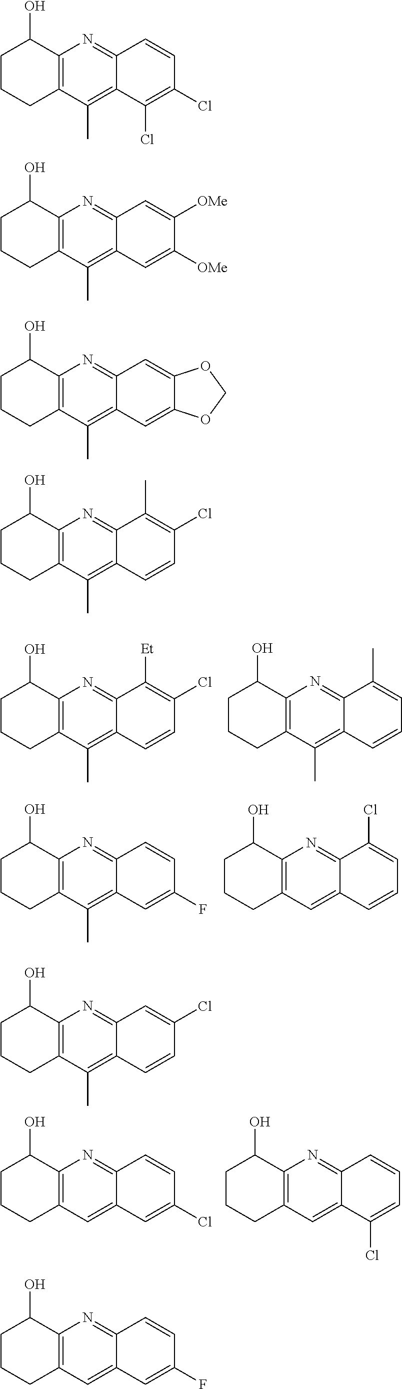 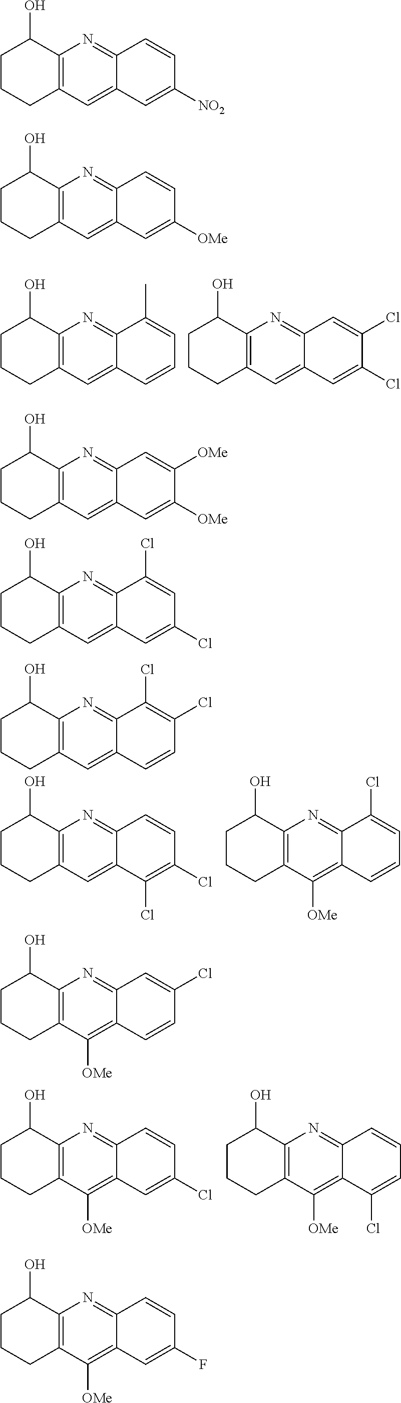 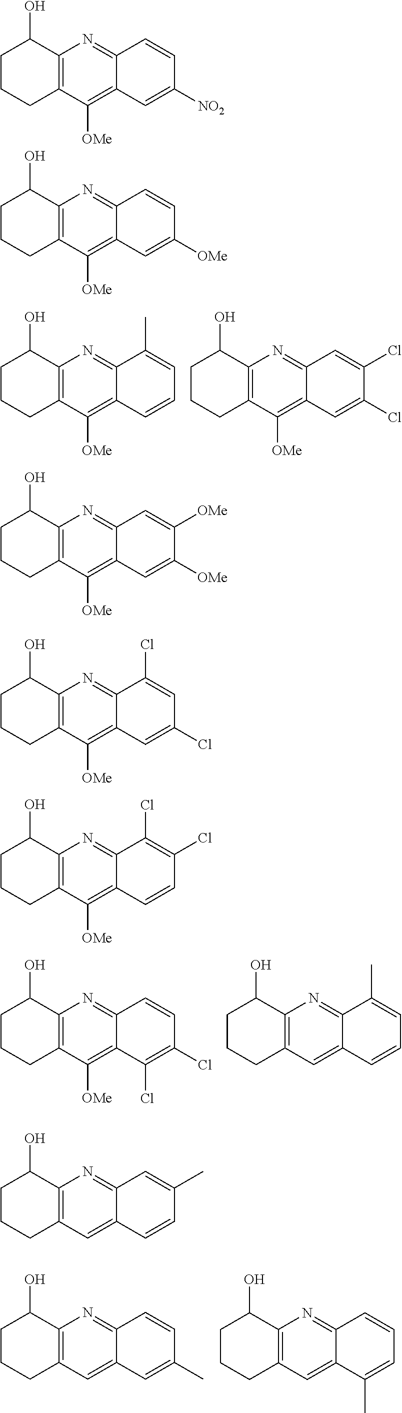 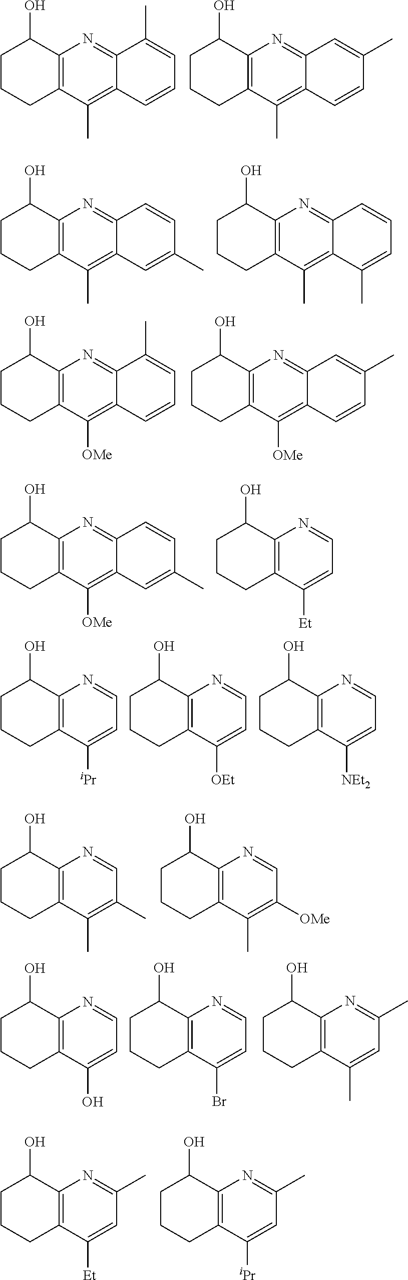 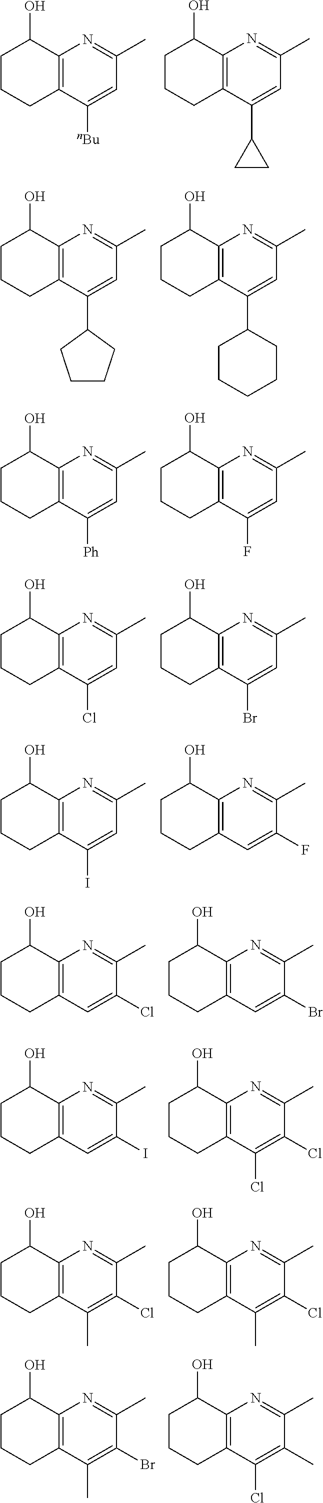 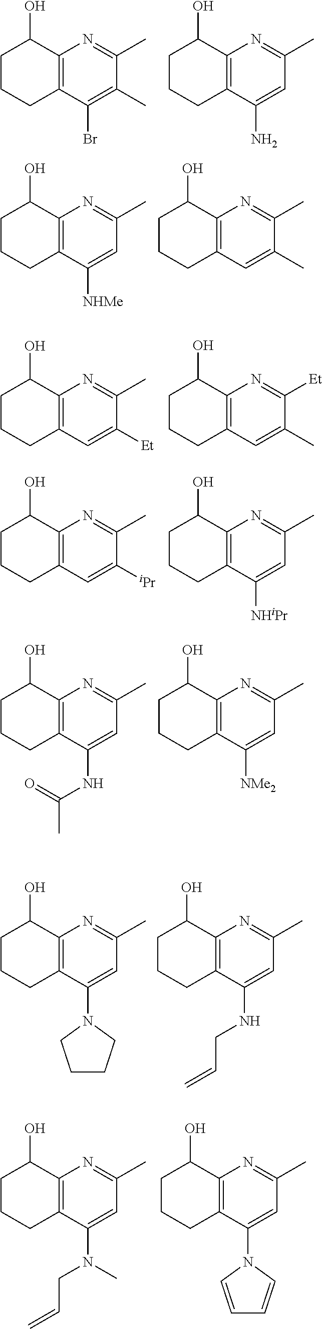 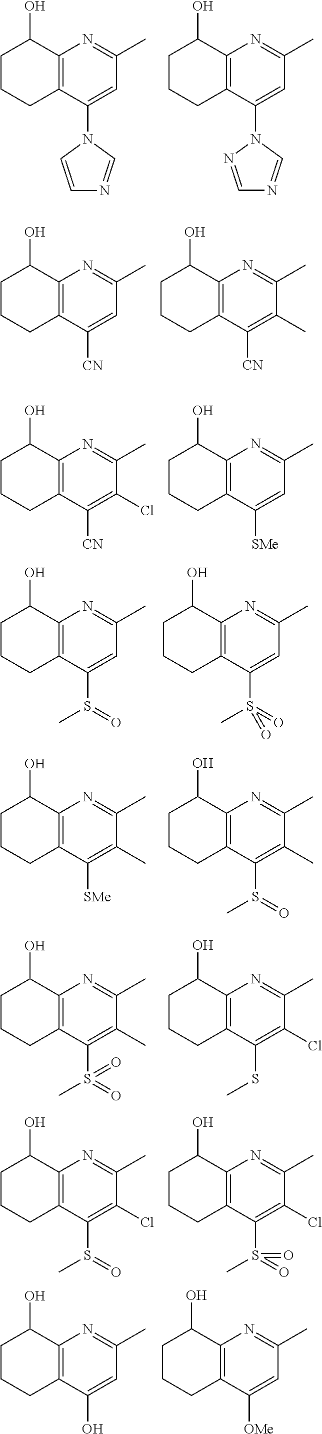 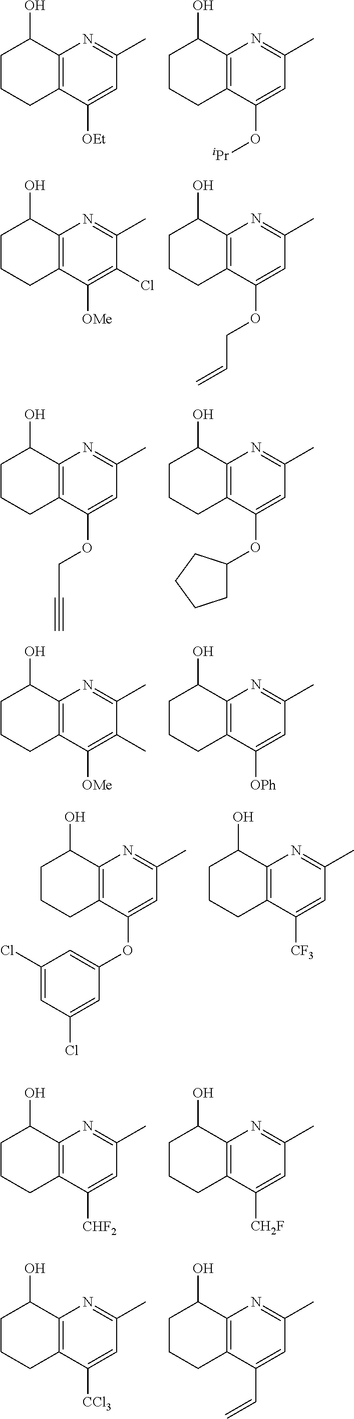 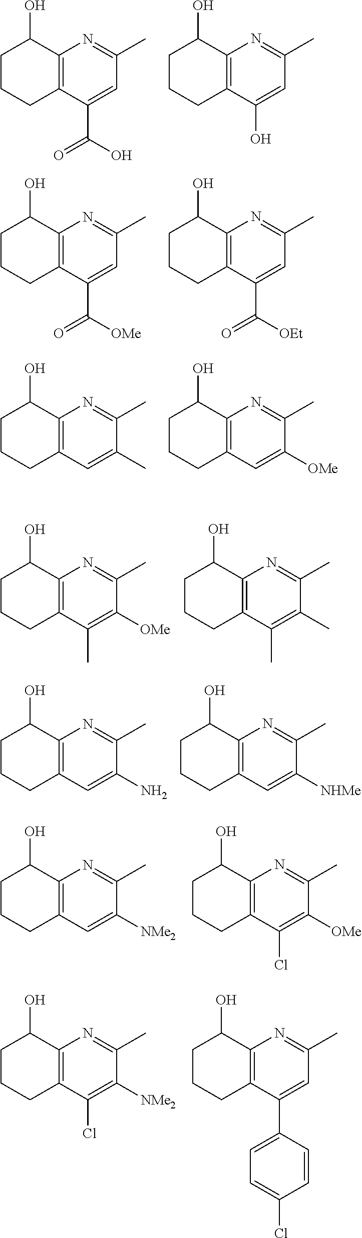 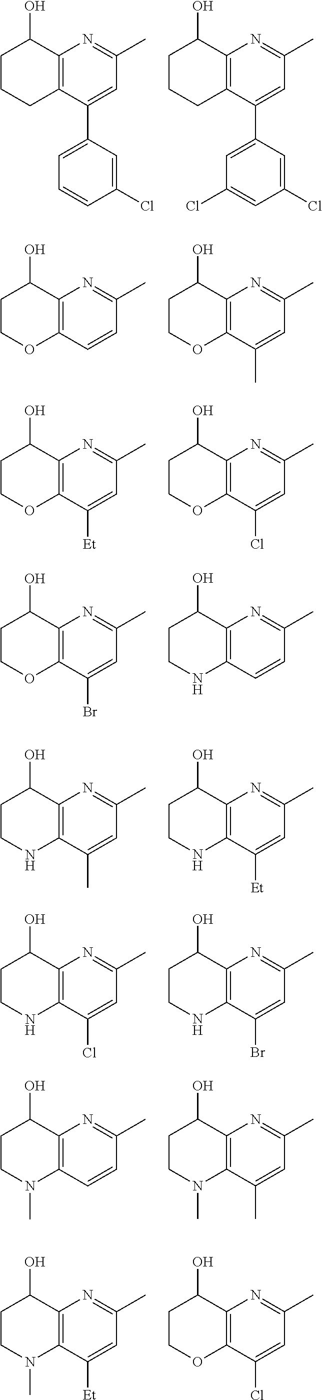 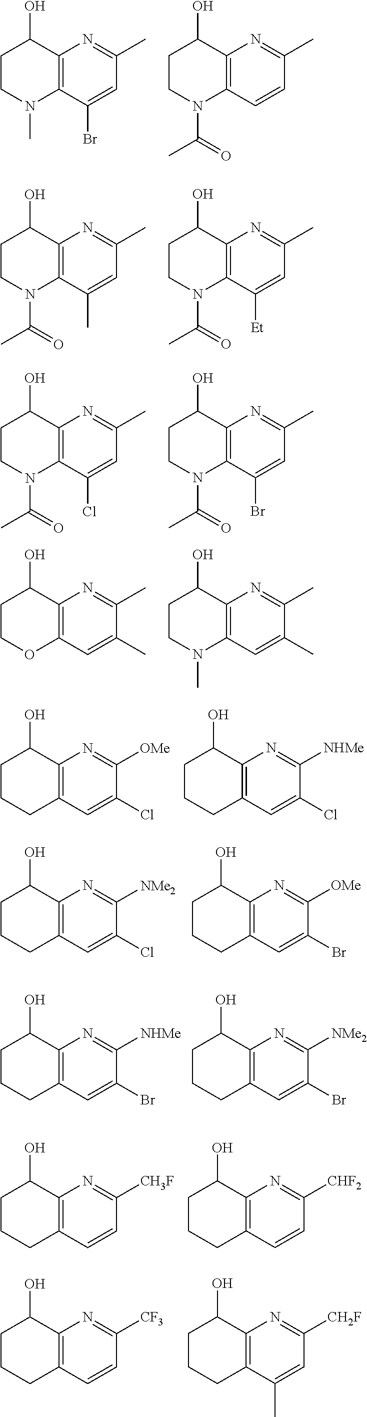 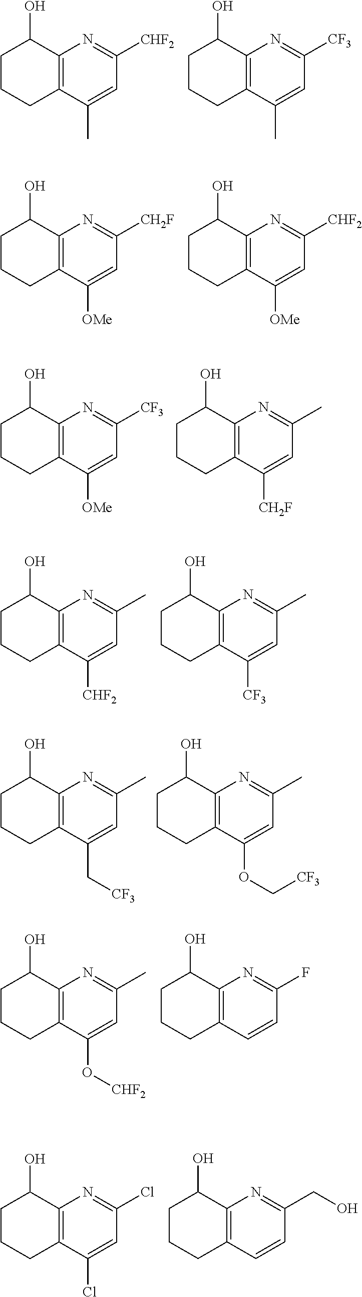 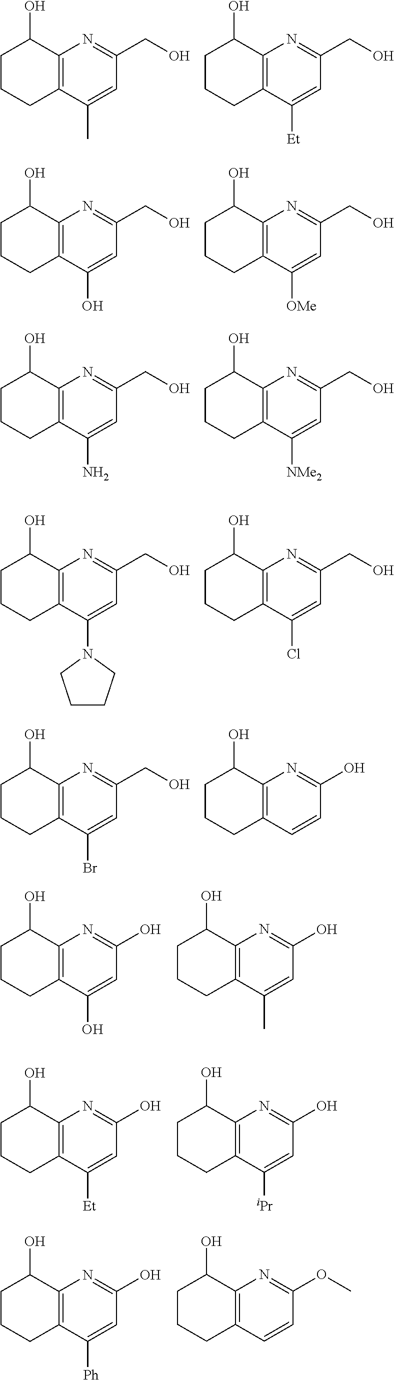 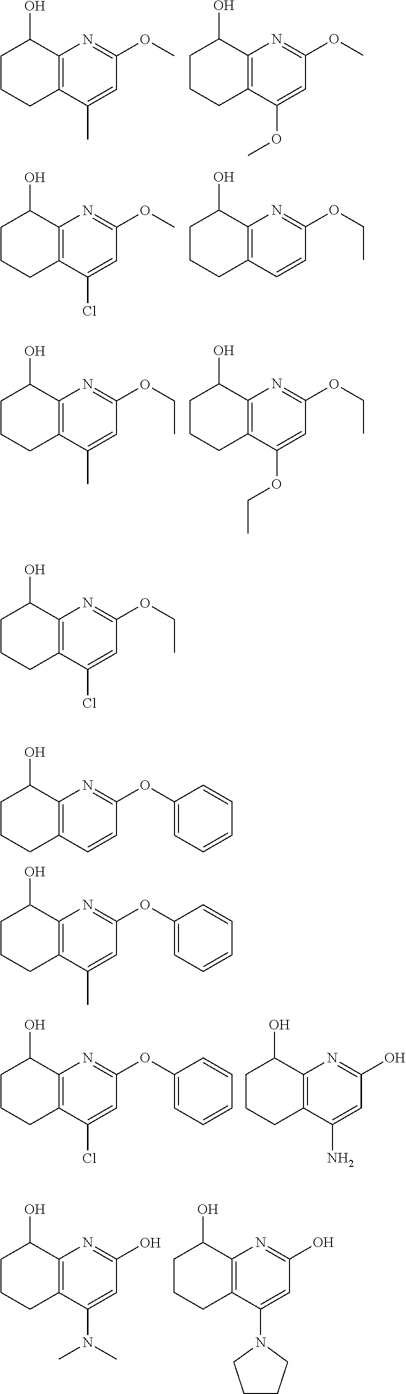 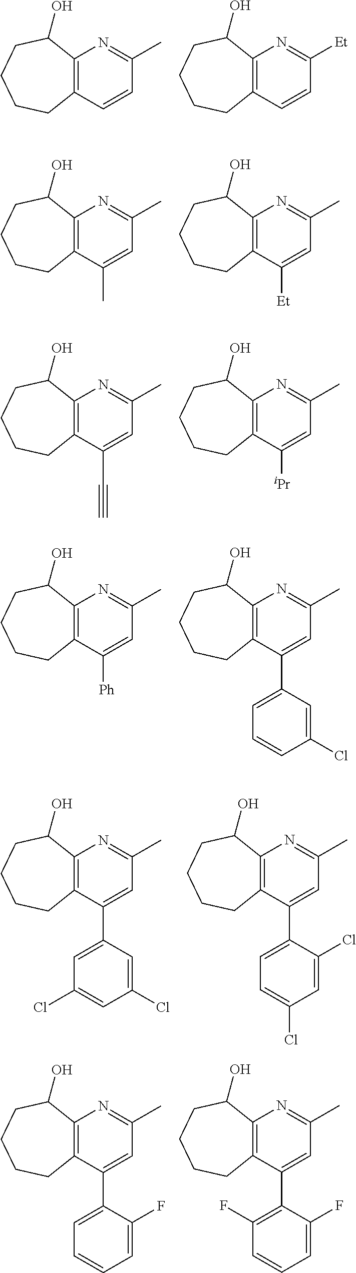 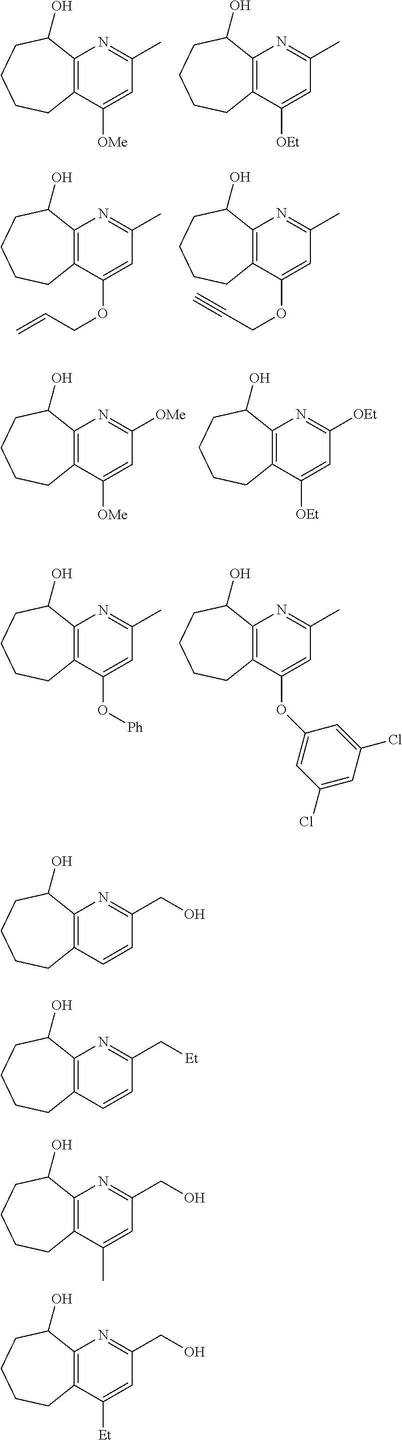 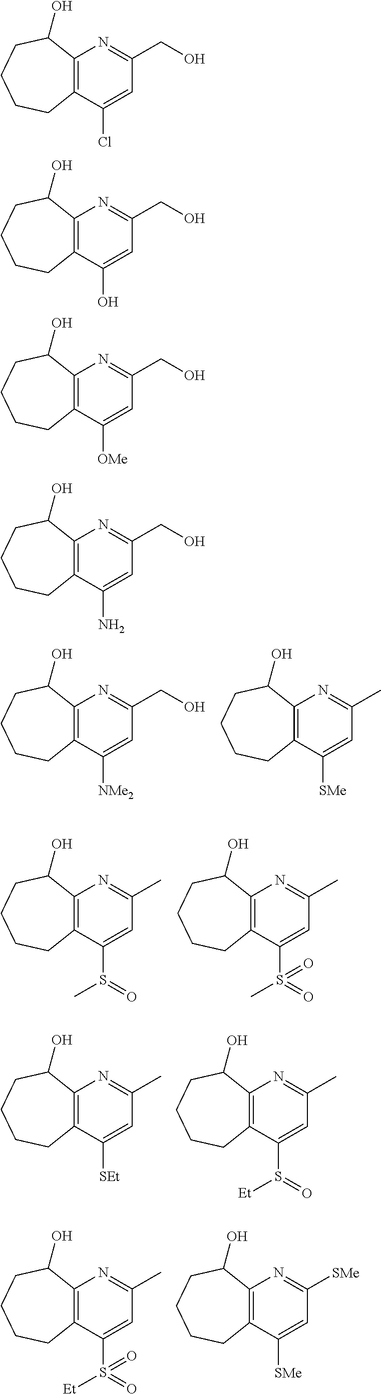 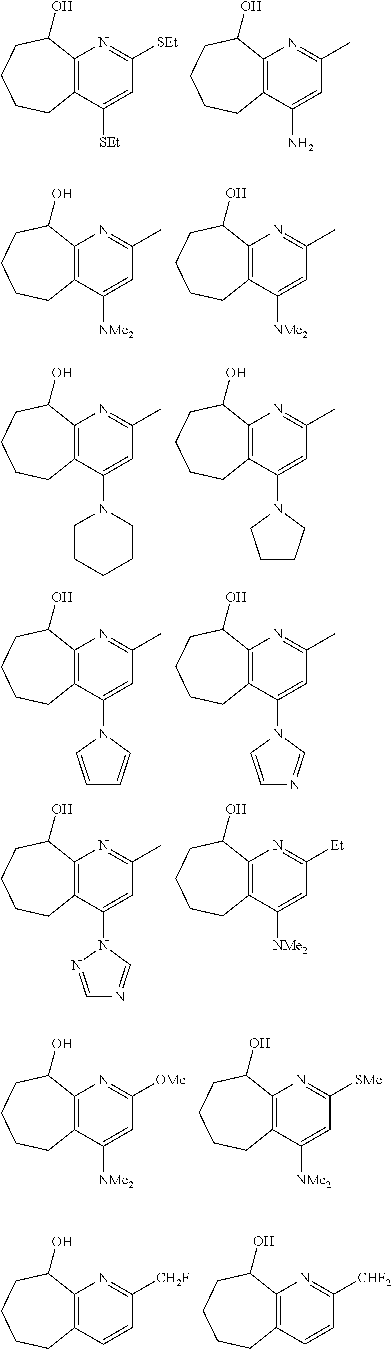 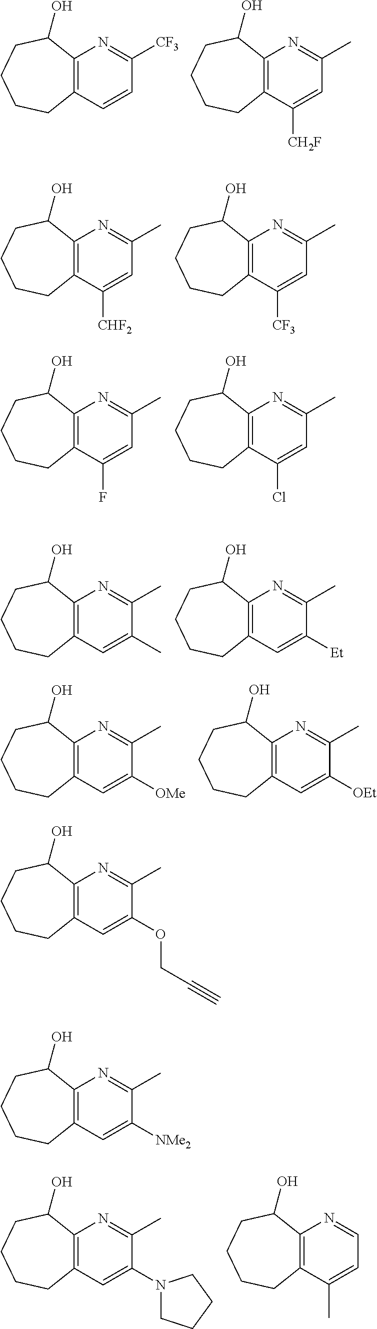 [chemical structure]
